(19) United States
(12) Patent Application Publication
(10) Pub. No.: US 2025/0122208 A1
(43) Pub. Date: Apr. 17, 2025

(54) CHEMOKINE RECEPTOR MODULATORS AND USES THEREOF

(71) Applicant: RAPT THERAPEUTICS, INC., South San Francisco, CA (US)

(72) Inventors: Blanca I. Gomez, San Mateo, CA (US); Michelle Yoo Min Ko, San Francisco, CA (US); Daniel Poon, Piedmont, CA (US); Omar Robles, San Mateo, CA (US); Grant M. Shibuya, South San Francisco, CA (US); Vi-Anh T. Vu, Sunnyvale, CA (US); Joel Aponte-Guzman, San Francisco, CA (US); David J. Wustrow, Los Gatos, CA (US); Mikhail Zibinsky, Redwood City, CA (US)

(21) Appl. No.: 18/907,350

(22) Filed: Oct. 4, 2024

Related U.S. Application Data
(60) Provisional application No. 63/542,981, filed on Oct. 6, 2023.

(51) Int. Cl. C07D 487/04; A61K 31/519; A61K 45/06

(52) U.S. Cl. CPC C07D 487/04; A61K 31/519; A61K 45/06

(57) ABSTRACT

Described herein, inter alia, are compounds and methods of use thereof for the modulation of certain chemokine receptor activity.

### CROSS REFERENCES TO RELATED APPLICATIONS

This application claims the benefit of priority of U.S. Provisional Application No. 63/542,981 filed on Oct. 6, 2023, which is incorporated herein by reference in its entirety and for all purposes.

### BACKGROUND

The successful operation of the host defense system is the result of several processes that work together to eliminate foreign pathogens. Coordinated innate and acquired immune responses are required, and many secreted and cell-associated factors have been identified as important mediators coordinating and regulating these two arms of host defense. Chemokines are a family of cytokines that act as chemoattractants to guide leukocyte migration. They are secreted by a wide variety of cells and can be functionally divided into two groups, hemostatic chemokines and inflammatory chemokines. Hemostatic chemokines are constituently produced in certain tissues and control cells of the immune system during processes of immune surveillance, such as directing lymphocytes to the lymph nodes to allow them to screen for invasion of pathogens. Inflammatory chemokines are released from cells in response to a pathological event (e.g., pro-inflammatory stimuli such as IL-1 or viruses). They function primarily as chemoattractants as part of the inflammatory response and serve to guide cells of both the innate and adaptive immune systems to the site of inflammation. The C-C chemokine receptor type 4 (CCR4), plays a role in the progression of a number of inflammation-related and other disorders. The identification of compounds that modulate CCR4 function is an ongoing challenge. Disclosed herein, inter alia, are solutions to these and other problems in the art.

### BRIEF SUMMARY

In an aspect is provided a compound having structural Formula (I):

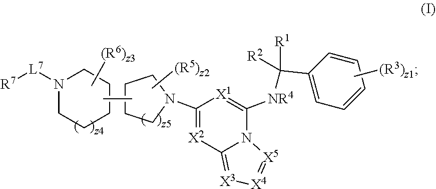

or a pharmaceutically acceptable salt thereof.

X¹ is CR⁸ or N. X² is CR⁹ or N. X³ is CR¹⁰ or N. X⁴ is CR¹¹ or N. X⁵ is CR¹² or N. In embodiments, at least one of X¹, X², X³, X⁴, and X⁵ is N.

The symbol z1 is an integer from 0 to 5. The symbol z2 is an integer from 0 to 4. The symbol z3 is an integer from 0 to 11. The symbol z4 is an integer from 0 to 2. The symbol z5 is 1 or 2.

L⁷ is a bond, —O—, —S—, —NR^{7.2B}—, —C(O)—, —C(O)O—, —S(O)—, —S(O)₂—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

R¹ is hydrogen, halogen, —CX^{1.1}₃, —CHX^{1.1}₂, —CH₂X^{1.1}, —CN, —N₃, —SOₙ₁R^{1A}, —SOᵥ₁NR^{1B}R^{1C}, —NHNR^{1B}R^{1C}, —ONR^{1B}R^{1C}, —NHC(O)NHNR^{1B}R^{1C}, —NHC(O)NR^{1B}R^{1C}, —N(O)ₘ₁, —NR^{1B}R^{1C}, —C(O)R^{1D}, —C(O)OR^{1D}, C(O)NR^{1B}R^{1C}, —OR^{1A}, NR^{1B}SO₂R^{1A}, —NR^{1B}C(O)R^{1D}, —NR^{1B}C(O)OR^{1D}, —NR^{1B}OR^{1D}, —OCX^{11.3}, —OCHX^{1.1}₂, —OCH₂X^{1.1}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R² is hydrogen, halogen, —CX^{2.1}₃, —CHX^{2.1}₂, —CH₂X^{2.1}, —CN, —N₃, —SOₙ₂R^{2A}, —SOᵥ₂NR^{2B}R^{2C}, —NHNR^{2B}R^{2C}, —ONR^{2B}R^{2C}, —NHC(O)NHNR^{2B}R^{2C}, —NHC(O)NR^{2B}R^{2C}, —N(O)ₘ₂, —NR^{2B}R^{2C}, —C(O)R^{2D}, —C(O)OR^{2D}, —C(O)NR^{2B}R^{2C}, —OR^{2A}, —NR^{2B}SO₂R^{2A}, —NR^{2B}C(O)R^{2D}, —NR^{2B}C(O)OR^{2D}, —NR^{2B}OR^{2D}, —OCX^{2.1}₃, —OCHX^{2.1}₂, —OCH₂X^{2.1}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R³ is independently halogen, —CX^{3.1}₃, —CHX^{3.1}₂, —CH₂X^{3.1}, —CN, —N₃, —SOₙ₃R^{3A}, —SOᵥ₃NR^{3B}R^{3C}, —NHNR^{3B}R^{3C}, —ONR^{3B}R^{3C}, —NHC(O)NHNR^{3B}R^{3C}, —NHC(O)NR^{3B}R^{3C}, —N(O)ₘ₃, —NR^{3B}R^{3C}, —C(O)R^{3D}, —C(O)OR^{3D}, —C(O)NR^{3B}R^{3C}, —OR^{3A}, —NR^{3B}SO₂R^{3A}, —NR^{3B}C(O)R^{3D}, —NR^{3B}C(O)OR^{3D}, —NR^{3B}OR^{3D}, —OCX^{3.1}₃, —OCHX^{3.1}₂, —OCH₂X^{3.1}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R⁴ is hydrogen, —CX^{4.1}₃, —CHX^{4.1}₂, —CH₂X^{4.1}, —SOₙ₄R^{4A}, —SOᵥ₄NR^{4B}R^{4C}, —C(O)R^{4D}, —C(O)OR^{4D}, —C(O)NR^{4B}R^{4C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R⁵ is independently halogen, oxo, —CX^{5.1}₃, —CHX^{5.1}₂, —CH₂X^{5.1}, —CN, —N₃, —SOₙ₅R^{5A}, —SOᵥ₅NR^{5B}R^{5C}, —NHNR^{5B}R^{5C}, —ONR^{5B}R^{5C}, —NHC(O)NHNR^{5B}R^{5C}, —NHC(O)NR^{5B}R^{5C}, —N(O)ₘ₅, —NR^{5B}R^{5C}, —C(O)R^{5D}, —C(O)OR^{5D}, —C(O)NR^{5B}R^{5C}, —OR^{5A}, —NR^{5B}SO₂R^{5A}, —NR^{5B}C(O)R^{5D}, —NR^{5B}C(O)OR^{5D}, —NR^{5B}OR^{5D}, —OCX^{5.1}₃, —OCHX^{5.1}₂, —OCH₂X^{5.1}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R⁶ is independently halogen, oxo, —CX^{6.1}₃, —CHX^{6.1}₂, —CH₂X^{6.1}, —CN, —N₃, —SOₙ₆R^{6A}, —SOᵥ₆NR^{6B}R^{6C}, —NHNR^{6B}R^{6C}, —ONR^{6B}R^{6C}, —NHC(O)NHNR^{6B}R^{6C}, —NHC(O)NR^{6B}R^{6C}, —N(O)ₘ₆, —NR^{6B}R^{6C}, —C(O)R^{6D}, —C(O)OR^{6D}, C(O)NR^{6B}R^{6C}, —OR^{6A}, NR^{6B}SO₂R^{6A}, —NR^{6B}C(O)R^{6D}, —NR^{6B}C(O)OR^{6D}, —NR^{6B}OR^{6D}, —OCX^{6.1}₃, —OCHX^{6.1}₂, —OCH₂X^{6.1}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R⁷ is hydrogen, halogen, —CX^{7.1}₃, —CHX^{7.1}₂, —CH₂X^{7.1}, —CN, —N₃, —SOₙ₇R^{7A}, —SOᵥ₇NR^{7B}R^{7C}, —NHNR^{7B}R^{7C}, —ONR^{7B}R^{7C}, —NHC(O)NHNR^{7B}R^{7C}, —NHC(O)NR^{7B}R^{7C}, —N(O)ₘ₇, —NR^{7B}R^{7C}, —C(O)R^{7D}, —C(O)OR^{7D}, —C(O)NR^{7B}R^{7C}, —OR^{7A}, —NR^{7B}SO₂R^{7A}, —NR^{7B}C(O)R^{7D}, —NR^{7B}C(O)OR^{7D}, —NR^{7B}OR^{7D}, —OCX^{7.1}₃, —OCHX^{7.1}₂, —OCH₂X^{7.1}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R⁸ is hydrogen, halogen, —CX^{8.1}₃, —CHX^{8.1}₂, —CH₂X^{8.1}, —CN, —N₃, —SOₙ₈R^{8A}, —SOᵥ₈NR^{8B}R^{8C}, —NHNR^{8B}R^{8C}, —ONR^{8B}R^{8C}, —NHC(O)NHNR^{8B}R^{8C}, —NHC(O)NR^{8B}R^{8C}, —N(O)ₘ₈, —NR^{8B}R^{8C}, —C(O)R^{8D}, —C(O)OR^{8D}, —C(O)NR^{8B}R^{8C}, —OR^{8A}, —NR^{8B}SO₂R^{8A}, —NR^{8B}C(O)R^{8D}, —NR^{8B}C(O)OR^{8D}, —NR^{8B}OR^{8D}, —OCX^{8.1}₃, —OCHX^{8.1}₂, —OCH₂X^{8.1}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R⁹ is hydrogen, halogen, —CX^{9.1}₃, —CHX^{9.1}₂, —CH₂X^{9.1}, —CN, —N₃, —SOₙ₉R^{9A}, —SOᵥ₉NR^{9B}R^{9C}, —NHNR^{9B}R^{9C}, —ONR^{9B}R^{9C}, —NHC(O)NHNR^{9B}R^{9C}, —NHC(O)NR^{9B}R^{9C}, —N(O)ₘ₉, —NR^{9B}R^{9C}, —C(O)R^{9D}, —C(O)OR^{9D}, —C(O)NR^{9B}R^{9C}, —OR^{9A}, —NR^{9B}SO₂R^{9A}, —NR^{9B}C(O)R^{9D}, —NR^{9B}C(O)OR^{9D}, —NR^{9B}OR^{9D}, —OCX^{9.1}₃, —OCHX^{9.1}₂, —OCH₂X^{9.1}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or when X² is CR⁹ and X³ is CR¹⁰, then R⁹ and R¹⁰ may optionally be joined to form a substituted or unsubstituted cycloalkyl, substituted or unsubstituted heterocycloalkyl, substituted or unsubstituted aryl, or substituted or unsubstituted heteroaryl.

R¹⁰ is hydrogen, halogen, —CX^{10.1}₃, —CHX^{10.1}₂, —CH₂X^{10.1}, —CN, —N₃, —SOₙ₁₀R^{10A}, —SOᵥ₁₀NR^{10B}R^{10C}, —NHNR^{10B}R^{10C}, —ONR^{10B}R^{10C}, —NHC(O)NHNR^{10B}R^{10C}, —NHC(O)NR^{10B}R^{10C}, —N(O)ₘ₁₀, —NR^{10B}R^{10C}, —C(O)R^{10D}, —C(O)OR^{10D}, —C(O)NR^{10B}R^{10C}, —OR^{10A}, —NR^{10B}SO₂R^{10A}, —NR^{10B}C(O)R^{10D}, —NR^{10B}C(O)OR^{10D}, —NR^{10B}OR^{10D}, —OCX^{10.1}₃, —OCHX^{10.1}₂, —OCH₂X^{10.1}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or when X³ is CR¹⁰ and X⁴ is CR¹¹, then R¹⁰ and R¹¹ may optionally be joined to form a substituted or unsubstituted cycloalkyl, substituted or unsubstituted heterocycloalkyl, substituted or unsubstituted aryl, or substituted or unsubstituted heteroaryl.

R¹¹ is hydrogen, halogen, —CX^{11.1}₃, —CHX^{11.1}₂, —CH₂X^{11.1}—, —CN, —N₃, —SOₙ₁₁R^{11A}, —SOᵥ₁₁NR^{11B}R^{11C}, —NHNR^{11B}R^{11C}, —ONR_{11B}R^{11C}, —NHC(O)NHNR^{11B}R^{11C}, —NHC(O)NR^{11B}BR^{11C}, —N(O)ₘ₁₁, —NR^{11B}R^{11C}, —C(O)R^{11D}—C(O)OR^{11D}, —C(O)NR^{11B}R^{11C}, —OR^{11A}—NR^{11B}SO₂R^{11A}—NR^{11B}C(O)R^{11D}—NR^{11B}C(O)OR^{11D}—NR^{11B}OR^{11D}—OCX^{11.1}₃, —OCHX^{11.1}₂, —OCH₂X^{11.1}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or when X⁴ is CR¹¹ and X⁵ is CR¹², then R¹¹ and R¹² may optionally be joined to form a substituted or unsubstituted cycloalkyl, substituted or unsubstituted heterocycloalkyl, substituted or unsubstituted aryl, or substituted or unsubstituted heteroaryl.

R¹² is hydrogen, halogen, —CX^{12.1}₃, —CHX^{12.1}₂, —CH₂X^{12.1}, —CN, —N₃, —SOₙ₁₂R^{12A}, —SOᵥ₁₂NR^{12B}R^{12C}, —NHNR^{12B}R^{12C}, —ONR^{12B}R^{12C}, —NHC(O)NHNR^{12B}R^{12C}, —NHC(O)NR^{12B}R^{12C}, —N(O)ₘ₁₂, —NR^{12B}R^{12C}, —C(O)R^{12D}, —C(O)OR^{12D}, —C(O)NR^{12B}R^{12C}, —OR^{12A}—NR¹²BSO₂R^{12A}—NR¹²BC(O)R^{12D}—NR^{12B}C(O)OR^{12D}—NR¹²BOR^{12D}, —OCX^{12.1}₃, —OCHX^{12.1}₂, —OCH₂X^{12.1}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R^{1A}, R^{1B}, R^{1C}, R^{1D}, R^{2A}, R^{2B}, R^{2C}, R^{2D}, R^{3A}, R^{3B}, R^{3C}, R^{3D}, R^{4A}, R^{4B}, R^{4C}, R^{4D}, R^{5A}, R^{5B}, R^{5C}, R^{5D}, R^{6A}, R^{6B}, R^{6C}, R^{6D}, R^{7A}, R^{7B}, R^{7C}, R^{7D}, R^{7B}, R^{8A}, R^{8B}, R^{8C}, R^{8D}, R^{9A}, R^{9B}, R^{9C}, R^{9D}, R^{10A}, R^{10B}, R^{10C}, R^{10D}, R^{11A}, R^{11B}, R^{11C}, R^{11D}, R^{12A}, R^{12B}, R^{12C}, and R^{12D} are independently hydrogen, halogen, —CF₃, —CCl₃, —CBr₃, —Cl₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1B} and R^{1C} substituents bonded to the same nitrogen atom may optionally be joined to form a substituted or unsubstituted heterocycloalkyl or substituted or unsubstituted heteroaryl; R^{2B} and R^{2C} substituents bonded to the same nitrogen atom may optionally be joined to form a substituted or unsubstituted heterocycloalkyl or substituted or unsubstituted heteroaryl; R^{3B} and R^{3C} substituents bonded to the same nitrogen atom may optionally be joined to form a substituted or unsubstituted heterocycloalkyl or substituted or unsubstituted heteroaryl; R^{4B} and R^{4C} substituents bonded to the same nitrogen atom may optionally be joined to form a substituted or unsubstituted heterocycloalkyl or substituted or unsubstituted heteroaryl; R^{5B} and R^{5C} substituents bonded to the same nitrogen atom may optionally be joined to form a substituted or unsubstituted heterocycloalkyl or substituted or unsubstituted heteroaryl; R^{6B} and R^{6C} substituents bonded to the same nitrogen atom may optionally be joined to form a substituted or unsubstituted heterocycloalkyl or substituted or unsubstituted heteroaryl; R^{7B} and R^{7C} substituents bonded to the same nitrogen atom may optionally be joined to form a substituted or unsubstituted heterocycloalkyl or substituted or unsubstituted heteroaryl; R^{8B} and R^{8C} substituents bonded to the same nitrogen atom may optionally be joined to form a substituted or unsubstituted heterocycloalkyl or substituted or unsubstituted heteroaryl; R^{9B} and R^{9C} substituents bonded to the same nitrogen atom may optionally be joined to form a substituted or unsubstituted heterocycloalkyl or substituted or unsubstituted heteroaryl; R^{10B} and R^{10C} substituents bonded to the same nitrogen atom may optionally be joined to form a substituted or unsubstituted heterocycloalkyl or substituted or unsubstituted heteroaryl; R^{11B} and R^{11C} substituents bonded to the same nitrogen atom may optionally be joined to form a substituted or unsubstituted heterocycloalkyl or substituted or unsubstituted heteroaryl; R^{12B} and R^{12C} substituents bonded to the same nitrogen atom may optionally be joined to form a substituted or unsubstituted heterocycloalkyl or substituted or unsubstituted heteroaryl.

X^{1.1}, X^{2.1}, X^{3.1}, X^{4.1}, X^{5.1}, X^{6.1}, X^{7.1}, X^{8.1}, X^{9.1}, X^{10.1}, X^{11.1}, and X^{12.1} are independently —Cl, —Br, —I, or —F. The symbols n1, n2, n3, n4, n5, n6, n7, n8, n9, n10, n11, and n12 are independently an integer from 0 to 4. The symbols m1, m2, m3, m5, m6, m7, m8, m9, m10, m11, m12, v1, v2, v3, v4, v5, v6, v7, v8, v9, v10, v11, and v12 are independently 1 or 2.

In an aspect is provided a pharmaceutical composition including a compound described herein, or a pharmaceutically acceptable salt thereof, and a pharmaceutically acceptable excipient.

In an aspect is provided a method of inhibiting C-C chemokine receptor type 4 (CCR4), including contacting CCR4 with a compound described herein, or a pharmaceutically acceptable salt thereof.

In an aspect is provided a method of treating or preventing a disease or disorder mediated by CCR4, including administering to a subject in need thereof a therapeutically effective amount of a compound described herein, or a pharmaceutically acceptable salt thereof.

In an aspect is provided a method of treating or preventing a disease or disorder mediated by CCR4, including administering to a subject in need thereof a therapeutically effective amount of a pharmaceutical composition described herein.

In an aspect is provided a kit including a compound described herein (e.g., a CCR4 inhibitor) or pharmaceutical compositions thereof.

### DETAILED DESCRIPTION

Provided herein are, for example, compounds and compositions for inhibition of C-C chemokine receptor type 4, and pharmaceutical compositions including the same. Also provided herein are, for example, methods of treating or preventing a disease, disorder or condition, or a symptom thereof, for example a disease, disorder or condition, or a symptom thereof, mediated by modulation (e.g., inhibition) of CCR4.

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di-, and multivalent radicals. The alkyl may include a designated number of carbons (e.g., C₁-C₁₀ means one to ten carbons). In embodiments, the alkyl is fully saturated. In embodiments, the alkyl is monounsaturated. In embodiments, the alkyl is polyunsaturated. Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkenyl includes one or more double bonds. An alkynyl includes one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene. The term “alkynylene” by itself or as part of another substituent, means, unless otherwise stated, a divalent radical derived from an alkyne. In embodiments, the alkylene is fully saturated. In embodiments, the alkylene is monounsaturated. In embodiments, the alkylene is polyunsaturated. An alkenylene includes one or more double bonds. An alkynylene includes one or more triple bonds. The term “unsubstituted methylene” refers to the moiety—CH₂—. The term “substituted methylene” refers to that moiety wherein one or both hydrogen atoms are replaced with a substituent.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —S—CH₂—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds. In embodiments, the heteroalkyl is fully saturated. In embodiments, the heteroalkyl is monounsaturated. In embodiments, the heteroalkyl is polyunsaturated.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like. The term “heteroalkenylene,” by itself or as part of another substituent, means, unless otherwise stated, a divalent radical derived from a heteroalkene. The term “heteroalkynylene” by itself or as part of another substituent, means, unless otherwise stated, a divalent radical derived from a heteroalkyne. In embodiments, the heteroalkylene is fully saturated. In embodiments, the heteroalkylene is monounsaturated. In embodiments, the heteroalkylene is polyunsaturated. A heteroalkenylene includes one or more double bonds. A heteroalkynylene includes one or more triple bonds.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. In embodiments, the cycloalkyl is fully saturated. In embodiments, the cycloalkyl is monounsaturated. In embodiments, the cycloalkyl is polyunsaturated. In embodiments, the heterocycloalkyl is fully saturated. In embodiments, the heterocycloalkyl is monounsaturated. In embodiments, the heterocycloalkyl is polyunsaturated.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. A bicyclic or multicyclic cycloalkyl ring system refers to multiple rings fused together wherein at least one of the fused rings is a cycloalkyl ring and wherein the multiple rings are attached to the parent molecular moiety through any carbon atom contained within a cycloalkyl ring of the multiple rings.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. A bicyclic or multicyclic cycloalkenyl ring system refers to multiple rings fused together wherein at least one of the fused rings is a cycloalkenyl ring and wherein the multiple rings are attached to the parent molecular moiety through any carbon atom contained within a cycloalkenyl ring of the multiple rings.

In embodiments, the term “heterocycloalkyl” means a monocyclic, bicyclic, or a multicyclic heterocycloalkyl ring system. In embodiments, heterocycloalkyl groups are fully saturated. A bicyclic or multicyclic heterocycloalkyl ring system refers to multiple rings fused together wherein at least one of the fused rings is a heterocycloalkyl ring and wherein the multiple rings are attached to the parent molecular moiety through any atom contained within a heterocycloalkyl ring of the multiple rings.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring and wherein the multiple rings are attached to the parent molecular moiety through any carbon atom contained within an aryl ring of the multiple rings. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring and wherein the multiple rings are attached to the parent molecular moiety through any atom contained within a heteroaromatic ring of the multiple rings). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g., substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g., all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylarylene” as an arylene moiety covalently bonded to an alkylene moiety (also referred to herein as an alkylene linker). In embodiments, the alkylarylene group has the formula:

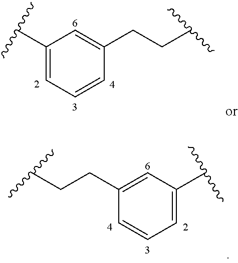 [SMILES: *CCc1cccc(*)c1 ; *CCc1cccc(*)c1]

An alkylarylene moiety may be substituted (e.g., with a substituent group) on the alkylene moiety or the arylene linker (e.g., at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃, —SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, halogen, —SiR′R″R″′, —OC(O)R′, —C(O)R′, —CO₂R′, —CONR′R″, —OC(O)NR′R″, —NR″C(O)R′, —NR′C(O)NR″R″′, —NR″C(O)₂R′, —NRC(NR′R″R″)═NR″″, —NRC(NR′R″)═NR″′, —S(O)R′, —S(O)₂R′, —S(O)₂NR′R″, —NRSO₂R′, —NR′NR″RWW, —ONR′R″, —NR′C(O)NR″NR″R″″, —CN, —NO₂, —NR′SO₂RWW, —NR′C(O)RWW, —NR′C(O)ORWW, —NR′OR″, in a number ranging from zero to (2m′+1), where m′ is the total number of carbon atoms in such radical. R, R′, R″, R″′, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R″′, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, halogen, —SiR′R″R″′, —OC(O)R′, —C(O)R′, —CO₂R′, —CONR′R″, —OC(O)NR′R″, —NR″C(O)R′, —NR′C(O)NR″R″′″, —NR″C(O)₂R′, —NR-C(NR′R″R′″)═NR″″, —NR-C(NR′R″)═NR″″, —S(O)R′, —S(O)₂R′, —S(O)₂NR′R″, —NRSO₂R′, —NR′NR″RWW, —ONR′R″, —NR′C(O)NR″NR″′R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)OR″, —NR′OR″, in a number ranging from zero to the total number of open valences on the aromatic ring system; and where R′, R″, R″′, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R″′, and R″″ groups when more than one of these groups is present.

Substituents for rings (e.g., cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g., a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}-U-, wherein T and U are independently —NR—, —O—, —CRR′-, or a single bond, and q is an integer of from 0 to 3. Alternatively, two of the substituents on adjacent atoms of the aryl or heteroaryl ring may optionally be replaced with a substituent of the formula -A-(CH₂)ᵣ-B-, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R″′)_{d}—, where s and d are independently integers of from 0 to 3, and X′ is —O—, —NR′—, —S—, —S(O)—, —S(O)₂—, or —S(O)₂NR′—. The substituents R, R′, R″, and R″′ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —CI₃, —CHCl₂, —CHBr₂, —CHF₂, —CHI₂, —CH₂Cl,
—CH₂Br, —CH₂F, —CH₂I, —OCCl₃, —OCF₃, —OCBr₃, —OC₃, —OCHCl₂, —OCHBr₂, —OCHI₂,
—OCHF₂, —OCH₂Cl, —OCH₂Br, —OCH₂I, —OCH₂F, —CN, —OH, —NH₂, —COOH, —CONH₂,
—NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃,
unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
(i) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(ii) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
   (a) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —N O₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(b) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₅ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted phenyl, and each substituted or unsubstituted heteroaryl is a substituted or unsubstituted 5 to 6 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

In a recited claim or chemical formula description herein, each R substituent or L linker that is described as being “substituted” without reference as to the identity of any chemical moiety that composes the “substituted” group (also referred to herein as an “open substitution” on an R substituent or L linker or an “openly substituted” R substituent or L linker), the recited R substituent or L linker may, in embodiments, be substituted with one or more first substituent groups as defined below.

The first substituent group is denoted with a corresponding first decimal point numbering system such that, for example, R¹ may be substituted with one or more first substituent groups denoted by R^{1.1}, R² may be substituted with one or more first substituent groups denoted by R^{2.1}, R³ may be substituted with one or more first substituent groups denoted by R^{3.1}, R⁴ may be substituted with one or more first substituent groups denoted by R^{4.1}, R⁵ may be substituted with one or more first substituent groups denoted by R^{5.1}, and the like up to or exceeding an R¹⁰⁰ that may be substituted with one or more first substituent groups denoted by R^{100.1}. As a further example, R^{1A} may be substituted with one or more first substituent groups denoted by R^{1A.1}, R^{2A} may be substituted with one or more first substituent groups denoted by R^{2A.1}, R^{3A} may be substituted with one or more first substituent groups denoted by R^{3A.1}, R^{4A} may be substituted with one or more first substituent groups denoted by R^{4A.1}, R^{5A} may be substituted with one or more first substituent groups denoted by R^{5A.1} and the like up to or exceeding an R^{100A} may be substituted with one or more first substituent groups denoted by R^{100A}. As a further example, L¹ may be substituted with one or more first substituent groups denoted by R^{L1.1}, L² may be substituted with one or more first substituent groups denoted by R^{L2.1}, L³ may be substituted with one or more first substituent groups denoted by R^{L3.1}, L⁴ may be substituted with one or more first substituent groups denoted by R^{L4.1}, L⁵ may be substituted with one or more first substituent groups denoted by R^{L5.1} and the like up to or exceeding an L¹⁰⁰ which may be substituted with one or more first substituent groups denoted by R^{L100.1}. Thus, each numbered R group or L group (alternatively referred to herein as R¹¹ or L″ wherein “WW” represents the stated superscript number of the subject R group or L group) described herein may be substituted with one or more first substituent groups referred to herein generally as R^{WW.1} or R^{LWW.1}, respectively. In turn, each first substituent group (e.g., R^{1.1}, R^{2.1}, R^{3.1}, R^{4.1}, R^{5.1}, . . . R^{100.1}; R^{1A.1}, R^{2A.1}, R^{3A.1}, R^{4A.1}, R^{5A.1}, . . . , R^{100A.1}; R^{L1.1}, R^{L2.2}, R^{L3.1}, R^{L4.1}, R^{L5.1} . . . , R^{L100.1}) may be further substituted with one or more second substituent groups (e.g., R^{1.2}, R^{2.2}, R^{3.2}, R^{4.2}, R^{5.2} . . . R^{100.2}; R^{1A.2}, R^{2A.2}, R^{3A.2}, R^{4A.2}, R^{5A.2} . . . R^{100A.2}; R^{L1.2}, R^{L2.2}, R^{L3.2}, R^{L4.2}, R^{L5.2} . . . R^{L100.2}, respectively). Thus, each first substituent group, which may alternatively be represented herein as R^{WW.1} as described above, may be further substituted with one or more second substituent groups, which may alternatively be represented herein as R^{WW.2}.

Finally, each second substituent group (e.g., R^{1.2}, R^{2.2}, R^{3.2}, R^{4.2}, R^{5.2} . . . R^{100.2}; R^{1A.2}, R^{2A.2}, R^{3A.2}, R^{4A.2}, R^{5A.2} . . . R^{100A.2}; R^{L1.2}, R^{L2.2}, R^{L3.2}, R^{L4.2}, R^{L5.2} . . . R^{100.2}) may be further substituted with one or more third substituent groups (e.g., R^{1.3}, R^{2.3}, R^{3.3}, R^{4.3}, R^{5.3} . . . R^{100.3}, R^{1A.3}, R^{2A.3}, R^{3A.3}, R^{4A.3}, R^{5A.3} . . . R^{100A.3}; R^{L1.3}, R^{L2.3}, R^{L3.3}, R^{L4.3}, R^{L5.3} . . . RL^{100.3}; respectively). Thus, each second substituent group, which may alternatively be represented herein as R^{WW.2} as described above, may be further substituted with one or more third substituent groups, which may alternatively be represented herein as R^{WW.3}. Each of the first substituent groups may be optionally different. Each of the second substituent groups may be optionally different. Each of the third substituent groups may be optionally different.

Thus, as used herein, R^{WW} represents a substituent recited in a claim or chemical formula description herein which is openly substituted. “WW” represents the stated superscript number of the subject R group (1, 2, 3, 1A, 2A, 3A, 1B, 2B, 3B, etc.). Likewise, L” is a linker recited in a claim or chemical formula description herein which is openly substituted. Again, “WW” represents the stated superscript number of the subject L group (1, 2, 3, 1A, 2A, 3A, 1B, 2B, 3B, etc.). As stated above, in embodiments, each R^{WW} may be unsubstituted or independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group may be unsubstituted or independently substituted with one or more third substituent groups, referred to herein as R^{WW.3}. Similarly, each L^{WW} linker may be unsubstituted or independently substituted with one or more first substituent groups, referred to herein as R^{LWW.1}; each first substituent group, R^{LWW.1}, may be unsubstituted or independently substituted with one or more second substituent groups, referred to herein as R^{LWW.2}; and each second substituent group may be unsubstituted or independently substituted with one or more third substituent groups, referred to herein as R^{LWW.3}. Each first substituent group is optionally different. Each second substituent group is optionally different. Each third substituent group is optionally different. For example, if R^{WW} is phenyl, the said phenyl group is optionally substituted by one or more R^{WW.1} groups as defined herein below, e.g., when R^{WW.1} is R^{WW.2}-substituted or unsubstituted alkyl, examples of groups so formed include but are not limited to itself optionally substituted by 1 or more R^{WW.2}, which R^{WW.2} is optionally substituted by one or more R^{WW.3}. By way of example when the R^{WW} group is phenyl substituted by R^{WW.1}, which is methyl, the methyl group may be further substituted to form groups including but not limited to:

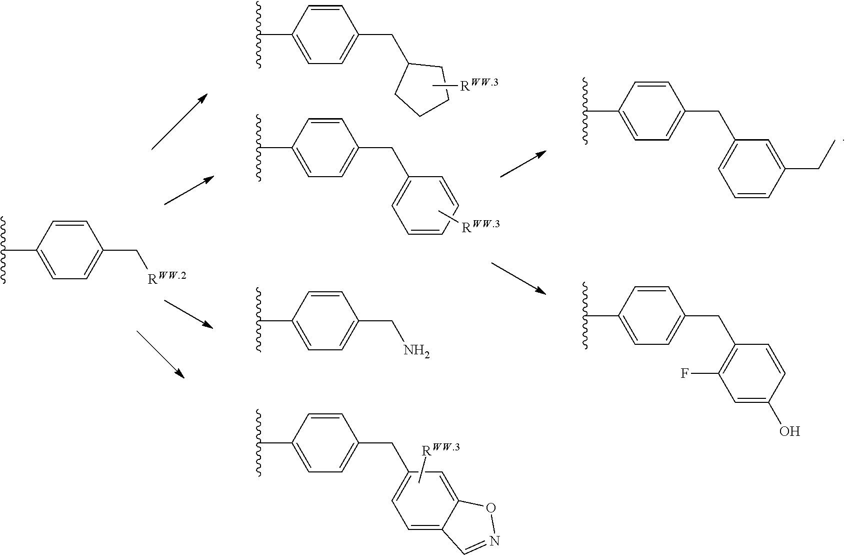

R^{WW.1} is independently oxo, halogen, —CX^{WW.1}₃, —CHX^{WW.1}₂, —CH₂X^{WW.1}, —OCX^{WW.1}₃, —OCH₂X^{WW.1}, —OCHX^{WW.2}, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.2}-substituted or unsubstituted alkyl (e.g., C₁-C₈, C₁-C₆, C₁-C₄, or C₁-C₂), R^{WW.2}-substituted or unsubstituted heteroalkyl (e.g., 2 to 8 membered, 2 to 6 membered, 4 to 6 membered, 2 to 3 membered, or 4 to 5 membered), R^{WW.2}-substituted or unsubstituted cycloalkyl (e.g., C₃-C₈, C₃-C₆, C₄-C₆, or C₅-C₆), R^{WW.2}-substituted or unsubstituted heterocycloalkyl (e.g., 3 to 8 membered, 3 to 6 membered, 4 to 6 membered, 4 to 5 membered, or 5 to 6 membered), R^{WW.2}-substituted or unsubstituted aryl (e.g., C₆-C₁₂, C₆-C₁₀, or phenyl), or R^{WW.2}-substituted or unsubstituted heteroaryl (e.g., 5 to 12 membered, 5 to 10 membered, 5 to 9 membered, or 5 to 6 membered). In embodiments, R^{WW.1} is independently oxo, halogen, —CX^{WW.1}₃, —CHX^{WW.1}₂, —CH₂X^{WW.1}, —OCX^{WW.1}₃, —OCH₂X^{WW.1}, —OCHX^{WW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.1} is independently —F, —Cl, —Br, or —I.

R^{WW.2} is independently oxo, halogen, —CX^{WW.2}₃, —CHX^{WW.2}₂, —CH₂X^{WW.2}, —OCX^{WW.2}₃, —OCH₂X^{WW.2}, —OCHX^{WW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.3}-substituted or unsubstituted alkyl (e.g., C₁-C₈, C₁-C₆, C₁-C₄, or C₁-C₂), R^{WW.3}-substituted or unsubstituted heteroalkyl (e.g., 2 to 8 membered, 2 to 6 membered, 4 to 6 membered, 2 to 3 membered, or 4 to 5 membered), R″-3-substituted or unsubstituted cycloalkyl (e.g., C₃-C₈, C₃-C₆, C₄-C₆, or C₅-C₆), R^{W}-3-substituted or unsubstituted heterocycloalkyl (e.g., 3 to 8 membered, 3 to 6 membered, 4 to 6 membered, 4 to 5 membered, or 5 to 6 membered), R^{WW.3}-substituted or unsubstituted aryl (e.g., C₆-C₁₂, C₆-C₁₀, or phenyl), or R^{WW.3}-substituted or unsubstituted heteroaryl (e.g., 5 to 12 membered, 5 to 10 membered, 5 to 9 membered, or 5 to 6 membered). In embodiments, R^{WW.2} is independently oxo, halogen, —CX^{WW.2}₃, —CHX^{WW.2}₂, —CH₂X^{WW.2}, —OCX^{WW.2}₃, —OCH₂X^{WW.2}, —OCHX^{WW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.2} is independently —F, —Cl, —Br, or —I.

R^{WW.3} is independently oxo, halogen, —CX^{WW.3}₃, —CHX^{WW.3}₂, —CH₂X^{WW.3}₃, —OCX^{WW.3}₃, —OCH₂X^{WW.3}, —OCHX^{WW.3}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.3} is independently —F, —Cl, —Br, or —I.

Where two different R^{WW} substituents are joined together to form an openly substituted ring (e.g., substituted cycloalkyl, substituted heterocycloalkyl, substituted aryl or substituted heteroaryl), in embodiments the openly substituted ring may be independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group, R^{WW.2} may be unsubstituted or independently substituted with one or more third substituent groups, referred to herein as R^{WW3}; and each third substituent group, R^{WW3}, is unsubstituted. Each first substituent group is optionally different. Each second substituent group is optionally different. Each third substituent group is optionally different. In the context of two different R¹substituents joined together to form an openly substituted ring, the “WW” symbol in the R^{WW.1}, R^{WW.2} and R^{WW.3} refers to the designated number of one of the two different R^{WW} substituents. For example, in embodiments where R^{100A} and R^{100B} are optionally joined together to form an openly substituted ring, R^{WW.1} is R^{100A.1}, R^{WW.2} is R^{100A.2}, and R^{WW.3} is R^{100A.3}. Alternatively, in embodiments where R^{100A} and R^{100B} are optionally joined together to form an openly substituted ring, R^{WW.1} is R^{100B}, R^{WW.2} is R^{100B.2}, and R^{WW.3} is R^{100B.3}. R^{WW.1}, R^{WW.2} and R^{WW.3} in this paragraph are as defined in the preceding paragraphs.

R^{LWW.1} is independently oxo, halogen, —CX^{LWW.1}₃, —CHX^{LWW.1}₂, —CH₂X^{LWW.1}, —OCX^{LWW.1}₃, —OCH₂X^{LWW.1}₂, —OCHX^{LWW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{LWW. 2}-substituted or unsubstituted alkyl (e.g., C₁-C₈, C₁-C₆, C₁-C₄, or C₁-C₂), R^{LWW.2}-substituted or unsubstituted heteroalkyl (e.g., 2 to 8 membered, 2 to 6 membered, 4 to 6 membered, 2 to 3 membered, or 4 to 5 membered), R^{LWW.2}-substituted or unsubstituted cycloalkyl (e.g., C₃-C₈, C₃-C₆, C₄-C₆, or C₅-C₆), R^{LWW.2}-substituted or unsubstituted heterocycloalkyl (e.g., 3 to 8 membered, 3 to 6 membered, 4 to 6 membered, 4 to 5 membered, or 5 to 6 membered), R^{LWW.2}-substituted or unsubstituted aryl (e.g., C₆-C₁₂, C₆-C₁₀, or phenyl), or R^{LWW.2}-substituted or unsubstituted heteroaryl (e.g., 5 to 12 membered, 5 to 10 membered, 5 to 9 membered, or 5 to 6 membered). In embodiments, R^{LWW.1} is independently oxo, halogen, —CX^{LWW.1}₃, —CHX^{LWW.1}₂, —CH₂X^{LWW.1}, —OCX^{LWW.1}₃, —OCH₂X^{LWW.1}, —OCHX^{LWW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.1} is independently —F, —Cl, —Br, or —I.

R^{LWW.2} is independently oxo, halogen, —CX^{LWW.2}₃, —CHX^{LWW.2}₂, —CH₂X^{LWW.2}, —OCX^{LWW.2}₃, —OCH₂X^{LWW.2}—OCHX^{LWW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{LWW.3}-substituted or unsubstituted alkyl (e.g., C₁-C₈, C₁-C₆, C₁-C₄, or C₁-C₂), R^{LWW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{LWW.3}-substituted or unsubstituted heterocycloalkyl (e.g., 3 to 8 membered, 3 to 6 membered, 4 to 6 membered, 4 to 5 membered, or 5 to 6 membered), R^{LWW.3}-substituted or unsubstituted aryl (e.g., C₆-C₁₂, C₆-C₁₀, or phenyl), or R^{LWW.3}-substituted or unsubstituted heteroaryl (e.g., 5 to 12 membered, 5 to 10 membered, 5 to 9 membered, or 5 to 6 membered). In embodiments, R^{LWW.2} is independently oxo, halogen, —CX^{LWW.2}₃, —CHX^{LWW.2}₂, —CH₂X^{LWW.2}, —OCX^{LWW.2}₃, —OCH₂XL^{WW.2}, OCHX^{LWW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.2} is independently —F, —Cl, —Br, or —I.

R^{LWW.3} is independently oxo, halogen, —CX^{LWW.3}₃, —CHX^{LWW.3}₂, —CH₂X^{LWW.3}—OCX^{LWW.3}₃, —OCH₂XL^{WW.3}, OCHX^{LWW.3}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW}0.3 is independently —F, —Cl, —Br, or —I.

In the event that any R group recited in a claim or chemical formula description set forth herein (R^{W} substituent) is not specifically defined in this disclosure, then that R group (R″ group) is hereby defined as independently oxo, halogen, —CX^{WW}₃, —CHX^{WW}₂, —CH₂X^{WW}, —OCX^{WW}₃, —OCH₂X^{WW}, —OCHX^{WW}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{11.1}-substituted or unsubstituted alkyl (e.g., C₁-C₈, C₁-C₆, C₁-C₄, or C₁-C₂), R^{WW.1}-substituted or unsubstituted heteroalkyl (e.g., 2 to 8 membered, 2 to 6 membered, 4 to 6 membered, 2 to 3 membered, or 4 to 5 membered), R^{WW.1}-substituted or unsubstituted cycloalkyl (e.g., C₃-C₈, C₃-C₆, C₄-C₆, or C₅-C₆), R^{WW.1}-substituted or unsubstituted heterocycloalkyl (e.g., 3 to 8 membered, 3 to 6 membered, 4 to 6 membered, 4 to 5 membered, or 5 to 6 membered), R^{WW.1}-substituted or unsubstituted aryl (e.g., C₆-C₁₂, C₆-C₁₀, or phenyl), or R^{WW.1}-substituted or unsubstituted heteroaryl (e.g., 5 to 12 membered, 5 to 10 membered, 5 to 9 membered, or 5 to 6 membered). X^{WW} is independently —F, —Cl, —Br, or —I. Again, “WW” represents the stated superscript number of the subject R group (e.g., 1, 2, 3, 1A, 2A, 3A, 1B, 2B, 3B, etc.). R^{WW.1}, R^{WW.2}, and R^{WW.3} are as defined above.

In the event that any L linker group recited in a claim or chemical formula description set forth herein (i.e., an L^{WW} substituent) is not explicitly defined, then that L group (L^{WW} group) is herein defined as independently a bond, —O—, —NH—, —C(O)—, —C(O)NH—, —NHC(O)—, —NHC(O)NH—, —NHC(NH)NH—, —C(O)O—, —OC(O)—, —S—, —SO₂—, —SO₂NH—, RLWW¹substituted or unsubstituted alkylene (e.g., C₁-C₈, C₁-C₆, C₁-C₄, or C₁-C₂), R^{LWW.1}-substituted or unsubstituted heteroalkylene (e.g., 2 to 8 membered, 2 to 6 membered, 4 to 6 membered, 2 to 3 membered, or 4 to 5 membered), R^{LWW.1}-substituted or unsubstituted cycloalkylene (e.g., C₃-C₈, C₃-C₆, C₄-C₆, or C₅-C₆), R^{LWW.1}substituted or unsubstituted heterocycloalkylene (e.g., 3 to 8 membered, 3 to 6 membered, 4 to 6 membered, 4 to 5 membered, or 5 to 6 membered), R^{LWW.1}-substituted or unsubstituted arylene (e.g., C₆-C₁₂, C₆-C₁₀, or phenyl), or R^{LWW.1}-substituted or unsubstituted heteroarylene (e.g., 5 to 12 membered, 5 to 10 membered, 5 to 9 membered, or 5 to 6 membered). Again, “WW” represents the stated superscript number of the subject L group (1, 2, 3, 1A, 2A, 3A, 1B, 2B, 3B, etc.). R^{LWW.1}, as well as R^{LWW.2} and R^{LWW.3} are as defined above.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)-for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

“Analog,” “analogue,” or “derivative” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an”, as used in herein means one or more. In addition, the phrase “substituted with a[n]”, as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl”, the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13A}, R^{13B}, R^{13C}, R^{13D}, etc., wherein each of R^{13A}, R^{13B}, R^{13C}, R^{13D}, etc. is defined within the scope of the definition of R¹³ and optionally differently. Where an R moiety, group, or substituent as disclosed herein is attached through the representation of a single bond and the R moiety, group, or substituent is oxo, a person having ordinary skill in the art will immediately recognize that the oxo is attached through a double bond in accordance with the normal rules of chemical valency.

A “detectable moiety” as used herein refers to a moiety that can be covalently or noncovalently attached to a compound or biomolecule that can be detected for instance, using techniques known in the art. In embodiments, the detectable moiety is covalently attached. The detectable moiety may provide for imaging of the attached compound or biomolecule. The detectable moiety may indicate the contacting between two compounds. Exemplary detectable moieties are fluorophores, antibodies, reactive dies, radio-labeled moieties, magnetic contrast agents, and quantum dots. Exemplary fluorophores include fluorescein, rhodamine, GFP, coumarin, FJTC, Alexa fluor, Cy3, Cy5, BODIPY, and cyanine dyes. Exemplary radionuclides include Fluorine-18, Gallium-68, and Copper-64. Exemplary magnetic contrast agents include gadolinium, iron oxide and iron platinum, and manganese.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1.19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g., methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents. In embodiments, compounds of the present disclosure contain both basic and acidic functionalities that allow the compounds to be converted into either base or acid addition salts. The neutral forms of the compounds may be regenerated by contacting the salt with a base or acid and isolating the parent compound in a conventional manner. The parent form of the compounds differs from the various salt forms in certain physical properties, such as solubility in polar solvents, but, unless specifically indicated, the salts disclosed herein are equivalent to the parent form of the compound for the purposes of the present disclosure.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

A “CCR4 inhibitor” refers to a compound (e.g., a compound described herein) capable of reducing the activity of CCR4 (e.g., by binding to CCR4) when compared to a control, such as absence of the compound or a compound with known inactivity.

The terms “polypeptide,” “peptide”, and “protein” are used interchangeably herein to refer to a polymer of amino acid residues, wherein the polymer may optionally be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymer. In embodiments, the terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified polypeptide backbones. The terms include fusion proteins, including, but not limited to, fusion proteins with a heterologous amino acid sequence; fusion proteins with heterologous and homologous leader sequences, with or without N-terminus methionine residues;
immunologically tagged proteins; and the like.

A polypeptide, or a cell is “recombinant” when it is artificial or engineered, or derived from or contains an artificial or engineered protein or nucleic acid (e.g., non-natural or not wild type). For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

The term “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, include prevention of an injury, pathology, condition, or disease. In embodiments, treating is preventing. In embodiments, treating does not include preventing. In embodiments, the treating or treatment is not prophylactic treatment.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms (e.g., ocular pain, seeing halos around lights, red eye, very high intraocular pressure), fully or partially remove the disease's underlying cause, shorten a disease's duration, or do a combination of these things.

“Treating” and “treatment” as used herein include prophylactic treatment. Treatment methods include administering to a subject a therapeutically effective amount of a compound described herein. The administering step may consist of a single administration or may include a series of administrations. The length of the treatment period depends on a variety of factors, such as the severity of the condition, the age of the patient, the concentration of the compound, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient.

The term “prevent” refers to a decrease in the occurrence of disease symptoms in a patient. As indicated above, the prevention may be complete (no detectable symptoms) or partial, such that fewer symptoms are observed than would likely occur absent treatment. In embodiments, prevent refers to slowing the progression of the disease, disorder or condition or inhibiting progression thereof to a harmful or otherwise undesired state.

“Contacting” is used in accordance with its plain ordinary meaning and refers to the process of allowing at least two distinct species (e.g., chemical compounds including biomolecules, or cells) to become sufficiently proximal to react, interact or physically touch. It should be appreciated; however, the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be a compound as described herein and a cellular component (e.g., protein, ion, lipid, nucleic acid, nucleotide, amino acid, protein, particle, organelle, cellular compartment, microorganism, virus, lipid droplet, vesicle, small molecule, protein complex, protein aggregate, or macromolecule). In some embodiments contacting includes allowing a compound described herein to interact with a cellular component (e.g., protein, ion, lipid, nucleic acid, nucleotide, amino acid, protein, particle, virus, lipid droplet, organelle, cellular compartment, microorganism, vesicle, small molecule, protein complex, protein aggregate, or macromolecule) that is involved in a signaling pathway.

As defined herein, the term “activation,” “activate,” “activating” and the like in reference to a protein refers to conversion of a protein into a biologically active derivative from an initial inactive or deactivated state. The terms reference activation, or activating, sensitizing, or up-regulating signal transduction or enzymatic activity or the amount of a protein decreased in a disease.

The terms “agonist,” “activator,” “upregulator,” etc. refer to a substance capable of detectably increasing the expression or activity of a given gene or protein. The agonist can increase expression or activity by at least 10%, 20%, 30%, 40%, 50%, 60%, 70%, 80%, 90%, 95%, 96%, 97%, 98%, or 99% in comparison to a control in the absence of the agonist. In certain instances, expression or activity is 1.5-fold, 2-fold, 3-fold, 4-fold, 5-fold, 10-fold or higher than the expression or activity in the absence of the agonist. In embodiments, an agonist is a molecule that interacts with a target to cause or promote an increase in the activation of the target. In embodiments, activators are molecules that increase, activate, facilitate, enhance activation, sensitize, or up-regulate, e.g., a gene, protein, ligand, receptor, or cell.

As defined herein, the term “inhibition,” “inhibit,” “inhibiting” and the like in reference to a cellular component-inhibitor interaction means negatively affecting (e.g., decreasing) the activity or function of the cellular component (e.g., decreasing the signaling pathway stimulated by a cellular component (e.g., protein, ion, lipid, virus, lipid droplet, nucleic acid, nucleotide, amino acid, protein, particle, organelle, cellular compartment, microorganism, vesicle, small molecule, protein complex, protein aggregate, or macromolecule)), relative to the activity or function of the cellular component in the absence of the inhibitor. In embodiments inhibition means negatively affecting (e.g., decreasing) the concentration or levels of the cellular component relative to the concentration or level of the cellular component in the absence of the inhibitor. In some embodiments, inhibition refers to reduction of a disease or symptoms of disease. In some embodiments, inhibition refers to a reduction in the activity of a signal transduction pathway or signaling pathway (e.g., reduction of a pathway involving the cellular component). Thus, inhibition includes, at least in part, partially or totally blocking stimulation, decreasing, preventing, or delaying activation, or inactivating, desensitizing, or down-regulating the signaling pathway or enzymatic activity or the amount of a cellular component.

The terms “inhibitor,” “repressor,” “antagonist,” or “downregulator” interchangeably refer to a substance capable of detectably decreasing the expression or activity of a given gene or protein. The antagonist can decrease expression or activity by at least 10%, 20%, 30%, 40%, 50%, 60%, 70%, 80%, 90%, 95%, 96%, 97%, 98%, or 99% in comparison to a control in the absence of the antagonist. In certain instances, expression or activity is 1.5-fold, 2-fold, 3-fold, 4-fold, 5-fold, 10-fold or lower than the expression or activity in the absence of the antagonist. An antagonist prevents, reduces, inhibits, or neutralizes the activity of an agonist, and an antagonist can also prevent, inhibit, or reduce constitutive activity of a target, e.g., a target receptor, even where there is no identified agonist. In embodiments, inhibitors are molecules that decrease, block, prevent, delay activation, inactivate, desensitize, or down-regulate, e.g., a gene, protein, ligand, receptor, or cell. An inhibitor may also be defined as a molecule that reduces, blocks, or inactivates a constitutive activity. An “antagonist” is a molecule that opposes the action(s) of an agonist.

The term “modulator” refers to a composition that increases or decreases the level of a target molecule or the function of a target molecule or the physical state of the target of the molecule (e.g., a target may be a cellular component (e.g., protein, ion, lipid, virus, lipid droplet, nucleic acid, nucleotide, amino acid, protein, particle, organelle, cellular compartment, microorganism, vesicle, small molecule, protein complex, protein aggregate, or macromolecule)) relative to the absence of the composition.

The terms “C-C chemokine receptor type 4” and “CCR4” refer to a protein (including homologs, isoforms, and functional fragments thereof) that is a high affinity receptor for the C-C—type chemokines (e.g., CCL2 (MCP-1), CCL4 (MIP-1), CCL5 (RANTES), CCL17 (TARC), and CCL22 (MDC)). It is referred to by a number of different names in the scientific literature, including “CC-CKR-4”, “C-C CKR-4”, “K5-5”, “CD194”, “CMIIKBR⁴”, “ChemR¹³”, “HGCN”, and “14099”. The term includes any recombinant or naturally-occurring form of CCR4 and/or variants thereof that maintain CCR4 activity (e.g., within at least 30%, 40%, 50%, 60%, 70%, 80%, 90%, 95%, or 100% activity compared to wildtype CCR4). The term includes any mutant form of CCR4 variants (e.g., frameshift mutations) thereof that maintain CCR4 activity (e.g., within at least 30%, 40%, 50%, 60%, 70%, 80%, 90%, 95%, or 100% activity compared to wildtype CCR4). In embodiments, the CCR4 protein encoded by the CCR4 gene has the amino acid sequence set forth in or corresponding to Entrez 1233, UniProt P51679, or RefSeq (protein) NP_005499.1. In embodiments, the CCR4 gene has the nucleic acid sequence set forth in RefSeq (mRNA) NM_005508. In embodiments, the amino acid sequence or nucleic acid sequence is the sequence known at the time of filing of the present application. In embodiments, the sequence corresponds to GI:5031627. In embodiments, the sequence corresponds to NP_005499.1. In embodiments, the sequence corresponds to NM_005508.4. In embodiments, the sequence corresponds to GI:48762930. In embodiments, the CCR4 is a human CCR4, such as a human cancer causing CCR4. Though frequently found on dendritic cells, macrophages, NK cells, platelets, and basophils, CCR4 is predominantly associated with T cells. It plays a role in the progression of multiple inflammation-related disorders, and, as described herein, has also been implicated in a number of other conditions. The genomic sequence of CCR4 is present on chromosome 3 (NC_000003.12), and the CCR4 gene is conserved in a number of species, including chimpanzee, Rhesus monkey, dog, cow, mouse, rat, chicken, and zebrafish. The CCR4 polypeptide comprises 360 amino acid residues (NP_005499.1), and, like other chemokine receptors, CCR4 is a G protein-coupled receptor that may be found on the surface of leukocytes (see, for example, Horuk (1994) Trends Pharm. Sci. 15:159.165).

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

“Patient”, “patient in need thereof”, “subject”, or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In embodiments, a patient is human. In embodiments, a patient in need thereof is human. In embodiments, a subject is human. In embodiments, a subject in need thereof is human.

The terms “disease” and “condition” refer to a state of being or health status of a patient or subject capable of being treated with the compounds or methods provided herein. The disease may be a cancer. The disease may be an autoimmune disease. The disease may be an inflammatory disease. The disease may be an infectious disease. In some further instances, “cancer” refers to human cancers and carcinomas, sarcomas, adenocarcinomas, lymphomas, leukemias, etc., including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including MDS, AML, ALL, ATLL and CML), or multiple myeloma.

As used herein, the term “inflammatory disease” refers to a disease or condition characterized by aberrant inflammation (e.g., an increased level of inflammation compared to a control such as a healthy person not suffering from a disease). Examples of inflammatory diseases include autoimmune diseases, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo, asthma, allergic asthma, acne vulgaris, celiac disease, chronic prostatitis, pelvic inflammatory disease, reperfusion injury, ischemia reperfusion injury, stroke, sarcoidosis, transplant rejection, interstitial cystitis, atherosclerosis, scleroderma, and atopic dermatitis. Such conditions are frequently inextricably intertwined with other diseases, disorders and conditions. A non-limiting list of inflammatory-related diseases, disorders and conditions which may, for example, be caused by inflammatory cytokines, include, arthritis, kidney failure, lupus, asthma, psoriasis, colitis, pancreatitis, allergies, fibrosis, surgical complications (e.g., where inflammatory cytokines prevent healing), anemia, and fibromyalgia. Other diseases and disorders which may be associated with chronic inflammation include Alzheimer's disease, congestive heart failure, stroke, aortic valve stenosis, arteriosclerosis, osteoporosis, Parkinson's disease, infections, inflammatory bowel disease (IBD), allergic contact dermatitis and other eczemas, systemic sclerosis, transplantation and multiple sclerosis. Some of the aforementioned diseases, disorders and conditions for which a compound (e.g., CCR4 inhibitor) described herein may be particularly efficacious (due to, for example, limitations of current therapies) are described in more detail hereafter. Examples of inflammatory diseases include traumatic brain injury, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo, asthma, allergic asthma, acne vulgaris, celiac disease, chronic prostatitis, inflammatory bowel disease, pelvic inflammatory disease, reperfusion injury, sarcoidosis, transplant rejection, interstitial cystitis, atherosclerosis, and atopic dermatitis.

As used herein, the term “cancer” refers to all types of cancer, neoplasm or malignant tumors found in mammals (e.g., humans), including leukemia, lymphoma, carcinomas and sarcomas. Exemplary cancers that may be treated with a compound or method provided herein include cancer of the thyroid, endocrine system, brain, breast, cervix, colon, head and neck, liver, kidney, lung, non-small cell lung, melanoma, mesothelioma, ovary, sarcoma, stomach, uterus medulloblastoma, colorectal cancer, or pancreatic cancer. Additional examples include Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, lymphomas, thyroid cancer, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

As used herein, the term “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin's disease. Hodgkin's disease represents approximately 15% of all diagnosed lymphomas. This is a cancer associated with Reed-Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma, Mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cutaneous T-cell lymphoma, peripheral T-cell lymphoma, anaplastic large cell lymphoma, mycosis fungoides, and precursor T-lymphoblastic lymphoma.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. “Metastatic cancer” is also called “Stage IV cancer.” Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The terms “cutaneous metastasis” or “skin metastasis” refer to secondary malignant cell growths in the skin, wherein the malignant cells originate from a primary cancer site (e.g., breast). In cutaneous metastasis, cancerous cells from a primary cancer site may migrate to the skin where they divide and cause lesions. Cutaneous metastasis may result from the migration of cancer cells from breast cancer tumors to the skin.

The term “visceral metastasis” refer to secondary malignant cell growths in the interal organs (e.g., heart, lungs, liver, pancreas, intestines) or body cavities (e.g., pleura, peritoneum), wherein the malignant cells originate from a primary cancer site (e.g., head and neck, liver, breast). In visceral metastasis, cancerous cells from a primary cancer site may migrate to the internal organs where they divide and cause lesions. Visceral metastasis may result from the migration of cancer cells from liver cancer tumors or head and neck tumors to internal organs.

As used herein, the term “autoimmune disease” refers to a disease or condition in which a subject's immune system has an aberrant immune response against a substance that does not normally elicit an immune response in a healthy subject. Examples of autoimmune diseases that may be treated with a compound, pharmaceutical composition, or method described herein include Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenic purpura (ATP), Autoimmune thyroid disease, Autoimmune urticaria, Axonal or neuronal neuropathies, Balo disease, Behcet's disease, Bullous pemphigoid, Cardiomyopathy, Castleman disease, Celiac disease, Chagas disease, Chronic fatigue syndrome, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, Lichen planus, Lichen sclerosus, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Type 1 diabetes, Ulcerative colitis, Undifferentiated connective tissue disease (UCTD), Uveitis, Vasculitis, Vesiculobullous dermatosis, Vitiligo, or Wegener's granulomatosis (i.e., Granulomatosis with Polyangiitis (GPA).

The term “drug” is used in accordance with its common meaning and refers to a substance which has a physiological effect (e.g., beneficial effect, is useful for treating a subject) when introduced into or to a subject (e.g., in or on the body of a subject or patient). A drug moiety is a radical of a drug.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

As used herein, the term “administering” is used in accordance with its plain and ordinary meaning and includes oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies (e.g., anti-cancer agent, chemotherapeutic, or treatment for a neurodegenerative disease). In embodiments, the administering does not include administration of any active agent other than the recited active agent. The compounds of the invention can be administered alone or can be co-administered to the patient. Co-administration is meant to include simultaneous or sequential administration of the compounds individually or in combination (more than one compound). Thus, the preparations can also be combined, when desired, with other active substances (e.g., to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In another embodiment, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576.1587, 1989). The compositions of the present invention can also be delivered as nanoparticles. In embodiments, the administering does not include administration of any active agent other than the recited active agent.

By “co-administer” it is meant that a composition described herein is administered at the same time, just prior to, or just after the administration of one or more additional therapies. The compounds of the invention can be administered alone or can be coadministered to the patient. Coadministration is meant to include simultaneous or sequential administration of the compounds individually or in combination (more than one compound). The compositions of the present invention can be delivered transdermally, by a topical route, or formulated as applicator sticks, solutions, suspensions, emulsions, gels, creams, ointments, pastes, jellies, paints, powders, and aerosols.

An “effective amount” is an amount sufficient for a compound to accomplish a stated purpose relative to the absence of the compound (e.g., achieve the effect for which it is administered, treat a disease, reduce enzyme activity, increase enzyme activity, reduce signaling pathway, reduce one or more symptoms of a disease or condition. An example of an “effective amount” is an amount sufficient to contribute to the treatment, prevention, or reduction of a symptom or symptoms of a disease, which could also be referred to as a “therapeutically effective amount” when referred to in this context. A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. An “activity increasing amount,” as used herein, refers to an amount of agonist required to increase the activity of an enzyme relative to the absence of the agonist. A “function increasing amount,” as used herein, refers to the amount of agonist required to increase the function of an enzyme or protein relative to the absence of the agonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). The therapeutically effective amount can be ascertained by measuring relevant physiological effects, and it can be adjusted in connection with the dosing regimen and diagnostic analysis of the subject's condition, and the like. By way of example, measurement of the serum level of a CCR4 inhibitor (or, e.g., a metabolite thereof) at a particular time post-administration may be indicative of whether a therapeutically effective amount has been administered.

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

In therapeutic use for the treatment of a disease, compound utilized in the pharmaceutical compositions of the present invention may be administered at the initial dosage of about 0.001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the compound or drug being employed. For example, dosages can be empirically determined considering the type and stage of disease (e.g., cancer or metabolic disorder) diagnosed in a particular patient. The dose administered to a patient, in the context of the present invention, should be sufficient to affect a beneficial therapeutic response in the patient over time. The size of the dose will also be determined by the existence, nature, and extent of any adverse side effects that accompany the administration of a compound in a particular patient. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired.

Dosages may be varied depending upon the requirements of the patient and the compound being employed. The dose administered to a patient, in the context of the present invention should be sufficient to affect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached.

Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

The compounds described herein can be used in combination with one another, with other active agents known to be useful in treating cancer (e.g., colon cancer), cardiovascular disease, metabolic disease, immune or inflammatory disease or disorder.

In some embodiments, co-administration includes administering one active agent within 0.5, 1, 2, 4, 6, 8, 10, 12, 16, 20, 24 hours, 2 days, 4 days, 1 week or 1 month of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another. In some embodiments, the compounds described herein may be combined with treatments for cancer (e.g., colon cancer), cardiovascular disease, metabolic disease, immune or inflammatory disease or disorder.

“Cardiovascular agent” is used in accordance with its plain ordinary meaning and refers to a composition (e.g., compound, drug, antagonist, inhibitor, modulator) used in any way to treat conditions of the heart or the circulatory or vascular system relative to a control. In some embodiments, a cardiovascular agent is an agent identified herein having utility in methods of treating cardiovascular disease or disorder. In some embodiments, a cardiovascular agent is an agent approved by the FDA or similar regulatory agency of a country other than the USA, for treating cardiovascular disease or disorder.

“Anti-inflammatory agent” is used in accordance with its plain ordinary meaning and refers to a composition (e.g., compound, drug, antagonist, inhibitor, modulator) used in any way to reduce inflammation or swelling, relative to a control (e.g, the absence of the agent), In some embodiments, an anti-inflammatory agent is an agent identified herein having utility in methods of treating an inflammatory disease or disorder. In some embodiments, an anti-inflammatory agent is an agent approved by the FDA or similar regulatory agency of a country other than the USA, for reducing swelling and inflammation.

The compounds described herein can be administered to treat an immune or inflammatory disease or disorder, a cardiovascular or metabolic disease or disorder and/or cancer. In this regard, the compounds disclosed herein may be administered either alone to treat such diseases or disorders or may be co-administered with another therapeutic agent to treat such diseases or disorders.

The compounds disclosed herein may be co-administered with a cytokine or agonist or antagonist of cytokine function, (including agents which act on cytokine signaling pathways such as modulators of the SOCS system) including alpha-, beta-, and gamma-interferons; insulin-like growth factor type I (IGF-1); interleukins (IL) including IL1 to 17, and interleukin antagonists or inhibitors such as analcinra; tumour necrosis factor alpha (TNF-.alpha.) inhibitors such as anti-TNF monoclonal antibodies (for example infliximab; adalimumab, and CDP-870) and TNF receptor antagonists including immunoglobulin molecules (such as etanercept) and low-molecular-weight agents such as pentoxyfylline.

The compounds disclosed herein may be co-administered with an anti-inflammatory agent, such as thalidomide or a derivative thereof, a retinoid, dithranol or calcipotriol, a non-steroidal anti-inflammatory agent (hereinafter NSAID) including non-selective cyclo-oxygenase COX-1/COX-2 inhibitors whether applied topically or systemically (such as piroxicam, diclofenac, propionic acids such as naproxen, flurbiprofen, fenoprofen, ketoprofen and ibuprofen, fenamates such as mefenamic acid, indomethacin, sulindac, azapropazone, pyrazolones such as phenylbutazone, salicylates such as aspirin); selective COX-2 inhibitors (such as meloxicam, celecoxib, rofecoxib, valdecoxib, lumarocoxib, parecoxib and etoricoxib); cyclo-oxygenase inhibiting nitric oxide donors (CINODs); glucocorticosteroids (whether administered by topical, oral, intramuscular, intravenous, or intra-articular routes); methotrexate; leflunomide; hydroxychloroquine; d-penicillamine; auranofin or other parenteral or oral gold preparations; analgesics; diacerein; intra-articular therapies such as hyaluronic acid derivatives; and nutritional supplements such as glucosamine.

The compounds disclosed herein may be co-administered with a calcium channel blocker, a beta-adrenoceptor blocker, an angiotensin-converting enzyme (ACE) inhibitor, an angiotensin-2 receptor antagonist; a lipid lowering agent such as a statin or a fibrate; a modulator of blood cell morphology such as pentoxyfylline; thrombolytic, or an anticoagulant such as a platelet aggregation inhibitor.

“Anti-cancer agent” and “anticancer agent” are used in accordance with their plain ordinary meaning and refers to a composition (e.g., compound, drug, antagonist, inhibitor, modulator) having antineoplastic properties or the ability to inhibit the growth or proliferation of cells. In some embodiments, an anti-cancer agent is a chemotherapeutic. In some embodiments, an anti-cancer agent is an agent identified herein having utility in methods of treating cancer. In some embodiments, an anti-cancer agent is an agent approved by the FDA or similar regulatory agency of a country other than the USA, for treating cancer. Examples of anti-cancer agents include, but are not limited to, MEK (e.g., MEK1, MEK2, or MEK1 and MEK2) inhibitors (e.g., XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g., cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g., U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec.RTM.), geldanamycin, 17-N-Allylamino-17-Demethoxygeldanamycin (17-AAG), flavopiridol, LY294002, bortezomib, trastuzumab, BAY 11-7082, PKC412, PD184352, 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin I1 (including recombinant interleukin II, or rlL.sub.2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g., Taxol.TM (i.e., paclitaxel), Taxotere.TM, compounds comprising the taxane skeleton, Erbulozole (i.e., R-55104), Dolastatin 10 (i.e., DLS-10 and NSC-376128), Mivobulin isethionate (i.e., as CI-980), Vincristine, NSC-639829, Discodermolide (i.e. as NVP-XX-A-296), ABT-751 (Abbott, i.e., E-7010), Altorhyrtins (e.g., Altorhyrtin A and Altorhyrtin C), Spongistatins (e.g., Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e., LU-103793 and NSC-D-669356), Epothilones (e.g., Epothilone A, Epothilone B, Epothilone C (i.e., desoxyepothilone A or dEpoA), Epothilone D (i.e., KOS-862, dEpoB, and desoxyepothilone B), Epothilone E, Epothilone F, Epothilone B N-oxide, Epothilone A N-oxide, 16-aza-epothilone B, 21-aminoepothilone B (i.e., BMS-310705), 21-hydroxyepothilone D (i.e., Desoxyepothilone F and dEpoF), 26-fluoroepothilone, Auristatin PE (i.e. NSC-654663), Soblidotin (i.e. TZT-1027), LS-4559-P (Pharmacia, i.e. LS-4577), LS-4578 (Pharmacia, i.e., LS-477-P), LS-4477 (Pharmacia), LS-4559 (Pharmacia), RPR-112378 (Aventis), Vincristine sulfate, DZ-3358 (Daiichi), FR-182877 (Fujisawa, i.e. WS-9885B), GS-164 (Takeda), GS-198 (Takeda), KAR-2 (Hungarian Academy of Sciences), BSF-223651 (BASF, i.e. ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (i.e., LY-355703), AC-7739 (Ajinomoto, i.e. AVE-8063A and CS-39.HCl), AC-7700 (Ajinomoto, i.e. AVE-8062, AVE-8062A, CS-39-L-Ser.HCl, and RPR-258062A), Vitilevuamide, Tubulysin A, Canadensol, Centaureidin (i.e., NSC-106969), T-138067 (Tularik, i.e., T-67, TL-138067 and TI-138067), COBRA-1 (Parker Hughes Institute, i.e. DDE-261 and WHI-261), H10 (Kansas State University), H16 (Kansas State University), Oncocidin A1 (i.e., BTO-956 and DIME), DDE-313 (Parker Hughes Institute), Fijianolide B, Laulimalide, SPA-2 (Parker Hughes Institute), SPA-1 (Parker Hughes Institute, i.e., SPIKET-P), 3-IAABU (Cytoskeleton/Mt. Sinai School of Medicine, i.e. MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, i.e. MF-191), TMPN (Arizona State University), Vanadocene acetylacetonate, T-138026 (Tularik), Monsatrol, lnanocine (i.e. NSC-698666), 3-IAABE (Cytoskeleton/Mt. Sinai School of Medicine), A-204197 (Abbott), T-607 (Tuiarik, i.e. T-900607), RPR-115781 (Aventis), Eleutherobins (such as Desmethyleleutherobin, Desaetyleleutherobin, lsoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (i.e. NSCL-96F037), D-68838 (Asta Medica), D-68836 (Asta Medica), Myoseverin B, D-43411 (Zentaris, i.e. D-81862), A-289099 (Abbott), A-318315 (Abbott), HTI-286 (i.e. SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi)), steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Gudrin (BCG), levamisole, interleukin-2, alpha-interferon, etc.), monoclonal antibodies (e.g., anti-CD20, anti-HER², anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD20 monoclonal antibody conjugated to ¹¹In, ⁹⁰Y, or ¹³¹L etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g., gefitinib (Iressa™), erlotinib (Tarceva™), cetuximab (Erbitux™), lapatinib (Tykerb™) panitumumab (Vectibix™), vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, or the like.

“Chemotherapeutic” or “chemotherapeutic agent” is used in accordance with its plain ordinary meaning and refers to a chemical composition or compound having antineoplastic properties or the ability to inhibit the growth or proliferation of cells.

Additionally, the compounds described herein can be co-administered with conventional immunotherapeutic agents including, but not limited to, immunostimulants (e.g., Bacillus Calmette-Guérin (BCG), levamisole, interleukin-2, alpha-interferon, etc.), monoclonal antibodies (e.g., anti-CD20, anti-HER², anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), and radioimmunotherapy (e.g., anti-CD20 monoclonal antibody conjugated to ¹¹¹In, ⁹⁰Y, or ¹³¹I, etc.).

The compounds disclosed herein may be co-administered with an antiproliferative/antineoplastic drug or a combination thereof, as used in medical oncology, such as an alkylating agent (for example cisplatin, carboplatin, cyclophosphamide, nitrogen mustard, melphalan, chlorambucil, busulphan or a nitrosourea); an antimetabolite (for example an antifolate such as a fluoropyrimidine like 5-fluorouracil or tegafur, raltitrexed, methotrexate, cytosine arabinoside, hydroxyurea, gemcitabine or paclitaxel); an antitumour antibiotic (for example an anthracycline such as adriamycin, bleomycin, doxorubicin, daunomycin, epirubicin, idarubicin, mitomycin-C, dactinomycin or mithramycin); an antimitotic agent (for example a vinca alkaloid such as vincristine, vinblastine, vindesine or vinorelbine, or a taxoid such as taxol or taxotere); or a topoisomerase inhibitor (for example an epipodophyllotoxin such as etoposide, teniposide, amsacrine, topotecan or a camptothecin); (ii) a cytostatic agent such as an antioestrogen (for example tamoxifen, toremifene, raloxifene, droloxifene or iodoxyfene), an oestrogen receptor down regulator (for example fulvestrant), an antiandrogen (for example bicalutamide, flutamide, nilutamide or cyproterone acetate), a LHRH antagonist or LHRH agonist (for example goserelin, leuprorelin or buserelin), a progestogen (for example megestrol acetate), an aromatase inhibitor (for example as anastrozole, letrozole, vorazole or exemestane) or an inhibitor of 5a-reductase such as finasteride; (iii) an agent which inhibits cancer cell invasion (for example a metalloproteinase inhibitor like marimastat or an inhibitor of urokinase plasminogen activator receptor function); (iv) an inhibitor of growth factor function, for example: a growth factor antibody (for example the anti-erbb2 antibody trastuzumab, or the anti-erbb1 antibody cetuximab [C225]), a farnesyl transferase inhibitor, a tyrosine kinase inhibitor or a serine/threonine kinase inhibitor, an inhibitor of the epidermal growth factor family (for example an EGFR family tyrosine kinase inhibitor such as N-(3-chloro-4-fluorophenyl)-7-methoxy-6-(3-morpholinopropoxy)quinazoli-n-4-amine (gefitinib, AZD 1839), N-(3-ethynylphenyl)-6,7-bis(2-methoxyethoxy)quinazolin-4-amine (erlotinib, OSI-774) or 6-acrylamido-N-(3-chloro-4-fluorophenyl)-7-(3-morpholinopropoxy)quinazoli-n-4-amine (CI 1033)), an inhibitor of the platelet-derived growth factor family, or an inhibitor of the hepatocyte growth factor family; (v) an antiangiogenic agent such as one which inhibits the effects of vascular endothelial growth factor (for example the anti-vascular endothelial cell growth factor antibody bevacizumab, a compound disclosed in WO 97/22596, WO 97/30035, WO 97/32856 or WO 98/13354), or a compound that works by another mechanism (for example linomide, an inhibitor of integrin.alpha.v.beta.3 function or an angiostatin); (vi) a vascular damaging agent such as combretastatin A4, or a compound disclosed in WO 99/02166, WO 00/40529, WO 00/41669, WO 01/92224, WO 02/04434 or WO 02/08213; (vii) an agent used in antisense therapy, for example one directed to one of the targets listed above, such as ISIS 2503, an anti-ras antisense; (viii) an agent used in a gene therapy approach, for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; or (ix) an agent used in an immunotherapeutic approach, for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

In embodiments, the compounds disclosed herein can be co-administered with an antibody, such as a monoclonal antibody targeting B-Lymphocytes (such as CD20 (rituximab), MRA-aIL16R and T-Lymphocytes, CTLA4-Ig, HuMax I1.15) or antibody modulating Ig function such as anti-IgE (for example omalizumab).

In embodiments, treatment of cancer includes administration of an effective amount of at least two of the following: a CCR4 inhibitor (e.g., compound described herein), an inhibitor of the PD-L1/PD-1 pathway, an inhibitor of CTLA-4, an agonistic antibody of CD137 (4.1BB). In some embodiments, the method may include the use of two or more combinations.

In embodiments, treatment of cancer includes an effective amount of at least two or more of the following: a CCR4 inhibitor (e.g., compound described herein) and any combination of agent that may be an immune modulator such as, but not limited to, those listed in Table 1, or described herein (e.g., an anti-cancer agent). These immune modulators can be depleting antibodies, neutralizing antibodies, blocking antibodies, agonistic antibodies, small molecule modulators (inhibitors or stimulators) or small molecule analogs.

TABLE 1

| Target | | |
|---|---|---|
| or Therapy | Examples of Agents | Regulatory Mechanism |
| TIM-3 | TSR-022, MGB453 | Checkpoint-receptor |
| LAG-3 | BMS-986016, IMP321 | Checkpoint-receptor |
| B7-H3 | MGA271, MGD-009 | Checkpoint-receptor |
| TIGIT | RG-6058 | Checkpoint-receptor |
| BTLA | | Checkpoint-receptor |
| CD28 | AMG 557 | Checkpoint-receptor |
| CD40 | SEA-CD40, dacetuzumab, CP- | Checkpoint-receptor |
| | 870,893, Chi Lob 7/4, | |
| | lucatumumab | |
| CD80 | galiximab | Checkpoint-receptor |
| GITR | INCAGN1876, TRX518 | Checkpoint-receptor |
| ICOS | MEDI-570 | Checkpoint-receptor |
| OX40 (CD134) | MEDI-6469, INCAGN1949, | Checkpoint-receptor |
| | huMab OX40L | |
| NKG2A | monalizumab | Checkpoint-receptor |
| TGF-beta | Galunisertib, luspatercept, YH- | Cytokines |
| | 14618, dalantercept, BG- | |
| | 00011, trabedersen, isth-0036, | |
| | ace-083 | |
| IL2 | NKTR-214, recombinant IL2, | Cytokines |
| | aldesleukin | |
| IL12 | EGEN-001, NHS-IL12 | Cytokines |
| IL7 | Recombinant IL-7 | Cytokines |
| IL15 | NIZ-985, ALT-803 | Cytokines |
| IL21 | Recombinant IL-21, anti- | Cytokines |
| | CD20.IL21 | |
| IL13 | Tralokinumab, dupilumab | |
| CSF1R | cabiralizumab | Cytokine |
| PI3K delta | INCB50465, idealisib, TGR- | Kinase |
| | 1202, AMG319 | |
| PI3K gamma | IPI-549 | Kinase |
| DNMT (DNA | Azacytidine, decitabine, | Epigenetic Regulator |
| methyl | guadecitabine | |
| transferase | | |
| inhibitor) | | |
| HDAC (histone | Vorinostat, Panobinostat, | Epigenetic Regulator |
| deacetylase) | belinostat, entinostat, | |
| | mocetinostat, givinostat, | |
| | chidamide, quisinostat, | |
| | abexinostat, chr-3996, ar-42 | |
| Brd4 | INCN54329, INCB57643, | Transcription |
| | birabresib, apabetalone, | regulator |
| | alvocidib, PLX-51107, FT- | |
| | 1101, RG-6146, AZD-8186, | |
| | CPI-0610, JQ1 | |
| HMT (histone | | Epigenetic Regulator |
| methyl | | |
| transferases) | | |
| LSD1 | INCB59872, IMG-7289, RG- | Epigenetic Regulator |
| | 6016, CC-90011, GSK- | |
| | 2879552, ORY-2001, 4SC- | |
| | 202, ORY-3001 | |
| TNFa | Recombinant TNFa, MEDI- | Cytokine |
| | 1873, FPA-154, LKZ-145 | |
| IL1 | Recombinant IL1 | Cytokine |
| IFNa | Recombinant interferon alpha- | Cytokine |
| | n1, Recombinant interferon | |
| | alpha-2b, Recombinant | |
| | interferon alpha-n3 | |
| IFNb | Recombinant IFN beta-1a | Cytokine |
| IFNg | actimmune | Cytokine |
| STING | Cyclic di-nucleotides | Signaling Molecule |
| TLR | Poly I:C, IMO-2055, TMX- | Pathogen Recognition |
| | 101, imiquimod, CpG, | Receptor |
| | MGN1703, glucopyranosyl | |
| | lipid A, CBLB502, BCG, | |
| | HILTONOL, AMPLIGEN, | |
| | MOTOLIMOD, DUK-CPG- | |
| | 001, AS15 | |
| IL10 | Recombinant IL-10 | Cytokine |
| CCR2 | CCX140, CCX872, BMS- | Chemokine |
| | 813160, CENICRIVIROC, | |
| | CNTX-6970. PF-4136309, | |
| | plozalizumab, INCB-9471, PF- | |
| | 04634817 | |
| CCR5 | Maraviroc, PRO-140, BMS- | Chemokine |
| | 813160, NIFEVIROC, OHR- | |
| | 118 | |
| CXCR4 | Ulocuplumab, plerixafor, x4p- | Chemokine |
| | 001, us1-311, ly-2510924, | |
| | APH-0812, BL-8040, | |
| | BURIXAFOR, | |
| | BALIXAFORTIDE, PTX- | |
| | 9908, GMI-1359, F-50067 | |
| LFA1 | | Adhesion Molecule |
| MICA/B | IPH-4301 | Immune Receptor |
| | | Ligand |
| VISTA | CA-170 | Checkpoint-Ligand |
| Adenosine | ISTRADEFYLLINE, | Nucleoside |
| | TOZADENANT, PBF-509, | |
| | PBF-999, CPI-444 | |
| CD39 | OREG-103. Anti-CD39 | Ecto-enzyme |
| | antibodies, | |
| CD73 | Oleclumab, PBF-1662, anti- | Ecto-Enzyme |
| | CD73 antibodies | |
| PD1 | Pembrolizumab, nivolumab, | Checkpoint-receptor |
| | INCSHR1210, CT-011, | |
| | AMP224 | |
| PD-L1 | Atezolizumab, avelumab | Checkpoint-Ligand |
| PD-L2 | | Checkpoint-Ligand |
| CTLA4 | Tremelimumab | Checkpoint-receptor |
| CD137 | Urelumab, utomilumab, BMS- | |
| | 663513, PF-05082566 | |
| AXL | BGB-324, BPI-9016M, S- | Kinase |
| | 49076 | |
| MERTK | BGB-324, BPI-9016M, S- | Kinase |
| | 49076 | |
| TYRO | BGB-324, BPI-9016M, S- | |
| | 49076 | |
| BTK | ibrutinib | Kinase |
| ITK | ibrutinib | Kinase |
| LCK | | Kinase |
| TET2 | | Enzyme |
| Arginase | Cb-1158 | Endo/ecto enzyme |
| GCN2 | | Kinase |
| B7-H4 | MDX-1140, AMP-110 | Checkpoint-receptor |
| HIF1alpha | PT2385 | Transcription Factor |
| LIGHT | | TNF Superfamily |
| (TNFSF14) | | |
| FLT3 | CDX-301, FLX925, | Kinase |
| | quizartinib, gilteritinib, | |
| | PKC412, midostaurin, | |
| | crenolanib | |
| CD158 | Lirlumab, IPH-2101 | |
| CD47 | Anti-CD47, TTI-621, NI-1701, | |
| | SRF-231, Effi-DEM, RCT- | |
| | 1938 | |
| IDO | Epacadostat, F287, | |
| | BMS983205, GDC-0919, | |
| | indoximod | |
| RORgamma | | |

In a further embodiment, the compounds described herein can be co-administered with conventional radiotherapeutic agents including, but not limited to, radionuclides such as ⁴⁷Sc, ⁶⁴Cu, ⁶⁷Cu, ⁸⁹Sr, ⁸⁶Y, ⁸⁷Y, ⁹⁰Y, ¹⁰⁵Rh, ¹¹¹Ag, ¹¹¹In, ¹¹⁷Sn, ¹⁴⁹Pm, ¹⁵³Sm, ¹⁶⁶Ho, ¹⁷⁷Lu, ¹⁸⁶Re, ¹⁸⁸Re, ²¹¹At, and ²¹²Bi, optionally conjugated to antibodies directed against tumor antigens.

In addition, a CCR4 inhibitor (e.g., compound described herein) may be combined with the therapeutic administration of immune cells, sometimes referred to as adoptive cell transfer. These cells may be cells from the patient, a genetically related or unrelated donor, they may be genetically modified (e.g., CAR-T cells, NK cells, etc), cell lines, genetically modified cell lines and live or dead versions of the above. CCR4 inhibitors may also be combined with vaccines of any kind (e.g., protein/peptide, viral, bacterial, cellular vaccines) to stimulate immune responses to cancer.

In embodiments, treatment is administration of an effective amount of a CCR4 inhibitor (e.g., compound described herein) in combination with an inhibitor of the PD-L1/PD-1 pathway, an inhibitor of CTLA-4, an agonistic antibody of CD137 (4.1BB) or in combination with another immune modulator as listed in Table 1.

In embodiments, treatment is therapeutic administration of an effective amount of a CCR4 inhibitor (e.g., compound described herein) in combination with an inhibitor of the PD-L1/PD-1 pathway, an inhibitor of CTLA-4, an agonistic antibody of CD137 (4.1BB) or in combination with another immune modulator as listed in Table 1. In embodiments, treatment starts when tumors reach a size of about 40-70 mm³.

The CCR4 inhibitors disclosed herein (e.g., compounds described herein) can be administered by any acceptable route, such oral, intraadiposal, intraarterial, intraarticular, intracranial, intradermal, intralesional, intramusculay, intranasal, intraocularal, intrapericardial, intraperitoneal, intrapleural, intraprostatical, intrarectal, intrathecal, intratracheal, intratumoral, intraumbilical, intravaginal, intravenousl, intravesicullar, intravitreal, liposomal, local, mucosal, parenteral, rectal, subconjunctival, subcutaneous, sublingual, topical, transbuccal, transdermal, vaginal, in cremes, in lipid compositions, via a catheter, via a lavage, via continuous infusion, via infusion, via inhalation, via injection, via local delivery, via localized perfusion, bathing target cells directly, or any combination thereof.

The immune modulators disclosed herein can be administered by any acceptable route, such oral, intraadiposal, intraarterial, intraarticular, intracranial, intradermal, intralesional, intramusculay, intranasal, intraocularal, intrapericardial, intraperitoneal, intrapleural, intraprostatical, intrarectal, intrathecal, intratracheal, intratumoral, intraumbilical, intravaginal, intravenousl, intravesicullar, intravitreal, liposomal, local, mucosal, parenteral, rectal, subconjunctival, subcutaneous, sublingual, topical, transbuccal, transdermal, vaginal, in cremes, in lipid compositions, via a catheter, via a lavage, via continuous infusion, via infusion, via inhalation, via injection, via local delivery, via localized perfusion, bathing target cells directly, or any combination thereof.

The CCR4 inhibitors disclosed herein (e.g., compounds described herein) may be administered once daily until study reached endpoint. The immune modulator disclosed herein may be administered at least three times but in some studies four or more times depending on the length of the study and/or the design of the study.

The methods disclosed herein may be used in combination with additional cancer therapy. In some embodiments, the distinct cancer therapy comprises surgery, radiotherapy, chemotherapy, toxin therapy, immunotherapy, cryotherapy, or gene therapy. In some embodiments, the cancer is a chemotherapy-resistant or radio-resistant cancer.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound as described herein (including embodiments and examples).

The term “modulator” refers to a composition that increases or decreases the level of a target molecule or the function of a target molecule or the physical state of the target of the molecule. In some embodiments, a CCR4 associated disease modulator is a compound that reduces the severity of one or more symptoms of a disease associated with CCR4 (e.g., cancer, inflammatory disease, autoimmune disease, or infectious disease). A CCR4 modulator is a compound that increases or decreases the activity or function or level of activity or level of function of CCR4. A modulator may act alone, or it may use a cofactor, e.g., a protein, metal ion, or small molecule. Examples of modulators include small molecule compounds and other bioorganic molecules. Numerous libraries of small molecule compounds (e.g., combinatorial libraries) are commercially available and can serve as a starting point for identifying a modulator. The skilled artisan is able to develop one or more assays (e.g., biochemical or cell-based assays) in which such compound libraries can be screened in order to identify one or more compounds having the desired properties; thereafter, the skilled medicinal chemist is able to optimize such one or more compounds by, for example, synthesizing and evaluating analogs and derivatives thereof. Synthetic and/or molecular modeling studies can also be utilized in the identification of an activator.

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule. In embodiments, the terms “modulate”, “modulation” and the like refer to the ability of a molecule (e.g., an activator or an inhibitor) to increase or decrease the function or activity of CCR4, either directly or indirectly, relative to the absence of the molecule.

The term “associated” or “associated with” or “mediated by” in the context of a substance or substance activity or function associated with a disease (e.g., a protein associated disease, a cancer associated with CCR4 activity, CCR4 associated cancer, CCR4 associated disease (e.g., cancer, inflammatory disease, autoimmune disease, or infectious disease)) or a disease mediated by a substance, substance activity, or substance function, means that the disease (e.g., cancer, inflammatory disease, autoimmune disease, or infectious disease) is caused by (in whole or in part), or a symptom of the disease is caused by (in whole or in part) the substance or substance activity or function. For example, a cancer associated with CCR4 activity or function may be a cancer that results (entirely or partially) from aberrant CCR4 function (e.g., enzyme activity, protein-protein interaction, signaling pathway) or a cancer wherein a particular symptom of the disease is caused (entirely or partially) by aberrant CCR4 activity or function. As used herein, what is described as being associated with a disease, if a causative agent, could be a target for treatment of the disease. For example, a cancer associated with CCR4 activity or function or a CCR4 associated disease (e.g., cancer, inflammatory disease, autoimmune disease, or infectious disease), may be treated with a compound described herein (e.g., CCR4 modulator or CCR4 inhibitor), in the instance where increased CCR4 activity or function (e.g., signaling pathway activity) causes the disease (e.g., cancer, inflammatory disease, autoimmune disease, or infectious disease). For example, an inflammatory disease associated with CCR4 activity or function or a CCR4 associated inflammatory disease, may be treated with a CCR4 modulator or CCR4 inhibitor, in the instance where increased CCR4 activity or function (e.g., signaling pathway activity) causes the disease.

The term “aberrant” as used herein refers to different from normal. When used to describe enzymatic activity or protein function, aberrant refers to activity or function that is greater or less than a normal control or the average of normal non-diseased control samples. Aberrant activity may refer to an amount of activity that results in a disease, wherein returning the aberrant activity to a normal or non-disease-associated amount (e.g., by administering a compound or using a method as described herein), results in reduction of the disease or one or more disease symptoms.

The term “signaling pathway” as used herein refers to a series of interactions between cellular and optionally extra-cellular components (e.g., proteins, nucleic acids, small molecules, ions, lipids) that conveys a change in one component to one or more other components, which in turn may convey a change to additional components, which is optionally propogated to other signaling pathway components. For example, binding of a CCR4 with a compound as described herein may reduce the level of a product of the CCR4 catalyzed reaction or the level of a downstream derivative of the product or binding may reduce the interactions between the CCR4 or a reaction product and downstream effectors or signaling pathway components (e.g., MAP kinase pathway), resulting in changes in cell growth, proliferation, or survival.

As used herein, the terms “CCR4 inhibitor”, “CCR4 antagonist”, “C-C chemokine receptor type 4 inhibitor”, “C-C chemokine receptor type 4 antagonist” and all other related art-accepted terms, many of which are set forth below, refer to a compound capable of detectably decreasing the expression or activity, either directly or indirectly, the CCR4 receptor in an in vitro assay, an in vivo model, and/or other means indicative of therapeutic efficacy. The terms may also refer to a compound that exhibits at least some therapeutic benefit (e.g., as determined via an objective parameter such as a serum concentration or subjective parameter, such as a subject's feeling of well-being) in a human subject.

The phrase “in a sufficient amount to effect a change” means that there is a detectable difference between a level of an indicator measured before (e.g., a baseline level or in the presence of a control) and after administration of a particular therapy. Indicators include any objective parameter (e.g., serum concentration) or subjective parameter (e.g., a subject's feeling of well-being).

The “activity” of a molecule may describe or refer to the binding of the molecule to a ligand or to a receptor; to catalytic activity; to the ability to stimulate gene expression or cell signaling, differentiation, or maturation; to antigenic activity; to the modulation of activities of other molecules; and the like. The term “proliferative activity” encompasses an activity that promotes, that is necessary for, or that is specifically associated with, for example, normal cell division, as well as cancer, tumors, dysplasia, cell transformation, metastasis, and angiogenesis.

“Substantially pure” indicates that a component makes up greater than about 50% of the total content of the composition, and typically greater than about 60% of the total content. More typically, “substantially pure” refers to compositions in which at least 75%, at least 85%, at least 90% or more of the total composition is the component of interest. In some cases, the component will make up greater than about 90%, or greater than about 95% of the total content of the composition (percentage in a weight per weight basis).

The terms “specifically binds” and “selectively binds,” when referring to a binder/target, ligand/receptor, antibody/antigen, or other binding pair, indicate a binding reaction which is determinative of the presence of the target (e.g., protein) in a heterogeneous population of components (e.g., proteins and other biologics). Thus, under designated conditions, a specified ligand binds to a particular receptor and does not bind in a significant amount to other proteins present in the sample. The antibody, or binding composition derived from the antigen-binding site of an antibody, of the contemplated method binds to its antigen, or a variant thereof, with an affinity that is at least two-fold greater, at least 10-times greater, at least 20-times greater, or at least 100-times greater than the affinity with any other target, or binding composition derived therefrom. In embodiments, the antibody will have an affinity that is greater than about 10⁹ liters/mol, as determined by, e.g., Scatchard analysis (Munsen, et al. (1980) Analyt. Biochem. 107:220-239).

The terms “nucleic acid,” “nucleic acid molecule,” “polynucleotide” and the like are used interchangeably herein to refer to a polymeric form of nucleotides of any length (e.g., either deoxyribonucleotides or ribonucleotides) or analogs thereof. Non-limiting examples of polynucleotides include linear and circular nucleic acids, messenger RNA (mRNA), complementary DNA (cDNA), recombinant polynucleotides, vectors, probes, primers and the like.

As used herein, the terms “variants” and “homologs” are used interchangeably to refer to amino acid or nucleic acid sequences that are similar to reference amino acid or nucleic acid sequences, respectively. The term encompasses naturally-occurring variants and non-naturally-occurring variants. Naturally-occurring variants include homologs (polypeptides and nucleic acids that differ in amino acid or nucleotide sequence, respectively, from one species to another), and allelic variants (polypeptides and nucleic acids that differ in amino acid or nucleotide sequence, respectively, from one individual to another within a species). Thus, variants and homologs encompass naturally occurring amino acid and nucleic acid sequences encoded thereby and their isoforms, as well as splice variants of a protein or gene. The terms also encompass nucleic acid sequences that vary in one or more bases from a naturally-occurring nucleic acid sequence but still translate into an amino acid sequence that corresponds to the naturally-occurring protein due to degeneracy of the genetic code. Non-naturally-occurring variants and homologs include polypeptides and nucleic acids that comprise a change in amino acid or nucleotide sequence, respectively, where the change in sequence is artificially introduced; for example, the change is generated in the laboratory by human intervention (“hand of man”). Therefore, non-naturally occurring variants and homologs may also refer to those that differ from the naturally occurring sequences by one or more conservative substitutions and/or tags and/or conjugates.

### II. Compounds

In an aspect is provided a compound having structural Formula (I):

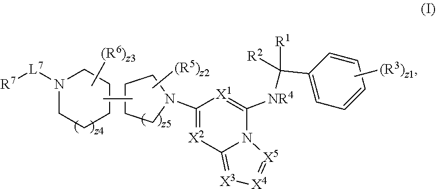

or a pharmaceutically acceptable salt thereof.

X¹ is CR⁸ or N.

X² is CR⁹ or N.

X³ is CR¹⁰ or N.

X⁴ is CR¹¹ or N.

X⁵ is CR¹² or N.

In embodiments, at least one of X¹, X², X³, X⁴, and X⁵ is N.

The symbol z1 is an integer from 0 to 5.

The symbol z2 is an integer from 0 to 4.

The symbol z3 is an integer from 0 to 11.

The symbol z4 is an integer from 0 to 2.

The symbol z5 is 1 or 2.

L⁷ is a bond, —O—, —S—, —NR^{7.2B}—, —C(O)—, —C(O)O—, —S(O)—, —S(O)₂—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

R¹ is hydrogen, halogen, —CX^{1.1}₃, —CHX^{1.1}₂, —CH₂X^{1.1}, —CN, —N₃, —SOₙ₁R^{1A}, —SOᵥ₁NR^{1B}R^{1C}, —NHNR^{1B}R^{1C}, —ONR^{1B}R^{1C}, —NHC(O)NHNR^{1B}R^{1C}, —NHC(O)NR^{1B}R^{1C}, —N(O)ₘ₁, —NR^{1B}R^{1C}, —C(O)R^{1D}, —C(O)OR^{1D}, —C(O)NR^{1B}R^{1C}, —OR^{1A}, —NR^{1B}SO₂R^{1A}, —NR^{1B}C(O)R^{1D}, —NR^{1B}C(O)OR^{1D}, —NR^{1B}OR^{1D}, —OCX^{11.3}, —OCHX^{1.1}₂, —OCH₂X^{1.1}, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R² is hydrogen, halogen, —CX^{2.1}₃, —CHX^{2.1}₂, —CH₂X^{2.1}, —CN, —N₃, —SOₙ₂R^{2A}, —SOᵥ₂NR^{2B}R^{2C}, —NHNR^{2B}R^{2C}, —ONR^{2B}R^{2C}, —NHC(O)NHNR^{2B}R^{2C}, —NHC(O)NR^{2B}R^{2C}, —N(O)ₘ₂, —NR^{2B}R^{2C}, —C(O)R^{2D}, —C(O)OR^{2D}, —C(O)NR^{2B}R^{2C}, —OR^{2A}, —NR^{2B}SO₂R^{2A}, —NR^{2B}C(O)R^{2D}, —NR^{2B}C(O)OR^{2D}, —NR^{2B}OR^{2D}, —OCX^{2.1}₃, —OCHX^{2.1}₂, —OCH₂X^{2.1}, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R³ is independently halogen, —CX^{3.1}₃, —CHX^{3.1}₂, —CH₂X^{3.1}, —CN, —N₃, —SOₙ₃R^{3A}, —SOᵥ₃NR^{3B}R^{3C}, —NHNR^{3B}R^{3C}, —ONR^{3B}R^{3C}, —NHC(O)NHNR^{3B}R^{3C}, —NHC(O)NR^{3B}R^{3C}, —N(O)ₘ₃, —NR^{3B}R^{3C}, —C(O)R^{3D}, —C(O)OR^{3D}, C(O)NR^{3B}R^{3C}, —OR^{3A}, NR^{3B}SO₂R^{3A}, —NR^{3B}C(O)R^{3D}, —NR^{3B}C(O)OR^{3D}, —NR^{3B}OR^{3D}, —OCX^{3.1}₃, —OCHX^{3.1}₂, —OCH₂X^{3.1}, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R⁴ is hydrogen, —CX^{4.1}₃, —CHX^{4.1}₂, —CH₂X^{4.1}, —SOₙ₄R^{4A}, —SOᵥ₄NR^{4B}R^{4C}, —C(O)R^{4D}, —C(O)OR^{4D}, —C(O)NR^{4B}R^{4C}, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R⁵ is independently halogen, oxo, —CX^{5.1}₃, —CHX^{5.1}₂, —CH₂X^{5.1}, —CN, —N₃, —SOₙ₅R^{5A}, —SOᵥ₅NR^{5B}R^{5C}, —NHNR^{5B}R^{5C}, —ONR^{5B}R^{5C}, —NHC(O)NHNR^{5B}R^{5C}, —NHC(O)NR^{5B}R^{5C}, —N(O)ₘ₅, —NR^{5B}R^{5C}, —C(O)R^{5D}, —C(O)OR^{5D}, —C(O)NR^{5B}R^{5C}, —OR^{5A}, —NR^{5B}SO₂R^{5A}, —NR^{5B}C(O)R^{5D}, —NR^{5B}C(O)OR^{5D}, —NR^{5B}OR^{5D}, —OCX^{5.1}₃, —OCHX^{5.1}₂, —OCH₂X^{5.1}, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R⁶ is independently halogen, oxo, —CX^{6.1}₃, —CHX^{6.1}₂, —CH₂X^{6.1}, —CN, —N₃, —SOₙ₆R^{6A}, —SOᵥ₆NR^{6B}R^{6C}, —NHNR^{6B}R^{6C}, —ONR^{6B}R^{6C}, —NHC(O)NHNR^{6B}R^{6C}, —NHC(O)NR^{6B}R^{6C}, —N(O)ₘ₆, —NR^{6B}R^{6C}, —C(O)R^{6D}, —C(O)OR^{6D}, —C(O)NR^{6B}R^{6C}, —OR^{6A}, —NR^{6B}SO₂R^{6A}, —NR^{6B}C(O)R^{6D}—NR^{6B}C(O)OR^{6D}, —NR^{6B}OR^{6D}, —OCX^{6.13}, —OCHX^{6.12}, —OCH₂X^{6.1}, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R⁷ is hydrogen, halogen, —CX^{7.1}₃, —CHX^{7.1}₂, —CH₂X^{7.1}, —CN, —N₃, —SOₙ₇R^{7A}, —SOᵥ₇NR^{7B}R^{7C}, —NHNR^{7B}R^{7C}, —ONR^{7B}R^{7C}, —NHC(O)NHNR^{7B}R^{7C}, —NHC(O)NR^{7B}R^{7C}, —N(O)ₘ₇, —NR^{7B}R^{7C}, —C(O)R^{7D}, —C(O)OR^{7D}, C(O)NR^{7B}R^{7C}, —OR^{7A}, NR^{7B}SO₂R^{7A}, —NR^{7B}C(O)R^{7D}, —NR^{7B}C(O)OR^{7D}, —NR^{7B}OR^{7D}, —OCX^{7.1}₃, —OCHX^{7.1}₂, —OCH₂X^{7.1}, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R⁸ is hydrogen, halogen, —CX^{8.1}₃, —CHX^{8.1}₂, —CH₂X^{8.1}, —CN, —N₃, —SOᵥ₈R^{8A}, —SOᵥ₈NR^{8B}R^{8C}, —NHNR^{8B}R^{8C}, —ONR^{8B}R^{8C}, —NHC(O)NHNR^{8B}R^{8C}, —NHC(O)NR^{8B}R^{8C}, —N(O)ₘ₈, —NR^{8B}R^{8C}, —C(O)R^{8D}, —C(O)OR^{8D}, —C(O)NR^{8B}R^{8C}, —OR^{8A}, —NR^{8B}SO₂R^{8A}, —NR^{8B}C(O)R^{8D}, —NR^{8B}C(O)OR^{8D}, —NR^{8B}OR^{8D}, —OCX^{8.1}₃, —OCHX^{8.1}₂, —OCH₂X^{8.1}, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R⁹ is hydrogen, halogen, —CX^{9.1}₃, —CHX^{9.1}₂, —CH₂X^{9.1}, —CN, —N₃, —SOₙ₉R^{9A}, —SOᵥ₉NR^{9B}R^{9C}, —NHNR^{9B}R^{9C}, —ONR^{9B}R^{9C}, —NHC(O)NHNR^{9B}R^{9C}, —NHC(O)NR^{9B}R^{9C}, —N(O)ₘ₉, —NR^{9B}R^{9C}, —C(O)R^{9D}, —C(O)OR^{9D}, C(O)NR^{9B}R^{9C}, —OR^{9A}, NR^{9B}SO₂R^{9A}, —NR^{9B}C(O)R^{9D}, —NR^{9B}C(O)OR^{9D}, —NR^{9B}OR^{9D}, —OCX^{9.1}₃, —OCHX^{9.1}₂, —OCH₂X^{9.1}, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); or when X² is CR⁹ and X³ is CR¹⁰, then R⁹ and R¹⁰ may optionally be joined to form a substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R¹⁰ is hydrogen, halogen, —CX^{10.1}₃, —CHX^{10.1}₂, —CH₂X^{10.1}, —CN, —N₃, —SOₙ₁₀R^{10A}, —SOᵥ₁₀NR^{10B}R^{10C}, —NHNR^{10B}R^{10C}, —ONR^{10B}R^{10C}, —NHC(O)NHNR^{10B}R^{10C}, —NHC(O)NR^{10B}R^{10C}, —N(O)ₘ₁₀, —NR^{10B}R^{10C}, —C(O)R^{10D}, —C(O)OR^{10D}, —C(O)NR^{10B}R^{10C}, —OR^{10A}, —NR^{10B}O₂R^{10A}, —NR¹⁰BC(O)R^{10D}, —NR¹⁰BC(O)OR^{10D}, —NR¹⁰BOR^{10D}, —OCX^{10B}, —OCHX^{10.1}₂, —OCH₂X^{10.1}, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); or when X³ is CR¹⁰ and X⁴ is CR¹¹, then R¹⁰ and R¹¹ may optionally be joined to form a substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R¹¹ is hydrogen, halogen, —CX^{11.1}₃, —CHX^{11.1}₂, —CH₂X^{11.1}, —CN, —N₃, —SOₙ₁₁R^{11A}, —SOᵥ₁₁NR^{11B}R^{11C}, —NHNR^{11B}R^{11C}, —ONR^{11B}R^{11C}, —NHC(O)NHNR^{11B}R^{11C}, —NHC(O)NR^{11B}R^{11C}, —N(O)ₘ₁₁, —NR^{11B}R^{11C}, —C(O)R^{11D}, —C(O)OR^{11D}, —C(O)NR^{11B}R^{11C}, —OR^{11A}, —NR^{11B}SO₂R^{11A}, —NR^{11B}C(O)R^{11D}, —NR^{11B}C(O)OR^{11D}, —NR^{11B}OR^{11D}, —OCX^{11.1}₃, —OCHX^{11.1}₂, —OCH₂X^{11.1}—, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); or when X⁴ is CR¹¹ and X⁵ is CR¹², then R¹¹ and R¹² may optionally be joined to form a substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R¹² is hydrogen, halogen, —CX^{12.1}₃, —CHX^{12.1}₂, —CH₂X^{12.1}, —CN, —N₃, —SOₙ₁₂R^{12A}, —SOᵥ₁₂NR^{12B}R^{12C}, —NHNR^{12B}R^{12C}, —ONR^{12B}R^{12C}, —NHC(O)NHNR^{12B}R^{12C}, —NHC(O)NR^{12B}R^{12C}, —N(O)ₘ₁₂, —NR^{12B}R^{12C}, —C(O)R^{12D}, —C(O)OR^{12D}, C(O)NR^{12B}R^{12C}, —OR^{12A}—NR¹²BSO₂R^{12A}, —NR¹²BC(O)R^{12D}, —NR¹²BC(O)OR^{12D}, —NR¹²BOR^{12D}, —OCX^{12.1}₃, —OCHX^{12.1}₂, —OCH₂X^{12.1}, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R^{1A}, R^{1B}, R^{1C}, R^{1D}, R^{2A}, R^{2B}, R^{2C}, R^{2D}, R^{3A}, R^{3B}, R^{3C}, R^{3D}, R^{4A}, R^{4B}, R^{4C}, R^{4D}, R^{5A}, R^{5B}, R^{5C}, R^{5D}, R^{6A}, R^{6B}, R^{6C}, R^{6D}, R^{7A}, R^{7B}, R^{7C}, R^{7D}, R^{7B}, R^{8A}, R^{8B}, R^{8C}, R^{8D}, R^{9A}, R^{9B}, R^{9C}, R^{9D}, R^{10A}, R^{10B}, R^{10C}, R^{10D}, R^{11A}, R^{11B}, R^{11C}, R^{11D}, R^{12A}, R^{12.3}, R^{12C}, and R^{12D} are independently hydrogen, halogen, —CF₃, —CCl₃, —CBr₃, —CI₃, —COOH, —CONH₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{1B} and R^{1C} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{2B} and R^{2C}substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{3B} and R^{3C} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{4B} and R^{4C} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{5B} and R^{5C} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{6B} and R^{6C} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{7B} and R^{7C} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{8B} and R^{8C} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{9B} and R^{9C} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{10B} and R^{10C} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{11B} and R^{11C} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{12B}and R^{12C} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered).

X^{1.1}, X^{2.1}, X^{3.1}, X^{4.1}, X^{5.1}, X^{6.1}, X^{7.1}, X^{8.1}, X^{9.1}, X^{10.1}, X^{11.1}, and X^{12.1} are independently —Cl, —Br, —I, or —F.

The symbols n1, n2, n3, n4, n5, n6, n7, n8, n9, n10, n11, and n12 are independently an integer from 0 to 4.

The symbols m1, m2, m3, m5, m6, m7, m8, m9, m10, m11, m12, v1, v2, v3, v4, v5, v6, v7, v8, v9, v10, v11, and v12 are independently 1 or 2.

In embodiments, X¹ is CR⁸. In embodiments, X¹ is CH. In embodiments, X¹ is N.

In embodiments, X² is CR⁹. In embodiments, X² is CH. In embodiments, X² is N.

In embodiments, X³ is CR¹⁰. In embodiments, X³ is CH. In embodiments, X³ is N.

In embodiments, X⁴ is CR¹¹. In embodiments, X⁴ is CH. In embodiments, X⁴ is N.

In embodiments, X⁵ is CR¹². In embodiments, X⁵ is CH. In embodiments, X⁵ is N.

In embodiments, at least one of X¹, X², X³, X⁴, and X⁵ is N. In embodiments, X¹ is CR⁸, X² is N, X³ is N, X⁴ is CR¹¹, and X⁵ is N. In embodiments, X¹ is N, X² is CR⁹, X³ is N, X⁴ is CR¹¹, and X⁵ is N.

In embodiments, z1 is 0. In embodiments, z1 is 1. In embodiments, z1 is 2. In embodiments, z1 is 3. In embodiments, z1 is 4. In embodiments, z1 is 5.

In embodiments, z2 is 0. In embodiments, z2 isl. In embodiments, z2 is 2. In embodiments, z2 is 3. In embodiments, z2 is 4.

In embodiments, z3 is 0. In embodiments, z3 is 1. In embodiments, z3 is 2. In embodiments, z3 is 3. In embodiments, z3 is 4. In embodiments, z3 is 5. In embodiments, z3 is 6. In embodiments, z3 is 7. In embodiments, z3 is 8. In embodiments, z3 is 9. In embodiments, z3 is 10. In embodiments, z3 is 11.

In embodiments, z4 is 0. In embodiments, z4 is 1. In embodiments, z4 is 2.

In embodiments, z5 is 1. In embodiments, z5 is 2.

In embodiments, the compound has the formula:

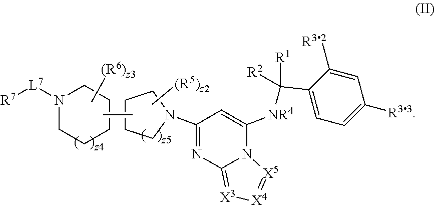

X³, X⁴, X⁵, z2, z3, z4, z5, L⁷, R¹, R², R⁴, R⁵, R⁶, and R⁷ are as described herein, including in embodiments.

R^{3.2} is hydrogen, halogen, —CX^{3.2}₃, —CHX^{3.2}₂, —CH₂X^{3.2}, —CN, —N₃, —SOₙ₃0.2R^{3.2A}, —SOᵥ₃0.2NR^{3.2B}R^{3.2C}, —NHNR^{3.2B}R^{3.2C}, —ONR^{3.2B}R^{3.2C}, —NHC(O)NHNR^{3.2B}R^{3.2C}, —NHC(O)NR^{3.2B}R^{3.2C}, —N(O)_{m3.2}, —NR^{3.2}R^{3.2C}, C(O)R^{3.2D}, —C(O)OR^{3.2D}—C(O)NR^{3.2B}R^{3.2C}, —OR^{3.2A}, —NR^{3.2B}SO₂R^{3.2A}—NR^{3.2C}(O)R^{3.2D}—NR^{3.2B}C(O)OR^{3.2D}—NR^{3.2B}OR^{3.2D}, —OCX^{3.2}₃, —OCHX^{3.2}₂, —OCH₂X^{3.2}, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R^{3.3} is hydrogen, halogen, —CX^{3.3}₃, —CHX^{3.3}₂, —CH₂X^{3.3}, —CN, —N₃, —SO_{n3.3}R^{3.3A}, —SO_{v3.3}NR^{3.3B}R^{3.3C}, —NHNR^{3.3B}R^{3.3C}, —ONR^{3.3B}R^{3.3C}, —NHC(O)NHNR^{3.3B}R^{3.3C}, —NHC(O)NR^{3.3B}R^{3.3C}, —N(O)_{m3.3}, —NR^{3.3B}R^{3.3C}, —C(O)R^{3.3D}, —C(O)OR^{3.3D}—C(O)NR^{3.3B}R^{3.3C}, —OR^{3.3A}, —NR^{3.3B}SO₂R^{3.3A}, —NR^{3.3B}C(O)R^{3.3D}, —NR^{3.3B}C(O)OR^{3.3D}, —NR^{3.3B}OR^{3.3D}, —OCX^{3.3B}, —OCHX^{3.3}₂, —OCH₂X^{3.3}, substituted or unsubstituted alkyl (e.g., C₁-C₈, C₁-C₆, C₁-C₄, or C₁-C₂) substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R^{3.2A}, R^{3.2B}, R^{3.2C}, R^{3.2D}, R^{3.3A}, R^{3.3B}, R^{3.3C}, and R^{3.3D} are independently hydrogen, halogen, —CF₃, —CCl₃, —CBr₃, —CI₃, —COOH, —CONH₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{3.2B}and R^{3.2c} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{3.3B} and R^{3.3c} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered).

The symbols n3.2 and n3.3 are independently an integer from 0 to 4.

The symbols m3.2, m3.3, v3.2, and v3.3 are independently 1 or 2.

X^{3.2} and X^{3.3} are independently —Cl, —Br, —I, or —F.

In embodiments, the compound has the formula:

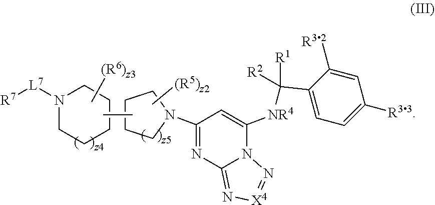

X⁴, z2, z3, z4, z5, L⁷, R¹, R², R^{3.2}, R^{3.3}, R⁴, R⁵, R⁶, and R⁷ are as described herein, including in embodiments. In embodiments, X⁴ is CR¹¹; and R¹¹ is hydrogen, —CX^{11.1}₃, —CHX^{11.1}₂, —CH₂X^{11.1}, —CN, or substituted or unsubstituted C₁-C₄ alkyl. In embodiments, R¹¹ is hydrogen.

In embodiments, the compound has the formula:

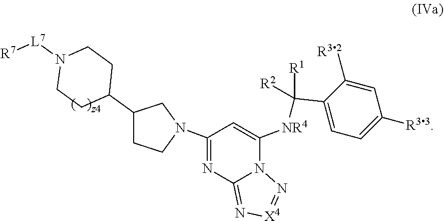

X⁴, z4, L⁷, R¹, R², R^{3.2}, R^{3.3}, R⁴, and R⁷ are as described herein, including in embodiments.

In embodiments, the compound has the formula:

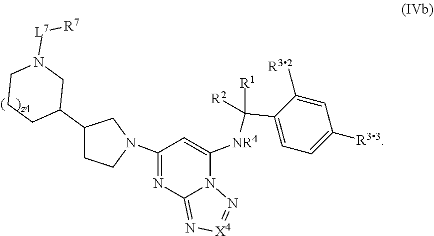

X⁴, z4, L⁷, R¹, R², R^{3.2}, R^{3.3}, R⁴, and R⁷ are as described herein, including in embodiments.

In embodiments, the compound has the formula:

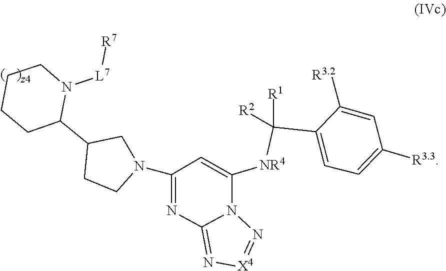

X⁴, z4, L⁷, R¹, R², R^{3.2}, R^{3.3}, R⁴, and R⁷ are as described herein, including in embodiments.

In embodiments, the compound has the formula:

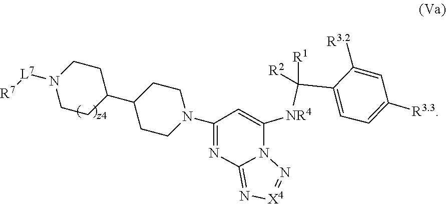

X⁴, z4, L⁷, R¹, R², R^{3.2}, R^{3.3}, R⁴, and R⁷ are as described herein, including in embodiments.

In embodiments, the compound has the formula:

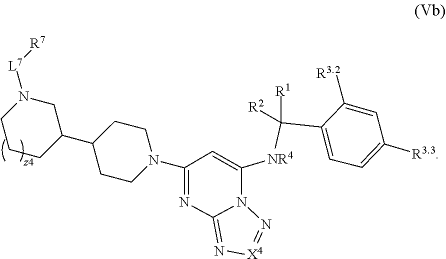

X⁴, z4, L⁷, R¹, R², R^{3.2}, R^{3.3}, R⁴, and R⁷ are as described herein, including in embodiments.

In embodiments, the compound has the formula:

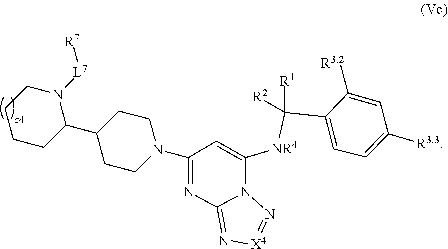

X⁴, z4, L⁷, R¹, R², R^{3.2}, R^{3.3}, R⁴, and R⁷ are as described herein, including in embodiments.

In embodiments, the compound has the formula:

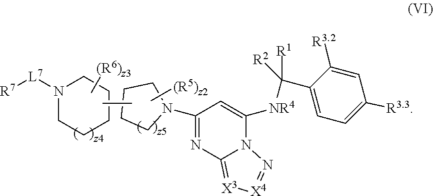

X³, X⁴, z2, z3, z4, z5, L⁷, R¹, R², R^{3.2}, R^{3.3}, R⁴, R⁵, R⁶, and R⁷ are as described herein, including in embodiments. In embodiments, X³ is CR¹⁰; X⁴ is CR¹¹; R¹⁰ is hydrogen, —CX^{10.1}₃, —CHX^{10.1}₂—CH₂X^{10.1}, —CN, or substituted or unsubstituted C₁-C₄ alkyl; and R¹¹ is hydrogen, —CX^{11.1}₃, —CHX^{11.1}₂, —CH₂X^{11.1}, —CN, or substituted or unsubstituted C₁-C₄ alkyl. In embodiments, R¹⁰ and R¹¹ are hydrogen.

In embodiments, the compound has the formula:

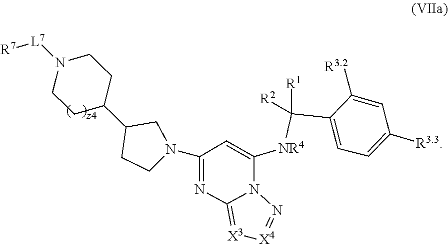

X³, X⁴, z4, L⁷, R¹, R², R^{3.2}, R^{3.3}, R⁴, and R⁷ are as described herein, including in embodiments.

In embodiments, the compound has the formula:

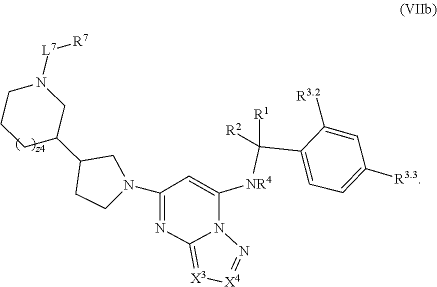

X³, X⁴, z4, L⁷, R¹, R², R^{3.2}, R^{3.3}, R⁴, and R⁷ are as described herein, including in embodiments.

In embodiments, the compound has the formula:

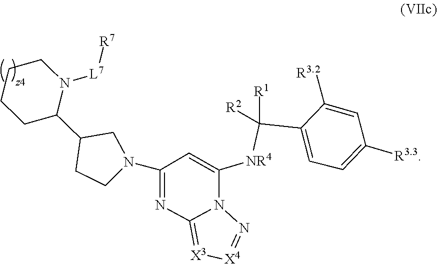

X³, X⁴, z4, L⁷, R¹, R², R^{3.2}, R^{3.3}, R⁴, and R⁷ are as described herein, including in embodiments.

In embodiments, the compound has the formula:

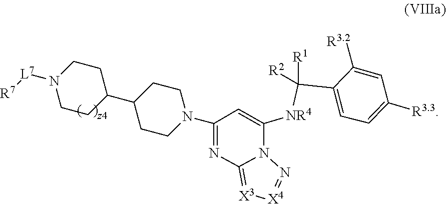

X³, X⁴, z4, L⁷, R¹, R², R^{3.2}, R^{3.3}, R⁴, and R⁷ are as described herein, including in embodiments.

In embodiments, the compound has the formula:

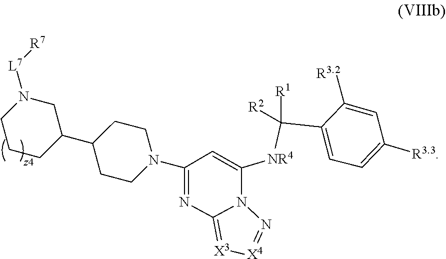

X³, X⁴, z4, L⁷, R¹, R², R^{3.2}, R^{3.3}, R⁴, and R⁷ are as described herein, including in embodiments.

In embodiments, the compound has the formula:

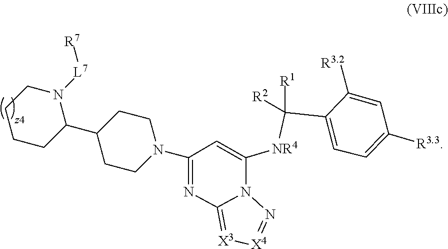

X³, X⁴, z4, L⁷, R¹, R², R^{3.2}, R^{3.3}, R⁴, and R⁷ are as described herein, including in embodiments.

In embodiments, a substituted R¹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹ is substituted with a plurality of groups selected from substituent groups, size-limited substituent groups, and lower substituent groups; each substituent group, size-limited substituent group, and/or lower substituent group may optionally be different. In embodiments, when R¹ is substituted, it is substituted with at least one substituent group. In embodiments, when R¹ is substituted, it is substituted with at least one size-limited substituent group. In embodiments, when R¹ is substituted, it is substituted with at least one lower substituent group.

In embodiments, R¹ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R¹ is hydrogen. In embodiments, R¹ is halogen. In embodiments, R¹ is —F. In embodiments, R¹ is —Cl. In embodiments, R¹ is —Br. In embodiments, R¹ is —I. In embodiments, R¹ is unsubstituted C₁-C₄ alkyl. In embodiments, R¹ is unsubstituted methyl. In embodiments, R¹ is unsubstituted ethyl. In embodiments, R¹ is unsubstituted propyl. In embodiments, R¹ is unsubstituted n-propyl. In embodiments, R¹ is unsubstituted isopropyl. In embodiments, R¹ is unsubstituted butyl. In embodiments, R¹ is unsubstituted n-butyl. In embodiments, R¹ is unsubstituted isobutyl. In embodiments, R¹ is unsubstituted tert-butyl.

In embodiments, R¹ is hydrogen, substituted or unsubstituted alkyl, or substituted or unsubstituted heteroalkyl. In embodiments, R¹ is hydrogen or substituted or unsubstituted C₁-C₄ alkyl.

In embodiments, a substituted R^{1A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1A} is substituted with a plurality of groups selected from substituent groups, size-limited substituent groups, and lower substituent groups; each substituent group, size-limited substituent group, and/or lower substituent group may optionally be different. In embodiments, when R^{1A} is substituted, it is substituted with at least one substituent group. In embodiments, when R^{1A} is substituted, it is substituted with at least one size-limited substituent group. In embodiments, when R^{1A} is substituted, it is substituted with at least one lower substituent group.

In embodiments, a substituted R^{1B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1B} is substituted with a plurality of groups selected from substituent groups, size-limited substituent groups, and lower substituent groups; each substituent group, size-limited substituent group, and/or lower substituent group may optionally be different. In embodiments, when R^{1B} is substituted, it is substituted with at least one substituent group. In embodiments, when R^{1B} is substituted, it is substituted with at least one size-limited substituent group. In embodiments, when R^{1B} is substituted, it is substituted with at least one lower substituent group.

In embodiments, a substituted R^{1C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1C} is substituted with a plurality of groups selected from substituent groups, size-limited substituent groups, and lower substituent groups; each substituent group, size-limited substituent group, and/or lower substituent group may optionally be different. In embodiments, when R^{1C} is substituted, it is substituted with at least one substituent group. In embodiments, when R^{1C} is substituted, it is substituted with at least one size-limited substituent group. In embodiments, when R^{1C} is substituted, it is substituted with at least one lower substituent group.

In embodiments, a substituted ring formed when R^{1B} and R^{1C} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{1B} and R^{1C} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{1B} and R^{1C} substituents bonded to the same nitrogen atom are joined is substituted, it is substituted with at least one substituent group. In embodiments, when the substituted ring formed when R^{1B} and R^{1C} substituents bonded to the same nitrogen atom are joined is substituted, it is substituted with at least one size-limited substituent group. In embodiments, when the substituted ring formed when R^{1B} and R^{1C} substituents bonded to the same nitrogen atom are joined is substituted, it is substituted with at least one lower substituent group.

In embodiments, a substituted R^{1D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1D} is substituted with a plurality of groups selected from substituent groups, size-limited substituent groups, and lower substituent groups; each substituent group, size-limited substituent group, and/or lower substituent group may optionally be different. In embodiments, when R^{1D} is substituted, it is substituted with at least one substituent group. In embodiments, when R^{1D} is substituted, it is substituted with at least one size-limited substituent group. In embodiments, when R^{1D} is substituted, it is substituted with at least one lower substituent group.

In embodiments, R^{1A} is hydrogen. In embodiments, R^{1A} is unsubstituted C₁-C₄ alkyl. In embodiments, R^{1A} is unsubstituted methyl. In embodiments, R^{1A} is unsubstituted ethyl. In embodiments, R^{1A} is unsubstituted propyl. In embodiments, R^{1A} is unsubstituted n-propyl. In embodiments, R^{1A} is unsubstituted isopropyl. In embodiments, R^{1A} is unsubstituted butyl. In embodiments, R^{1A} is unsubstituted n-butyl. In embodiments, R^{1A} is unsubstituted isobutyl. In embodiments, R^{1A} is unsubstituted tert-butyl.

In embodiments, R^{1B} is hydrogen. In embodiments, R^{1B} is unsubstituted C₁-C₄ alkyl. In embodiments, R^{1B} is unsubstituted methyl. In embodiments, R^{1B} is unsubstituted ethyl. In embodiments, R^{1B} is unsubstituted propyl. In embodiments, R^{1B} is unsubstituted n-propyl. In embodiments, R^{1B} is unsubstituted isopropyl. In embodiments, R^{1B} is unsubstituted butyl. In embodiments, R^{1B} is unsubstituted n-butyl. In embodiments, R^{1B} is unsubstituted isobutyl. In embodiments, R^{1B} is unsubstituted tert-butyl.

In embodiments, R^{1C} is hydrogen. In embodiments, R^{1C} is unsubstituted C₁-C₄ alkyl. In embodiments, R^{1C} is unsubstituted methyl. In embodiments, R^{1C} is unsubstituted ethyl. In embodiments, R^{1C} is unsubstituted propyl. In embodiments, R^{1C} is unsubstituted n-propyl. In embodiments, R^{1C} is unsubstituted isopropyl. In embodiments, R^{1C} is unsubstituted butyl. In embodiments, R^{1C} is unsubstituted n-butyl. In embodiments, R^{1C} is unsubstituted isobutyl. In embodiments, R^{1C} is unsubstituted tert-butyl.

In embodiments, R^{1D} is hydrogen. In embodiments, R^{1D} is unsubstituted C₁-C₄ alkyl. In embodiments, R^{1D} is unsubstituted methyl. In embodiments, R^{1D} is unsubstituted ethyl. In embodiments, R^{1D} is unsubstituted propyl. In embodiments, R^{1D} is unsubstituted n-propyl. In embodiments, R^{1D} is unsubstituted isopropyl. In embodiments, R^{1D} is unsubstituted butyl. In embodiments, R^{1D} is unsubstituted n-butyl. In embodiments, R^{1D} is unsubstituted isobutyl. In embodiments, R^{1D} is unsubstituted tert-butyl.

In embodiments, a substituted R² (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R² is substituted with a plurality of groups selected from substituent groups, size-limited substituent groups, and lower substituent groups; each substituent group, size-limited substituent group, and/or lower substituent group may optionally be different. In embodiments, when R² is substituted, it is substituted with at least one substituent group. In embodiments, when R² is substituted, it is substituted with at least one size-limited substituent group. In embodiments, when R² is substituted, it is substituted with at least one lower substituent group.

In embodiments, R² is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R² is hydrogen. In embodiments, R² is halogen. In embodiments, R² is —F. In embodiments, R² is —Cl. In embodiments, R² is —Br. In embodiments, R² is —I. In embodiments, R² is unsubstituted C₁-C₄ alkyl. In embodiments, R² is unsubstituted methyl. In embodiments, R² is unsubstituted ethyl. In embodiments, R² is unsubstituted propyl. In embodiments, R² is unsubstituted n-propyl. In embodiments, R² is unsubstituted isopropyl. In embodiments, R² is unsubstituted butyl. In embodiments, R² is unsubstituted n-butyl. In embodiments, R² is unsubstituted isobutyl. In embodiments, R² is unsubstituted tert-butyl.

In embodiments, R² is hydrogen, substituted or unsubstituted alkyl, or substituted or unsubstituted heteroalkyl. In embodiments, R² is hydrogen or substituted or unsubstituted C₁-C₄ alkyl.

In embodiments, a substituted R^{2A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2A} is substituted with a plurality of groups selected from substituent groups, size-limited substituent groups, and lower substituent groups; each substituent group, size-limited substituent group, and/or lower substituent group may optionally be different. In embodiments, when R^{2A} is substituted, it is substituted with at least one substituent group. In embodiments, when R^{2A} is substituted, it is substituted with at least one size-limited substituent group. In embodiments, when R^{2A} is substituted, it is substituted with at least one lower substituent group.

In embodiments, a substituted R²¹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R²¹ is substituted with a plurality of groups selected from substituent groups, size-limited substituent groups, and lower substituent groups; each substituent group, size-limited substituent group, and/or lower substituent group may optionally be different. In embodiments, when R²¹ is substituted, it is substituted with at least one substituent group. In embodiments, when R²¹ is substituted, it is substituted with at least one size-limited substituent group. In embodiments, when R²¹ is substituted, it is substituted with at least one lower substituent group.

In embodiments, a substituted R^{2C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2C} is substituted with a plurality of groups selected from substituent groups, size-limited substituent groups, and lower substituent groups; each substituent group, size-limited substituent group, and/or lower substituent group may optionally be different. In embodiments, when R^{2C} is substituted, it is substituted with at least one substituent group. In embodiments, when R^{2C} is substituted, it is substituted with at least one size-limited substituent group. In embodiments, when R^{2C} is substituted, it is substituted with at least one lower substituent group.

In embodiments, a substituted ring formed when R^{2B} and R^{2C} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{2B} and R^{2C} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{2B} and R^{2C} substituents bonded to the same nitrogen atom are joined is substituted, it is substituted with at least one substituent group. In embodiments, when the substituted ring formed when R^{2B} and R^{2C}substituents bonded to the same nitrogen atom are joined is substituted, it is substituted with at least one size-limited substituent group. In embodiments, when the substituted ring formed when R^{2B} and R^{2C} substituents bonded to the same nitrogen atom are joined is substituted, it is substituted with at least one lower substituent group.

In embodiments, a substituted R^{2D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2D} is substituted with a plurality of groups selected from substituent groups, size-limited substituent groups, and lower substituent groups; each substituent group, size-limited substituent group, and/or lower substituent group may optionally be different. In embodiments, when R^{2D} is substituted, it is substituted with at least one substituent group. In embodiments, when R^{2D} is substituted, it is substituted with at least one size-limited substituent group. In embodiments, when R^{2D} is substituted, it is substituted with at least one lower substituent group.

In embodiments, R^{2A} is hydrogen. In embodiments, R^{2A} is unsubstituted C₁-C₄ alkyl. In embodiments, R^{2A} is unsubstituted methyl. In embodiments, R^{2A} is unsubstituted ethyl. In embodiments, R^{2A} is unsubstituted propyl. In embodiments, R^{2A} is unsubstituted n-propyl. In embodiments, R^{2A} is unsubstituted isopropyl. In embodiments, R^{2A} is unsubstituted butyl. In embodiments, R^{2A} is unsubstituted n-butyl. In embodiments, R^{2A} is unsubstituted isobutyl. In embodiments, R^{2A} is unsubstituted tert-butyl.

In embodiments, R^{2B} is hydrogen. In embodiments, R^{2B} is unsubstituted C₁-C₄ alkyl. In embodiments, R^{2B} is unsubstituted methyl. In embodiments, R^{2B} is unsubstituted ethyl. In embodiments, R^{2B} is unsubstituted propyl. In embodiments, R^{2B} is unsubstituted n-propyl. In embodiments, R^{2B} is unsubstituted isopropyl. In embodiments, R^{2B} is unsubstituted butyl. In embodiments, R^{2B} is unsubstituted n-butyl. In embodiments, R^{2B} is unsubstituted isobutyl. In embodiments, R^{2B} is unsubstituted tert-butyl.

In embodiments, R^{2C} is hydrogen. In embodiments, R^{2C} is unsubstituted C₁-C₄ alkyl. In embodiments, R^{2C} is unsubstituted methyl. In embodiments, R^{2C} is unsubstituted ethyl. In embodiments, R^{2C} is unsubstituted propyl. In embodiments, R^{2C} is unsubstituted n-propyl. In embodiments, R^{2C} is unsubstituted isopropyl. In embodiments, R^{2C} is unsubstituted butyl. In embodiments, R^{2C} is unsubstituted n-butyl. In embodiments, R^{2C} is unsubstituted isobutyl. In embodiments, R^{2C} is unsubstituted tert-butyl.

In embodiments, R^{2D} is hydrogen. In embodiments, R^{2D} is unsubstituted C₁-C₄ alkyl. In embodiments, R^{2D} is unsubstituted methyl. In embodiments, R^{2D} is unsubstituted ethyl. In embodiments, R^{2D} is unsubstituted propyl. In embodiments, R^{2D} is unsubstituted n-propyl. In embodiments, R^{2D} is unsubstituted isopropyl. In embodiments, R^{2D} is unsubstituted butyl. In embodiments, R^{2D} is unsubstituted n-butyl. In embodiments, R^{2D} is unsubstituted isobutyl. In embodiments, R^{2D} is unsubstituted tert-butyl.

In embodiments, a substituted R³ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R³ is substituted with a plurality of groups selected from substituent groups, size-limited substituent groups, and lower substituent groups; each substituent group, size-limited substituent group, and/or lower substituent group may optionally be different. In embodiments, when R³ is substituted, it is substituted with at least one substituent group. In embodiments, when R³ is substituted, it is substituted with at least one size-limited substituent group. In embodiments, when R³ is substituted, it is substituted with at least one lower substituent group.

In embodiments, R³ is independently halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R³ is independently halogen. In embodiments, R³ is independently —F. In embodiments, R³ is independently —Cl. In embodiments, R³ is independently —Br. In embodiments, R³ is independently —I. In embodiments, R³ is independently —CCl₃. In embodiments, R³ is independently —CBr₃. In embodiments, R³ is independently —CF₃. In embodiments, R³ is independently —CI₃. In embodiments, R³ is independently —CH₂C₁. In embodiments, R³ is independently —CH₂Br. In embodiments, R³ is independently —CH₂F. In embodiments, R³ is independently —CH₂I. In embodiments, R³ is independently —CHCl₂. In embodiments, R³ is independently —CHBr₂. In embodiments, R³ is independently —CHF₂. In embodiments, R³ is independently —CHI₂. In embodiments, R³ is independently —CN. In embodiments, R³ is independently —OH. In embodiments, R³ is independently —NH₂. In embodiments, R³ is independently —COOH. In embodiments, R³ is independently —CONH₂. In embodiments, R³ is independently —NO₂. In embodiments, R³ is independently —SH. In embodiments, R³ is independently —SO₃H. In embodiments, R³ is independently —OSO₃H. In embodiments, R³ is independently —SO₂NH₂. In embodiments, R³ is independently —NHNH₂. In embodiments, R³ is independently —ONH₂. In embodiments, R³ is independently —NHC(O)NH₂. In embodiments, R³ is independently —NHSO₂H. In embodiments, R³ is independently —NHC(O)H. In embodiments, R³ is independently —NHC(O)OH. In embodiments, R³ is independently —NHOH. In embodiments, R³ is independently —OCCl₃. In embodiments, R³ is independently —OCBr₃. In embodiments, R³ is independently —OCF₃. In embodiments, R³ is independently —OCI₃. In embodiments, R³ is independently —OCH₂Cl. In embodiments, R³ is independently —OCH₂Br. In embodiments, R³ is independently —OCH₂F. In embodiments, R³ is independently —OCH₂I. In embodiments, R³ is independently —OCHCl₂. In embodiments, R³ is independently —OCHBr₂. In embodiments, R³ is independently —OCHF₂. In embodiments, R³ is independently —OCHI₂. In embodiments, R³ is independently —N3. In embodiments, R³ is independently unsubstituted C₁-C₄ alkyl. In embodiments, R³ is independently unsubstituted methyl. In embodiments, R³ is independently unsubstituted ethyl. In embodiments, R³ is independently unsubstituted propyl. In embodiments, R³ is independently unsubstituted n-propyl. In embodiments, R³ is independently unsubstituted isopropyl. In embodiments, R³ is independently unsubstituted butyl. In embodiments, R³ is independently unsubstituted n-butyl. In embodiments, R³ is independently unsubstituted isobutyl. In embodiments, R³ is independently unsubstituted tert-butyl. In embodiments, R³ is independently unsubstituted 2 to 6 membered heteroalkyl. In embodiments, R³ is independently unsubstituted methoxy. In embodiments, R³ is independently unsubstituted ethoxy. In embodiments, R³ is independently unsubstituted propoxy. In embodiments, R³ is independently unsubstituted n-propoxy. In embodiments, R³ is independently unsubstituted isopropoxy. In embodiments, R³ is independently unsubstituted butoxy.

In embodiments, R³ is independently halogen, —CF₃, —CHF₂, —CH₂F, —CN, —N₃, —SO₂R^{3A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, a substituted R^{3A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3A} is substituted with a plurality of groups selected from substituent groups, size-limited substituent groups, and lower substituent groups; each substituent group, size-limited substituent group, and/or lower substituent group may optionally be different. In embodiments, when R^{3A} is substituted, it is substituted with at least one substituent group. In embodiments, when R^{3A} is substituted, it is substituted with at least one size-limited substituent group. In embodiments, when R^{3A} is substituted, it is substituted with at least one lower substituent group.

In embodiments, a substituted R^{3B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3B} is substituted with a plurality of groups selected from substituent groups, size-limited substituent groups, and lower substituent groups; each substituent group, size-limited substituent group, and/or lower substituent group may optionally be different. In embodiments, when R^{3B} is substituted, it is substituted with at least one substituent group. In embodiments, when R^{3B} is substituted, it is substituted with at least one size-limited substituent group. In embodiments, when R^{3B} is substituted, it is substituted with at least one lower substituent group.

In embodiments, a substituted R^{3C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3C} is substituted with a plurality of groups selected from substituent groups, size-limited substituent groups, and lower substituent groups; each substituent group, size-limited substituent group, and/or lower substituent group may optionally be different. In embodiments, when R^{3C} is substituted, it is substituted with at least one substituent group. In embodiments, when R^{3C} is substituted, it is substituted with at least one size-limited substituent group. In embodiments, when R^{3C} is substituted, it is substituted with at least one lower substituent group.

In embodiments, a substituted ring formed when R^{3B} and R^{3C} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{3B} and R^{3C} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{3B} and R^{3C} substituents bonded to the same nitrogen atom are joined is substituted, it is substituted with at least one substituent group. In embodiments, when the substituted ring formed when R^{3B} and R^{3C}substituents bonded to the same nitrogen atom are joined is substituted, it is substituted with at least one size-limited substituent group. In embodiments, when the substituted ring formed when R^{3B} and R^{3C} substituents bonded to the same nitrogen atom are joined is substituted, it is substituted with at least one lower substituent group.

In embodiments, a substituted R^{3D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3D} is substituted with a plurality of groups selected from substituent groups, size-limited substituent groups, and lower substituent groups; each substituent group, size-limited substituent group, and/or lower substituent group may optionally be different. In embodiments, when R^{3D} is substituted, it is substituted with at least one substituent group. In embodiments, when R^{3D} is substituted, it is substituted with at least one size-limited substituent group. In embodiments, when R^{3D} is substituted, it is substituted with at least one lower substituent group.

In embodiments, R^{3A} is hydrogen. In embodiments, R^{3A} is unsubstituted C₁-C₄ alkyl. In embodiments, R^{3A} is unsubstituted methyl. In embodiments, R^{3A} is unsubstituted ethyl. In embodiments, R^{3A} is unsubstituted propyl. In embodiments, R^{3A} is unsubstituted n-propyl. In embodiments, R^{3A} is unsubstituted isopropyl. In embodiments, R^{3A} is unsubstituted butyl. In embodiments, R^{3A} is unsubstituted n-butyl. In embodiments, R^{3A} is unsubstituted isobutyl. In embodiments, R^{3A} is unsubstituted tert-butyl.

In embodiments, R^{3B} is hydrogen. In embodiments, R^{3B} is unsubstituted C₁-C₄ alkyl. In embodiments, R^{3B} is unsubstituted methyl. In embodiments, R^{3B} is unsubstituted ethyl. In embodiments, R^{3B} is unsubstituted propyl. In embodiments, R^{3B} is unsubstituted n-propyl. In embodiments, R^{3B} is unsubstituted isopropyl. In embodiments, R^{3B} is unsubstituted butyl. In embodiments, R^{3B} is unsubstituted n-butyl. In embodiments, R^{3B} is unsubstituted isobutyl. In embodiments, R^{3B} is unsubstituted tert-butyl.

In embodiments, R^{3C}, is hydrogen. In embodiments, R^{3C}, is unsubstituted C₁-C₄ alkyl. In embodiments, R^{3C} is unsubstituted methyl. In embodiments, R^{3C} is unsubstituted ethyl. In embodiments, R^{3C} is unsubstituted propyl. In embodiments, R^{3C} is unsubstituted n-propyl. In embodiments, R^{3C} is unsubstituted isopropyl. In embodiments, R^{3C} is unsubstituted butyl. In embodiments, R^{3C} is unsubstituted n-butyl. In embodiments, R^{3C} is unsubstituted isobutyl. In embodiments, R^{3C} is unsubstituted tert-butyl.

In embodiments, R^{3D} is hydrogen. In embodiments, R^{3D} is unsubstituted C₁-C₄ alkyl. In embodiments, R^{3D} is unsubstituted methyl. In embodiments, R^{3D} is unsubstituted ethyl. In embodiments, R^{3D} is unsubstituted propyl. In embodiments, R^{3D} is unsubstituted n-propyl. In embodiments, R^{3D} is unsubstituted isopropyl. In embodiments, R^{3D} is unsubstituted butyl. In embodiments, R^{3D} is unsubstituted n-butyl. In embodiments, R^{3D} is unsubstituted isobutyl. In embodiments, R^{3D} is unsubstituted tert-butyl.

In embodiments, a substituted R^{3.2} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3.2} is substituted with a plurality of groups selected from substituent groups, size-limited substituent groups, and lower substituent groups; each substituent group, size-limited substituent group, and/or lower substituent group may optionally be different. In embodiments, when R^{3.2} is substituted, it is substituted with at least one substituent group. In embodiments, when R^{3.2} is substituted, it is substituted with at least one size-limited substituent group. In embodiments, when R^{3.2} is substituted, it is substituted with at least one lower substituent group.

In embodiments, R^{3.2} is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R^{3.2} is hydrogen. In embodiments, R^{3.2} is halogen. In embodiments, R^{3.2} is —F. In embodiments, R^{3.2} is —Cl. In embodiments, R^{3.2} is —Br. In embodiments, R^{3.2} is —I. In embodiments, R^{3.2} is unsubstituted C₁-C₄ alkyl. In embodiments, R^{3.2} is unsubstituted methyl. In embodiments, R^{3.2} is unsubstituted ethyl. In embodiments, R^{3.2} is unsubstituted propyl. In embodiments, R^{3.2} is unsubstituted n-propyl. In embodiments, R^{3.2} is unsubstituted isopropyl. In embodiments, R^{3.2} is unsubstituted butyl. In embodiments, R^{3.2} is unsubstituted n-butyl. In embodiments, R^{3.2} is unsubstituted isobutyl. In embodiments, R^{3.2} is unsubstituted tert-butyl. In embodiments, R^{3.2} is unsubstituted 2 to 6 membered heteroalkyl. In embodiments, R^{3.2} is unsubstituted methoxy. In embodiments, R^{3.2} is unsubstituted ethoxy. In embodiments, R^{3.2} is unsubstituted propoxy. In embodiments, R^{3.2} is unsubstituted n-propoxy. In embodiments, R^{3.2} is unsubstituted isopropoxy. In embodiments, R^{3.2} is unsubstituted butoxy.

In embodiments, a substituted R^{3.3} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3.3} is substituted with a plurality of groups selected from substituent groups, size-limited substituent groups, and lower substituent groups; each substituent group, size-limited substituent group, and/or lower substituent group may optionally be different. In embodiments, when R^{3.3} is substituted, it is substituted with at least one substituent group. In embodiments, when R^{3.3} is substituted, it is substituted with at least one size-limited substituent group. In embodiments, when R^{3.3} is substituted, it is substituted with at least one lower substituent group.

In embodiments, R^{3.3} is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R^{3.3} is hydrogen. In embodiments, R^{3.3} is halogen. In embodiments, R^{3.3} is —F. In embodiments, R^{3.3} is —Cl. In embodiments, R^{3.3} is —Br. In embodiments, R^{3.3} is —I. In embodiments, R^{3.3} is unsubstituted C₁-C₄ alkyl. In embodiments, R^{3.3} is unsubstituted methyl. In embodiments, R^{3.3} is unsubstituted ethyl. In embodiments, R^{3.3} is unsubstituted propyl. In embodiments, R^{3.3} is unsubstituted n-propyl. In embodiments, R^{3.3} is unsubstituted isopropyl. In embodiments, R^{3.3} is unsubstituted butyl. In embodiments, R^{3.3} is unsubstituted n-butyl. In embodiments, R^{3.3} is unsubstituted isobutyl. In embodiments, R^{3.3} is unsubstituted tert-butyl. In embodiments, R^{3.3} is unsubstituted 2 to 6 membered heteroalkyl. In embodiments, R^{3.3} is unsubstituted methoxy. In embodiments, R^{3.3} is unsubstituted ethoxy. In embodiments, R^{3.3} is unsubstituted propoxy. In embodiments, R^{3.3} is unsubstituted n-propoxy. In embodiments, R^{3.3} is unsubstituted isopropoxy. In embodiments, R^{3.3} is unsubstituted butoxy.

In embodiments, R^{3.2} and R^{3.3} are independently hydrogen or any value of R³ as described herein, including in embodiments. In embodiments, R^{3.2} and R^{3.3} are independently halogen. In embodiments, R^{3.2} and R^{3.3} are —Cl.

In embodiments, a substituted R^{3.2A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3.2A} is substituted with a plurality of groups selected from substituent groups, size-limited substituent groups, and lower substituent groups; each substituent group, size-limited substituent group, and/or lower substituent group may optionally be different. In embodiments, when R^{3.2A} is substituted, it is substituted with at least one substituent group. In embodiments, when R^{3.2A} is substituted, it is substituted with at least one size-limited substituent group. In embodiments, when R^{3.2A} is substituted, it is substituted with at least one lower substituent group.

In embodiments, a substituted R^{3.2B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3.2B} is substituted with a plurality of groups selected from substituent groups, size-limited substituent groups, and lower substituent groups; each substituent group, size-limited substituent group, and/or lower substituent group may optionally be different. In embodiments, when R^{3.2B} is substituted, it is substituted with at least one substituent group. In embodiments, when R^{3.2B} is substituted, it is substituted with at least one size-limited substituent group. In embodiments, when R^{3.2B} is substituted, it is substituted with at least one lower substituent group.

In embodiments, a substituted R^{3.2c} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3.2c} is substituted with a plurality of groups selected from substituent groups, size-limited substituent groups, and lower substituent groups; each substituent group, size-limited substituent group, and/or lower substituent group may optionally be different. In embodiments, when R^{3.2c} is substituted, it is substituted with at least one substituent group. In embodiments, when R^{3.2c} is substituted, it is substituted with at least one size-limited substituent group. In embodiments, when R^{3.2c} is substituted, it is substituted with at least one lower substituent group.

In embodiments, a substituted ring formed when R^{3.2B} and R^{3.2c} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{3.2B} and R^{3.2c} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{3.2B} and R^{3.2c}substituents bonded to the same nitrogen atom are joined is substituted, it is substituted with at least one substituent group. In embodiments, when the substituted ring formed when R^{3.2B} and R^{3.2c} substituents bonded to the same nitrogen atom are joined is substituted, it is substituted with at least one size-limited substituent group. In embodiments, when the substituted ring formed when R^{3.2B} and R^{3.2c} substituents bonded to the same nitrogen atom are joined is substituted, it is substituted with at least one lower substituent group.

In embodiments, a substituted R^{3.2D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3.2D} is substituted with a plurality of groups selected from substituent groups, size-limited substituent groups, and lower substituent groups; each substituent group, size-limited substituent group, and/or lower substituent group may optionally be different. In embodiments, when R^{3.2D} is substituted, it is substituted with at least one substituent group. In embodiments, when R^{3.2D} is substituted, it is substituted with at least one size-limited substituent group. In embodiments, when R^{3.2D} is substituted, it is substituted with at least one lower substituent group.

In embodiments, R^{3.2A} is hydrogen. In embodiments, R^{3.2A} is unsubstituted C₁-C₄ alkyl. In embodiments, R^{3.2A} is unsubstituted methyl. In embodiments, R^{3.2A} is unsubstituted ethyl. In embodiments, R^{3.2A} is unsubstituted propyl. In embodiments, R^{3.2A} is unsubstituted n-propyl. In embodiments, R^{3.2A} is unsubstituted isopropyl. In embodiments, R^{3.2A} is unsubstituted butyl. In embodiments, R^{3.2A} is unsubstituted n-butyl. In embodiments, R^{3.2A} is unsubstituted isobutyl. In embodiments, R^{3.2A} is unsubstituted tert-butyl.

In embodiments, R^{3.21} is hydrogen. In embodiments, R^{3.21} is unsubstituted C₁-C₄ alkyl. In embodiments, R^{3.21} is unsubstituted methyl. In embodiments, R^{3.2B} is unsubstituted ethyl. In embodiments, R^{3.21} is unsubstituted propyl. In embodiments, R^{3.21} is unsubstituted n-propyl. In embodiments, R^{3.21} is unsubstituted isopropyl. In embodiments, R^{3.21} is unsubstituted butyl. In embodiments, R^{3.21} is unsubstituted n-butyl. In embodiments, R^{3.2B} is unsubstituted isobutyl. In embodiments, R^{3.21} is unsubstituted tert-butyl.

In embodiments, R^{3.2c} is hydrogen. In embodiments, R^{3.2c} is unsubstituted C₁-C₄ alkyl. In embodiments, R^{3.2c} is unsubstituted methyl. In embodiments, R^{3.2c} is unsubstituted ethyl. In embodiments, R^{3.2c} is unsubstituted propyl. In embodiments, R^{3.2c} is unsubstituted n-propyl. In embodiments, R^{3.2c} is unsubstituted isopropyl. In embodiments, R^{3.2c} is unsubstituted butyl. In embodiments, R^{3.2}c is unsubstituted n-butyl. In embodiments, R^{3.2C} is unsubstituted isobutyl. In embodiments, R^{3.2C} is unsubstituted tert-butyl.

In embodiments, R^{3.2D} is hydrogen. In embodiments, R^{3.2D} is unsubstituted C₁-C₄ alkyl. In embodiments, R^{3.2D} is unsubstituted methyl. In embodiments, R^{3.2D} is unsubstituted ethyl. In embodiments, R^{3.2D} is unsubstituted propyl. In embodiments, R^{3.2D} is unsubstituted n propyl. In embodiments, R^{3.2D} is unsubstituted isopropyl. In embodiments, R^{3.2D} is unsubstituted butyl. In embodiments, R^{3.2D} is unsubstituted n-butyl. In embodiments, R^{3.2D} is unsubstituted isobutyl. In embodiments, R^{3.2D} is unsubstituted tert-butyl.

In embodiments, a substituted R^{3.3A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3.3A} is substituted with a plurality of groups selected from substituent groups, size-limited substituent groups, and lower substituent groups; each substituent group, size-limited substituent group, and/or lower substituent group may optionally be different. In embodiments, when R^{3.3A} is substituted, it is substituted with at least one substituent group. In embodiments, when R^{3.3A} is substituted, it is substituted with at least one size-limited substituent group. In embodiments, when R^{3.3A} is substituted, it is substituted with at least one lower substituent group.

In embodiments, a substituted R^{3.3B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3.3B} is substituted with a plurality of groups selected from substituent groups, size-limited substituent groups, and lower substituent groups; each substituent group, size-limited substituent group, and/or lower substituent group may optionally be different. In embodiments, when R^{3.3B} is substituted, it is substituted with at least one substituent group. In embodiments, when R^{3.3B} is substituted, it is substituted with at least one size-limited substituent group. In embodiments, when R^{3.3B} is substituted, it is substituted with at least one lower substituent group.

In embodiments, a substituted R^{3.3C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3.3C} is substituted with a plurality of groups selected from substituent groups, size-limited substituent groups, and lower substituent groups; each substituent group, size-limited substituent group, and/or lower substituent group may optionally be different. In embodiments, when R^{3.3C} is substituted, it is substituted with at least one substituent group. In embodiments, when R^{3.3C} is substituted, it is substituted with at least one size-limited substituent group. In embodiments, when R^{3.3C} is substituted, it is substituted with at least one lower substituent group.

In embodiments, a substituted ring formed when R^{3.3B} and R^{3.3C} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{3.3B} and R^{3.3C} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{3.3B} and R^{3.3}c substituents bonded to the same nitrogen atom are joined is substituted, it is substituted with at least one substituent group. In embodiments, when the substituted ring formed when R^{3.3B} and R^{3.3C} substituents bonded to the same nitrogen atom are joined is substituted, it is substituted with at least one size-limited substituent group. In embodiments, when the substituted ring formed when R^{3.3B} and R^{3.3C} substituents bonded to the same nitrogen atom are joined is substituted, it is substituted with at least one lower substituent group.

In embodiments, a substituted R^{3.3D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3.3D} is substituted with a plurality of groups selected from substituent groups, size-limited substituent groups, and lower substituent groups; each substituent group, size-limited substituent group, and/or lower substituent group may optionally be different. In embodiments, when R^{3.3D} is substituted, it is substituted with at least one substituent group. In embodiments, when R^{3.3D} is substituted, it is substituted with at least one size-limited substituent group. In embodiments, when R^{3.3D} is substituted, it is substituted with at least one lower substituent group.

In embodiments, R^{3.3A} is hydrogen. In embodiments, R^{3.3A} is unsubstituted C₁-C₄ alkyl. In embodiments, R^{3.3A} is unsubstituted methyl. In embodiments, R^{3.3A} is unsubstituted ethyl. In embodiments, R^{3.3A} is unsubstituted propyl. In embodiments, R^{3.3A} is unsubstituted n propyl. In embodiments, R^{3.3A} is unsubstituted isopropyl. In embodiments, R^{3.3A} is unsubstituted butyl. In embodiments, R^{3.3A} is unsubstituted n-butyl. In embodiments, R^{3.3A} is unsubstituted isobutyl. In embodiments, R^{3.3A} is unsubstituted tert-butyl.

In embodiments, R^{3.3B} is hydrogen. In embodiments, R^{3.3B} is unsubstituted C₁-C₄ alkyl. In embodiments, R^{3.3B} is unsubstituted methyl. In embodiments, R^{3.3B} is unsubstituted ethyl. In embodiments, R^{3.3B} is unsubstituted propyl. In embodiments, R^{3.3B} is unsubstituted n-propyl. In embodiments, R^{3.3B} is unsubstituted isopropyl. In embodiments, R^{3.31} is unsubstituted butyl. In embodiments, R^{3.3B} is unsubstituted n-butyl. In embodiments, R^{3.3B} is unsubstituted isobutyl. In embodiments, R^{3.3B} is unsubstituted tert-butyl.

In embodiments, R^{3.3C} is hydrogen. In embodiments, R^{3.3C} is unsubstituted C₁-C₄ alkyl. In embodiments, R^{3.3C} is unsubstituted methyl. In embodiments, R^{3.3C} is unsubstituted ethyl. In embodiments, R^{3.3C} is unsubstituted propyl. In embodiments, R^{3.3C} is unsubstituted n-propyl. In embodiments, R^{3.3C} is unsubstituted isopropyl. In embodiments, R^{3.3C} is unsubstituted butyl. In embodiments, R^{3.3C} is unsubstituted n-butyl. In embodiments, R^{3.3C} is unsubstituted isobutyl. In embodiments, R^{3.3C} is unsubstituted tert-butyl.

In embodiments, R^{3.3D} is hydrogen. In embodiments, R^{3.3D} is unsubstituted C₁-C₄ alkyl. In embodiments, R^{3.3D} is unsubstituted methyl. In embodiments, R^{3.3D} is unsubstituted ethyl. In embodiments, R^{3.3D} is unsubstituted propyl. In embodiments, R^{3.3D} is unsubstituted n propyl. In embodiments, R^{3.3D} is unsubstituted isopropyl. In embodiments, R^{3.3D} is unsubstituted butyl. In embodiments, R^{3.3D} is unsubstituted n-butyl. In embodiments, R^{3.3D} is unsubstituted isobutyl. In embodiments, R^{3.3D} is unsubstituted tert-butyl.

In embodiments, a substituted R⁴ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁴ is substituted with a plurality of groups selected from substituent groups, size-limited substituent groups, and lower substituent groups; each substituent group, size-limited substituent group, and/or lower substituent group may optionally be different. In embodiments, when R⁴ is substituted, it is substituted with at least one substituent group. In embodiments, when R⁴ is substituted, it is substituted with at least one size-limited substituent group. In embodiments, when R⁴ is substituted, it is substituted with at least one lower substituent group.

In embodiments, R⁴ is hydrogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —COOH, —CONH₂, —SH, —SO₃H, —OSO₃H, —SO₂NH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁴ is hydrogen. In embodiments, R⁴ is unsubstituted C₁-C₄ alkyl. In embodiments, R⁴ is unsubstituted methyl. In embodiments, R⁴ is unsubstituted ethyl. In embodiments, R⁴ is unsubstituted propyl. In embodiments, R⁴ is unsubstituted n-propyl. In embodiments, R⁴ is unsubstituted isopropyl. In embodiments, R⁴ is unsubstituted butyl. In embodiments, R⁴ is unsubstituted n-butyl. In embodiments, R⁴ is unsubstituted isobutyl. In embodiments, R⁴ is unsubstituted tert-butyl.

In embodiments, a substituted R^{4A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4A} is substituted with a plurality of groups selected from substituent groups, size-limited substituent groups, and lower substituent groups; each substituent group, size-limited substituent group, and/or lower substituent group may optionally be different. In embodiments, when R^{4A} is substituted, it is substituted with at least one substituent group. In embodiments, when R^{4A} is substituted, it is substituted with at least one size-limited substituent group. In embodiments, when R^{4A} is substituted, it is substituted with at least one lower substituent group.

In embodiments, a substituted R^{4B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4B} is substituted with a plurality of groups selected from substituent groups, size-limited substituent groups, and lower substituent groups; each substituent group, size-limited substituent group, and/or lower substituent group may optionally be different. In embodiments, when R^{4B} is substituted, it is substituted with at least one substituent group. In embodiments, when R^{4B} is substituted, it is substituted with at least one size-limited substituent group. In embodiments, when R^{4B} is substituted, it is substituted with at least one lower substituent group.

In embodiments, a substituted R^{4C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4C} is substituted with a plurality of groups selected from substituent groups, size-limited substituent groups, and lower substituent groups; each substituent group, size-limited substituent group, and/or lower substituent group may optionally be different. In embodiments, when R^{4C} is substituted, it is substituted with at least one substituent group. In embodiments, when R^{4C} is substituted, it is substituted with at least one size-limited substituent group. In embodiments, when R^{4C} is substituted, it is substituted with at least one lower substituent group.

In embodiments, a substituted ring formed when R^{4B} and R^{4C} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{4B} and R^{4C} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{4B} and R^{4C} substituents bonded to the same nitrogen atom are joined is substituted, it is substituted with at least one substituent group. In embodiments, when the substituted ring formed when R^{4B} and R^{4C}substituents bonded to the same nitrogen atom are joined is substituted, it is substituted with at least one size-limited substituent group. In embodiments, when the substituted ring formed when R^{4B} and R^{4C} substituents bonded to the same nitrogen atom are joined is substituted, it is substituted with at least one lower substituent group.

In embodiments, a substituted R^{4D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4D} is substituted with a plurality of groups selected from substituent groups, size-limited substituent groups, and lower substituent groups; each substituent group, size-limited substituent group, and/or lower substituent group may optionally be different. In embodiments, when R^{4D} is substituted, it is substituted with at least one substituent group. In embodiments, when R^{4D} is substituted, it is substituted with at least one size-limited substituent group. In embodiments, when R^{4D} is substituted, it is substituted with at least one lower substituent group.

In embodiments, R^{4A} is hydrogen. In embodiments, R^{4A} is unsubstituted C₁-C₄ alkyl. In embodiments, R^{4A} is unsubstituted methyl. In embodiments, R^{4A} is unsubstituted ethyl. In embodiments, R^{4A} is unsubstituted propyl. In embodiments, R^{4A} is unsubstituted n-propyl. In embodiments, R^{4A} is unsubstituted isopropyl. In embodiments, R^{4A} is unsubstituted butyl. In embodiments, R^{4A} is unsubstituted n-butyl. In embodiments, R^{4A} is unsubstituted isobutyl. In embodiments, R^{4A} is unsubstituted tert-butyl.

In embodiments, R^{4B} is hydrogen. In embodiments, R^{4B} is unsubstituted C₁-C₄ alkyl. In embodiments, R^{4B} is unsubstituted methyl. In embodiments, R^{4B} is unsubstituted ethyl. In embodiments, R^{4B} is unsubstituted propyl. In embodiments, R^{4B} is unsubstituted n-propyl. In embodiments, R^{4B} is unsubstituted isopropyl. In embodiments, R^{4B} is unsubstituted butyl. In embodiments, R^{4B} is unsubstituted n-butyl. In embodiments, R^{4B} is unsubstituted isobutyl. In embodiments, R^{4B} is unsubstituted tert-butyl.

In embodiments, R^{4C} is hydrogen. In embodiments, R^{4C} is unsubstituted C₁-C₄ alkyl. In embodiments, R^{4C} is unsubstituted methyl. In embodiments, R^{4C} is unsubstituted ethyl. In embodiments, R^{4C} is unsubstituted propyl. In embodiments, R^{4C} is unsubstituted n-propyl. In embodiments, R^{4C} is unsubstituted isopropyl. In embodiments, R^{4C} is unsubstituted butyl. In embodiments, R^{4C} is unsubstituted n-butyl. In embodiments, R^{4C} is unsubstituted isobutyl. In embodiments, R^{4C} is unsubstituted tert-butyl.

In embodiments, R^{4D} is hydrogen. In embodiments, R^{4D} is unsubstituted C₁-C₄ alkyl. In embodiments, R^{4D} is unsubstituted methyl. In embodiments, R^{4D} is unsubstituted ethyl. In embodiments, R^{4D} is unsubstituted propyl. In embodiments, R^{4D} is unsubstituted n-propyl. In embodiments, R^{4D} is unsubstituted isopropyl. In embodiments, R^{4D} is unsubstituted butyl. In embodiments, R^{4D} is unsubstituted n-butyl. In embodiments, R^{4D} is unsubstituted isobutyl. In embodiments, R^{4D} is unsubstituted tert-butyl.

In embodiments, a substituted R⁵(e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁵ is substituted with a plurality of groups selected from substituent groups, size-limited substituent groups, and lower substituent groups; each substituent group, size-limited substituent group, and/or lower substituent group may optionally be different. In embodiments, when R⁵ is substituted, it is substituted with at least one substituent group. In embodiments, when R⁵ is substituted, it is substituted with at least one size-limited substituent group. In embodiments, when R⁵ is substituted, it is substituted with at least one lower substituent group.

In embodiments, R⁵ is independently halogen, oxo, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁵ is independently halogen. In embodiments, R⁵ is independently oxo. In embodiments, R⁵ is independently —F. In embodiments, R⁵ is independently —Cl. In embodiments, R⁵ is independently —Br. In embodiments, R⁵ is independently —I. In embodiments, R⁵ is independently —CCl₃. In embodiments, R⁵ is independently —CBr₃. In embodiments, R⁵ is independently —CF₃. In embodiments, R⁵ is independently —CI₃. In embodiments, R⁵ is independently —CH₂Cl. In embodiments, R⁵ is independently —CH₂Br. In embodiments, R⁵ is independently —CH₂F. In embodiments, R⁵ is independently —CH₂I. In embodiments, R⁵ is independently —CHCl₂. In embodiments, R⁵ is independently —CHBr₂. In embodiments, R⁵ is independently —CHF₂. In embodiments, R⁵ is independently —CHI₂. In embodiments, R⁵ is independently —CN. In embodiments, R⁵ is independently —OH. In embodiments, R⁵ is independently —NH₂. In embodiments, R⁵ is independently —COOH. In embodiments, R⁵ is independently —CONH₂. In embodiments, R⁵ is independently —NO₂. In embodiments, R⁵ is independently —SH. In embodiments, R⁵ is independently —SO₃H. In embodiments, R⁵ is independently —OSO₃H. In embodiments, R⁵ is independently —SO₂NH₂. In embodiments, R⁵ is independently —NHNH₂. In embodiments, R⁵ is independently —ONH₂. In embodiments, R⁵ is independently —NHC(O)NH₂. In embodiments, R⁵ is independently —NHSO₂H. In embodiments, R⁵ is independently —NHC(O)H. In embodiments, R⁵ is independently —NHC(O)OH. In embodiments, R⁵ is independently —NHOH. In embodiments, R⁵ is independently —OCCl₃. In embodiments, R⁵ is independently —OCBr₃. In embodiments, R⁵ is independently —OCF₃. In embodiments, R⁵ is independently —OCI₃. In embodiments, R⁵ is independently —OCH₂Cl. In embodiments, R⁵ is independently —OCH₂Br. In embodiments, R⁵ is independently —OCH₂F. In embodiments, R⁵ is independently —OCH₂I. In embodiments, R⁵ is independently —OCHCl₂. In embodiments, R⁵ is independently —OCHBr₂. In embodiments, R⁵ is independently —OCHF₂. In embodiments, R⁵ is independently —OCHI₂. In embodiments, R⁵ is independently —N₃. In embodiments, R⁵ is independently unsubstituted C₁-C₄ alkyl. In embodiments, R⁵ is independently unsubstituted methyl. In embodiments, R⁵ is independently unsubstituted ethyl. In embodiments, R⁵ is independently unsubstituted propyl. In embodiments, R⁵ is independently unsubstituted n-propyl. In embodiments, R⁵ is independently unsubstituted isopropyl. In embodiments, R⁵ is independently unsubstituted butyl. In embodiments, R⁵ is independently unsubstituted n-butyl. In embodiments, R⁵ is independently unsubstituted isobutyl. In embodiments, R⁵ is independently unsubstituted tert-butyl. In embodiments, R⁵ is independently unsubstituted 2 to 6 membered heteroalkyl. In embodiments, R⁵ is independently unsubstituted methoxy. In embodiments, R⁵ is independently unsubstituted ethoxy. In embodiments, R⁵ is independently unsubstituted propoxy. In embodiments, R⁵ is independently unsubstituted n-propoxy. In embodiments, R⁵ is independently unsubstituted isopropoxy. In embodiments, R⁵ is independently unsubstituted butoxy.

In embodiments, a substituted R^{5A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{5A} is substituted with a plurality of groups selected from substituent groups, size-limited substituent groups, and lower substituent groups; each substituent group, size-limited substituent group, and/or lower substituent group may optionally be different. In embodiments, when R^{5A} is substituted, it is substituted with at least one substituent group. In embodiments, when R^{5A} is substituted, it is substituted with at least one size-limited substituent group. In embodiments, when R^{5A} is substituted, it is substituted with at least one lower substituent group.

In embodiments, a substituted R^{5B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{5B} is substituted with a plurality of groups selected from substituent groups, size-limited substituent groups, and lower substituent groups; each substituent group, size-limited substituent group, and/or lower substituent group may optionally be different. In embodiments, when R^{5B} is substituted, it is substituted with at least one substituent group. In embodiments, when R^{5B} is substituted, it is substituted with at least one size-limited substituent group. In embodiments, when R^{5B} is substituted, it is substituted with at least one lower substituent group.

In embodiments, a substituted R^{5C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{5C} is substituted with a plurality of groups selected from substituent groups, size-limited substituent groups, and lower substituent groups; each substituent group, size-limited substituent group, and/or lower substituent group may optionally be different. In embodiments, when R^{5C} is substituted, it is substituted with at least one substituent group. In embodiments, when R^{5C} is substituted, it is substituted with at least one size-limited substituent group. In embodiments, when R^{5C} is substituted, it is substituted with at least one lower substituent group.

In embodiments, a substituted ring formed when R^{5B} and R^{5C} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{5B} and R^{5C} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{5B} and R^{5C} substituents bonded to the same nitrogen atom are joined is substituted, it is substituted with at least one substituent group. In embodiments, when the substituted ring formed when R^{5B} and R^{5C} substituents bonded to the same nitrogen atom are joined is substituted, it is substituted with at least one size-limited substituent group. In embodiments, when the substituted ring formed when R^{5B} and R^{5C} substituents bonded to the same nitrogen atom are joined is substituted, it is substituted with at least one lower substituent group.

In embodiments, a substituted R^{5D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{5D} is substituted with a plurality of groups selected from substituent groups, size-limited substituent groups, and lower substituent groups; each substituent group, size-limited substituent group, and/or lower substituent group may optionally be different. In embodiments, when R^{5D} is substituted, it is substituted with at least one substituent group. In embodiments, when R^{5D} is substituted, it is substituted with at least one size-limited substituent group. In embodiments, when R^{5D} is substituted, it is substituted with at least one lower substituent group.

In embodiments, R^{5A} is hydrogen. In embodiments, R^{5A} is unsubstituted C₁-C₄ alkyl. In embodiments, R^{5A} is unsubstituted methyl. In embodiments, R^{5A} is unsubstituted ethyl. In embodiments, R^{5A} is unsubstituted propyl. In embodiments, R^{5A} is unsubstituted n-propyl. In embodiments, R^{5A} is unsubstituted isopropyl. In embodiments, R^{5A} is unsubstituted butyl. In embodiments, R^{5A} is unsubstituted n-butyl. In embodiments, R^{5A} is unsubstituted isobutyl. In embodiments, R^{5A} is unsubstituted tert-butyl.

In embodiments, R^{5B} is hydrogen. In embodiments, R^{5B} is unsubstituted C₁-C₄ alkyl. In embodiments, R^{5B} is unsubstituted methyl. In embodiments, R^{5B} is unsubstituted ethyl. In embodiments, R^{5B} is unsubstituted propyl. In embodiments, R^{5B} is unsubstituted n-propyl. In embodiments, R^{5B} is unsubstituted isopropyl. In embodiments, R^{5B} is unsubstituted butyl. In embodiments, R^{5B} is unsubstituted n-butyl. In embodiments, R^{5B} is unsubstituted isobutyl. In embodiments, R^{5B} is unsubstituted tert-butyl.

In embodiments, R^{5C} is hydrogen. In embodiments, R^{5C} is unsubstituted C₁-C₄ alkyl. In embodiments, R^{5C} is unsubstituted methyl. In embodiments, R^{5C} is unsubstituted ethyl. In embodiments, R^{5C} is unsubstituted propyl. In embodiments, R^{5C} is unsubstituted n-propyl. In embodiments, R^{5C} is unsubstituted isopropyl. In embodiments, R^{5C} is unsubstituted butyl. In embodiments, R^{5C} is unsubstituted n-butyl. In embodiments, R^{5C} is unsubstituted isobutyl. In embodiments, R^{5C} is unsubstituted tert-butyl.

In embodiments, R^{5D} is hydrogen. In embodiments, R^{5D} is unsubstituted C₁-C₄ alkyl. In embodiments, R^{5D} is unsubstituted methyl. In embodiments, R^{5D} is unsubstituted ethyl. In embodiments, R^{5D} is unsubstituted propyl. In embodiments, R^{5D} is unsubstituted n-propyl. In embodiments, R^{5D} is unsubstituted isopropyl. In embodiments, R^{5D} is unsubstituted butyl. In embodiments, R^{5D} is unsubstituted n-butyl. In embodiments, R^{5D} is unsubstituted isobutyl. In embodiments, R^{5D} is unsubstituted tert-butyl.

In embodiments, a substituted R⁶ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁶ is substituted with a plurality of groups selected from substituent groups, size-limited substituent groups, and lower substituent groups; each substituent group, size-limited substituent group, and/or lower substituent group may optionally be different. In embodiments, when R⁶ is substituted, it is substituted with at least one substituent group. In embodiments, when R⁶ is substituted, it is substituted with at least one size-limited substituent group. In embodiments, when R⁶ is substituted, it is substituted with at least one lower substituent group.

In embodiments, R⁶ is independently halogen, oxo, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁶ is independently halogen. In embodiments, R⁶ is independently oxo. In embodiments, R⁶ is independently —F. In embodiments, R⁶ is independently —Cl. In embodiments, R⁶ is independently —Br. In embodiments, R⁶ is independently —I. In embodiments, R⁶ is independently —CCl₃. In embodiments, R⁶ is independently —CBr₃. In embodiments, R⁶ is independently —CF₃. In embodiments, R⁶ is independently —CI₃. In embodiments, R⁶ is independently —CH₂C₁. In embodiments, R⁶ is independently —CH₂Br. In embodiments, R⁶ is independently —CH₂F. In embodiments, R⁶ is independently —CH₂I. In embodiments, R⁶ is independently —CHCl₂. In embodiments, R⁶ is independently —CHBr₂. In embodiments, R⁶ is independently —CHF₂. In embodiments, R⁶ is independently —CHI₂. In embodiments, R⁶ is independently —CN. In embodiments, R⁶ is independently —OH. In embodiments, R⁶ is independently —NH₂. In embodiments, R⁶ is independently —COOH. In embodiments, R⁶ is independently —CONH₂. In embodiments, R⁶ is independently —NO₂. In embodiments, R⁶ is independently —SH. In embodiments, R⁶ is independently —SO₃H. In embodiments, R⁶ is independently —OSO₃H. In embodiments, R⁶ is independently —SO₂NH₂. In embodiments, R⁶ is independently —NHNH₂. In embodiments, R⁶ is independently —ONH₂. In embodiments, R⁶ is independently —NHC(O)NH₂. In embodiments, R⁶ is independently —NHSO₂H. In embodiments, R⁶ is independently —NHC(O)H. In embodiments, R⁶ is independently —NHC(O)OH. In embodiments, R⁶ is independently —NHOH. In embodiments, R⁶ is independently —OCCl₃. In embodiments, R⁶ is independently —OCBr₃. In embodiments, R⁶ is independently —OCF₃. In embodiments, R⁶ is independently —OCI₃. In embodiments, R⁶ is independently —OCH₂Cl. In embodiments, R⁶ is independently —OCH₂Br. In embodiments, R⁶ is independently —OCH₂F. In embodiments, R⁶ is independently —OCH₂I. In embodiments, R⁶ is independently —OCHCl₂. In embodiments, R⁶ is independently —OCHBr₂. In embodiments, R⁶ is independently —OCHF₂. In embodiments, R⁶ is independently —OCHI₂. In embodiments, R⁶ is independently —N₃. In embodiments, R⁶ is independently unsubstituted C₁-C₄ alkyl. In embodiments, R⁶ is independently unsubstituted methyl. In embodiments, R⁶ is independently unsubstituted ethyl. In embodiments, R⁶ is independently unsubstituted propyl. In embodiments, R⁶ is independently unsubstituted n-propyl. In embodiments, R⁶ is independently unsubstituted isopropyl. In embodiments, R⁶ is independently unsubstituted butyl. In embodiments, R⁶ is independently unsubstituted n-butyl. In embodiments, R⁶ is independently unsubstituted isobutyl. In embodiments, R⁶ is independently unsubstituted tert-butyl. In embodiments, R⁶ is independently unsubstituted 2 to 6 membered heteroalkyl. In embodiments, R⁶ is independently unsubstituted methoxy. In embodiments, R⁶ is independently unsubstituted ethoxy. In embodiments, R⁶ is independently unsubstituted propoxy. In embodiments, R⁶ is independently unsubstituted n-propoxy. In embodiments, R⁶ is independently unsubstituted isopropoxy. In embodiments, R⁶ is independently unsubstituted butoxy.

In embodiments, a substituted R^{6A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{6A} is substituted with a plurality of groups selected from substituent groups, size-limited substituent groups, and lower substituent groups; each substituent group, size-limited substituent group, and/or lower substituent group may optionally be different. In embodiments, when R^{6A} is substituted, it is substituted with at least one substituent group. In embodiments, when R^{6A} is substituted, it is substituted with at least one size-limited substituent group. In embodiments, when R^{6A} is substituted, it is substituted with at least one lower substituent group.

In embodiments, a substituted R^{6B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{6B} is substituted with a plurality of groups selected from substituent groups, size-limited substituent groups, and lower substituent groups; each substituent group, size-limited substituent group, and/or lower substituent group may optionally be different. In embodiments, when R^{6B} is substituted, it is substituted with at least one substituent group. In embodiments, when R^{6B} is substituted, it is substituted with at least one size-limited substituent group. In embodiments, when R^{6B} is substituted, it is substituted with at least one lower substituent group.

In embodiments, a substituted R^{6C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{6C} is substituted with a plurality of groups selected from substituent groups, size-limited substituent groups, and lower substituent groups; each substituent group, size-limited substituent group, and/or lower substituent group may optionally be different. In embodiments, when R^{6C} is substituted, it is substituted with at least one substituent group. In embodiments, when R^{6C} is substituted, it is substituted with at least one size-limited substituent group. In embodiments, when R^{6C} is substituted, it is substituted with at least one lower substituent group.

In embodiments, a substituted ring formed when R^{6B} and R^{6C} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{6B} and R^{6C} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{6B} and R^{6C} substituents bonded to the same nitrogen atom are joined is substituted, it is substituted with at least one substituent group. In embodiments, when the substituted ring formed when R^{6B} and R^{6C}substituents bonded to the same nitrogen atom are joined is substituted, it is substituted with at least one size-limited substituent group. In embodiments, when the substituted ring formed when R^{6B} and R^{6C} substituents bonded to the same nitrogen atom are joined is substituted, it is substituted with at least one lower substituent group.

In embodiments, a substituted R^{6D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{6D} is substituted with a plurality of groups selected from substituent groups, size-limited substituent groups, and lower substituent groups; each substituent group, size-limited substituent group, and/or lower substituent group may optionally be different. In embodiments, when R^{6D} is substituted, it is substituted with at least one substituent group. In embodiments, when R^{6D} is substituted, it is substituted with at least one size-limited substituent group. In embodiments, when R^{6D} is substituted, it is substituted with at least one lower substituent group.

In embodiments, R^{6A} is hydrogen. In embodiments, R^{6A} is unsubstituted C_—C₄ alkyl. In embodiments, R^{6A} is unsubstituted methyl. In embodiments, R^{6A} is unsubstituted ethyl. In embodiments, R^{6A} is unsubstituted propyl. In embodiments, R^{6A} is unsubstituted n-propyl. In embodiments, R^{6A} is unsubstituted isopropyl. In embodiments, R^{6A} is unsubstituted butyl. In embodiments, R^{6A} is unsubstituted n-butyl. In embodiments, R^{6A} is unsubstituted isobutyl. In embodiments, R^{6A} is unsubstituted tert-butyl.

In embodiments, R^{6B} is hydrogen. In embodiments, R^{6B} is unsubstituted C₁-C₄ alkyl. In embodiments, R^{6B} is unsubstituted methyl. In embodiments, R^{6B} is unsubstituted ethyl. In embodiments, R^{6B} is unsubstituted propyl. In embodiments, R^{6B} is unsubstituted n-propyl. In embodiments, R^{6B} is unsubstituted isopropyl. In embodiments, R^{6B} is unsubstituted butyl. In embodiments, R^{6B} is unsubstituted n-butyl. In embodiments, R^{6B} is unsubstituted isobutyl. In embodiments, R^{6B} is unsubstituted tert-butyl.

In embodiments, R^{6C} is hydrogen. In embodiments, RC is unsubstituted C₁-C₄ alkyl. In embodiments, R^{6C} is unsubstituted methyl. In embodiments, R^{6C} is unsubstituted ethyl. In embodiments, R^{6C} is unsubstituted propyl. In embodiments, R^{6C} is unsubstituted n-propyl. In embodiments, R^{6C} is unsubstituted isopropyl. In embodiments, R^{6C} is unsubstituted butyl. In embodiments, R^{6C} is unsubstituted n-butyl. In embodiments, R^{6C} is unsubstituted isobutyl. In embodiments, R^{6C} is unsubstituted tert-butyl.

In embodiments, R^{6D} is hydrogen. In embodiments, R^{6D} is unsubstituted C₁-C₄ alkyl. In embodiments, R^{6D} is unsubstituted methyl. In embodiments, R^{6D} is unsubstituted ethyl. In embodiments, R^{6D} is unsubstituted propyl. In embodiments, R^{6D} is unsubstituted n-propyl. In embodiments, R^{6D} is unsubstituted isopropyl. In embodiments, R^{6D} is unsubstituted butyl. In embodiments, R^{6D} is unsubstituted n-butyl. In embodiments, R^{6D} is unsubstituted isobutyl. In embodiments, R^{6D} is unsubstituted tert-butyl.

In embodiments, a substituted L⁷ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L⁷ is substituted with a plurality of groups selected from substituent groups, size-limited substituent groups, and lower substituent groups; each substituent group, size-limited substituent group, and/or lower substituent group may optionally be different. In embodiments, when L⁷ is substituted, it is substituted with at least one substituent group. In embodiments, when L⁷ is substituted, it is substituted with at least one size-limited substituent group. In embodiments, when L⁷ is substituted, it is substituted with at least one lower substituent group.

In embodiments, L⁷ is a bond, —O—, —S—, —NH—, —C(O)—, —C(O)O—, —S(O)—, —S(O)₂—, substituted or unsubstituted C₁-C₆ alkylene, substituted or unsubstituted 2 to 6 membered heteroalkylene, substituted or unsubstituted C₃-C₆ cycloalkylene, substituted or unsubstituted 3 to 6 membered heterocycloalkylene, substituted or unsubstituted phenylene, or substituted or unsubstituted 5 to 6 membered heteroarylene.

In embodiments, L⁷ is a bond. In embodiments, L⁷ is —O—. In embodiments, L⁷ is —S—. In embodiments, L⁷ is —NR^{7.2B}—, wherein R^{7.2B} is as described herein, including in embodiments. In embodiments, L⁷ is —NH—. In embodiments, L⁷ is —C(O)—. In embodiments, L⁷ is —C(O)O—. In embodiments, L⁷ is —S(O)—. In embodiments, L⁷ is —S(O)₂—.

In embodiments, L⁷ is a bond, substituted or unsubstituted alkylene, or substituted or unsubstituted cycloalkylene. In embodiments, L⁷ is a bond, unsubstituted C₁-C₆ alkylene, or substituted or unsubstituted C₃-C₆ cycloalkylene. In embodiments, L⁷ is a bond, unsubstituted methylene, unsubstituted ethylene, unsubstituted n-propylene,

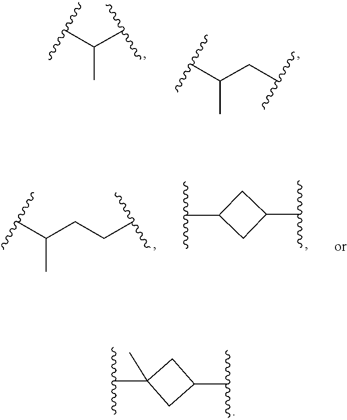

In embodiments, L7 is unsubstituted methylene. In embodiments, L7 is unsubstituted ethylene. In embodiments, L7 is unsubstituted propylene. In embodiments, L7 is unsubstituted n-propylene. In embodiments, L7 is unsubstituted isopropylene. In embodiments, L7 is unsubstituted butylene. In embodiments, L7 is unsubstituted n-butylene. In embodiments, L7 is unsubstituted tert-butylene. In embodiments, L7 is unsubstituted cyclopropylene. In embodiments, L7 is unsubstituted cyclobutylene. In embodiments, L7 is unsubstituted cyclopentylene. In embodiments, L7 is unsubstituted cyclohexylene. In embodiments, L7 is

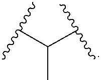 [SMILES: *C(*)C]

In embodiments, L7 is

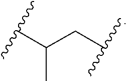 [SMILES: *CC(*)C]

In embodiments, L7 is

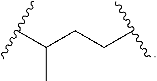 [SMILES: *CCC(*)C]

In embodiments, L7 is

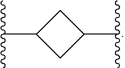 [SMILES: *C1CC(*)C1]

In embodiments, L7 is

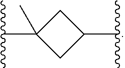 [SMILES: *C1CC(*)(C)C1]

In embodiments, a substituted R^{72B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{7.2B} is substituted with a plurality of groups selected from substituent groups, size-limited substituent groups, and lower substituent groups; each substituent group, size-limited substituent group, and/or lower substituent group may optionally be different. In embodiments, when R^{7.2B} is substituted, it is substituted with at least one substituent group. In embodiments, when R^{7.2B} is substituted, it is substituted with at least one size-limited substituent group. In embodiments, when R^{7.2B} is substituted, it is substituted with at least one lower substituent group.

In embodiments, R^{7.2B} is hydrogen. In embodiments, R^{7.2B} is unsubstituted C₁-C₄ alkyl. In embodiments, R^{7.2B} is unsubstituted methyl. In embodiments, R^{7.2B} is unsubstituted ethyl. In embodiments, R^{7.2B} is unsubstituted propyl. In embodiments, R^{7.2B} is unsubstituted n-propyl. In embodiments, R^{7.2B} is unsubstituted isopropyl. In embodiments, R^{7.2B} is unsubstituted butyl. In embodiments, R^{7.2B} is unsubstituted n-butyl. In embodiments, R^{7.2B} is unsubstituted isobutyl. In embodiments, R^{7.2B} is unsubstituted tert-butyl.

In embodiments, a substituted R⁷ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁷ is substituted with a plurality of groups selected from substituent groups, size-limited substituent groups, and lower substituent groups; each substituent group, size-limited substituent group, and/or lower substituent group may optionally be different. In embodiments, when R⁷ is substituted, it is substituted with at least one substituent group. In embodiments, when R⁷ is substituted, it is substituted with at least one size-limited substituent group. In embodiments, when R⁷ is substituted, it is substituted with at least one lower substituent group.

In embodiments, R⁷ is hydrogen, halogen, —CX^{7.1}₃, —CN, —SOₙ₇R^{7A}, —SOᵥ₇NR^{7B}R^{7C}, —NHC(O)NR^{7B}R^{7C}, —NR^{7B}R^{7C}, —C(O)R^{7D}, —C(O)OR^{7D}, —C(O)NR^{7B}R^{7C}, —OR^{7A}, —NR^{7B}SO₂R^{7A}, —NR^{7B}C(O)R^{7D}, —NR^{7B}C(O)OR^{7D}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R⁷ is hydrogen, —CX^{7.1}₃, —CN, —OR^{7A}, —C(O)OR^{7D}, —C(O)NR^{7B}R^{7C}, —NR^{7B}C(O)R^{7D}, —SO₇R^{7A}, —SOᵥ₇NR^{7B}R^{7C}, substituted or unsubstituted C₁-C₄ alkyl, unsubstituted 2 to 6 membered heteroalkyl, substituted or unsubstituted C₃-C₆ cycloalkyl, or substituted or unsubstituted 5 to 6 membered heteroaryl.

In embodiments, R⁷ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁷ is hydrogen. In embodiments, R⁷ is halogen. In embodiments, R⁷ is —F. In embodiments, R⁷ is —Cl. In embodiments, R⁷ is —Br. In embodiments, R⁷ is —I. In embodiments, R⁷ is —CCl₃. In embodiments, R⁷ is —CBr₃. In embodiments, R⁷ is —CF₃. In embodiments, R⁷ is —CI₃. In embodiments, R⁷ is —CH₂C₁. In embodiments, R⁷ is —CH₂Br. In embodiments, R⁷ is —CH₂F. In embodiments, R⁷ is —CH₂I. In embodiments, R⁷ is —CHCl₂. In embodiments, R⁷ is —CHBr₂. In embodiments, R⁷ is —CHF₂. In embodiments, R⁷ is —CHI₂. In embodiments, R⁷ is —CN. In embodiments, R⁷ is —OH. In embodiments, R⁷ is —NH₂. In embodiments, R⁷ is —COOH. In embodiments, R⁷ is —CONH₂. In embodiments, R⁷ is —C(O)NHCH₃. In embodiments, R⁷ is —C(O)N(CH₃)₂. In embodiments, R⁷ is —NO₂. In embodiments, R⁷ is —SH. In embodiments, R⁷ is —SO₃H. In embodiments, R⁷ is —OSO₃H. In embodiments, R⁷ is —SO₂CH₃. In embodiments, R⁷ is —SO₂NH₂. In embodiments, R⁷ is —SO₂NHCH₃. In embodiments, R⁷ is —NHNH₂. In embodiments, R⁷ is —ONH₂. In embodiments, R⁷ is —NHC(O)CH₃. In embodiments, R⁷ is —NHC(O)NH₂. In embodiments, R⁷ is —NHSO₂H. In embodiments, R⁷ is —NHC(O)H. In embodiments, R⁷ is —NHC(O)OH. In embodiments, R⁷ is —NHOH. In embodiments, R⁷ is —OCCl₃. In embodiments, R⁷ is —OCBr₃. In embodiments, R⁷ is —OCF₃. In embodiments, R⁷ is —OCI₃. In embodiments, R⁷ is —OCH₂Cl. In embodiments, R⁷ is —OCH₂Br. In embodiments, R⁷ is —OCH₂F. In embodiments, R⁷ is —OCH₂I. In embodiments, R⁷ is —OCHCl₂. In embodiments, R⁷ is —OCHBr₂. In embodiments, R⁷ is —OCHF₂. In embodiments, R⁷ is —OCHI₂. In embodiments, R⁷ is —N₃. In embodiments, R⁷ is unsubstituted C₁-C₄ alkyl. In embodiments, R⁷ is unsubstituted methyl. In embodiments, R⁷ is unsubstituted ethyl. In embodiments, R⁷ is unsubstituted propyl. In embodiments, R⁷ is unsubstituted n-propyl. In embodiments, R⁷ is unsubstituted isopropyl. In embodiments, R⁷ is unsubstituted butyl. In embodiments, R⁷ is unsubstituted n-butyl. In embodiments, R⁷ is unsubstituted isobutyl. In embodiments, R⁷ is unsubstituted tert-butyl. In embodiments, R⁷ is unsubstituted 2 to 6 membered heteroalkyl. In embodiments, R⁷ is unsubstituted methoxy. In embodiments, R⁷ is unsubstituted ethoxy. In embodiments, R⁷ is unsubstituted propoxy. In embodiments, R⁷ is unsubstituted n-propoxy. In embodiments, R⁷ is unsubstituted isopropoxy. In embodiments, R⁷ is unsubstituted butoxy. In embodiments, R⁷ is unsubstituted C₃-C₆ cycloalkyl. In embodiments, R⁷ is unsubstituted cyclopropyl. In embodiments, R⁷ is unsubstituted cyclobutyl. In embodiments, R⁷ is unsubstituted cyclopentyl. In embodiments, R⁷ is unsubstituted cyclohexyl.

In embodiments, R⁷ is hydrogen, —CF₃, —CN, —OH, —OCH₃, —C(O)OH, —SO₂CH₃, —SO₂NH₂, —SO₂NHCH₃, —NHC(O)CH₃, —C(O)NHCH₃, unsubstituted methyl, unsubstituted cyclopentyl,

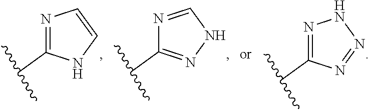 [SMILES: *c1ncc[nH]1 ; *c1nc[nH]n1 ; *c1nn[nH]n1]

In embodiments R7 is

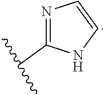 [SMILES: *c1ncc[nH]1]

In embodiments, R7 is

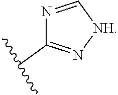 [SMILES: *c1nc[nH]n1]

In embodiments, R⁷ is

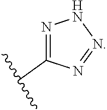 [SMILES: *c1nn[nH]n1]

In embodiments, R⁷ is substituted or unsubstituted aziridinyl. In embodiments, R⁷ is substituted or unsubstituted oxiranyl. In embodiments, R⁷ is substituted or unsubstituted thiiranyl. In embodiments, R⁷ is substituted or unsubstituted azetidinyl. In embodiments, R⁷ is substituted or unsubstituted oxetanyl. In embodiments, R⁷ is substituted or unsubstituted thietanyl. In embodiments, R⁷ is substituted or unsubstituted pyrrolidinyl. In embodiments, R⁷ is substituted or unsubstituted pyrrolyl. In embodiments, R⁷ is substituted or unsubstituted imidazolyl. In embodiments, R⁷ is substituted or unsubstituted imidazolinyl. In embodiments, R⁷ is substituted or unsubstituted pyrazolinyl. In embodiments, R⁷ is substituted or unsubstituted tetrahydrofuranyl. In embodiments, R⁷ is substituted or unsubstituted thiolanyl. In embodiments, R⁷ is substituted or unsubstituted piperidinyl. In embodiments, R⁷ is substituted or unsubstituted piperazinyl. In embodiments, R⁷ is substituted or unsubstituted pyranyl. In embodiments, R⁷ is substituted or unsubstituted morpholinyl. In embodiments, R⁷ is substituted or unsubstituted 1,4-dioxanyl. In embodiments, R⁷ is substituted or unsubstituted tetrahydropyranyl. In embodiments, R⁷ is substituted or unsubstituted thianyl. In embodiments, R⁷ is substituted or unsubstituted dithianyl. In embodiments, R⁷ is substituted or unsubstituted phenyl. In embodiments, R⁷ is substituted or unsubstituted thiofuranyl. In embodiments, R⁷ is substituted or unsubstituted imidazolyl. In embodiments, R⁷ is substituted or unsubstituted pyrazolyl. In embodiments, R⁷ is substituted or unsubstituted triazolyl. In embodiments, R⁷ is substituted or unsubstituted tetrazolyl. In embodiments, R⁷ is substituted or unsubstituted furanyl. In embodiments, R⁷ is substituted or unsubstituted oxazolyl. In embodiments, R⁷ is substituted or unsubstituted isooxazolyl. In embodiments, R⁷ is substituted or unsubstituted oxadiazolyl. In embodiments, R⁷ is substituted or unsubstituted oxatriazolyl. In embodiments, R⁷ is substituted or unsubstituted thienyl. In embodiments, R⁷ is substituted or unsubstituted thiazolyl. In embodiments, R⁷ is substituted or unsubstituted isothiazolyl. In embodiments, R⁷ is substituted or unsubstituted pyridinyl. In embodiments, R⁷ is substituted or unsubstituted pyrazinyl. In embodiments, R⁷ is substituted or unsubstituted pyrimidinyl. In embodiments, R⁷ is substituted or unsubstituted pyridazinyl. In embodiments, R⁷ is substituted or unsubstituted triazinyl. In embodiments, R⁷ is substituted or unsubstituted 1,3,5-triazinyl. In embodiments, R⁷ is substituted or unsubstituted 1,2,3-triazinyl. In embodiments, R⁷ is substituted or unsubstituted 1,2,4-triazinyl. In embodiments, R⁷ is substituted or unsubstituted indolyl. In embodiments, R⁷ is substituted or unsubstituted benzimidazolyl. In embodiments, R⁷ is substituted or unsubstituted indazolyl. In embodiments, R⁷ is substituted or unsubstituted benzotriazolyl. In embodiments, R⁷ is substituted or unsubstituted pyrrolopyrimidinyl. In embodiments, R⁷ is substituted or unsubstituted purinyl. In embodiments, R⁷ is substituted or unsubstituted indolizinyl. In embodiments, R⁷ is substituted or unsubstituted pyrrolopyriazinyl. In embodiments, R⁷ is substituted or unsubstituted pyrrolopyrimidinyl. In embodiments, R⁷ is substituted or unsubstituted imidazopyridazinyl. In embodiments, R⁷ is substituted or unsubstituted imidazopyridinyl. In embodiments, R⁷ is substituted or unsubstituted imidazopyrimidinyl. In embodiments, R⁷ is substituted or unsubstituted cinnolinyl. In embodiments, R⁷ is substituted or unsubstituted quinazolinyl. In embodiments, R⁷ is substituted or unsubstituted quinoxalinyl. In embodiments, R⁷ is substituted or unsubstituted phthalazinyl. In embodiments, R⁷ is substituted or unsubstituted pyridopyrazinyl. In embodiments, R⁷ is substituted or unsubstituted pteridinyl. In embodiments, R⁷ is substituted or unsubstituted pyrazolopyridinyl. In embodiments, R⁷ is substituted or unsubstituted quinolinyl. In embodiments, R⁷ is substituted or unsubstituted isoquinolinyl. In embodiments, R⁷ is substituted or unsubstituted naphthyridinyl. In embodiments, R⁷ is substituted or unsubstituted carbazolyl.

In embodiments, R⁷ is unsubstituted aziridinyl. In embodiments, R⁷ is unsubstituted oxiranyl. In embodiments, R⁷ is unsubstituted thiiranyl. In embodiments, R⁷ is unsubstituted azetidinyl. In embodiments, R⁷ is unsubstituted oxetanyl. In embodiments, R⁷ is unsubstituted thietanyl. In embodiments, R⁷ is unsubstituted pyrrolidinyl. In embodiments, R⁷ is unsubstituted pyrrolyl. In embodiments, R⁷ is unsubstituted imidazolyl. In embodiments, R⁷ is unsubstituted imidazolinyl. In embodiments, R⁷ is unsubstituted pyrazolinyl. In embodiments, R⁷ is unsubstituted tetrahydrofuranyl. In embodiments, R⁷ is unsubstituted thiolanyl. In embodiments, R⁷ is unsubstituted piperidinyl. In embodiments, R⁷ is unsubstituted piperazinyl. In embodiments, R⁷ is unsubstituted pyranyl. In embodiments, R⁷ is unsubstituted morpholinyl. In embodiments, R⁷ is unsubstituted 1,4-dioxanyl. In embodiments, R⁷ is unsubstituted tetrahydropyranyl. In embodiments, R⁷ is unsubstituted thianyl. In embodiments, R⁷ is unsubstituted dithianyl. In embodiments, R⁷ is unsubstituted phenyl. In embodiments, R⁷ is unsubstituted thiofuranyl. In embodiments, R⁷ is unsubstituted imidazolyl. In embodiments, R⁷ is unsubstituted pyrazolyl. In embodiments, R⁷ is unsubstituted triazolyl. In embodiments, R⁷ is unsubstituted tetrazolyl. In embodiments, R⁷ is unsubstituted furanyl. In embodiments, R⁷ is unsubstituted oxazolyl. In embodiments, R⁷ is unsubstituted isooxazolyl. In embodiments, R⁷ is unsubstituted oxadiazolyl. In embodiments, R⁷ is unsubstituted oxatriazolyl. In embodiments, R⁷ is unsubstituted thienyl. In embodiments, R⁷ is unsubstituted thiazolyl. In embodiments, R⁷ is unsubstituted isothiazolyl. In embodiments, R⁷ is unsubstituted pyridinyl. In embodiments, R⁷ is unsubstituted pyrazinyl. In embodiments, R⁷ is unsubstituted pyrimidinyl. In embodiments, R⁷ is unsubstituted pyridazinyl. In embodiments, R⁷ is unsubstituted triazinyl. In embodiments, R⁷ is unsubstituted 1,3,5-triazinyl. In embodiments, R⁷ is unsubstituted 1,2,3-triazinyl. In embodiments, R⁷ is unsubstituted 1,2,4-triazinyl. In embodiments, R⁷ is unsubstituted indolyl. In embodiments, R⁷ is unsubstituted benzimidazolyl. In embodiments, R⁷ is unsubstituted indazolyl. In embodiments, R⁷ is unsubstituted benzotriazolyl. In embodiments, R⁷ is unsubstituted pyrrolopyrimidinyl. In embodiments, R⁷ is unsubstituted purinyl. In embodiments, R⁷ is unsubstituted indolizinyl. In embodiments, R⁷ is unsubstituted pyrrolopyriazinyl. In embodiments, R⁷ is unsubstituted pyrrolopyrimidinyl. In embodiments, R⁷ is unsubstituted imidazopyridazinyl. In embodiments, R⁷ is unsubstituted imidazopyridinyl. In embodiments, R⁷ is unsubstituted imidazopyrimidinyl. In embodiments, R⁷ is unsubstituted cinnolinyl. In embodiments, R⁷ is unsubstituted quinazolinyl. In embodiments, R⁷ is unsubstituted quinoxalinyl. In embodiments, R⁷ is unsubstituted phthalazinyl. In embodiments, R⁷ is unsubstituted pyridopyrazinyl. In embodiments, R⁷ is unsubstituted pteridinyl. In embodiments, R⁷ is unsubstituted pyrazolopyridinyl. In embodiments, R⁷ is unsubstituted quinolinyl. In embodiments, R⁷ is unsubstituted isoquinolinyl. In embodiments, R⁷ is unsubstituted naphthyridinyl. In embodiments, R⁷ is unsubstituted carbazolyl.

In embodiments, a substituted R^{7A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{7A} is substituted with a plurality of groups selected from substituent groups, size-limited substituent groups, and lower substituent groups; each substituent group, size-limited substituent group, and/or lower substituent group may optionally be different. In embodiments, when R^{7A} is substituted, it is substituted with at least one substituent group. In embodiments, when R^{7A} is substituted, it is substituted with at least one size-limited substituent group. In embodiments, when R^{7A} is substituted, it is substituted with at least one lower substituent group.

In embodiments, a substituted R^{7B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{7B} is substituted with a plurality of groups selected from substituent groups, size-limited substituent groups, and lower substituent groups; each substituent group, size-limited substituent group, and/or lower substituent group may optionally be different. In embodiments, when R^{7B} is substituted, it is substituted with at least one substituent group. In embodiments, when R^{7B} is substituted, it is substituted with at least one size-limited substituent group. In embodiments, when R^{7B} is substituted, it is substituted with at least one lower substituent group.

In embodiments, a substituted R^{7C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{7C} is substituted with a plurality of groups selected from substituent groups, size-limited substituent groups, and lower substituent groups; each substituent group, size-limited substituent group, and/or lower substituent group may optionally be different. In embodiments, when R^{7C} is substituted, it is substituted with at least one substituent group. In embodiments, when R^{7C} is substituted, it is substituted with at least one size-limited substituent group. In embodiments, when R^{7C} is substituted, it is substituted with at least one lower substituent group.

In embodiments, a substituted ring formed when R^{7B} and R^{7C} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{7B} and R^{7C} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{7B} and R^{7C} substituents bonded to the same nitrogen atom are joined is substituted, it is substituted with at least one substituent group. In embodiments, when the substituted ring formed when R^{7B} and R^{7C}substituents bonded to the same nitrogen atom are joined is substituted, it is substituted with at least one size-limited substituent group. In embodiments, when the substituted ring formed when R^{7B} and R^{7C} substituents bonded to the same nitrogen atom are joined is substituted, it is substituted with at least one lower substituent group.

In embodiments, a substituted R^{7D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{7D} is substituted with a plurality of groups selected from substituent groups, size-limited substituent groups, and lower substituent groups; each substituent group, size-limited substituent group, and/or lower substituent group may optionally be different. In embodiments, when R^{7D} is substituted, it is substituted with at least one substituent group. In embodiments, when R^{7D} is substituted, it is substituted with at least one size-limited substituent group. In embodiments, when R^{7D} is substituted, it is substituted with at least one lower substituent group.

In embodiments, R^{7A} is hydrogen. In embodiments, R^{7A} is unsubstituted C₁-C₄ alkyl. In embodiments, R^{7A} is unsubstituted methyl. In embodiments, R^{7A} is unsubstituted ethyl. In embodiments, R^{7A} is unsubstituted propyl. In embodiments, R^{7A} is unsubstituted n-propyl. In embodiments, R^{7A} is unsubstituted isopropyl. In embodiments, R^{7A} is unsubstituted butyl. In embodiments, R^{7A} is unsubstituted n-butyl. In embodiments, R^{7A} is unsubstituted isobutyl. In embodiments, R^{7A} is unsubstituted tert-butyl.

In embodiments, R^{7B} is hydrogen. In embodiments, R^{7B} is unsubstituted C₁-C₄ alkyl. In embodiments, R^{7B} is unsubstituted methyl. In embodiments, R^{7B} is unsubstituted ethyl. In embodiments, R^{7B} is unsubstituted propyl. In embodiments, R^{7B} is unsubstituted n-propyl. In embodiments, R^{7B} is unsubstituted isopropyl. In embodiments, R^{7B} is unsubstituted butyl. In embodiments, R^{7B} is unsubstituted n-butyl. In embodiments, R^{7B} is unsubstituted isobutyl. In embodiments, R^{7B} is unsubstituted tert-butyl.

In embodiments, R^{7C} is hydrogen. In embodiments, R^{7C} is unsubstituted C₁-C₄ alkyl. In embodiments, R^{7C} is unsubstituted methyl. In embodiments, R^{7C} is unsubstituted ethyl. In embodiments, R^{7C} is unsubstituted propyl. In embodiments, R^{7C} is unsubstituted n-propyl. In embodiments, R^{7C} is unsubstituted isopropyl. In embodiments, R^{7C} is unsubstituted butyl. In embodiments, R^{7C} is unsubstituted n-butyl. In embodiments, R^{7C} is unsubstituted isobutyl. In embodiments, R^{7C} is unsubstituted tert-butyl.

In embodiments, R^{7D} is hydrogen. In embodiments, R^{7D} is unsubstituted C₁-C₄ alkyl. In embodiments, R^{7D} is unsubstituted methyl. In embodiments, R^{7D} is unsubstituted ethyl. In embodiments, R^{7D} is unsubstituted propyl. In embodiments, R^{7D} is unsubstituted n-propyl. In embodiments, R^{7D} is unsubstituted isopropyl. In embodiments, R^{7D} is unsubstituted butyl. In embodiments, R^{7D} is unsubstituted n-butyl. In embodiments, R^{7D} is unsubstituted isobutyl. In embodiments, R^{7D} is unsubstituted tert-butyl.

In embodiments, -L⁷-R⁷ is hydrogen, —CH₃, —COOH, —CH₂CF₃,

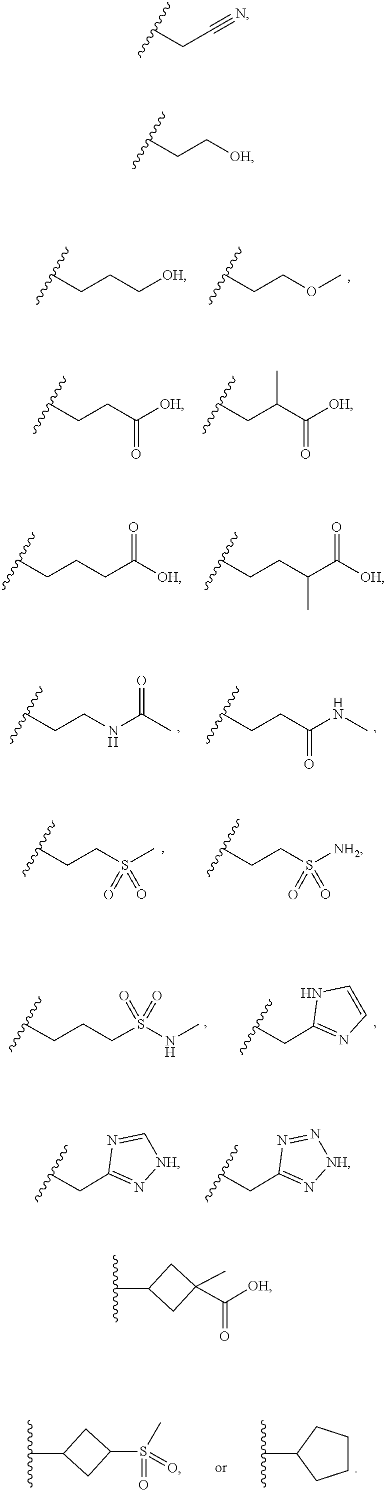

In embodiments, -L⁷-R⁷ is hydrogen. In embodiments, -L⁷-R⁷ is —CH₃. In embodiments, -L⁷-R⁷ is —COOH. In embodiments, -L⁷-R⁷ is —CH₂CF₃. In embodiments, -L⁷-R⁷ is

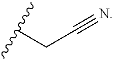 [SMILES: *CC#N]

In embodiments, -L⁷-R⁷ is

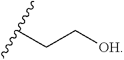 [SMILES: *CCO]

In embodiments, -L⁷-R⁷ is

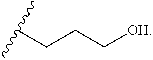 [SMILES: *CCCO]

In embodiments, -L⁷-R⁷ is

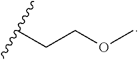 [SMILES: *CCOC]

In embodiments, -L⁷-R⁷ is

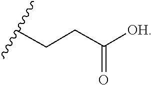 [SMILES: *CCC(=O)O]

In embodiments, -L⁷-R⁷ is

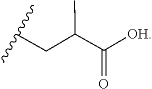 [SMILES: *CC(C)C(=O)O]

In embodiments, -L⁷-R⁷ is

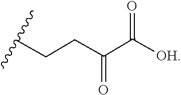 [SMILES: *CCC(=O)C(=O)O]

In embodiments, -L⁷-R⁷ is

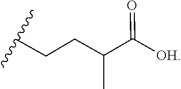 [SMILES: *CCC(C)C(=O)O]

In embodiments, -L⁷-R⁷ is

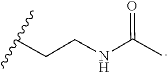 [SMILES: *CCNC(C)=O]

In embodiments, -L⁷-R⁷ is

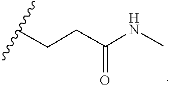 [SMILES: *CCC(=O)NC]

In embodiments, -L⁷-R⁷ is

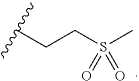 [SMILES: *CCS(C)(=O)=O]

In embodiments, -L⁷-R⁷ is

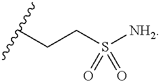 [SMILES: *CCS(N)(=O)=O]

In embodiments, -L⁷-R⁷ is

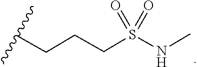 [SMILES: *CCCS(=O)(=O)NC]

In embodiments, -L⁷-R⁷ is

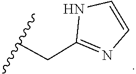 [SMILES: *Cc1ncc[nH]1]

In embodiments, -L⁷-R⁷ is

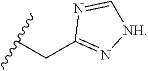 [SMILES: *Cc1nc[nH]n1]

In embodiments, -L⁷-R⁷ is

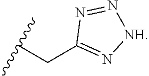 [SMILES: *Cc1nn[nH]n1]

In embodiments, -L⁷-R⁷ is

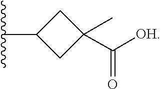 [SMILES: *C1CC(C)(C(=O)O)C1]

In embodiments, -L⁷-R⁷ is

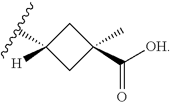 [SMILES: *[C@H]1C[C@](C)(C(=O)O)C1]

In embodiments, -L⁷-R⁷ is

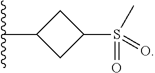 [SMILES: *C1CC(S(C)(=O)=O)C1]

In embodiments, -L⁷-R⁷ is

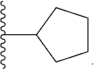 [SMILES: *C1CCCC1]

In embodiments, a substituted R⁸ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁸ is substituted with a plurality of groups selected from substituent groups, size-limited substituent groups, and lower substituent groups; each substituent group, size-limited substituent group, and/or lower substituent group may optionally be different. In embodiments, when R⁸ is substituted, it is substituted with at least one substituent group. In embodiments, when R⁸ is substituted, it is substituted with at least one size-limited substituent group. In embodiments, when R⁸ is substituted, it is substituted with at least one lower substituent group.

In embodiments, R⁸ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁸ is hydrogen. In embodiments, R⁸ is halogen. In embodiments, R⁸ is —F. In embodiments, R⁸ is —Cl. In embodiments, R⁸ is —Br. In embodiments, R⁸ is —I. In embodiments, R⁸ is —CCl₃. In embodiments, R⁸ is —CBr₃. In embodiments, R⁸ is —CF₃. In embodiments, R⁸ is —CI₃. In embodiments, R⁸ is —CH₂C₁. In embodiments, R⁸ is —CH₂Br. In embodiments, R⁸ is —CH₂F. In embodiments, R⁸ is —CH₂I. In embodiments, R⁸ is —CHCl₂. In embodiments, R′ is —CHBr₂. In embodiments, R′ is —CHF₂. In embodiments, R′ is —CHI₂. In embodiments, R¹ is —CN. In embodiments, R¹ is —OH. In embodiments, R¹ is —NH₂. In embodiments, R¹ is —COOH. In embodiments, R¹ is —CONH₂. In embodiments, R¹ is —NO₂. In embodiments, R′ is —SH. In embodiments, R′ is —SO₃H. In embodiments, R′ is —OSO₃H. In embodiments, R¹ is —SO₂NH₂. In embodiments, R¹ is —NHNH₂. In embodiments, R¹ is —ONH₂. In embodiments, R′ is —NHC(O)NH₂. In embodiments, R′ is —NHSO₂H. In embodiments, R⁸ is —NHC(O)H. In embodiments, R⁸ is —NHC(O)OH. In embodiments, R⁸ is —NHOH. In embodiments, R⁸ is —OCCl₃. In embodiments, R⁸ is —OCBr₃. In embodiments, R⁸ is —OCF₃. In embodiments, R⁸ is —OCI₃. In embodiments, R⁸ is —OCH₂Cl. In embodiments, R⁸ is —OCH₂Br. In embodiments, R⁸ is —OCH₂F. In embodiments, R⁸ is —OCH₂I. In embodiments, R⁸ is —OCHCl₂. In embodiments, R⁸ is —OCHBr₂. In embodiments, R⁸ is —OCHF₂. In embodiments, R⁸ is —OCHI₂. In embodiments, R⁸ is —N₃. In embodiments, R⁸ is unsubstituted C₁-C₄ alkyl. In embodiments, R⁸ is unsubstituted methyl. In embodiments, R⁸ is unsubstituted ethyl. In embodiments, R⁸ is unsubstituted propyl. In embodiments, R⁸ is unsubstituted n-propyl. In embodiments, R⁸ is unsubstituted isopropyl. In embodiments, R⁸ is unsubstituted butyl. In embodiments, R⁸ is unsubstituted n-butyl. In embodiments, R⁸ is unsubstituted isobutyl. In embodiments, R⁸ is unsubstituted tert-butyl. In embodiments, R⁸ is unsubstituted 2 to 6 membered heteroalkyl. In embodiments, R⁸ is unsubstituted methoxy. In embodiments, R⁸ is unsubstituted ethoxy. In embodiments, R⁸ is unsubstituted propoxy. In embodiments, R⁸ is unsubstituted n-propoxy. In embodiments, R⁸ is unsubstituted isopropoxy. In embodiments, R⁸ is unsubstituted butoxy.

In embodiments, a substituted R^{8A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{8A} is substituted with a plurality of groups selected from substituent groups, size-limited substituent groups, and lower substituent groups; each substituent group, size-limited substituent group, and/or lower substituent group may optionally be different. In embodiments, when R^{8A} is substituted, it is substituted with at least one substituent group. In embodiments, when R^{8A} is substituted, it is substituted with at least one size-limited substituent group. In embodiments, when R^{8A} is substituted, it is substituted with at least one lower substituent group.

In embodiments, a substituted R^{8B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{8B} is substituted with a plurality of groups selected from substituent groups, size-limited substituent groups, and lower substituent groups; each substituent group, size-limited substituent group, and/or lower substituent group may optionally be different. In embodiments, when R^{8B} is substituted, it is substituted with at least one substituent group. In embodiments, when R^{8B} is substituted, it is substituted with at least one size-limited substituent group. In embodiments, when R^{8B} is substituted, it is substituted with at least one lower substituent group.

In embodiments, a substituted R^{8C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{8C} is substituted with a plurality of groups selected from substituent groups, size-limited substituent groups, and lower substituent groups; each substituent group, size-limited substituent group, and/or lower substituent group may optionally be different. In embodiments, when R^{8C} is substituted, it is substituted with at least one substituent group. In embodiments, when R^{8C} is substituted, it is substituted with at least one size-limited substituent group. In embodiments, when R^{8C} is substituted, it is substituted with at least one lower substituent group.

In embodiments, a substituted ring formed when R^{8B} and R^{8C} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{8B} and R^{8C} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{8B} and R^{8C} substituents bonded to the same nitrogen atom are joined is substituted, it is substituted with at least one substituent group. In embodiments, when the substituted ring formed when R^{8B} and R^{8C} substituents bonded to the same nitrogen atom are joined is substituted, it is substituted with at least one size-limited substituent group. In embodiments, when the substituted ring formed when R^{8B} and R^{8C} substituents bonded to the same nitrogen atom are joined is substituted, it is substituted with at least one lower substituent group.

In embodiments, a substituted R^{8D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{8D} is substituted with a plurality of groups selected from substituent groups, size-limited substituent groups, and lower substituent groups; each substituent group, size-limited substituent group, and/or lower substituent group may optionally be different. In embodiments, when R^{8D} is substituted, it is substituted with at least one substituent group. In embodiments, when R^{8D} is substituted, it is substituted with at least one size-limited substituent group. In embodiments, when R^{8D} is substituted, it is substituted with at least one lower substituent group.

In embodiments, R^{8A} is hydrogen. In embodiments, R^{8A} is unsubstituted C₁-C₄ alkyl. In embodiments, R^{8A} is unsubstituted methyl. In embodiments, R^{8A} is unsubstituted ethyl. In embodiments, R^{8A} is unsubstituted propyl. In embodiments, R^{8A} is unsubstituted n-propyl. In embodiments, R^{8A} is unsubstituted isopropyl. In embodiments, R^{8A} is unsubstituted butyl. In embodiments, R^{8A} is unsubstituted n-butyl. In embodiments, R^{8A} is unsubstituted isobutyl. In embodiments, R^{8A} is unsubstituted tert-butyl.

In embodiments, R^{8B} is hydrogen. In embodiments, R^{8B} is unsubstituted C₁-C₄ alkyl. In embodiments, R^{8B} is unsubstituted methyl. In embodiments, R^{8B} is unsubstituted ethyl. In embodiments, R^{8B} is unsubstituted propyl. In embodiments, R^{8B} is unsubstituted n-propyl. In embodiments, R^{8B} is unsubstituted isopropyl. In embodiments, R^{8B} is unsubstituted butyl. In embodiments, R^{8B} is unsubstituted n-butyl. In embodiments, R^{8B} is unsubstituted isobutyl. In embodiments, R^{8B} is unsubstituted tert-butyl.

In embodiments, R^{8C} is hydrogen. In embodiments, R^{8C} is unsubstituted C₁-C₄ alkyl. In embodiments, R^{8C} is unsubstituted methyl. In embodiments, R^{8C} is unsubstituted ethyl. In embodiments, R^{8C} is unsubstituted propyl. In embodiments, R^{8C} is unsubstituted n-propyl. In embodiments, R^{8C} is unsubstituted isopropyl. In embodiments, R^{8C} is unsubstituted butyl. In embodiments, R^{8C} is unsubstituted n-butyl. In embodiments, R^{8C} is unsubstituted isobutyl. In embodiments, R^{8C} is unsubstituted tert-butyl.

In embodiments, R^{8D} is hydrogen. In embodiments, R^{8D} is unsubstituted C₁-C₄ alkyl. In embodiments, R^{8D} is unsubstituted methyl. In embodiments, R^{8D} is unsubstituted ethyl. In embodiments, R^{8D} is unsubstituted propyl. In embodiments, R^{8D} is unsubstituted n-propyl. In embodiments, R^{8D} is unsubstituted isopropyl. In embodiments, R^{8D} is unsubstituted butyl. In embodiments, R^{8D} is unsubstituted n-butyl. In embodiments, R^{8D} is unsubstituted isobutyl. In embodiments, R^{8D} is unsubstituted tert-butyl.

In embodiments, a substituted R⁹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁹ is substituted with a plurality of groups selected from substituent groups, size-limited substituent groups, and lower substituent groups; each substituent group, size-limited substituent group, and/or lower substituent group may optionally be different. In embodiments, when R⁹ is substituted, it is substituted with at least one substituent group. In embodiments, when R⁹ is substituted, it is substituted with at least one size-limited substituent group. In embodiments, when R⁹ is substituted, it is substituted with at least one lower substituent group.

In embodiments, R⁹ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁹ is hydrogen. In embodiments, R⁹ is halogen. In embodiments, R⁹ is —F. In embodiments, R⁹ is —Cl. In embodiments, R⁹ is —Br. In embodiments, R⁹ is —I. In embodiments, R⁹ is —CCl₃. In embodiments, R⁹ is —CBr₃. In embodiments, R⁹ is —CF₃. In embodiments, R⁹ is —CI₃. In embodiments, R⁹ is —CH₂C₁. In embodiments, R⁹ is —CH₂Br. In embodiments, R⁹ is —CH₂F. In embodiments, R⁹ is —CH₂I. In embodiments, R⁹ is —CHCl₂. In embodiments, R⁹ is —CHBr₂. In embodiments, R⁹ is —CHF₂. In embodiments, R⁹ is —CHI₂. In embodiments, R⁹ is —CN. In embodiments, R⁹ is —OH. In embodiments, R⁹ is —NH₂. In embodiments, R⁹ is —COOH. In embodiments, R⁹ is —CONH₂. In embodiments, R⁹ is —NO₂. In embodiments, R⁹ is —SH. In embodiments, R⁹ is —SO₃H. In embodiments, R⁹ is —OSO₃H. In embodiments, R⁹ is —SO₂NH₂. In embodiments, R⁹ is —NHNH₂. In embodiments, R⁹ is —ONH₂. In embodiments, R⁹ is —NHC(O)NH₂. In embodiments, R⁹ is —NHSO₂H. In embodiments, R⁹ is —NHC(O)H. In embodiments, R⁹ is —NHC(O)OH. In embodiments, R⁹ is —NHOH. In embodiments, R⁹ is —OCCl₃. In embodiments, R⁹ is —OCBr₃. In embodiments, R⁹ is —OCF₃. In embodiments, R⁹ is —OCI₃. In embodiments, R⁹ is —OCH₂Cl. In embodiments, R⁹ is —OCH₂Br. In embodiments, R⁹ is —OCH₂F. In embodiments, R⁹ is —OCH₂I. In embodiments, R⁹ is —OCHCl₂. In embodiments, R⁹ is —OCHBr₂. In embodiments, R⁹ is —OCHF₂. In embodiments, R⁹ is —OCHI₂. In embodiments, R⁹ is —N₃. In embodiments, R⁹ is unsubstituted C₁-C₄ alkyl. In embodiments, R⁹ is unsubstituted methyl. In embodiments, R⁹ is unsubstituted ethyl. In embodiments, R⁹ is unsubstituted propyl. In embodiments, R⁹ is unsubstituted n-propyl. In embodiments, R⁹ is unsubstituted isopropyl. In embodiments, R⁹ is unsubstituted butyl. In embodiments, R⁹ is unsubstituted n-butyl. In embodiments, R⁹ is unsubstituted isobutyl. In embodiments, R⁹ is unsubstituted tert-butyl. In embodiments, R⁹ is unsubstituted 2 to 6 membered heteroalkyl. In embodiments, R⁹ is unsubstituted methoxy. In embodiments, R⁹ is unsubstituted ethoxy. In embodiments, R⁹ is unsubstituted propoxy. In embodiments, R⁹ is unsubstituted n-propoxy. In embodiments, R⁹ is unsubstituted isopropoxy. In embodiments, R⁹ is unsubstituted butoxy.

In embodiments, a substituted R^{9A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{9A} is substituted with a plurality of groups selected from substituent groups, size-limited substituent groups, and lower substituent groups; each substituent group, size-limited substituent group, and/or lower substituent group may optionally be different. In embodiments, when R^{9A} is substituted, it is substituted with at least one substituent group. In embodiments, when R^{9A} is substituted, it is substituted with at least one size-limited substituent group. In embodiments, when R^{9A} is substituted, it is substituted with at least one lower substituent group.

In embodiments, a substituted R^{9B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{9B} is substituted with a plurality of groups selected from substituent groups, size-limited substituent groups, and lower substituent groups; each substituent group, size-limited substituent group, and/or lower substituent group may optionally be different. In embodiments, when R^{9B} is substituted, it is substituted with at least one substituent group. In embodiments, when R^{9B} is substituted, it is substituted with at least one size-limited substituent group. In embodiments, when R^{9B} is substituted, it is substituted with at least one lower substituent group.

In embodiments, a substituted R^{9C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{9C} is substituted with a plurality of groups selected from substituent groups, size-limited substituent groups, and lower substituent groups; each substituent group, size-limited substituent group, and/or lower substituent group may optionally be different. In embodiments, when R^{9C} is substituted, it is substituted with at least one substituent group. In embodiments, when R^{9C} is substituted, it is substituted with at least one size-limited substituent group. In embodiments, when R^{9C} is substituted, it is substituted with at least one lower substituent group.

In embodiments, a substituted ring formed when R^{9B} and R^{9C} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{9B} and R^{9C} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{9B} and R^{9C} substituents bonded to the same nitrogen atom are joined is substituted, it is substituted with at least one substituent group. In embodiments, when the substituted ring formed when R^{9B} and R^{9C}substituents bonded to the same nitrogen atom are joined is substituted, it is substituted with at least one size-limited substituent group. In embodiments, when the substituted ring formed when R^{9B} and R^{9C} substituents bonded to the same nitrogen atom are joined is substituted, it is substituted with at least one lower substituent group.

In embodiments, a substituted R^{9D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{9D} is substituted with a plurality of groups selected from substituent groups, size-limited substituent groups, and lower substituent groups; each substituent group, size-limited substituent group, and/or lower substituent group may optionally be different. In embodiments, when R^{9D} is substituted, it is substituted with at least one substituent group. In embodiments, when R^{9D} is substituted, it is substituted with at least one size-limited substituent group. In embodiments, when R^{9D} is substituted, it is substituted with at least one lower substituent group.

In embodiments, R^{9A} is hydrogen. In embodiments, R^{9A} is unsubstituted C₁-C₄ alkyl. In embodiments, R^{9A} is unsubstituted methyl. In embodiments, R^{9A} is unsubstituted ethyl. In embodiments, R^{9A} is unsubstituted propyl. In embodiments, R^{9A} is unsubstituted n-propyl. In embodiments, R^{9A} is unsubstituted isopropyl. In embodiments, R^{9A} is unsubstituted butyl. In embodiments, R^{9A} is unsubstituted n-butyl. In embodiments, R^{9A} is unsubstituted isobutyl. In embodiments, R^{9A} is unsubstituted tert-butyl.

In embodiments, R^{9B} is hydrogen. In embodiments, R^{9B} is unsubstituted C₁-C₄ alkyl. In embodiments, R^{9B} is unsubstituted methyl. In embodiments, R^{9B} is unsubstituted ethyl. In embodiments, R^{9B} is unsubstituted propyl. In embodiments, R^{9B} is unsubstituted n-propyl. In embodiments, R^{9B} is unsubstituted isopropyl. In embodiments, R^{9B} is unsubstituted butyl. In embodiments, R^{9B} is unsubstituted n-butyl. In embodiments, R^{9B} is unsubstituted isobutyl. In embodiments, R^{9B} is unsubstituted tert-butyl.

In embodiments, R^{9C} is hydrogen. In embodiments, R^{9C} is unsubstituted C₁-C₄ alkyl. In embodiments, R^{9C} is unsubstituted methyl. In embodiments, R^{9C} is unsubstituted ethyl. In embodiments, R^{9C} is unsubstituted propyl. In embodiments, R^{9C} is unsubstituted n-propyl. In embodiments, R^{9C} is unsubstituted isopropyl. In embodiments, R^{9C} is unsubstituted butyl. In embodiments, R^{9C} is unsubstituted n-butyl. In embodiments, R^{9C} is unsubstituted isobutyl. In embodiments, R^{9C} is unsubstituted tert-butyl.

In embodiments, R^{9D} is hydrogen. In embodiments, R^{9D} is unsubstituted C₁-C₄ alkyl. In embodiments, R^{9D} is unsubstituted methyl. In embodiments, R^{9D} is unsubstituted ethyl. In embodiments, R^{9D} is unsubstituted propyl. In embodiments, R^{9D} is unsubstituted n-propyl. In embodiments, R^{9D} is unsubstituted isopropyl. In embodiments, R^{9D} is unsubstituted butyl. In embodiments, R^{9D} is unsubstituted n-butyl. In embodiments, R^{9D} is unsubstituted isobutyl. In embodiments, R^{9D} is unsubstituted tert-butyl.

In embodiments, a substituted R¹⁰ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁰ is substituted with a plurality of groups selected from substituent groups, size-limited substituent groups, and lower substituent groups; each substituent group, size-limited substituent group, and/or lower substituent group may optionally be different. In embodiments, when R¹⁰ is substituted, it is substituted with at least one substituent group. In embodiments, when R¹⁰ is substituted, it is substituted with at least one size-limited substituent group. In embodiments, when R¹⁰ is substituted, it is substituted with at least one lower substituent group.

In embodiments, R¹⁰ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂C₁, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R¹⁰ is hydrogen. In embodiments, R¹⁰ is halogen. In embodiments, R¹⁰ is —F. In embodiments, R¹⁰ is —Cl. In embodiments, R¹⁰ is —Br. In embodiments, R¹⁰ is —I. In embodiments, R¹⁰ is —CCl₃. In embodiments, R¹⁰ is —CBr₃. In embodiments, R¹⁰ is —CF₃. In embodiments, R¹⁰ is —CI₃. In embodiments, R¹⁰ is —CH₂C₁. In embodiments, R¹⁰ is —CH₂Br. In embodiments, R¹⁰ is —CH₂F. In embodiments, R¹⁰ is —CH₂J. In embodiments, R¹⁰ is —CHCl₂. In embodiments, R¹⁰ is —CHBr₂. In embodiments, R¹⁰ is —CHF₂. In embodiments, R¹⁰ is —CHI₂. In embodiments, R¹⁰ is —CN. In embodiments, R¹⁰ is —OH. In embodiments, R¹⁰ is —NH₂. In embodiments, R¹⁰ is —COOH. In embodiments, R¹⁰ is —CONH₂. In embodiments, R¹⁰ is —NO₂. In embodiments, R¹⁰ is —SH. In embodiments, R¹⁰ is —SO₃H. In embodiments, R¹⁰ is —OSO₃H. In embodiments, R¹⁰ is —SO₂NH₂. In embodiments, R¹⁰ is —NHNH₂. In embodiments, R¹⁰ is —ONH₂. In embodiments, R¹⁰ is —NHC(O)NH₂. In embodiments, R¹⁰ is —NHSO₂H. In embodiments, R¹⁰ is —NHC(O)H. In embodiments, R¹⁰ is —NHC(O)OH. In embodiments, R¹⁰ is —NHOH. In embodiments, R¹⁰ is —OCCl₃. In embodiments, R¹⁰ is —OCBr₃. In embodiments, R¹⁰ is —OCF₃. In embodiments, R¹⁰ is —OCI₃. In embodiments, R¹⁰ is —OCH₂Cl. In embodiments, R¹⁰ is —OCH₂Br. In embodiments, R¹⁰ is —OCH₂F. In embodiments, R¹⁰ is —OCH₂I. In embodiments, R¹⁰ is —OCHCl₂. In embodiments, R¹⁰ is —OCHBr₂. In embodiments, R¹⁰ is —OCHF₂. In embodiments, R¹⁰ is —OCHI₂. In embodiments, R¹⁰ is —N₃. In embodiments, R¹⁰ is unsubstituted C₁-C₄ alkyl. In embodiments, R¹⁰ is unsubstituted methyl. In embodiments, R¹⁰ is unsubstituted ethyl. In embodiments, R¹⁰ is unsubstituted propyl. In embodiments, R¹⁰ is unsubstituted n-propyl. In embodiments, R¹⁰ is unsubstituted isopropyl. In embodiments, R¹⁰ is unsubstituted butyl. In embodiments, R¹⁰ is unsubstituted n-butyl. In embodiments, R¹⁰ is unsubstituted isobutyl. In embodiments, R¹⁰ is unsubstituted tert-butyl. In embodiments, R¹⁰ is unsubstituted 2 to 6 membered heteroalkyl. In embodiments, R¹⁰ is unsubstituted methoxy. In embodiments, R¹⁰ is unsubstituted ethoxy. In embodiments, R¹⁰ is unsubstituted propoxy. In embodiments, R¹⁰ is unsubstituted n-propoxy. In embodiments, R¹⁰ is unsubstituted isopropoxy. In embodiments, R¹⁰ is unsubstituted butoxy.

In embodiments, a substituted R^{10A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{10A} is substituted with a plurality of groups selected from substituent groups, size-limited substituent groups, and lower substituent groups; each substituent group, size-limited substituent group, and/or lower substituent group may optionally be different. In embodiments, when R^{10A} is substituted, it is substituted with at least one substituent group. In embodiments, when R^{10A} is substituted, it is substituted with at least one size-limited substituent group. In embodiments, when R^{10A} is substituted, it is substituted with at least one lower substituent group.

In embodiments, a substituted R^{10B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{10B} is substituted with a plurality of groups selected from substituent groups, size-limited substituent groups, and lower substituent groups; each substituent group, size-limited substituent group, and/or lower substituent group may optionally be different. In embodiments, when R^{10B} is substituted, it is substituted with at least one substituent group. In embodiments, when R^{10B} is substituted, it is substituted with at least one size-limited substituent group. In embodiments, when R^{10B} is substituted, it is substituted with at least one lower substituent group.

In embodiments, a substituted RiOc (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{10C} is substituted with a plurality of groups selected from substituent groups, size-limited substituent groups, and lower substituent groups; each substituent group, size-limited substituent group, and/or lower substituent group may optionally be different. In embodiments, when RiOc is substituted, it is substituted with at least one substituent group. In embodiments, when R^{10C} is substituted, it is substituted with at least one size-limited substituent group. In embodiments, when R^{10C} is substituted, it is substituted with at least one lower substituent group.

In embodiments, a substituted ring formed when R^{10B} and R^{10c} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{10B} and R^{10c} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{10B} and R^{10c} substituents bonded to the same nitrogen atom are joined is substituted, it is substituted with at least one substituent group. In embodiments, when the substituted ring formed when R^{10B} and R^{10c} substituents bonded to the same nitrogen atom are joined is substituted, it is substituted with at least one size-limited substituent group. In embodiments, when the substituted ring formed when R^{10B} and R^{10c} substituents bonded to the same nitrogen atom are joined is substituted, it is substituted with at least one lower substituent group.

In embodiments, a substituted R^{10D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{10D} is substituted with a plurality of groups selected from substituent groups, size-limited substituent groups, and lower substituent groups; each substituent group, size-limited substituent group, and/or lower substituent group may optionally be different. In embodiments, when R^{10D} is substituted, it is substituted with at least one substituent group. In embodiments, when R^{10D} is substituted, it is substituted with at least one size-limited substituent group. In embodiments, when R^{10D} is substituted, it is substituted with at least one lower substituent group.

In embodiments, R^{10A} is hydrogen. In embodiments, R^{10A} is unsubstituted C₁-C₄ alkyl. In embodiments, R^{10A} is unsubstituted methyl. In embodiments, R^{10A} is unsubstituted ethyl. In embodiments, R^{10A} is unsubstituted propyl. In embodiments, R^{10A} is unsubstituted n-propyl. In embodiments, R^{10A} is unsubstituted isopropyl. In embodiments, R^{10A} is unsubstituted butyl. In embodiments, R^{10A} is unsubstituted n-butyl. In embodiments, R^{10A} is unsubstituted isobutyl. In embodiments, R^{10A} is unsubstituted tert-butyl.

In embodiments, R^{10B} is hydrogen. In embodiments, R^{10B} is unsubstituted C₁-C₄ alkyl. In embodiments, R^{10B} is unsubstituted methyl. In embodiments, R^{10B} is unsubstituted ethyl. In embodiments, R^{10B} is unsubstituted propyl. In embodiments, R^{10B} is unsubstituted n-propyl. In embodiments, R^{10B} is unsubstituted isopropyl. In embodiments, R^{0B} is unsubstituted butyl. In embodiments, R^{10B} is unsubstituted n-butyl. In embodiments, R^{0B} is unsubstituted isobutyl. In embodiments, R^{10B} is unsubstituted tert-butyl.

In embodiments, R^{10C} is hydrogen. In embodiments, RiOC is unsubstituted C₁-C₄ alkyl. In embodiments, R^{10C} is unsubstituted methyl. In embodiments, R^{10C} is unsubstituted ethyl. In embodiments, R^{10C} is unsubstituted propyl. In embodiments, R^{10C} is unsubstituted n-propyl. In embodiments, R^{10C} is unsubstituted isopropyl. In embodiments, R^{10C} is unsubstituted butyl. In embodiments, R^{10C} is unsubstituted n-butyl. In embodiments, R^{10C} is unsubstituted isobutyl. In embodiments, R^{10C} is unsubstituted tert-butyl.

In embodiments, R^{10D} is hydrogen. In embodiments, R^{10D} is unsubstituted C₁-C₄ alkyl. In embodiments, R^{10D} is unsubstituted methyl. In embodiments, R^{10D} is unsubstituted ethyl. In embodiments, R^{10D} is unsubstituted propyl. In embodiments, R^{10D} is unsubstituted n-propyl. In embodiments, R^{10D} is unsubstituted isopropyl. In embodiments, R^{10D} is unsubstituted butyl. In embodiments, R^{10D} is unsubstituted n-butyl. In embodiments, R^{10D} is unsubstituted isobutyl. In embodiments, R^{10D} is unsubstituted tert-butyl.

In embodiments, a substituted R¹¹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹¹ is substituted with a plurality of groups selected from substituent groups, size-limited substituent groups, and lower substituent groups; each substituent group, size-limited substituent group, and/or lower substituent group may optionally be different. In embodiments, when R¹¹ is substituted, it is substituted with at least one substituent group. In embodiments, when R¹¹ is substituted, it is substituted with at least one size-limited substituent group. In embodiments, when R¹¹ is substituted, it is substituted with at least one lower substituent group.

In embodiments, R¹¹ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R¹¹ is hydrogen. In embodiments, R¹¹ is halogen. In embodiments, R″ is —F. In embodiments, R¹¹ is —Cl. In embodiments, R¹¹ is —Br. In embodiments, R¹¹ is —I. In embodiments, R¹¹ is —CCl₃. In embodiments, R¹¹ is —CBr₃. In embodiments, R¹¹ is —CF₃. In embodiments, R¹¹ is —CI₃. In embodiments, R¹¹ is —CH₂C₁. In embodiments, R¹¹ is —CH₂Br. In embodiments, R¹¹ is —CH₂F. In embodiments, R¹¹ is —CH₂I. In embodiments, R¹¹ is —CHCl₂. In embodiments, R¹¹ is —CHBr₂. In embodiments, R¹¹ is —CHF₂. In embodiments, R¹¹ is —CHI₂. In embodiments, R¹¹ is —CN. In embodiments, R¹¹ is —OH. In embodiments, R¹¹ is —NH₂. In embodiments, R¹¹ is —COOH. In embodiments, R¹¹ is —CONH₂. In embodiments, R¹¹ is —NO₂. In embodiments, R¹¹ is —SH. In embodiments, R¹¹ is —SO₃H. In embodiments, R¹¹ is —OSO₃H. In embodiments, R¹¹ is —SO₂NH₂. In embodiments, R¹¹ is —NHNH₂. In embodiments, R¹¹ is —ONH₂. In embodiments, R¹¹ is —NHC(O)NH₂. In embodiments, R¹¹ is —NHSO₂H. In embodiments, R¹¹ is —NHC(O)H. In embodiments, R¹¹ is —NHC(O)OH. In embodiments, R¹¹ is —NHOH. In embodiments, R¹¹ is —OCCl₃. In embodiments, R¹¹ is —OCBr₃. In embodiments, R″ is —OCF₃. In embodiments, R¹¹ is —OCT₃. In embodiments, R¹¹ is —OCH₂Cl. In embodiments, R¹¹ is —OCH₂Br. In embodiments, R¹¹ is —OCH₂F. In embodiments, R″ is —OCH₂I. In embodiments, R¹¹ is —OCHCl₂. In embodiments, R¹¹ is —OCHBr₂. In embodiments, R¹¹ is —OCHF₂. In embodiments, R¹¹ is —OCHI₂. In embodiments, R¹¹ is —N₃. In embodiments, R¹¹ is unsubstituted C₁-C₄ alkyl. In embodiments, R¹¹ is unsubstituted methyl. In embodiments, R¹¹ is unsubstituted ethyl. In embodiments, R¹¹ is unsubstituted propyl. In embodiments, R¹¹ is unsubstituted n-propyl. In embodiments, R¹¹ is unsubstituted isopropyl. In embodiments, R¹¹ is unsubstituted butyl. In embodiments, R¹¹ is unsubstituted n-butyl. In embodiments, R¹¹ is unsubstituted isobutyl. In embodiments, R¹¹ is unsubstituted tert-butyl. In embodiments, R¹¹ is unsubstituted 2 to 6 membered heteroalkyl. In embodiments, R¹¹ is unsubstituted methoxy. In embodiments, R¹¹ is unsubstituted ethoxy. In embodiments, R¹¹ is unsubstituted propoxy. In embodiments, R¹¹ is unsubstituted n-propoxy. In embodiments, R¹¹ is unsubstituted isopropoxy. In embodiments, R¹¹ is unsubstituted butoxy.

In embodiments, a substituted R^{11A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{11A} is substituted with a plurality of groups selected from substituent groups, size-limited substituent groups, and lower substituent groups; each substituent group, size-limited substituent group, and/or lower substituent group may optionally be different. In embodiments, when R^{11A} is substituted, it is substituted with at least one substituent group. In embodiments, when R^{11A} is substituted, it is substituted with at least one size-limited substituent group. In embodiments, when R^{11A} is substituted, it is substituted with at least one lower substituent group.

In embodiments, a substituted R^{11B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1B} is substituted with a plurality of groups selected from substituent groups, size-limited substituent groups, and lower substituent groups; each substituent group, size-limited substituent group, and/or lower substituent group may optionally be different. In embodiments, when R^{1B} is substituted, it is substituted with at least one substituent group. In embodiments, when R^{11B} is substituted, it is substituted with at least one size-limited substituent group. In embodiments, when R^{1B} is substituted, it is substituted with at least one lower substituent group.

In embodiments, a substituted R^{11C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{11C} is substituted with a plurality of groups selected from substituent groups, size-limited substituent groups, and lower substituent groups; each substituent group, size-limited substituent group, and/or lower substituent group may optionally be different. In embodiments, when R^{11C} is substituted, it is substituted with at least one substituent group. In embodiments, when R^{11C} is substituted, it is substituted with at least one size-limited substituent group. In embodiments, when R^{11C} is substituted, it is substituted with at least one lower substituent group.

In embodiments, a substituted ring formed when R^{11B} and R^{11C} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{11B} and R^{11C} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{11B} and R^{11C} substituents bonded to the same nitrogen atom are joined is substituted, it is substituted with at least one substituent group. In embodiments, when the substituted ring formed when R^{11B} and R^{11C} substituents bonded to the same nitrogen atom are joined is substituted, it is substituted with at least one size-limited substituent group. In embodiments, when the substituted ring formed when R^{11B} and R^{11C} substituents bonded to the same nitrogen atom are joined is substituted, it is substituted with at least one lower substituent group.

In embodiments, a substituted R^{11D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{11D} is substituted with a plurality of groups selected from substituent groups, size-limited substituent groups, and lower substituent groups; each substituent group, size-limited substituent group, and/or lower substituent group may optionally be different. In embodiments, when R^{11D} is substituted, it is substituted with at least one substituent group. In embodiments, when R^{11D} is substituted, it is substituted with at least one size-limited substituent group. In embodiments, when R^{11D} is substituted, it is substituted with at least one lower substituent group.

In embodiments, R^{11A} is hydrogen. In embodiments, R^{11A} is unsubstituted C₁-C₄ alkyl. In embodiments, R^{11A} is unsubstituted methyl. In embodiments, R^{11A} is unsubstituted ethyl. In embodiments, R^{11A} is unsubstituted propyl. In embodiments, R^{11A} is unsubstituted n-propyl. In embodiments, R^{11A} is unsubstituted isopropyl. In embodiments, R^{11A} is unsubstituted butyl. In embodiments, R^{11A} is unsubstituted n-butyl. In embodiments, R^{11A} is unsubstituted isobutyl. In embodiments, R^{11A} is unsubstituted tert-butyl.

In embodiments, R^{1B} is hydrogen. In embodiments, R^{1B} is unsubstituted C₁-C₄ alkyl. In embodiments, R^{1B} is unsubstituted methyl. In embodiments, R^{1B} is unsubstituted ethyl. In embodiments, R^{11B} is unsubstituted propyl. In embodiments, R^{11B} is unsubstituted n-propyl. In embodiments, R^{1B} is unsubstituted isopropyl. In embodiments, R^{1B} is unsubstituted butyl. In embodiments, R^{1B} is unsubstituted n-butyl. In embodiments, R^{1B} is unsubstituted isobutyl. In embodiments, R^{1B} is unsubstituted tert-butyl.

In embodiments, R^{11C} is hydrogen. In embodiments, R^{11C} is unsubstituted C₁-C₄ alkyl. In embodiments, R^{11C} is unsubstituted methyl. In embodiments, R^{11C} is unsubstituted ethyl. In embodiments, R^{11C} is unsubstituted propyl. In embodiments, R^{11C} is unsubstituted n-propyl. In embodiments, R^{11C} is unsubstituted isopropyl. In embodiments, R^{11C} is unsubstituted butyl. In embodiments, R^{11C} is unsubstituted n-butyl. In embodiments, R^{11C} is unsubstituted isobutyl. In embodiments, R^{11C} is unsubstituted tert-butyl.

In embodiments, R^{11D} is hydrogen. In embodiments, R^{11D} is unsubstituted C₁-C₄ alkyl. In embodiments, R^{11D} is unsubstituted methyl. In embodiments, R^{11D} is unsubstituted ethyl. In embodiments, R^{11D} is unsubstituted propyl. In embodiments, R^{11D} is unsubstituted n-propyl. In embodiments, R^{11D} is unsubstituted isopropyl. In embodiments, R^{11D} is unsubstituted butyl. In embodiments, R^{11D} is unsubstituted n-butyl. In embodiments, R^{1ID} is unsubstituted isobutyl. In embodiments, R^{11D} is unsubstituted tert-butyl.

In embodiments, a substituted R¹² (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹² is substituted with a plurality of groups selected from substituent groups, size-limited substituent groups, and lower substituent groups; each substituent group, size-limited substituent group, and/or lower substituent group may optionally be different. In embodiments, when R¹² is substituted, it is substituted with at least one substituent group. In embodiments, when R¹² is substituted, it is substituted with at least one size-limited substituent group. In embodiments, when R¹² is substituted, it is substituted with at least one lower substituent group.

In embodiments, R¹² is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R¹² is hydrogen. In embodiments, R¹² is halogen. In embodiments, R¹² is —F. In embodiments, R¹² is —Cl. In embodiments, R¹² is —Br. In embodiments, R¹² is —I. In embodiments, R¹² is —CCl₃. In embodiments, R¹² is —CBr₃. In embodiments, R¹² is —CF₃. In embodiments, R¹² is —CI₃. In embodiments, R¹² is —CH₂C₁. In embodiments, R¹² is —CH₂Br. In embodiments, R¹² is —CH₂F. In embodiments, R¹² is —CH₂I. In embodiments, R¹² is —CHCl₂. In embodiments, R¹² is —CHBr₂. In embodiments, R¹² is —CHF₂. In embodiments, R¹² is —CHI₂. In embodiments, R¹² is —CN. In embodiments, R¹² is —OH. In embodiments, R¹² is —NH₂. In embodiments, R¹² is —COOH. In embodiments, R¹² is —CONH₂. In embodiments, R¹² is —NO₂. In embodiments, R¹² is —SH. In embodiments, R¹² is —SO₃H. In embodiments, R¹² is —OSO₃H. In embodiments, R¹² is —SO₂NH₂. In embodiments, R¹² is —NHNH₂. In embodiments, R¹² is —ONH₂. In embodiments, R¹² is —NHC(O)NH₂. In embodiments, R¹² is —NHSO₂H. In embodiments, R¹² is —NHC(O)H. In embodiments, R¹² is —NHC(O)OH. In embodiments, R¹² is —NHOH. In embodiments, R¹² is —OCCl₃. In embodiments, R¹² is —OCBr₃. In embodiments, R¹² is —OCF₃. In embodiments, R¹² is —OCI₃. In embodiments, R¹² is —OCH₂Cl. In embodiments, R¹² is —OCH₂Br. In embodiments, R¹² is —OCH₂F. In embodiments, R¹² is —OCH₂I. In embodiments, R¹² is —OCHCl₂. In embodiments, R¹² is —OCHBr₂. In embodiments, R¹² is —OCHF₂. In embodiments, R¹² is —OCHI₂. In embodiments, R¹² is —N₃. In embodiments, R¹² is unsubstituted C₁-C₄ alkyl. In embodiments, R¹² is unsubstituted methyl. In embodiments, R¹² is unsubstituted ethyl. In embodiments, R¹² is unsubstituted propyl. In embodiments, R¹² is unsubstituted n-propyl. In embodiments, R¹² is unsubstituted isopropyl. In embodiments, R¹² is unsubstituted butyl. In embodiments, R¹² is unsubstituted n-butyl. In embodiments, R¹² is unsubstituted isobutyl. In embodiments, R¹² is unsubstituted tert-butyl. In embodiments, R¹² is unsubstituted 2 to 6 membered heteroalkyl. In embodiments, R¹² is unsubstituted methoxy. In embodiments, R¹² is unsubstituted ethoxy. In embodiments, R¹² is unsubstituted propoxy. In embodiments, R¹² is unsubstituted n-propoxy. In embodiments, R¹² is unsubstituted isopropoxy. In embodiments, R¹² is unsubstituted butoxy.

In embodiments, a substituted R^{12A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{12A} is substituted with a plurality of groups selected from substituent groups, size-limited substituent groups, and lower substituent groups; each substituent group, size-limited substituent group, and/or lower substituent group may optionally be different. In embodiments, when R^{12A} is substituted, it is substituted with at least one substituent group. In embodiments, when R^{12A} is substituted, it is substituted with at least one size-limited substituent group. In embodiments, when R^{12A} is substituted, it is substituted with at least one lower substituent group.

In embodiments, a substituted R^{12.1} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{12.1} is substituted with a plurality of groups selected from substituent groups, size-limited substituent groups, and lower substituent groups; each substituent group, size-limited substituent group, and/or lower substituent group may optionally be different. In embodiments, when R^{12.1} is substituted, it is substituted with at least one substituent group. In embodiments, when R^{12B} is substituted, it is substituted with at least one size-limited substituent group. In embodiments, when R^{12.1} is substituted, it is substituted with at least one lower substituent group.

In embodiments, a substituted R^{12C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{12C} is substituted with a plurality of groups selected from substituent groups, size-limited substituent groups, and lower substituent groups; each substituent group, size-limited substituent group, and/or lower substituent group may optionally be different. In embodiments, when R^{12C} is substituted, it is substituted with at least one substituent group. In embodiments, when R^{12C} is substituted, it is substituted with at least one size-limited substituent group. In embodiments, when R^{12C} is substituted, it is substituted with at least one lower substituent group.

In embodiments, a substituted ring formed when R^{12B} and R^{12C} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{12B} and R^{12C} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{12B} and R^{12C} substituents bonded to the same nitrogen atom are joined is substituted, it is substituted with at least one substituent group. In embodiments, when the substituted ring formed when R^{12B} and R^{12C}substituents bonded to the same nitrogen atom are joined is substituted, it is substituted with at least one size-limited substituent group. In embodiments, when the substituted ring formed when R^{12B} and R^{12C} substituents bonded to the same nitrogen atom are joined is substituted, it is substituted with at least one lower substituent group.

In embodiments, a substituted R^{12D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{12D} is substituted with a plurality of groups selected from substituent groups, size-limited substituent groups, and lower substituent groups; each substituent group, size-limited substituent group, and/or lower substituent group may optionally be different. In embodiments, when R^{12D} is substituted, it is substituted with at least one substituent group. In embodiments, when R^{12D} is substituted, it is substituted with at least one size-limited substituent group. In embodiments, when R^{12D} is substituted, it is substituted with at least one lower substituent group.

In embodiments, R^{12A} is hydrogen. In embodiments, R^{12A} is unsubstituted C₁-C₄ alkyl. In embodiments, R^{12A} is unsubstituted methyl. In embodiments, R^{12A} is unsubstituted ethyl. In embodiments, R^{12A} is unsubstituted propyl. In embodiments, R^{2A} is unsubstituted n-propyl. In embodiments, R^{12A} is unsubstituted isopropyl. In embodiments, R^{12A} is unsubstituted butyl. In embodiments, R^{12A} is unsubstituted n-butyl. In embodiments, R^{12A} is unsubstituted isobutyl. In embodiments, R^{2A} is unsubstituted tert-butyl.

In embodiments, R^{12.1} is hydrogen. In embodiments, R^{12.1} is unsubstituted C₁-C₄ alkyl. In embodiments, R^{12.1} is unsubstituted methyl. In embodiments, R^{12.1} is unsubstituted ethyl. In embodiments, R^{12.1} is unsubstituted propyl. In embodiments, R^{12.1} is unsubstituted n-propyl. In embodiments, R^{12.1} is unsubstituted isopropyl. In embodiments, R^{12.1} is unsubstituted butyl. In embodiments, R^{12.1} is unsubstituted n-butyl. In embodiments, R^{12.1} is unsubstituted isobutyl. In embodiments, R^{12.1} is unsubstituted tert-butyl.

In embodiments, R^{12C} is hydrogen. In embodiments, R^{12C} is unsubstituted C₁-C₄ alkyl. In embodiments, R^{12C} is unsubstituted methyl. In embodiments, R^{12C} is unsubstituted ethyl. In embodiments, R^{12C} is unsubstituted propyl. In embodiments, R^{12C} is unsubstituted n-propyl. In embodiments, R^{12C} is unsubstituted isopropyl. In embodiments, R^{12C} is unsubstituted butyl. In embodiments, R^{12C} is unsubstituted n-butyl. In embodiments, R^{12C} is unsubstituted isobutyl. In embodiments, R^{12C} is unsubstituted tert-butyl.

In embodiments, R^{12D} is hydrogen. In embodiments, R^{12D} is unsubstituted C₁-C₄ alkyl. In embodiments, R^{12D} is unsubstituted methyl. In embodiments, R^{12D} is unsubstituted ethyl. In embodiments, R^{12D} is unsubstituted propyl. In embodiments, R^{12D} is unsubstituted n-propyl. In embodiments, R^{12D} is unsubstituted isopropyl. In embodiments, R^{12D} is unsubstituted butyl. In embodiments, R^{12D} is unsubstituted n-butyl. In embodiments, R^{12D} is unsubstituted isobutyl. In embodiments, R^{12D} is unsubstituted tert-butyl.

In embodiments, a substituted ring formed when R⁹ and R¹⁰ substituents bonded to the same nitrogen atom are joined (e.g., substituted cycloalkyl, substituted heterocycloalkyl, substituted aryl, and/or substituted heteroaryl) is substituted with at least one substituent group, size-limited substituent group, or lower substituent group; wherein if the substituted ring formed when R⁹ and R¹⁰ substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R⁹ and R¹⁰ substituents bonded to the same nitrogen atom are joined is substituted, it is substituted with at least one substituent group. In embodiments, when the substituted ring formed when R⁹ and R¹⁰ substituents bonded to the same nitrogen atom are joined is substituted, it is substituted with at least one size-limited substituent group. In embodiments, when the substituted ring formed when R⁹ and R¹⁰ substituents bonded to the same nitrogen atom are joined is substituted, it is substituted with at least one lower substituent group.

In embodiments, when X² is CR⁹ and X³ is CR¹⁰, then R⁹ and R¹⁰ may optionally be joined to form a substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl. In embodiments, when X² is CR⁹ and X³ is CR¹⁰, then R⁹ and R¹⁰ may optionally be joined to form a substituted or unsubstituted C₃-C₆ cycloalkyl. In embodiments, when X² is CR⁹ and X³ is CR¹⁰, then R⁹ and R¹⁰ may optionally be joined to form a substituted or unsubstituted 3 to 6 membered heterocycloalkyl. In embodiments, when X² is CR⁹ and X³ is CR¹⁰, then R⁹ and R¹⁰ may optionally be joined to form a substituted or unsubstituted phenyl. In embodiments, when X² is CR⁹ and X³ is CR¹⁰, then R⁹ and R¹⁰ may optionally be joined to form a substituted or unsubstituted 5 to 6 membered heteroaryl.

In embodiments, a substituted ring formed when R¹⁰ and R¹¹ substituents bonded to the same nitrogen atom are joined (e.g., substituted cycloalkyl, substituted heterocycloalkyl, substituted aryl, and/or substituted heteroaryl) is substituted with at least one substituent group, size-limited substituent group, or lower substituent group; wherein if the substituted ring formed when R¹⁰ and R¹¹ substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R¹⁰ and R¹¹ substituents bonded to the same nitrogen atom are joined is substituted, it is substituted with at least one substituent group. In embodiments, when the substituted ring formed when R¹⁰ and R¹¹ substituents bonded to the same nitrogen atom are joined is substituted, it is substituted with at least one size-limited substituent group. In embodiments, when the substituted ring formed when R¹⁰ and R¹¹ substituents bonded to the same nitrogen atom are joined is substituted, it is substituted with at least one lower substituent group.

In embodiments, when X³ is CR¹⁰ and X⁴ is CR¹¹, then R¹⁰ and R¹¹ may optionally be joined to form a substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl. In embodiments, when X³ is CR¹⁰ and X⁴ is CR¹¹, then R¹⁰ and R″ may optionally be joined to form a substituted or unsubstituted C₃-C₆ cycloalkyl. In embodiments, when X³ is CR¹⁰ and X⁴ is CR¹¹, then R¹⁰ and R¹¹ may optionally be joined to form a substituted or unsubstituted 3 to 6 membered heterocycloalkyl. In embodiments, when X³ is CR¹⁰ and X⁴ is CR¹¹, then R¹⁰ and R¹¹ may optionally be joined to form a substituted or unsubstituted phenyl. In embodiments, when X³ is CR¹⁰ and X⁴ is CR¹¹, then R¹⁰ and R¹¹ may optionally be joined to form a substituted or unsubstituted 5 to 6 membered heteroaryl.

In embodiments, a substituted ring formed when R¹¹ and R¹² substituents bonded to the same nitrogen atom are joined (e.g., substituted cycloalkyl, substituted heterocycloalkyl, substituted aryl, and/or substituted heteroaryl) is substituted with at least one substituent group, size-limited substituent group, or lower substituent group; wherein if the substituted ring formed when R¹¹ and R¹² substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R¹¹ and R¹² substituents bonded to the same nitrogen atom are joined is substituted, it is substituted with at least one substituent group. In embodiments, when the substituted ring formed when R¹¹ and R¹² substituents bonded to the same nitrogen atom are joined is substituted, it is substituted with at least one size-limited substituent group. In embodiments, when the substituted ring formed when R¹¹ and R¹² substituents bonded to the same nitrogen atom are joined is substituted, it is substituted with at least one lower substituent group.

In embodiments, when X⁴ is CR¹¹ and X⁵ is CR¹², then R¹¹ and R¹² may optionally be joined to form a substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl. In embodiments, when X⁴ is CR¹¹ and X⁵ is CR¹², then R¹¹ and R¹² may optionally be joined to form a substituted or unsubstituted C₃-C₆ cycloalkyl. In embodiments, when X⁴ is CR¹¹ and X⁵ is CR¹², then R^{11.1} and R¹² may optionally be joined to form a substituted or unsubstituted 3 to 6 membered heterocycloalkyl. In embodiments, when X⁴ is CR¹¹ and X⁵ is CR¹², then R¹¹ and R¹² may optionally be joined to form a substituted or unsubstituted phenyl. In embodiments, when X⁴ is CR¹¹ and X⁵ is CR¹², then R^{11.1} and R¹² may optionally be joined to form a substituted or unsubstituted 5 to 6 membered heteroaryl.

In embodiments, when R¹ is substituted, R¹ is substituted with one or more first substituent groups denoted by R^{1.1} as explained in the definitions section above in the description of “first substituent group(s)”. In embodiments, when an R^{1.1} substituent group is substituted, the R^{1.1} substituent group is substituted with one or more second substituent groups denoted by R^{1.2} as explained in the definitions section above in the description of “first substituent group(s)”. In embodiments, when an R^{1.2} substituent group is substituted, the R^{1.2} substituent group is substituted with one or more third substituent groups denoted by R^{1.3} as explained in the definitions section above in the description of “first substituent group(s)”. In the above embodiments, R¹, R^{1.1}, R^{1.2}, and R^{1.3} have values corresponding to the values of RWW, R^{WW.1}, R^{WW.2}, and R^{WW.3}, respectively, as explained in the definitions section above in the description of “first substituent group(s)”, wherein RWW, R^{W.1W}, R^{WW.2}, and R^{WW.3} correspond to R¹, R^{1.1}, R^{1.2}, and R^{1.3}, respectively.

In embodiments, when R^{1A} is substituted, R^{1A} is substituted with one or more first substituent groups denoted by R^{1A.1} as explained in the definitions section above in the description of “first substituent group(s)”. In embodiments, when an R^{1A.1} substituent group is substituted, the R^{1A.1} substituent group is substituted with one or more second substituent groups denoted by R^{1A.2} as explained in the definitions section above in the description of “first substituent group(s)”. In embodiments, when an R^{1A.2} substituent group is substituted, the R^{1A.2} substituent group is substituted with one or more third substituent groups denoted by R^{1A.3} as explained in the definitions section above in the description of “first substituent group(s)”. In the above embodiments, R^{1A}, R^{1A.1}, R^{1A.2}, and R^{1A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1A}, R^{1A.1}, R^{1A.2}, and R^{1A.3}, respectively.

In embodiments, when R^{1B} is substituted, R^{1B} is substituted with one or more first substituent groups denoted by R^{1B} as explained in the definitions section above in the description of “first substituent group(s)”. In embodiments, when an R^{1B.1} substituent group is substituted, the R^{1B.1} substituent group is substituted with one or more second substituent groups denoted by R^{1B.2} as explained in the definitions section above in the description of “first substituent group(s)”. In embodiments, when an R^{1B.2} substituent group is substituted, the R^{1B.2} substituent group is substituted with one or more third substituent groups denoted by R^{1B.3} as explained in the definitions section above in the description of “first substituent group(s)”. In the above embodiments, R^{1B}, R^{1B.1}, R^{1B.2}, and R^{1B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1B}, R^{1B.1}, R^{1B.2}, and R^{1B.3}, respectively.

In embodiments, when R^{1C} is substituted, R^{1C} is substituted with one or more first substituent groups denoted by R^{1c.1} as explained in the definitions section above in the description of “first substituent group(s)”. In embodiments, when an R^{1C.1} substituent group is substituted, the R^{1C.1} substituent group is substituted with one or more second substituent groups denoted by R^{1C.2} as explained in the definitions section above in the description of “first substituent group(s)”. In embodiments, when an R^{1c.2} substituent group is substituted, the R^{1C.2} substituent group is substituted with one or more third substituent groups denoted by R^{1C.3} as explained in the definitions section above in the description of “first substituent group(s)”. In the above embodiments, R^{1C}, R^{1C.1}, R^{1C.2}, and R^{1C.3} have values corresponding to the values of R^{WW}, R^{WW.1}, R^{WW.2}, and R^{WW.3}, respectively, as explained in the definitions section above in the description of “first substituent group(s)”, wherein R^{W}, R^{WW.1}, R^{WW.2}, and R^{WW3} correspond to R^{1C}, R^{1C}, R^{1C.2}, and R^{C.3}, respectively.

In embodiments, when R^{1B} and R^{1C} substituents bonded to the same nitrogen atom are optionally joined to form a moiety that is substituted (e.g., a substituted heterocycloalkyl or substituted heteroaryl), the moiety is substituted with one or more first substituent groups denoted by R^{1B.1} as explained in the definitions section above in the description of “first substituent group(s)”. In embodiments, when an R^{1B.1} substituent group is substituted, the R^{1B.1}substituent group is substituted with one or more second substituent groups denoted by R^{1B.2} as explained in the definitions section above in the description of “first substituent group(s)”. In embodiments, when an R^{1B.2} substituent group is substituted, the R^{1B2} substituent group is substituted with one or more third substituent groups denoted by R^{1B.3} as explained in the definitions section above in the description of “first substituent group(s)”. In the above embodiments, R^{1B.1}, R^{1B.2}, and R^{1B.3} have values corresponding to the values of R^{WW}, R^{WW.2} and R^{WW.3}, respectively, as explained in the definitions section above in the description of “first substituent group(s)”, wherein R^{WW.1}, R^{WW.2}, and R^{WW.3} correspond to R^{1B}0.1, R^{1B.2}, and R^{1B.3} respectively.

In embodiments, when R^{1B} and R^{1C} substituents bonded to the same nitrogen atom are optionally joined to form a moiety that is substituted (e.g., a substituted heterocycloalkyl or substituted heteroaryl), the moiety is substituted with one or more first substituent groups denoted by R^{1C.1} as explained in the definitions section above in the description of “first substituent group(s)”. In embodiments, when an R^{1C.1} substituent group is substituted, the R^{1C.1} substituent group is substituted with one or more second substituent groups denoted by R^{1C.2} as explained in the definitions section above in the description of “first substituent group(s)”. In embodiments, when an R^{1C.2} substituent group is substituted, the R^{1C.2} substituent group is substituted with one or more third substituent groups denoted by R^{1C.3} as explained in the definitions section above in the description of “first substituent group(s)”. In the above embodiments, R^{1C.1}, R^{1C.2}, and R^{1C.3} have values corresponding to the values of R^{1.1}, R^{WW.2}, and R^{WW.3}, respectively, as explained in the definitions section above in the description of “first substituent group(s)”, wherein R^{WW.1}, R^{WW.2}, and R^{WW.3} correspond to R^{1C.1}, R^{1C.2}, and R^{1C.3}, respectively.

In embodiments, when R^{1D} is substituted, R^{1D} is substituted with one or more first substituent groups denoted by R^{1D.1} as explained in the definitions section above in the description of “first substituent group(s)”. In embodiments, when an R^{1D.1} substituent group is substituted, the R^{1D.1} substituent group is substituted with one or more second substituent groups denoted by R^{1D.2} as explained in the definitions section above in the description of “first substituent group(s)”. In embodiments, when an R^{1D.2} substituent group is substituted, the R^{1D.2} substituent group is substituted with one or more third substituent groups denoted by R^{1D3} as explained in the definitions section above in the description of “first substituent group(s)”. In the above embodiments, R^{1D}, R^{1D.1}, R^{1D.2}, and R^{1D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1D}, R^{1D.1}, R^{1D.2} and R^{1D3}, respectively.

In embodiments, when R² is substituted, R² is substituted with one or more first substituent groups denoted by R²1 as explained in the definitions section above in the description of “first substituent group(s)”. In embodiments, when an R^{2.1} substituent group is substituted, the R^{2.1} substituent group is substituted with one or more second substituent groups denoted by R^{2.2} as explained in the definitions section above in the description of “first substituent group(s)”. In embodiments, when an R^{2.2} substituent group is substituted, the R^{2.2} substituent group is substituted with one or more third substituent groups denoted by R^{2.3} as explained in the definitions section above in the description of “first substituent group(s)”. In the above embodiments, R², R^{2.1}, R^{2.2}, and R^{2.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R², R^{2.1}, R^{2.2}, and R^{2.3}, respectively.

In embodiments, when R^{2A} is substituted, R^{2A} is substituted with one or more first substituent groups denoted by R^{2A.1} as explained in the definitions section above in the description of “first substituent group(s)”. In embodiments, when an R^{2A.1} substituent group is substituted, the R^{2A.1} substituent group is substituted with one or more second substituent groups denoted by R^{2A.2} as explained in the definitions section above in the description of “first substituent group(s)”. In embodiments, when an R^{2A.2} substituent group is substituted, the R^{2A.2} substituent group is substituted with one or more third substituent groups denoted by R^{2A.3} as explained in the definitions section above in the description of “first substituent group(s)”. In the above embodiments, R^{2A}, R^{2A.1}, R^{2A.2}, and R^{2A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2A}, R^{2A.1}, R^{2A.2}, and R^{2A}0.3, respectively.

In embodiments, when R^{2B} is substituted, R^{2B} is substituted with one or more first substituent groups denoted by R^{2B.1} as explained in the definitions section above in the description of “first substituent group(s)”. In embodiments, when an R^{2B.1} substituent group is substituted, the R^{2B.1} substituent group is substituted with one or more second substituent groups denoted by R^{2B.2} as explained in the definitions section above in the description of “first substituent group(s)”. In embodiments, when an R^{2B.2} substituent group is substituted, the R^{2B.2} substituent group is substituted with one or more third substituent groups denoted by R^{2B.3} as explained in the definitions section above in the description of “first substituent group(s)”. In the above embodiments, R^{2B}, R^{2B.1}, R^{2B.2}, and R^{2B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2B}, R^{2B.1}, R^{2B.2}, and R^{2B.3}, respectively.

In embodiments, when R^{2C} is substituted, R^{2C} is substituted with one or more first substituent groups denoted by R^{2C.1} as explained in the definitions section above in the description of “first substituent group(s)”. In embodiments, when an R^{2C.1} substituent group is substituted, the R^{2C.1} substituent group is substituted with one or more second substituent groups denoted by R^{2C.2} as explained in the definitions section above in the description of “first substituent group(s)”. In embodiments, when an R^{2C.2} substituent group is substituted, the R^{2C.2} substituent group is substituted with one or more third substituent groups denoted by R^{2C.3} as explained in the definitions section above in the description of “first substituent group(s)”. In the above embodiments, R^{2C}, R^{2C.1}, R^{2C.2}, and R^{2C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2C}, R^{2C.1}, R^{2C.2}, and R^{2C.3}, respectively.

In embodiments, when R^{2B} and R^{2C} substituents bonded to the same nitrogen atom are optionally joined to form a moiety that is substituted (e.g., a substituted heterocycloalkyl or substituted heteroaryl), the moiety is substituted with one or more first substituent groups denoted by R^{2B.1} as explained in the definitions section above in the description of “first substituent group(s)”. In embodiments, when an R^{2B.1} substituent group is substituted, the R^{2B.1} substituent group is substituted with one or more second substituent groups denoted by R^{2B.2} as explained in the definitions section above in the description of “first substituent group(s)”. In embodiments, when an R^{2B.2} substituent group is substituted, the R^{2B.2} substituent group is substituted with one or more third substituent groups denoted by R^{2B.3} as explained in the definitions section above in the description of “first substituent group(s)”. In the above embodiments, R^{2B}.1, R^{2B.2}, and R^{2B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{2B.1}, R^{2B.2}, and R^{2B.3}, respectively.

In embodiments, when R^{2B} and R^{2C} substituents bonded to the same nitrogen atom are optionally joined to form a moiety that is substituted (e.g., a substituted heterocycloalkyl or substituted heteroaryl), the moiety is substituted with one or more first substituent groups denoted by R^{2C.1} as explained in the definitions section above in the description of “first substituent group(s)”. In embodiments, when an R^{2C.1} substituent group is substituted, the R^{2C.1} substituent group is substituted with one or more second substituent groups denoted by R^{2C.2} as explained in the definitions section above in the description of “first substituent group(s)”. In embodiments, when an R^{2C.2} substituent group is substituted, the R^{2C.2} substituent group is substituted with one or more third substituent groups denoted by R^{2C.3} as explained in the definitions section above in the description of “first substituent group(s)”. In the above embodiments, R^{2C.1}, R^{2C.2}, and R^{2C.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{2C}, R^{2C.2}, and R^{2C.3}, respectively.

In embodiments, when R^{2D} is substituted, R^{2D} is substituted with one or more first substituent groups denoted by R^{2D.1} as explained in the definitions section above in the description of “first substituent group(s)”. In embodiments, when an R^{2D.1} substituent group is substituted, the R^{2D.1} substituent group is substituted with one or more second substituent groups denoted by R^{2D.2} as explained in the definitions section above in the description of “first substituent group(s)”. In embodiments, when an R^{2D.2} substituent group is substituted, the R^{2D.2} substituent group is substituted with one or more third substituent groups denoted by R^{2D3} as explained in the definitions section above in the description of “first substituent group(s)”. In the above embodiments, R^{2D}, R^{2D.1}, R^{2D.2}, and R^{2D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2D}, R^{2D.1}, R^{2D.2}, and R^{2D.3}, respectively.

In embodiments, when R³ is substituted, R³ is substituted with one or more first substituent groups denoted by R^{3.1} as explained in the definitions section above in the description of “first substituent group(s)”. In embodiments, when an R^{3.1} substituent group is substituted, the R^{3.1} substituent group is substituted with one or more second substituent groups denoted by R^{3.2} as explained in the definitions section above in the description of “first substituent group(s)”. In embodiments, when an R^{3.2} substituent group is substituted, the R^{3.2} substituent group is substituted with one or more third substituent groups denoted by R^{3.3} as explained in the definitions section above in the description of “first substituent group(s)”. In the above embodiments, R³, R^{3.1}, R^{3.2}, and R^{3.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R³, R³¹, R^{3.2}, and R^{3.3}, respectively.

In embodiments, when R^{3A} is substituted, R^{3A} is substituted with one or more first substituent groups denoted by R^{3A.1} as explained in the definitions section above in the description of “first substituent group(s)”. In embodiments, when an R^{3A.1} substituent group is substituted, the R^{3A.1} substituent group is substituted with one or more second substituent groups denoted by R^{3A.2} as explained in the definitions section above in the description of “first substituent group(s)”. In embodiments, when an R^{3A.2} substituent group is substituted, the R^{3A.2} substituent group is substituted with one or more third substituent groups denoted by R^{3A3} as explained in the definitions section above in the description of “first substituent group(s)”. In the above embodiments, R^{3A}, R^{3A.1}, R^{3A.2}, and R^{3A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3A}, R^{3A.1}, R^{3A.2}, and R^{3A.3}, respectively.

In embodiments, when R^{3B} is substituted, R^{3B} is substituted with one or more first substituent groups denoted by R^{3B.1} as explained in the definitions section above in the description of “first substituent group(s)”. In embodiments, when an R^{3B.1} substituent group is substituted, the R^{3B.1} substituent group is substituted with one or more second substituent groups denoted by R^{3B.2} as explained in the definitions section above in the description of “first substituent group(s)”. In embodiments, when an R^{3B.2} substituent group is substituted, the R^{3B.2} substituent group is substituted with one or more third substituent groups denoted by R^{3B3} as explained in the definitions section above in the description of “first substituent group(s)”. In the above embodiments, R^{3B}, R^{3B.1}, R^{3B.2}, and R^{3B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3B}, R^{3B.1}, R^{3B.2}, and R^{3B.3}, respectively.

In embodiments, when R^{3C} is substituted, R^{3C} is substituted with one or more first substituent groups denoted by R^{3C.1} as explained in the definitions section above in the description of “first substituent group(s)”. In embodiments, when an R^{3C.1} substituent group is substituted, the R^{3C.1} substituent group is substituted with one or more second substituent groups denoted by R^{3C.2} as explained in the definitions section above in the description of “first substituent group(s)”. In embodiments, when an R^{3C.2} substituent group is substituted, the R^{3C.2} substituent group is substituted with one or more third substituent groups denoted by R^{3C.3} as explained in the definitions section above in the description of “first substituent group(s)”. In the above embodiments, R^{3C}, R^{3C.1}, R^{3C.2}, and R^{3C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3C}, R^{3C.1}, R^{3C.2}, and R^{3C.3}, respectively.

In embodiments, when R^{3B} and R^{3C}, substituents bonded to the same nitrogen atom are optionally joined to form a moiety that is substituted (e.g., a substituted heterocycloalkyl or substituted heteroaryl), the moiety is substituted with one or more first substituent groups denoted by R^{3B.1} as explained in the definitions section above in the description of “first substituent group(s)”. In embodiments, when an R^{3B.1} substituent group is substituted, the R^{3B}1 substituent group is substituted with one or more second substituent groups denoted by R^{3B.2} as explained in the definitions section above in the description of “first substituent group(s)”. In embodiments, when an R^{3B.2} substituent group is substituted, the R^{3B.2} substituent group is substituted with one or more third substituent groups denoted by R^{3B.3} as explained in the definitions section above in the description of “first substituent group(s)”. In the above embodiments, R^{3B.1}, R^{3B.2}, and R^{3B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{3B.1}, R^{3B.2}, and R^{3B.3} respectively.

In embodiments, when R^{3B} and R^{3C}, substituents bonded to the same nitrogen atom are optionally joined to form a moiety that is substituted (e.g., a substituted heterocycloalkyl or substituted heteroaryl), the moiety is substituted with one or more first substituent groups denoted by R^{3C.1} as explained in the definitions section above in the description of “first substituent group(s)”. In embodiments, when an R^{3C.1} substituent group is substituted, the R^{3C.1} substituent group is substituted with one or more second substituent groups denoted by R^{3C.2} as explained in the definitions section above in the description of “first substituent group(s)”. In embodiments, when an R^{3C.2} substituent group is substituted, the R^{3C.2} substituent group is substituted with one or more third substituent groups denoted by R^{3C.3} as explained in the definitions section above in the description of “first substituent group(s)”. In the above embodiments, R^{3C.1}, R^{3C.2}, and R^{3C.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{3C.1}, R^{3C.2}, and R^{3C.3}, respectively.

In embodiments, when R^{3D} is substituted, R^{3D} is substituted with one or more first substituent groups denoted by R^{3D.1} as explained in the definitions section above in the description of “first substituent group(s)”. In embodiments, when an R^{3D.1} substituent group is substituted, the R^{3D.1} substituent group is substituted with one or more second substituent groups denoted by R^{3D.2} as explained in the definitions section above in the description of “first substituent group(s)”. In embodiments, when an R^{3D.2} substituent group is substituted, the R^{3D.2} substituent group is substituted with one or more third substituent groups denoted by R^{3D.3} as explained in the definitions section above in the description of “first substituent group(s)”. In the above embodiments, R^{3D}, R^{3D.1}, R^{3D.2}, and R^{3D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3D}, R^{3D.1}, R^{3D.2}, and R^{3D.3}, respectively.

In embodiments, when R^{3.2} is substituted, R^{3.2} is substituted with one or more first substituent groups denoted by R^{3.2.1} as explained in the definitions section above in the description of “first substituent group(s)”. In embodiments, when an R^{3.2.1} substituent group is substituted, the R^{3.2.1} substituent group is substituted with one or more second substituent groups denoted by R^{3.2.2} as explained in the definitions section above in the description of “first substituent group(s)”. In embodiments, when an R^{3.2.2} substituent group is substituted, the R^{3.2.2} substituent group is substituted with one or more third substituent groups denoted by R^{3.2.3} as explained in the definitions section above in the description of “first substituent group(s)”. In the above embodiments, R^{3.2}, R^{3.2.1}, R^{3.2.2}, and R^{3.2.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3.2}, R^{3.2.1}, R^{3.2.2}, and R^{3.2.3}, respectively.

In embodiments, when R^{3.2A} is substituted, R^{3.2A} is substituted with one or more first substituent groups denoted by R^{3.2A.1} as explained in the definitions section above in the description of “first substituent group(s)”. In embodiments, when an R^{3.2A.1} substituent group is substituted, the R^{3.2A.1} substituent group is substituted with one or more second substituent groups denoted by R^{3.2A.2} as explained in the definitions section above in the description of “first substituent group(s)”. In embodiments, when an R^{3.2A.2} substituent group is substituted, the R^{3.2A.2} substituent group is substituted with one or more third substituent groups denoted by R^{3.2A.3} as explained in the definitions section above in the description of “first substituent group(s)”. In the above embodiments, R^{3.2A}, R^{3.2A.1}, R^{3.2A.2}, and R^{3.2A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3.2A}, R^{3.2A}.1, R^{3.2A.2}, and R^{3.2A.3}, respectively.

In embodiments, when R^{3.2B} is substituted, R^{3.2B} is substituted with one or more first substituent groups denoted by R^{3.2B.1} as explained in the definitions section above in the description of “first substituent group(s)”. In embodiments, when an R^{3.2B.1} substituent group is substituted, the R^{3.2B.1} substituent group is substituted with one or more second substituent groups denoted by R^{3.2B.2} as explained in the definitions section above in the description of “first substituent group(s)”. In embodiments, when an R^{3.2B.2} substituent group is substituted, the R^{3.2B.2} substituent group is substituted with one or more third substituent groups denoted by R^{3.2B.3} as explained in the definitions section above in the description of “first substituent group(s)”. In the above embodiments, R^{3.2B}, R^{3.2B.1}, R^{3.2B.2}, and R^{3.2B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3.2B}, R^{3.2B}.1, R^{3.2B.2}, and R^{3.2B.3}, respectively.

In embodiments, when R^{3.2C} is substituted, R^{3.2C} is substituted with one or more first substituent groups denoted by R^{3.2C.1} as explained in the definitions section above in the description of “first substituent group(s)”. In embodiments, when an R^{3.2C.1} substituent group is substituted, the R^{3.2C.1} substituent group is substituted with one or more second substituent groups denoted by R^{3.2C.2} as explained in the definitions section above in the description of “first substituent group(s)”. In embodiments, when an R^{3.2C.2} substituent group is substituted, the R^{3.2C.2} substituent group is substituted with one or more third substituent groups denoted by R^{3.2C.3} as explained in the definitions section above in the description of “first substituent group(s)”. In the above embodiments, R^{3.2C}, R^{3.2C.1}, R^{3.2C.2}, and R^{3.2C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3.2C}, R^{3.2C.1}, R^{3.2C.2}, and R^{3.2C.3}, respectively.

In embodiments, when R^{3.2B} and R^{3.2C} substituents bonded to the same nitrogen atom are optionally joined to form a moiety that is substituted (e.g., a substituted heterocycloalkyl or substituted heteroaryl), the moiety is substituted with one or more first substituent groups denoted by R^{3.2B.1} as explained in the definitions section above in the description of “first substituent group(s)”. In embodiments, when an R^{3.2B.1} substituent group is substituted, the R^{3.2B.1} substituent group is substituted with one or more second substituent groups denoted by R^{3.2B.2} as explained in the definitions section above in the description of “first substituent group(s)”. In embodiments, when an R^{3.2B.2} substituent group is substituted, the R^{3.2B.2} substituent group is substituted with one or more third substituent groups denoted by R^{3.2B.3} as explained in the definitions section above in the description of “first substituent group(s)”. In the above embodiments, R^{3.2B.1}, R^{3.2B.2}, and R^{3.2B.3} have values corresponding to the values of R^{WW}, R^{WW.2}, and R^{WW.3}, respectively, as explained in the definitions section above in the description of “first substituent group(s)”, wherein R^{WW.1}, R^{WW.2}, and R^{WW.3} correspond to R^{3.2B.1}, R^{3.2.2}, and R^{3.2B.3}, respectively.

In embodiments, when R^{3.2B} and R^{3.2C} substituents bonded to the same nitrogen atom are optionally joined to form a moiety that is substituted (e.g., a substituted heterocycloalkyl or substituted heteroaryl), the moiety is substituted with one or more first substituent groups denoted by R^{3.2C.1} as explained in the definitions section above in the description of “first substituent group(s)”. In embodiments, when an R^{3.2C.1} substituent group is substituted, the R^{3.2C.1} substituent group is substituted with one or more second substituent groups denoted by R^{3.2C.2} as explained in the definitions section above in the description of “first substituent group(s)”. In embodiments, when an R^{3.2C.2} substituent group is substituted, the R^{3.2C.2} substituent group is substituted with one or more third substituent groups denoted by R^{3.2c.3} as explained in the definitions section above in the description of “first substituent group(s)”. In the above embodiments, R^{3.2C.1}, R^{3.2C.2}, and R^{3.2C.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{3.2C}, R^{3.2C.2}, and R^{3.2C.3}, respectively.

In embodiments, when R^{3.2D} is substituted, R^{3.2D} is substituted with one or more first substituent groups denoted by R^{3.2D.1} as explained in the definitions section above in the description of “first substituent group(s)”. In embodiments, when an R^{3.2D.1} substituent group is substituted, the R^{3.2D.1} substituent group is substituted with one or more second substituent groups denoted by R^{3.2D.2} as explained in the definitions section above in the description of “first substituent group(s)”. In embodiments, when an R^{3.2D.2} substituent group is substituted, the R^{3.2D.2} substituent group is substituted with one or more third substituent groups denoted by R^{3.2D.3} as explained in the definitions section above in the description of “first substituent group(s)”. In the above embodiments, R^{3.2D}, R^{3.2D.1}, R^{3.2D.2}, and R^{3.2D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3.2D}, R^{3.2D.1}, R^{3.2D.2}, and R^{3.2D.3}, respectively.

In embodiments, when R^{3.3} is substituted, R^{3.3} is substituted with one or more first substituent groups denoted by R^{3.31} as explained in the definitions section above in the description of “first substituent group(s)”. In embodiments, when an R^{3.31} substituent group is substituted, the R^{3.31} substituent group is substituted with one or more second substituent groups denoted by R^{3.32} as explained in the definitions section above in the description of “first substituent group(s)”. In embodiments, when an R^{3.32} substituent group is substituted, the R^{3.32} substituent group is substituted with one or more third substituent groups denoted by R^{3.33} as explained in the definitions section above in the description of “first substituent group(s)”. In the above embodiments, R^{3.3}, R^{3.31}, R^{3.32}, and R^{3.33} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3.3}, R^{3.31}, R^{3.3.2}, and R^{3.3}, respectively.

In embodiments, when R^{3.3A} is substituted, R^{3.3A} is substituted with one or more first substituent groups denoted by R^{3.3A.1} as explained in the definitions section above in the description of “first substituent group(s)”. In embodiments, when an R^{3.3A.1} substituent group is substituted, the R^{3.3A.1} substituent group is substituted with one or more second substituent groups denoted by R^{3.3A.2} as explained in the definitions section above in the description of “first substituent group(s)”. In embodiments, when an R^{3.3A.2} substituent group is substituted, the R^{3.3A.2} substituent group is substituted with one or more third substituent groups denoted by R^{3.3A.3} as explained in the definitions section above in the description of “first substituent group(s)”. In the above embodiments, R^{3.3A}, R^{3.3A.1}, R^{3.3A.2}, and R^{3.3A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3.3}A, R^{3.3A.1}, R^{3.3A.2}, and R^{3.3A.3}, respectively.

In embodiments, when R^{3.3B} is substituted, R^{3.3B} is substituted with one or more first substituent groups denoted by R^{3.3B.1} as explained in the definitions section above in the description of “first substituent group(s)”. In embodiments, when an R^{3.3B.1} substituent group is substituted, the R^{3.3B.1} substituent group is substituted with one or more second substituent groups denoted by R^{3.3B.2} as explained in the definitions section above in the description of “first substituent group(s)”. In embodiments, when an R^{3.3B.2} substituent group is substituted, the R^{3.3B.2} substituent group is substituted with one or more third substituent groups denoted by R^{3.3B.3} as explained in the definitions section above in the description of “first substituent group(s)”. In the above embodiments, R^{3.3B}, R^{3.3B.1}, R^{3.3B.2}, and R^{3.3B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3.3B}, R^{3.3B.1}, R^{3.3B.2}, and R^{3.3B.3}, respectively.

In embodiments, when R^{3.3C} is substituted, R^{3.3C} is substituted with one or more first substituent groups denoted by R^{3.3C.1} as explained in the definitions section above in the description of “first substituent group(s)”. In embodiments, when an R^{3.3C.1} substituent group is substituted, the R^{3.3C.1} substituent group is substituted with one or more second substituent groups denoted by R^{3.3C.2} as explained in the definitions section above in the description of “first substituent group(s)”. In embodiments, when an R^{3.3C.2} substituent group is substituted, the R^{3.3C.2} substituent group is substituted with one or more third substituent groups denoted by R^{3.3C.3} as explained in the definitions section above in the description of “first substituent group(s)”. In the above embodiments, R^{3.3C}, R^{3.3C.1}, R^{3.3C.2}, and R^{3.3C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3.3C}, R^{3.3C.1}, R^{3.3C.2}, and R^{3.3C.3}, respectively.

In embodiments, when R^{3.3B} and R^{3.3C} substituents bonded to the same nitrogen atom are optionally joined to form a moiety that is substituted (e.g., a substituted heterocycloalkyl or substituted heteroaryl), the moiety is substituted with one or more first substituent groups denoted by R^{3.3B.1} as explained in the definitions section above in the description of “first substituent group(s)”. In embodiments, when an R^{3.3B.1} substituent group is substituted, the R^{3.3B.1} substituent group is substituted with one or more second substituent groups denoted by R^{3.3B.2} as explained in the definitions section above in the description of “first substituent group(s)”. In embodiments, when an R^{3.3B.2} substituent group is substituted, the R^{3.3B.2} substituent group is substituted with one or more third substituent groups denoted by R^{3.3B.3} as explained in the definitions section above in the description of “first substituent group(s)”. In the above embodiments, R^{3.3B1}, R^{3.3B.2}, and R^{3.3B.3} have values corresponding to the values of R^{WW}, R^{WW.2}, and R^{WW.3}, respectively, as explained in the definitions section above in the description of “first substituent group(s)”, wherein R^{WW.1}, R^{WW.2}, and R^{WW.3} correspond to R^{3.3B1}, R^{3.3B2} and R^{3.3B.3}, respectively.

In embodiments, when R^{3.3B} and R^{3.3C} substituents bonded to the same nitrogen atom are optionally joined to form a moiety that is substituted (e.g., a substituted heterocycloalkyl or substituted heteroaryl), the moiety is substituted with one or more first substituent groups denoted by R^{3.3C.1} as explained in the definitions section above in the description of “first substituent group(s)”. In embodiments, when an R^{3.3C.1} substituent group is substituted, the R^{3.3C.1} substituent group is substituted with one or more second substituent groups denoted by R^{3.3C.2} as explained in the definitions section above in the description of “first substituent group(s)”. In embodiments, when an R^{3.3C.2} substituent group is substituted, the R^{3.3C.2} substituent group is substituted with one or more third substituent groups denoted by R^{3.3C.3} as explained in the definitions section above in the description of “first substituent group(s)”. In the above embodiments, R^{3.3C}, R^{3.3C.2}, and R^{3.3C.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{3.3C}, R^{3.3C.2}, and R^{3.3C.3}, respectively.

In embodiments, when R^{3.3D} is substituted, R^{3.3D} is substituted with one or more first substituent groups denoted by R^{3.3D.1} as explained in the definitions section above in the description of “first substituent group(s)”. In embodiments, when an R^{3.3D.1} substituent group is substituted, the R^{3.3D.1} substituent group is substituted with one or more second substituent groups denoted by R^{3.3D.2} as explained in the definitions section above in the description of “first substituent group(s)”. In embodiments, when an R^{3.3D.2} substituent group is substituted, the R^{3.3D.2} substituent group is substituted with one or more third substituent groups denoted by R^{3.3D.3} as explained in the definitions section above in the description of “first substituent group(s)”. In the above embodiments, R^{3.3D}, R^{3.3D.1}, R^{3.3D.2}, and R^{3.3D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3.3D}, R^{3.3D.1}, R^{3.3D.2}, and R^{3.3D.3}, respectively.

In embodiments, when R⁴ is substituted, R⁴ is substituted with one or more first substituent groups denoted by R^{4.1} as explained in the definitions section above in the description of “first substituent group(s)”. In embodiments, when an R^{4.1} substituent group is substituted, the R^{4.1} substituent group is substituted with one or more second substituent groups denoted by R^{4.2} as explained in the definitions section above in the description of “first substituent group(s)”. In embodiments, when an R^{4.2} substituent group is substituted, the R^{4.2} substituent group is substituted with one or more third substituent groups denoted by R^{4.3} as explained in the definitions section above in the description of “first substituent group(s)”. In the above embodiments, R⁴, R^{4.1}, R^{4.2}, and R^{4.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁴, R^{4.1}, R^{4.2}, and R^{4.3}, respectively.

In embodiments, when R^{4A} is substituted, R^{4A} is substituted with one or more first substituent groups denoted by R^{4A.1} as explained in the definitions section above in the description of “first substituent group(s)”. In embodiments, when an R^{4A.1} substituent group is substituted, the R^{4A.1} substituent group is substituted with one or more second substituent groups denoted by R^{4A.2} as explained in the definitions section above in the description of “first substituent group(s)”. In embodiments, when an R^{4A.2} substituent group is substituted, the R^{4A.2} substituent group is substituted with one or more third substituent groups denoted by R^{4A.3} as explained in the definitions section above in the description of “first substituent group(s)”. In the above embodiments, R^{4A}, R^{4A.1}, R^{4A.2}, and R^{4A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{4A}, R^{4A.1}, R^{4A.2}, and R^{4A.3}, respectively.

In embodiments, when R^{4B} is substituted, R^{4B} is substituted with one or more first substituent groups denoted by R^{4B.1} as explained in the definitions section above in the description of “first substituent group(s)”. In embodiments, when an R^{4B.1} substituent group is substituted, the R^{4B.1} substituent group is substituted with one or more second substituent groups denoted by R^{4B.2} as explained in the definitions section above in the description of “first substituent group(s)”. In embodiments, when an R^{4B.2} substituent group is substituted, the R^{4B.2} substituent group is substituted with one or more third substituent groups denoted by R^{4B.3} as explained in the definitions section above in the description of “first substituent group(s)”. In the above embodiments, R^{4B}, R^{4B.1}, R^{4B.2}, and R^{4B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{4B}, R^{4B.1}, R^{4B.2}, and R^{4B.3}, respectively.

In embodiments, when R^{4C} is substituted, R^{4C} is substituted with one or more first substituent groups denoted by R^{4C.1} as explained in the definitions section above in the description of “first substituent group(s)”. In embodiments, when an R^{4C.1} substituent group is substituted, the R^{4C.1} substituent group is substituted with one or more second substituent groups denoted by R^{4C.2} as explained in the definitions section above in the description of “first substituent group(s)”. In embodiments, when an R^{4C.2} substituent group is substituted, the R^{4C.2} substituent group is substituted with one or more third substituent groups denoted by R^{4C.3} as explained in the definitions section above in the description of “first substituent group(s)”. In the above embodiments, R^{4C}, R^{4C.1}, R^{4C.2}, and R^{4C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{4C}, R^{4C.1}, R^{4C.2}, and R^{4C.3}, respectively.

In embodiments, when R^{4B} and R^{4C} substituents bonded to the same nitrogen atom are optionally joined to form a moiety that is substituted (e.g., a substituted heterocycloalkyl or substituted heteroaryl), the moiety is substituted with one or more first substituent groups denoted by R^{4B.1} as explained in the definitions section above in the description of “first substituent group(s)”. In embodiments, when an R^{4B.1} substituent group is substituted, the R^{4B.1} substituent group is substituted with one or more second substituent groups denoted by R^{4B.2} as explained in the definitions section above in the description of “first substituent group(s)”. In embodiments, when an R^{4B.2} substituent group is substituted, the R^{4B.2} substituent group is substituted with one or more third substituent groups denoted by R^{4B.3} as explained in the definitions section above in the description of “first substituent group(s)”. In the above embodiments, R^{4B.1}, R^{4B.2}, and R^{4B.3} have values corresponding to the values of R^{WW.1}, R^{WW.2}, and R^{WW.3}, respectively, as explained in the definitions section above in the description of “first substituent group(s)”, wherein R^{WW}, R^{WW.2}, and R^{WW.3} correspond to R^{4B.1}, R^{4B.2}, and R^{4B.3} respectively.

In embodiments, when R^{4B} and R^{4C} substituents bonded to the same nitrogen atom are optionally joined to form a moiety that is substituted (e.g., a substituted heterocycloalkyl or substituted heteroaryl), the moiety is substituted with one or more first substituent groups denoted by R^{4C}.1 as explained in the definitions section above in the description of “first substituent group(s)”. In embodiments, when an R^{4C.1} substituent group is substituted, the R^{4C.1} substituent group is substituted with one or more second substituent groups denoted by R^{4C.2} as explained in the definitions section above in the description of “first substituent group(s)”. In embodiments, when an R^{4C.2} substituent group is substituted, the R^{4C.2} substituent group is substituted with one or more third substituent groups denoted by R^{4C.3} as explained in the definitions section above in the description of “first substituent group(s)”. In the above embodiments, R^{4C.1}, R^{4C.2}, and R^{4C.3} have values corresponding to the values of R^{WW.1}, R^{WW.2}, and R^{WW.3}, respectively, as explained in the definitions section above in the description of “first substituent group(s)”, wherein R^{WW}, R^{WW.2}, and R^{WW.3} correspond to R^{4C.1}, R^{4C.2}, and R^{4C.3}, respectively.

In embodiments, when R^{4D} is substituted, R^{4D} is substituted with one or more first substituent groups denoted by R^{4D.1} as explained in the definitions section above in the description of “first substituent group(s)”. In embodiments, when an R^{4D.1} substituent group is substituted, the R^{4D.1} substituent group is substituted with one or more second substituent groups denoted by R^{4D.2} as explained in the definitions section above in the description of “first substituent group(s)”. In embodiments, when an R^{4D.2} substituent group is substituted, the R^{4D.2} substituent group is substituted with one or more third substituent groups denoted by R^{4D3} as explained in the definitions section above in the description of “first substituent group(s)”. In the above embodiments, R^{4D}, R^{4D.1}, R^{4D.2}, and R^{4D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{4D}, R^{4D}, R^{4D.2}, and R^{4D.3}, respectively.

In embodiments, when R⁵ is substituted, R⁵ is substituted with one or more first substituent groups denoted by R^{5.1} as explained in the definitions section above in the description of “first substituent group(s)”. In embodiments, when an R^{5.1} substituent group is substituted, the R^{5.1} substituent group is substituted with one or more second substituent groups denoted by R^{5.2} as explained in the definitions section above in the description of “first substituent group(s)”. In embodiments, when an R^{5.2} substituent group is substituted, the R^{5.2} substituent group is substituted with one or more third substituent groups denoted by R5^{.3} as explained in the definitions section above in the description of “first substituent group(s)”. In the above embodiments, R⁵, R^{5.1}, R^{5.2}, and R^{5.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁵, R^{5.1}, R^{5.2}, and R^{5.3}, respectively.

In embodiments, when R^{5A} is substituted, R^{5A} is substituted with one or more first substituent groups denoted by R^{5A.1} as explained in the definitions section above in the description of “first substituent group(s)”. In embodiments, when an R^{5A.1} substituent group is substituted, the R^{5A.1} substituent group is substituted with one or more second substituent groups denoted by R^{5A.2} as explained in the definitions section above in the description of “first substituent group(s)”. In embodiments, when an R^{5A.2} substituent group is substituted, the R^{5A.2} substituent group is substituted with one or more third substituent groups denoted by R^{5A.3} as explained in the definitions section above in the description of “first substituent group(s)”. In the above embodiments, R^{5A}, R^{5A.1}, R^{5A.2}, and R^{5A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{5A}, R^{5A}.1, R^{5A.2}, and R^{5A.3}, respectively.

In embodiments, when R^{5B} is substituted, R^{5B} is substituted with one or more first substituent groups denoted by R^{5B.1} as explained in the definitions section above in the description of “first substituent group(s)”. In embodiments, when an R^{5B.1} substituent group is substituted, the R^{5B.1} substituent group is substituted with one or more second substituent groups denoted by R^{5B.2} as explained in the definitions section above in the description of “first substituent group(s)”. In embodiments, when an R^{5B.2} substituent group is substituted, the R^{5B.2} substituent group is substituted with one or more third substituent groups denoted by R^{5B3} as explained in the definitions section above in the description of “first substituent group(s)”. In the above embodiments, R^{5B}, R^{5B.1}, R^{5B.2}, and R^{5B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{5B}, R^{5B.1}, R^{5B.2}, and R^{5B.3}, respectively.

In embodiments, when R^{5C} is substituted, R^{5C} is substituted with one or more first substituent groups denoted by R^{5C.1} as explained in the definitions section above in the description of “first substituent group(s)”. In embodiments, when an R^{5C.1} substituent group is substituted, the R^{5C.1} substituent group is substituted with one or more second substituent groups denoted by R^{5C.2} as explained in the definitions section above in the description of “first substituent group(s)”. In embodiments, when an R^{5C.2} substituent group is substituted, the R^{5C.2} substituent group is substituted with one or more third substituent groups denoted by R^{5C.3} as explained in the definitions section above in the description of “first substituent group(s)”. In the above embodiments, R^{5C}, R^{5C.1}, R^{5C.2}, and R^{5C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{5C}, R^{5C.1}, R^{5C.2}, and R^{5C.3}, respectively.

In embodiments, when R^{5B} and R^{5C} substituents bonded to the same nitrogen atom are optionally joined to form a moiety that is substituted (e.g., a substituted heterocycloalkyl or substituted heteroaryl), the moiety is substituted with one or more first substituent groups denoted by R^{5B.1} as explained in the definitions section above in the description of “first substituent group(s)”. In embodiments, when an R^{5B.1} substituent group is substituted, the R^{5B.1} substituent group is substituted with one or more second substituent groups denoted by R^{5B.2} as explained in the definitions section above in the description of “first substituent group(s)”. In embodiments, when an R^{5B.2} substituent group is substituted, the R^{5B.2} substituent group is substituted with one or more third substituent groups denoted by R^{5B.3} as explained in the definitions section above in the description of “first substituent group(s)”. In the above embodiments, R^{5B}.1, R^{5B.2}, and R^{5B.3} have values corresponding to the values of R^{WW.1}, R^{WW.2}, and R^{WW.3}, respectively, as explained in the definitions section above in the description of “first substituent group(s)”, wherein R^{WW}, R^{WW.2}, and R^{WW.3} correspond to R^{5B.1}, R^{5B.2}, and R^{5B.3} respectively.

In embodiments, when R^{5B} and R^{5C} substituents bonded to the same nitrogen atom are optionally joined to form a moiety that is substituted (e.g., a substituted heterocycloalkyl or substituted heteroaryl), the moiety is substituted with one or more first substituent groups denoted by R^{5C.1} as explained in the definitions section above in the description of “first substituent group(s)”. In embodiments, when an R^{5C.1} substituent group is substituted, the R^{5C}.1 substituent group is substituted with one or more second substituent groups denoted by R^{5C.2} as explained in the definitions section above in the description of “first substituent group(s)”. In embodiments, when an R^{5C.2} substituent group is substituted, the R^{5C.2} substituent group is substituted with one or more third substituent groups denoted by R^{5C.3} as explained in the definitions section above in the description of “first substituent group(s)”. In the above embodiments, R^{5C}.1, R^{5C.2}, and R^{5C.3} have values corresponding to the values of R^{WW.1}, R^{WW.2}, and R^{WW.3}, respectively, as explained in the definitions section above in the description of “first substituent group(s)”, wherein R^{WW}, R^{WW.2}, and R^{WW.3} correspond to R^{5C}, R^{5C.2}, and R^{5C.3}, respectively.

In embodiments, when R^{5D} is substituted, R^{5D} is substituted with one or more first substituent groups denoted by R^{5D.1} as explained in the definitions section above in the description of “first substituent group(s)”. In embodiments, when an R^{5D.1} substituent group is substituted, the R^{5D.1} substituent group is substituted with one or more second substituent groups denoted by R^{5D.2} as explained in the definitions section above in the description of “first substituent group(s)”. In embodiments, when an R^{5D.2} substituent group is substituted, the R^{5D.2} substituent group is substituted with one or more third substituent groups denoted by R^{5D3} as explained in the definitions section above in the description of “first substituent group(s)”. In the above embodiments, R^{5D}, R^{5D}, R^{5D.2}, and R^{5D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{5D}, R^{5D.1}, R^{5D.2}, and R^{5D.3}, respectively.

In embodiments, when R⁶ is substituted, R⁶ is substituted with one or more first substituent groups denoted by R^{6.1} as explained in the definitions section above in the description of “first substituent group(s)”. In embodiments, when an R^{6.1} substituent group is substituted, the R^{6.1} substituent group is substituted with one or more second substituent groups denoted by R^{6.2} as explained in the definitions section above in the description of “first substituent group(s)”. In embodiments, when an R^{6.2} substituent group is substituted, the R^{6.2} substituent group is substituted with one or more third substituent groups denoted by R6^{.3} as explained in the definitions section above in the description of “first substituent group(s)”. In the above embodiments, R⁶, R^{6.1}, R⁶0.2, and R6^{.3} have values corresponding to the values of R^{WW}, R^{WW}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁶, R⁶¹, R⁶0.2, and R⁶-3, respectively.

In embodiments, when R^{6A} is substituted, R^{6A} is substituted with one or more first substituent groups denoted by R^{6A}0.1 as explained in the definitions section above in the description of “first substituent group(s)”. In embodiments, when an R^{6A}0.1 substituent group is substituted, the R^{6A}0.1 substituent group is substituted with one or more second substituent groups denoted by R^{6A.2} as explained in the definitions section above in the description of “first substituent group(s)”. In embodiments, when an R6A² substituent group is substituted, the R^{6A}0.2 substituent group is substituted with one or more third substituent groups denoted by R^{6A3} as explained in the definitions section above in the description of “first substituent group(s)”. In the above embodiments, R^{6A}, R^{6A}0.1, R^{6A}0.2, and R^{6A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{6A}, R^{6A}0.1, R^{6A}0.2, and R^{6A}0.3, respectively.

In embodiments, when R^{6B} is substituted, R^{6B} is substituted with one or more first substituent groups denoted by R^{6B}0.1 as explained in the definitions section above in the description of “first substituent group(s)”. In embodiments, when an R^{6B}0.1 substituent group is substituted, the R^{6B}0.1 substituent group is substituted with one or more second substituent groups denoted by R^{6B.2} as explained in the definitions section above in the description of “first substituent group(s)”. In embodiments, when an R^{6B.2} substituent group is substituted, the R^{6B}0.2 substituent group is substituted with one or more third substituent groups denoted by R^{6B3} as explained in the definitions section above in the description of “first substituent group(s)”. In the above embodiments, R^{6B}, R^{6B}0.1, R^{6B}0.2, and R^{6B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{6B}, R^{6B}0.1, R^{6B}0.2, and R^{6B}0.3, respectively.

In embodiments, when R^{6C} is substituted, R^{6C} is substituted with one or more first substituent groups denoted by R^{6c}0.1 as explained in the definitions section above in the description of “first substituent group(s)”. In embodiments, when an R^{6C}.1 substituent group is substituted, the R^{6c}0.1 substituent group is substituted with one or more second substituent groups denoted by R^{6c.2} as explained in the definitions section above in the description of “first substituent group(s)”. In embodiments, when an R^{6c.2} substituent group is substituted, the R^{6C}.2 substituent group is substituted with one or more third substituent groups denoted by R^{6c.3} as explained in the definitions section above in the description of “first substituent group(s)”. In the above embodiments, R^{6C}, R^{6C}.1, R^{6C.2}, and R^{6C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{6C}, R^{6c}0.1, R^{6c}0.2, and R^{6c}0.3, respectively.

In embodiments, when R^{6B} and R^{6C} substituents bonded to the same nitrogen atom are optionally joined to form a moiety that is substituted (e.g., a substituted heterocycloalkyl or substituted heteroaryl), the moiety is substituted with one or more first substituent groups denoted by R^{6B}.1 as explained in the definitions section above in the description of “first substituent group(s)”. In embodiments, when an R^{6B}.1 substituent group is substituted, the R^{6B}.1 substituent group is substituted with one or more second substituent groups denoted by R^{6B.2} as explained in the definitions section above in the description of “first substituent group(s)”. In embodiments, when an R^{6B.2} substituent group is substituted, the R^{6B.2} substituent group is substituted with one or more third substituent groups denoted by R^{6B.3} as explained in the definitions section above in the description of “first substituent group(s)”. In the above embodiments, R^{6B}0.1, R^{6B}0.2, and R^{6B.3} have values corresponding to the values of R^{WW.1}, R^{WW.2}, and R^{WW.3}, respectively, as explained in the definitions section above in the description of “first substituent group(s)”, wherein R^{WW}, R^{WW.2}, and R^{WW.3} correspond to R^{6B}0.1, R^{6B}0.2, and R^{6B}0.3 respectively.

In embodiments, when R^{6B} and R^{6C} substituents bonded to the same nitrogen atom are optionally joined to form a moiety that is substituted (e.g., a substituted heterocycloalkyl or substituted heteroaryl), the moiety is substituted with one or more first substituent groups denoted by R^{6C}.1 as explained in the definitions section above in the description of “first substituent group(s)”. In embodiments, when an R^{6C}.1 substituent group is substituted, the R^{6C}.1 substituent group is substituted with one or more second substituent groups denoted by R^{6C.2} as explained in the definitions section above in the description of “first substituent group(s)”. In embodiments, when an R^{6C.2} substituent group is substituted, the R^{6C.2} substituent group is substituted with one or more third substituent groups denoted by R^{6C.3} as explained in the definitions section above in the description of “first substituent group(s)”. In the above embodiments, R^{6C}.1, R^{6C.2}, and R^{6C.3} have values corresponding to the values of R^{WW.1}, R^{WW.2}, and R^{WW.3}, respectively, as explained in the definitions section above in the description of “first substituent group(s)”, wherein R^{WW}, R^{WW.2}, and R^{WW.3} correspond to R^{6C}.1, R^{6C.2}, and R^{6C.3}, respectively.

In embodiments, when R^{6D} is substituted, R^{6D} is substituted with one or more first substituent groups denoted by R^{6D.1} as explained in the definitions section above in the description of “first substituent group(s)”. In embodiments, when an R^{6D.1} substituent group is substituted, the R^{6D.1} substituent group is substituted with one or more second substituent groups denoted by R^{6D.2} as explained in the definitions section above in the description of “first substituent group(s)”. In embodiments, when an R^{6D.2} substituent group is substituted, the R^{6D.2} substituent group is substituted with one or more third substituent groups denoted by R^{6D3} as explained in the definitions section above in the description of “first substituent group(s)”. In the above embodiments, R^{6D}, R^{6D.1}, R^{6D.2}, and R^{6D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{6D}, R^{6D}, R^{6D.2}, and R^{6D.3}, respectively.

In embodiments, when R⁷ is substituted, R⁷ is substituted with one or more first substituent groups denoted by R^{7.1} as explained in the definitions section above in the description of “first substituent group(s)”. In embodiments, when an R^{7.1} substituent group is substituted, the R^{7.1} substituent group is substituted with one or more second substituent groups denoted by R^{7.2} as explained in the definitions section above in the description of “first substituent group(s)”. In embodiments, when an R^{7.2} substituent group is substituted, the R^{7.2} substituent group is substituted with one or more third substituent groups denoted by R^{7.3} as explained in the definitions section above in the description of “first substituent group(s)”. In the above embodiments, R⁷, R^{7.1}, R^{7.2}, and R^{7.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁷, R^{7.1}, R^{7.2}, and R⁷-3, respectively.

In embodiments, when R^{7A} is substituted, R^{7A} is substituted with one or more first substituent groups denoted by R^{7A.1} as explained in the definitions section above in the description of “first substituent group(s)”. In embodiments, when an R^{7A.1} substituent group is substituted, the R^{7A.1} substituent group is substituted with one or more second substituent groups denoted by R^{7A.2} as explained in the definitions section above in the description of “first substituent group(s)”. In embodiments, when an R^{7A.2} substituent group is substituted, the R^{7A.2} substituent group is substituted with one or more third substituent groups denoted by R^{7A.3} as explained in the definitions section above in the description of “first substituent group(s)”. In the above embodiments, R^{7A}, R^{7A.1}, R^{7A.2}, and R^{7A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{7A}, R^{7A.1}, R^{7A.2}, and R^{7A.3}, respectively.

In embodiments, when R^{7B} is substituted, R^{7B} is substituted with one or more first substituent groups denoted by R^{7B.1} as explained in the definitions section above in the description of “first substituent group(s)”. In embodiments, when an R^{7B.1} substituent group is substituted, the R^{7B.1} substituent group is substituted with one or more second substituent groups denoted by R^{7B.2} as explained in the definitions section above in the description of “first substituent group(s)”. In embodiments, when an R^{7B.2} substituent group is substituted, the R^{7B.2} substituent group is substituted with one or more third substituent groups denoted by R^{7B.3} as explained in the definitions section above in the description of “first substituent group(s)”. In the above embodiments, R^{7B}, R^{7B.1}, R^{7B.2}, and R^{7B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{7B}, R^{7B.1}, R^{7B.2}, and R^{7B.3}, respectively.

In embodiments, when R^{7C} is substituted, R^{7C} is substituted with one or more first substituent groups denoted by R^{7C.1} as explained in the definitions section above in the description of “first substituent group(s)”. In embodiments, when an R^{7C.1} substituent group is substituted, the R^{7C.1} substituent group is substituted with one or more second substituent groups denoted by R^{7C.2} as explained in the definitions section above in the description of “first substituent group(s)”. In embodiments, when an R^{7C.2} substituent group is substituted, the R^{7C.2} substituent group is substituted with one or more third substituent groups denoted by R^{7C.3} as explained in the definitions section above in the description of “first substituent group(s)”. In the above embodiments, R^{7C}, R^{7C.1}, R^{7C.2}, and R^{7C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{7C}, R^{7C.1}, R^{7C.2}, and R^{7C.3}, respectively.

In embodiments, when R^{7B} and R^{7C} substituents bonded to the same nitrogen atom are optionally joined to form a moiety that is substituted (e.g., a substituted heterocycloalkyl or substituted heteroaryl), the moiety is substituted with one or more first substituent groups denoted by R^{7B.1} as explained in the definitions section above in the description of “first substituent group(s)”. In embodiments, when an R^{7B.1} substituent group is substituted, the R^{7B.1} substituent group is substituted with one or more second substituent groups denoted by R^{7B.2} as explained in the definitions section above in the description of “first substituent group(s)”. In embodiments, when an R^{7B.2} substituent group is substituted, the R^{7B.2} substituent group is substituted with one or more third substituent groups denoted by R^{7B.3} as explained in the definitions section above in the description of “first substituent group(s)”. In the above embodiments, R^{7B.1}, R^{7B.2}, and R^{7B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{7B.1}, R^{7B.2}, and R^{7B.3} respectively.

In embodiments, when R^{7B} and R^{7C} substituents bonded to the same nitrogen atom are optionally joined to form a moiety that is substituted (e.g., a substituted heterocycloalkyl or substituted heteroaryl), the moiety is substituted with one or more first substituent groups denoted by R^{7C.1} as explained in the definitions section above in the description of “first substituent group(s)”. In embodiments, when an R^{7C.1} substituent group is substituted, the R^{7C.1} substituent group is substituted with one or more second substituent groups denoted by R^{7C.2} as explained in the definitions section above in the description of “first substituent group(s)”. In embodiments, when an R^{7C.2} substituent group is substituted, the R^{7C.2} substituent group is substituted with one or more third substituent groups denoted by R^{7C.3} as explained in the definitions section above in the description of “first substituent group(s)”. In the above embodiments, R^{7C.1}, R^{7C.2}, and R^{7C.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{7C.1}, R^{7C.2}, and R^{7C.3}, respectively.

In embodiments, when R^{7D} is substituted, R^{7D} is substituted with one or more first substituent groups denoted by R^{7D.1} as explained in the definitions section above in the description of “first substituent group(s)”. In embodiments, when an R^{7D.1} substituent group is substituted, the R^{7D.1} substituent group is substituted with one or more second substituent groups denoted by R^{7D.2} as explained in the definitions section above in the description of “first substituent group(s)”. In embodiments, when an R^{7D.2} substituent group is substituted, the R^{7D.2} substituent group is substituted with one or more third substituent groups denoted by R^{7D3} as explained in the definitions section above in the description of “first substituent group(s)”. In the above embodiments, R^{7D}, R^{7D.1}, R^{7D.2}, and R^{7D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{7D}, R^{7D.1}, R^{7D.2}, and R^{7D.3}, respectively.

In embodiments, when R^{7.2B} is substituted, R^{7.2B} is substituted with one or more first substituent groups denoted by R^{7.2B.1} as explained in the definitions section above in the description of “first substituent group(s)”. In embodiments, when an R^{7.2B.1} substituent group is substituted, the R^{7.2B.1} substituent group is substituted with one or more second substituent groups denoted by R^{7.2B.2} as explained in the definitions section above in the description of “first substituent group(s)”. In embodiments, when an R^{7.2B.2} substituent group is substituted, the R^{7.2B.2} substituent group is substituted with one or more third substituent groups denoted by R^{7.2B.3} as explained in the definitions section above in the description of “first substituent group(s)”. In the above embodiments, R^{7.2B}, R^{7.2B.1}, R^{7.2B.2}, and R^{7.2B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{7.2B}, R^{7.2B.1}, R^{7.2B.2}, and R^{7.2B.3}, respectively.

In embodiments, when R⁸ is substituted, R⁸ is substituted with one or more first substituent groups denoted by R^{8.1} as explained in the definitions section above in the description of “first substituent group(s)”. In embodiments, when an R^{8.1} substituent group is substituted, the R^{8.1} substituent group is substituted with one or more second substituent groups denoted by R^{8.2} as explained in the definitions section above in the description of “first substituent group(s)”. In embodiments, when an R^{8.2} substituent group is substituted, the R^{8.2} substituent group is substituted with one or more third substituent groups denoted by R^{8.3} as explained in the definitions section above in the description of “first substituent group(s)”. In the above embodiments, R⁸, R^{8.1}, R^{8.2}, and R^{8.3} have values corresponding to the values of R^{WW}, R^{WW.1}, R^{WW.2}, and R^{WW3}, respectively, as explained in the definitions section above in the description of “first substituent group(s)”, wherein R^{WW}, R^{WW.1}, R^{WW.2}, and R^{WW.3} correspond to R⁸, R^{8.1}, R^{8.2}, and R⁸-3, respectively.

In embodiments, when R^{8A} is substituted, R^{8A} is substituted with one or more first substituent groups denoted by R^{8A.1} as explained in the definitions section above in the description of “first substituent group(s)”. In embodiments, when an R^{8A.1} substituent group is substituted, the R^{8A.1} substituent group is substituted with one or more second substituent groups denoted by R^{8A.2} as explained in the definitions section above in the description of “first substituent group(s)”. In embodiments, when an R^{8A.2} substituent group is substituted, the R^{8A.2} substituent group is substituted with one or more third substituent groups denoted by R^{8A.3} as explained in the definitions section above in the description of “first substituent group(s)”. In the above embodiments, R^{8A}, R^{8A.1}, R^{8A.2}, and R^{8A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{8A}, R^{8A.1}, R^{8A.2}, and R^{8A.3}, respectively.

In embodiments, when R^{8B} is substituted, R^{8B} is substituted with one or more first substituent groups denoted by R^{8B.1} as explained in the definitions section above in the description of “first substituent group(s)”. In embodiments, when an R^{8B}l substituent group is substituted, the R^{8B.1} substituent group is substituted with one or more second substituent groups denoted by R^{8B.2} as explained in the definitions section above in the description of “first substituent group(s)”. In embodiments, when an R^{8B.2} substituent group is substituted, the R^{8B.2} substituent group is substituted with one or more third substituent groups denoted by R^{8B.3} as explained in the definitions section above in the description of “first substituent group(s)”. In the above embodiments, R^{8B}, R^{8B.1}, R^{8B.2}, and R^{8B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{8B}, R^{8B.1}, R^{8B.2}, and R^{8B.3}, respectively.

In embodiments, when R^{8C} is substituted, R^{8C} is substituted with one or more first substituent groups denoted by R^{8C.1} as explained in the definitions section above in the description of “first substituent group(s)”. In embodiments, when an R^{8C.1} substituent group is substituted, the R^{8C.1} substituent group is substituted with one or more second substituent groups denoted by R^{8C.2} as explained in the definitions section above in the description of “first substituent group(s)”. In embodiments, when an R^{8C.2} substituent group is substituted, the R^{8C.2} substituent group is substituted with one or more third substituent groups denoted by R^{8C.3} as explained in the definitions section above in the description of “first substituent group(s)”. In the above embodiments, R^{8C}, R^{8C.1}, R^{8C.2}, and R^{8C.3}have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{8C}, R^{8C.1}, R^{8C.2}, and R^{8C.3}, respectively.

In embodiments, when R^{8B} and R^{8C} substituents bonded to the same nitrogen atom are optionally joined to form a moiety that is substituted (e.g., a substituted heterocycloalkyl or substituted heteroaryl), the moiety is substituted with one or more first substituent groups denoted by R^{8B.1} as explained in the definitions section above in the description of “first substituent group(s)”. In embodiments, when an R^{8B.1} substituent group is substituted, the R^{8B.1} substituent group is substituted with one or more second substituent groups denoted by R^{8B.2} as explained in the definitions section above in the description of “first substituent group(s)”. In embodiments, when an R^{8B.2} substituent group is substituted, the R^{8B2} substituent group is substituted with one or more third substituent groups denoted by R^{8B.3} as explained in the definitions section above in the description of “first substituent group(s)”. In the above embodiments, R^{8B.1}, R^{8B.2}, and R^{8B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{8B.1}, R^{8B.2}, and R^{8B.3} respectively.

In embodiments, when R^{8B} and R^{8C} substituents bonded to the same nitrogen atom are optionally joined to form a moiety that is substituted (e.g., a substituted heterocycloalkyl or substituted heteroaryl), the moiety is substituted with one or more first substituent groups denoted by R^{8c} as explained in the definitions section above in the description of “first substituent group(s)”. In embodiments, when an R^{8C.1} substituent group is substituted, the R^{8C.1} substituent group is substituted with one or more second substituent groups denoted by R^{8C.2} as explained in the definitions section above in the description of “first substituent group(s)”. In embodiments, when an R^{8C.2} substituent group is substituted, the R^{8C.2} substituent group is substituted with one or more third substituent groups denoted by R^{8C.3} as explained in the definitions section above in the description of “first substituent group(s)”. In the above embodiments, R^{8c}1, R^{8C.2}, and R^{8C.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{8C.1}, R^{8C.2}, and R^{8C.3} respectively.

In embodiments, when R^{8D} is substituted, R^{8D} is substituted with one or more first substituent groups denoted by R^{8D.1} as explained in the definitions section above in the description of “first substituent group(s)”. In embodiments, when an R^{8D.1} substituent group is substituted, the R^{8D.1} substituent group is substituted with one or more second substituent groups denoted by R^{8D.2} as explained in the definitions section above in the description of “first substituent group(s)”. In embodiments, when an R^{8D.2} substituent group is substituted, the R^{8D.2} substituent group is substituted with one or more third substituent groups denoted by R^{8D.3} as explained in the definitions section above in the description of “first substituent group(s)”. In the above embodiments, R^{8D}, R^{8D.1}, R^{8D.2}, and R^{8D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{8D}, R^{8D.1}, R^{8D.2}, and R^{8D.3}, respectively.

In embodiments, when R⁹ is substituted, R⁹ is substituted with one or more first substituent groups denoted by R^{9.1} as explained in the definitions section above in the description of “first substituent group(s)”. In embodiments, when an R^{9.1} substituent group is substituted, the R^{9.1} substituent group is substituted with one or more second substituent groups denoted by R^{9.2} as explained in the definitions section above in the description of “first substituent group(s)”. In embodiments, when an R^{9.2} substituent group is substituted, the R^{9.2} substituent group is substituted with one or more third substituent groups denoted by R^{9.3} as explained in the definitions section above in the description of “first substituent group(s)”. In the above embodiments, R⁹, R^{9.1}, R^{9.2}, and R^{9.3} have values corresponding to the values of R^{WW}, R^{WW.1}, R^{WW.2}, and R^{WW3}, respectively, as explained in the definitions section above in the description of “first substituent group(s)”, wherein R^{WW}, R^{WW.1}, R^{WW.2}, and R^{WW.3} correspond to R⁹, R^{9.1}, R^{9.2}, and R^{9.3}, respectively.

In embodiments, when R^{9A} is substituted, R^{9A} is substituted with one or more first substituent groups denoted by R^{9A.1} as explained in the definitions section above in the description of “first substituent group(s)”. In embodiments, when an R^{9A.1} substituent group is substituted, the R^{9A.1} substituent group is substituted with one or more second substituent groups denoted by R^{9A.2} as explained in the definitions section above in the description of “first substituent group(s)”. In embodiments, when an R^{9A.2} substituent group is substituted, the R^{9A.2} substituent group is substituted with one or more third substituent groups denoted by R^{9A3} as explained in the definitions section above in the description of “first substituent group(s)”. In the above embodiments, R^{9A}, R^{9A.1}, R^{9A.2}, and R^{9A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{9A}, R^{9A}.1, R^{9A.2}, and R^{9A.3}, respectively.

In embodiments, when R^{9B} is substituted, R^{9B} is substituted with one or more first substituent groups denoted by R^{9B.1} as explained in the definitions section above in the description of “first substituent group(s)”. In embodiments, when an R^{9B.1} substituent group is substituted, the R^{9B.1} substituent group is substituted with one or more second substituent groups denoted by R^{9B.2} as explained in the definitions section above in the description of “first substituent group(s)”. In embodiments, when an R^{9B.2} substituent group is substituted, the R^{9B.2} substituent group is substituted with one or more third substituent groups denoted by R^{9B3} as explained in the definitions section above in the description of “first substituent group(s)”. In the above embodiments, R^{9B}, R^{9B.1}, R^{9B.2}, and R^{9B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{9B}, R^{9B.1}, R^{9B.2}, and R^{9B.3}, respectively.

In embodiments, when R^{9C} is substituted, R^{9C} is substituted with one or more first substituent groups denoted by R^{9C.1} as explained in the definitions section above in the description of “first substituent group(s)”. In embodiments, when an R^{9C.1} substituent group is substituted, the R^{9C.1} substituent group is substituted with one or more second substituent groups denoted by R^{9C.2} as explained in the definitions section above in the description of “first substituent group(s)”. In embodiments, when an R^{9C.2} substituent group is substituted, the R^{9C.2} substituent group is substituted with one or more third substituent groups denoted by R^{9C.3} as explained in the definitions section above in the description of “first substituent group(s)”. In the above embodiments, R^{9C}, R^{9C}1, R^{9C.2}, and R^{9C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{9C}, R^{9C.1}, R^{9C.2}, and R^{9C.3}, respectively.

In embodiments, when R^{9B} and R^{9C} substituents bonded to the same nitrogen atom are optionally joined to form a moiety that is substituted (e.g., a substituted heterocycloalkyl or substituted heteroaryl), the moiety is substituted with one or more first substituent groups denoted by R^{9B.1} as explained in the definitions section above in the description of “first substituent group(s)”. In embodiments, when an R^{9B.1} substituent group is substituted, the R^{9B.1} substituent group is substituted with one or more second substituent groups denoted by R^{9B.2} as explained in the definitions section above in the description of “first substituent group(s)”. In embodiments, when an R^{9B.2} substituent group is substituted, the R^{9B.2} substituent group is substituted with one or more third substituent groups denoted by R^{9B.3} as explained in the definitions section above in the description of “first substituent group(s)”. In the above embodiments, R^{9B.1}, R^{9B.2}, and R^{9B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{9B.1}, R^{9B.2}, and R^{9B.3} respectively.

In embodiments, when R^{9B} and R^{9C} substituents bonded to the same nitrogen atom are optionally joined to form a moiety that is substituted (e.g., a substituted heterocycloalkyl or substituted heteroaryl), the moiety is substituted with one or more first substituent groups denoted by R^{9C.1} as explained in the definitions section above in the description of “first substituent group(s)”. In embodiments, when an R^{9C.1} substituent group is substituted, the R^{9C.1} substituent group is substituted with one or more second substituent groups denoted by R^{9C.2} as explained in the definitions section above in the description of “first substituent group(s)”. In embodiments, when an R^{9C.2} substituent group is substituted, the R^{9C.2} substituent group is substituted with one or more third substituent groups denoted by R^{9C.3} as explained in the definitions section above in the description of “first substituent group(s)”. In the above embodiments, R^{9C.1}, R^{9C}2, and R^{9C.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{9C.1}, R^{9C.2}, and R^{9C.3} respectively.

In embodiments, when R^{9D} is substituted, R^{9D} is substituted with one or more first substituent groups denoted by R^{9D.1} as explained in the definitions section above in the description of “first substituent group(s)”. In embodiments, when an R^{9D.1} substituent group is substituted, the R^{9D.1} substituent group is substituted with one or more second substituent groups denoted by R^{9D.2} as explained in the definitions section above in the description of “first substituent group(s)”. In embodiments, when an R^{9D.2} substituent group is substituted, the R^{9D.2} substituent group is substituted with one or more third substituent groups denoted by R^{9D3} as explained in the definitions section above in the description of “first substituent group(s)”. In the above embodiments, R^{9D}, R^{9D.1}, R^{9D.2}, and R^{9D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{9D}, R^{9D.1}, R^{9D.2}, and R^{9D.3}, respectively.

In embodiments, when R¹⁰ is substituted, R¹⁰ is substituted with one or more first substituent groups denoted by R^{10.1} as explained in the definitions section above in the description of “first substituent group(s)”. In embodiments, when an R^{10.1} substituent group is substituted, the R^{10.1} substituent group is substituted with one or more second substituent groups denoted by R^{10.2} as explained in the definitions section above in the description of “first substituent group(s)”. In embodiments, when an R^{10.2} substituent group is substituted, the R^{10.2} substituent group is substituted with one or more third substituent groups denoted by R^{10.3} as explained in the definitions section above in the description of “first substituent group(s)”. In the above embodiments, R¹⁰, R^{10.1}, R^{10.2}, and R^{10.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹⁰, R^{10.1}, R^{10.2}, and R^{10.3}, respectively.

In embodiments, when R^{10A} is substituted, R^{10A} is substituted with one or more first substituent groups denoted by R^{10A.1} as explained in the definitions section above in the description of “first substituent group(s)”. In embodiments, when an R^{10A.1} substituent group is substituted, the R^{10A.1} substituent group is substituted with one or more second substituent groups denoted by R^{10A.2} as explained in the definitions section above in the description of “first substituent group(s)”. In embodiments, when an R^{10A.2} substituent group is substituted, the R^{10A.2} substituent group is substituted with one or more third substituent groups denoted by R^{10A}.3 as explained in the definitions section above in the description of “first substituent group(s)”. In the above embodiments, R^{10A}, R^{10A.1}, R^{10A.2}, and R^{10A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{10A}, R^{10A}, R^{10A.2}, and R^{10A.3}, respectively.

In embodiments, when R^{10B} is substituted, R^{10B} is substituted with one or more first substituent groups denoted by R^{10B.1} as explained in the definitions section above in the description of “first substituent group(s)”. In embodiments, when an R^{10B.1} substituent group is substituted, the R^{10B.1} substituent group is substituted with one or more second substituent groups denoted by R^{10B.2} as explained in the definitions section above in the description of “first substituent group(s)”. In embodiments, when an R^{10B.2} substituent group is substituted, the R^{10.2} substituent group is substituted with one or more third substituent groups denoted by R^{10B.3} as explained in the definitions section above in the description of “first substituent group(s)”. In the above embodiments, R^{10B}, R^{10B.1}, R^{10B.2}, and R^{10B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{10B}, R^{10B.1}, R^{10B.2}, and R^{10B.3}, respectively.

In embodiments, when R^{10C} is substituted, R^{10C} is substituted with one or more first substituent groups denoted by R^{10C}.i as explained in the definitions section above in the description of “first substituent group(s)”. In embodiments, when an R^{10C.1} substituent group is substituted, the R^{10C.1} substituent group is substituted with one or more second substituent groups denoted by R^{10C.2} as explained in the definitions section above in the description of “first substituent group(s)”. In embodiments, when an R^{10C.2} substituent group is substituted, the R^{10C.2} substituent group is substituted with one or more third substituent groups denoted by R^{10C.3} as explained in the definitions section above in the description of “first substituent group(s)”. In the above embodiments, R^{10C}, R^{10C.1}, R^{10C.2}, and R^{10C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{10C}, R^{10C.1}, R^{10C.2}, and R^{10C.3}, respectively.

In embodiments, when R^{10B} and R^{10C} substituents bonded to the same nitrogen atom are optionally joined to form a moiety that is substituted (e.g., a substituted heterocycloalkyl or substituted heteroaryl), the moiety is substituted with one or more first substituent groups denoted by R^{10.1} as explained in the definitions section above in the description of “first substituent group(s)”. In embodiments, when an R^{10B.1} substituent group is substituted, the R^{10B.1} substituent group is substituted with one or more second substituent groups denoted by R^{10B.2} as explained in the definitions section above in the description of “first substituent group(s)”. In embodiments, when an R^{10B.2} substituent group is substituted, the R^{10B.2} substituent group is substituted with one or more third substituent groups denoted by R^{10B.3} as explained in the definitions section above in the description of “first substituent group(s)”. In the above embodiments, R^{10B.1}, R^{10B.2}, and R^{10B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{10B.1}, R^{10B.2}, and R^{10.3} respectively.

In embodiments, when R^{10B} and R^{10C} substituents bonded to the same nitrogen atom are optionally joined to form a moiety that is substituted (e.g., a substituted heterocycloalkyl or substituted heteroaryl), the moiety is substituted with one or more first substituent groups denoted by R^{10C.1} as explained in the definitions section above in the description of “first substituent group(s)”. In embodiments, when an R^{10C.1} substituent group is substituted, the R^{10C.1} substituent group is substituted with one or more second substituent groups denoted by R^{10C.2} as explained in the definitions section above in the description of “first substituent group(s)”. In embodiments, when an R^{10C.2} substituent group is substituted, the R^{10C.2} substituent group is substituted with one or more third substituent groups denoted by R^{10C.3} as explained in the definitions section above in the description of “first substituent group(s)”. In the above embodiments, R^{10C.1}, R^{10C.2}, and R^{10C.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{10C.1}, R^{10C.2}, and R^{10C.3} respectively.

In embodiments, when R^{10D} is substituted, R^{10D} is substituted with one or more first substituent groups denoted by R^{10D.1} as explained in the definitions section above in the description of “first substituent group(s)”. In embodiments, when an R^{10D.1} substituent group is substituted, the R^{10D.1} substituent group is substituted with one or more second substituent groups denoted by R^{10D.2} as explained in the definitions section above in the description of “first substituent group(s)”. In embodiments, when an R^{10D.2} substituent group is substituted, the R^{10D.2} substituent group is substituted with one or more third substituent groups denoted by R^{10D.3} as explained in the definitions section above in the description of “first substituent group(s)”. In the above embodiments, R^{10D}, R^{10D.1}, R^{10D.2}, and R^{10D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{10D}, R^{10D.1}, R^{10D.2}, and R^{10D.3}, respectively.

In embodiments, when R¹¹ is substituted, R¹¹ is substituted with one or more first substituent groups denoted by R^{11.1} as explained in the definitions section above in the description of “first substituent group(s)”. In embodiments, when an R^{11.1} substituent group is substituted, the R^{11.1} substituent group is substituted with one or more second substituent groups denoted by R^{11.2} as explained in the definitions section above in the description of “first substituent group(s)”. In embodiments, when an R^{11.2} substituent group is substituted, the R^{11.2} substituent group is substituted with one or more third substituent groups denoted by R^{11.3} as explained in the definitions section above in the description of “first substituent group(s)”. In the above embodiments, R¹¹, R^{11.1}, R^{11.2}, and R^{11.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹¹, R¹¹, R^{11.2}, and R^{11.3}, respectively.

In embodiments, when R^{11A} is substituted, R^{11A} is substituted with one or more first substituent groups denoted by R^{11A.1} as explained in the definitions section above in the description of “first substituent group(s)”. In embodiments, when an R^{11A.1} substituent group is substituted, the R^{11A.1} substituent group is substituted with one or more second substituent groups denoted by R^{11A.2} as explained in the definitions section above in the description of “first substituent group(s)”. In embodiments, when an R^{11A.2} substituent group is substituted, the R^{11A.2} substituent group is substituted with one or more third substituent groups denoted by R^{11A.3} as explained in the definitions section above in the description of “first substituent group(s)”. In the above embodiments, R^{11A}, R^{11A.1}, R^{11A.2}, and R^{11A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{11A}, R^{11A.1}, R^{11A.2}, and R^{11A.3}, respectively.

In embodiments, when R^{11B} is substituted, R^{11B} is substituted with one or more first substituent groups denoted by R^{1B.1} as explained in the definitions section above in the description of “first substituent group(s)”. In embodiments, when an Ru¹¹ substituent group is substituted, the R^{11B.1} substituent group is substituted with one or more second substituent groups denoted by R^{11B.2} as explained in the definitions section above in the description of “first substituent group(s)”. In embodiments, when an R^{11B.2} substituent group is substituted, the R^{11B.2} substituent group is substituted with one or more third substituent groups denoted by R^{11.3} as explained in the definitions section above in the description of “first substituent group(s)”. In the above embodiments, R^{11B}, R^{11B.1}, R^{11B.2}, and R^{11B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{11B}, R^{11B.1}, R^{11B.2} and R^{11B.3}, respectively.

In embodiments, when R^{11C} is substituted, R^{11C} is substituted with one or more first substituent groups denoted by R¹¹ as explained in the definitions section above in the description of “first substituent group(s)”. In embodiments, when an R^{11C.1} substituent group is substituted, the R^{11C.1} substituent group is substituted with one or more second substituent groups denoted by R^{11C.2} as explained in the definitions section above in the description of “first substituent group(s)”. In embodiments, when an R^{11.C2} substituent group is substituted, the R^{11C.2} substituent group is substituted with one or more third substituent groups denoted by R^{11C.3} as explained in the definitions section above in the description of “first substituent group(s)”. In the above embodiments, R^{11C}, R^{11.1}, R^{11C.2}, and R^{11C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{11C}, R^{11.1}, R^{11C.2} and R^{11C.3}, respectively.

In embodiments, when R^{11B} and R^{11C} substituents bonded to the same nitrogen atom are optionally joined to form a moiety that is substituted (e.g., a substituted heterocycloalkyl or substituted heteroaryl), the moiety is substituted with one or more first substituent groups denoted by R^{11B.1} as explained in the definitions section above in the description of “first substituent group(s)”. In embodiments, when an R^{11B.1} substituent group is substituted, the R^{11B.1} substituent group is substituted with one or more second substituent groups denoted by R^{11B.2} as explained in the definitions section above in the description of “first substituent group(s)”. In embodiments, when an R^{11B.2} substituent group is substituted, the R^{11B.2} substituent group is substituted with one or more third substituent groups denoted by R^{11B.3} as explained in the definitions section above in the description of “first substituent group(s)”. In the above embodiments, R^{1B.1}, R^{11B.2}, and R^{11B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{1B.1}, R^{1B.2}, and R^{11B.3} respectively.

In embodiments, when R^{11B} and R^{11C} substituents bonded to the same nitrogen atom are optionally joined to form a moiety that is substituted (e.g., a substituted heterocycloalkyl or substituted heteroaryl), the moiety is substituted with one or more first substituent groups denoted by R^{11C.1} as explained in the definitions section above in the description of “first substituent group(s)”. In embodiments, when an R^{11C.1} substituent group is substituted, the R^{11C.1} substituent group is substituted with one or more second substituent groups denoted by R^{11C.2} as explained in the definitions section above in the description of “first substituent group(s)”. In embodiments, when an R^{11C.2} substituent group is substituted, the R^{11.2} substituent group is substituted with one or more third substituent groups denoted by R^{11C.3} as explained in the definitions section above in the description of “first substituent group(s)”. In the above embodiments, R^{11.1}, R^{11C.2}, and R^{11C.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{11C}, R^{11C.2}, and R^{11C.3} respectively.

In embodiments, when R^{11D} is substituted, R^{11D} is substituted with one or more first substituent groups denoted by R^{11D.1} as explained in the definitions section above in the description of “first substituent group(s)”. In embodiments, when an R^{11D.1} substituent group is substituted, the R^{11D.1} substituent group is substituted with one or more second substituent groups denoted by R^{11D.2} as explained in the definitions section above in the description of “first substituent group(s)”. In embodiments, when an R^{11D.2} substituent group is substituted, the R^{11D.2} substituent group is substituted with one or more third substituent groups denoted by R^{11D.3} as explained in the definitions section above in the description of “first substituent group(s)”. In the above embodiments, R^{11D}, R^{11D.1}, R^{11D.2}, and R^{11D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{11D}, R^{11D.1}, R^{11D.2}, and R^{11D.3}, respectively.

In embodiments, when R¹² is substituted, R¹² is substituted with one or more first substituent groups denoted by R^{12.1} as explained in the definitions section above in the description of “first substituent group(s)”. In embodiments, when an R^{12.1} substituent group is substituted, the R^{12.1} substituent group is substituted with one or more second substituent groups denoted by R^{12.2} as explained in the definitions section above in the description of “first substituent group(s)”. In embodiments, when an R^{12.2} substituent group is substituted, the R^{12.2} substituent group is substituted with one or more third substituent groups denoted by R^{12.3} as explained in the definitions section above in the description of “first substituent group(s)”. In the above embodiments, R¹², R^{12.1}, R^{12.2}, and R^{12.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹², R^{12.1}, R^{12.2}, and R^{12.3}, respectively.

In embodiments, when R^{12A} is substituted, R^{12A} is substituted with one or more first substituent groups denoted by R^{12A.1} as explained in the definitions section above in the description of “first substituent group(s)”. In embodiments, when an R^{12A.1} substituent group is substituted, the R^{12A.1} substituent group is substituted with one or more second substituent groups denoted by R^{12A.2} as explained in the definitions section above in the description of “first substituent group(s)”. In embodiments, when an R^{12A.2} substituent group is substituted, the R^{12A.2} substituent group is substituted with one or more third substituent groups denoted by R^{12A}.3 as explained in the definitions section above in the description of “first substituent group(s)”. In the above embodiments, R^{12A}, R^{12A}.1, R^{12A.2}, and R^{12A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{12A}, R^{12A}.1, R^{12A.2}, and R^{12A.3}, respectively.

In embodiments, when R^{12B} is substituted, R^{12B} is substituted with one or more first substituent groups denoted by R^{12B.1} as explained in the definitions section above in the description of “first substituent group(s)”. In embodiments, when an R^{12B.1} substituent group is substituted, the R^{12B.1} substituent group is substituted with one or more second substituent groups denoted by R^{12B.2} as explained in the definitions section above in the description of “first substituent group(s)”. In embodiments, when an R^{12B.2} substituent group is substituted, the R^{12B.2} substituent group is substituted with one or more third substituent groups denoted by R^{12B.3} as explained in the definitions section above in the description of “first substituent group(s)”. In the above embodiments, R^{12B}, R^{12B.1}, R^{12B.2}, and R^{12B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{12B}, R^{12B.1}, R^{12B.2}, and R^{12B.3}, respectively.

In embodiments, when R^{12C} is substituted, R^{12C} is substituted with one or more first substituent groups denoted by R^{12C.1} as explained in the definitions section above in the description of “first substituent group(s)”. In embodiments, when an R^{12C.1} substituent group is substituted, the R^{12C.1} substituent group is substituted with one or more second substituent groups denoted by R^{12C.2} as explained in the definitions section above in the description of “first substituent group(s)”. In embodiments, when an R^{12C.2} substituent group is substituted, the R^{12C.2} substituent group is substituted with one or more third substituent groups denoted by R^{12C.3} as explained in the definitions section above in the description of “first substituent group(s)”. In the above embodiments, R^{12C}, R^{12C.1}, R^{12C.2}, and R^{12C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{12C}, R^{12C.1}, R^{12C}.2, and R^{12C.3}, respectively.

In embodiments, when R^{12B} and R^{12C} substituents bonded to the same nitrogen atom are optionally joined to form a moiety that is substituted (e.g., a substituted heterocycloalkyl or substituted heteroaryl), the moiety is substituted with one or more first substituent groups denoted by R^{12B.1} as explained in the definitions section above in the description of “first substituent group(s)”. In embodiments, when an R^{12B.1} substituent group is substituted, the R^{12B.1} substituent group is substituted with one or more second substituent groups denoted by R^{12B.2} as explained in the definitions section above in the description of “first substituent group(s)”. In embodiments, when an R^{12B.2} substituent group is substituted, the R^{12B.2} substituent group is substituted with one or more third substituent groups denoted by R^{12B.3} as explained in the definitions section above in the description of “first substituent group(s)”. In the above embodiments, R^{12B.1}, R^{12B.2}, and R^{12B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{12B.1}, R^{12B.2}, and R^{12B.3}, respectively.

In embodiments, when R^{12B} and R^{12C} substituents bonded to the same nitrogen atom are optionally joined to form a moiety that is substituted (e.g., a substituted heterocycloalkyl or substituted heteroaryl), the moiety is substituted with one or more first substituent groups denoted by R^{12C.1} as explained in the definitions section above in the description of “first substituent group(s)”. In embodiments, when an R^{12C.1} substituent group is substituted, the R^{12C.1} substituent group is substituted with one or more second substituent groups denoted by R^{12C.2} as explained in the definitions section above in the description of “first substituent group(s)”. In embodiments, when an R^{12C.2} substituent group is substituted, the R^{12C.2} substituent group is substituted with one or more third substituent groups denoted by R^{12C.3} as explained in the definitions section above in the description of “first substituent group(s)”. In the above embodiments, R^{12C.1}, R^{12C.2}, and R^{12C.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{12C.1}, R^{12C.2}, and R^{12C.3} respectively.

In embodiments, when R^{12D} is substituted, R^{12D} is substituted with one or more first substituent groups denoted by R^{12D.1} as explained in the definitions section above in the description of “first substituent group(s)”. In embodiments, when an R^{12D.1} substituent group is substituted, the R^{12D.1} substituent group is substituted with one or more second substituent groups denoted by R^{12D.2} as explained in the definitions section above in the description of “first substituent group(s)”. In embodiments, when an R^{12D.2} substituent group is substituted, the R^{12D.2} substituent group is substituted with one or more third substituent groups denoted by R^{12D.3} as explained in the definitions section above in the description of “first substituent group(s)”. In the above embodiments, R^{12D}, R^{12D.1}, R^{12D.2}, and R^{12D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{12D}, R^{12D.1}, R^{12D.2}, and R^{12D.3}, respectively.

In embodiments, when R⁹ and R¹⁰ substituents are optionally joined to form a moiety that is substituted (e.g., a substituted cycloalkyl, substituted heterocycloalkyl, substituted aryl, or substituted heteroaryl), the moiety is substituted with one or more first substituent groups denoted by R^{9.1} as explained in the definitions section above in the description of “first substituent group(s)”. In embodiments, when an R^{9.1} substituent group is substituted, the R^{9.1} substituent group is substituted with one or more second substituent groups denoted by R^{9.2} as explained in the definitions section above in the description of “first substituent group(s)”. In embodiments, when an R^{9.2} substituent group is substituted, the R^{9.2} substituent group is substituted with one or more third substituent groups denoted by R^{9.3} as explained in the definitions section above in the description of “first substituent group(s)”. In the above embodiments, R^{9.1}, R^{9.2}, and R^{9.3} have values corresponding to the values of R^{11.1}, R^{WW.2}, and R^{WW.3}, respectively, as explained in the definitions section above in the description of “first substituent group(s)”, wherein R^{WW.1}, R^{WW.2}, and R^{WW.3} correspond to R^{9.1}, R^{9.2}, and R^{9.3}, respectively.

In embodiments, when R⁹ and R¹⁰ substituents are optionally joined to form a moiety that is substituted (e.g., a substituted cycloalkyl, substituted heterocycloalkyl, substituted aryl, or substituted heteroaryl), the moiety is substituted with one or more first substituent groups denoted by R^{10.1} as explained in the definitions section above in the description of “first substituent group(s)”. In embodiments, when an R^{10.1} substituent group is substituted, the R^{10.1} substituent group is substituted with one or more second substituent groups denoted by R^{10.2} as explained in the definitions section above in the description of “first substituent group(s)”. In embodiments, when an R^{10.2} substituent group is substituted, the R^{10.2} substituent group is substituted with one or more third substituent groups denoted by R^{10.3} as explained in the definitions section above in the description of “first substituent group(s)”. In the above embodiments, R^{10.1}, R¹², and R^{10.3} have values corresponding to the values of R^{11.1}, R^{WW.2}, and R^{WW.3}, respectively, as explained in the definitions section above in the description of “first substituent group(s)”, wherein R^{WW.1}, R^{WW.2}, and R^{WW.3} correspond to R^{10.1}, R^{10.2}, and R^{10.3}, respectively.

In embodiments, when R¹⁰ and R¹¹ substituents are optionally joined to form a moiety that is substituted (e.g., a substituted cycloalkyl, substituted heterocycloalkyl, substituted aryl, or substituted heteroaryl), the moiety is substituted with one or more first substituent groups denoted by R^{10.1} as explained in the definitions section above in the description of “first substituent group(s)”. In embodiments, when an R^{10.1} substituent group is substituted, the R^{10.1} substituent group is substituted with one or more second substituent groups denoted by R^{10.2} as explained in the definitions section above in the description of “first substituent group(s)”. In embodiments, when an R^{10.2} substituent group is substituted, the R^{10.2} substituent group is substituted with one or more third substituent groups denoted by R^{10.3} as explained in the definitions section above in the description of “first substituent group(s)”. In the above embodiments, R^{10.1}, R^{10.2}, and R^{10.3} have values corresponding to the values of R^{11.1}, R^{WW.2}, and R^{WW.3}, respectively, as explained in the definitions section above in the description of “first substituent group(s)”, wherein R^{WW.1}, R^{WW.2}, and R^{WW.3} correspond to R^{10.1}, R^{10.2}, and R^{10.3}, respectively.

In embodiments, when R¹⁰ and R¹¹ substituents are optionally joined to form a moiety that is substituted (e.g., a substituted cycloalkyl, substituted heterocycloalkyl, substituted aryl, or substituted heteroaryl), the moiety is substituted with one or more first substituent groups denoted by R^{11.1} as explained in the definitions section above in the description of “first substituent group(s)”. In embodiments, when an R^{11.11} substituent group is substituted, the R^{11.11} substituent group is substituted with one or more second substituent groups denoted by R^{11.2} as explained in the definitions section above in the description of “first substituent group(s)”. In embodiments, when an R^{11.2} substituent group is substituted, the R^{11.2} substituent group is substituted with one or more third substituent groups denoted by R^{11.3} as explained in the definitions section above in the description of “first substituent group(s)”. In the above embodiments, R^{11.1}, R^{11.2}, and R^{11.3} have values corresponding to the values of R^{11.1}, R^{WW.2}, and R^{WW.3}, respectively, as explained in the definitions section above in the description of “first substituent group(s)”, wherein R^{WW.1}, R^{WW.2}, and R^{WW.3} correspond to R¹¹, R^{11.2}, and R^{11.3}, respectively.

In embodiments, when R¹¹ and R¹² substituents are optionally joined to form a moiety that is substituted (e.g., a substituted cycloalkyl, substituted heterocycloalkyl, substituted aryl, or substituted heteroaryl), the moiety is substituted with one or more first substituent groups denoted by R^{11.1} as explained in the definitions section above in the description of “first substituent group(s)”. In embodiments, when an R^{11.1} substituent group is substituted, the R^{11.1} substituent group is substituted with one or more second substituent groups denoted by R^{11.2} as explained in the definitions section above in the description of “first substituent group(s)”. In embodiments, when an R^{11.2} substituent group is substituted, the R^{11.2} substituent group is substituted with one or more third substituent groups denoted by R^{11.3} as explained in the definitions section above in the description of “first substituent group(s)”. In the above embodiments, R¹¹, R^{11.2}, and R^{11.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{11.1}, R^{11.2}, and R^{11.3}, respectively.

In embodiments, when R¹¹ and R¹² substituents are optionally joined to form a moiety that is substituted (e.g., a substituted cycloalkyl, substituted heterocycloalkyl, substituted aryl, or substituted heteroaryl), the moiety is substituted with one or more first substituent groups denoted by R^{12.1} as explained in the definitions section above in the description of “first substituent group(s)”. In embodiments, when an R^{12.1} substituent group is substituted, the R^{12.1} substituent group is substituted with one or more second substituent groups denoted by R^{12.2} as explained in the definitions section above in the description of “first substituent group(s)”. In embodiments, when an R^{12.2} substituent group is substituted, the R^{12.2} substituent group is substituted with one or more third substituent groups denoted by R^{12.3} as explained in the definitions section above in the description of “first substituent group(s)”. In the above embodiments, R^{12.1}, R^{12.2}, and R^{12.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{12.1}, R^{12.2}, and R^{12.3} respectively.

In embodiments, when L⁷ is substituted, L⁷ is substituted with one or more first substituent groups denoted by R^{L7.1} as explained in the definitions section above in the description of “first substituent group(s)”. In embodiments, when an R^{L7.1} substituent group is substituted, the R^{L7.1} substituent group is substituted with one or more second substituent groups denoted by R^{L7.2} as explained in the definitions section above in the description of “first substituent group(s)”. In embodiments, when an R^{L7.2} substituent group is substituted, the R^{L7.2} substituent group is substituted with one or more third substituent groups denoted by R^{L73} as explained in the definitions section above in the description of “first substituent group(s)”. In the above embodiments, L⁷, R^{L7.1}, R^{L7.2}, and R^{L7.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L⁷, R^{L7.1}, R^{L7.2}, and R^{L7.3}, respectively.

In embodiments, the compound has the formula:

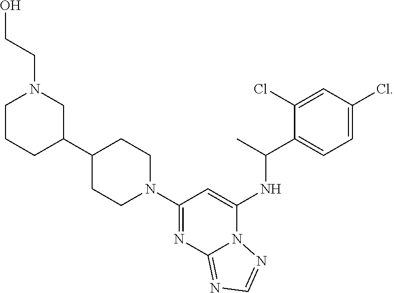 [SMILES: CC(Nc1cc(N2CCC(C3CCCN(CCO)C3)CC2)nc2ncnn12)c1ccc(Cl)cc1Cl]

In embodiments, the compound has the formula:

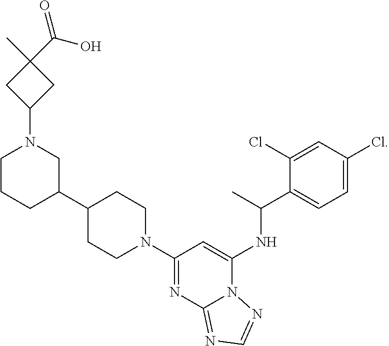 [SMILES: CC(Nc1cc(N2CCC(C3CCCN(C4CC(C)(C(=O)O)C4)C3)CC2)nc2ncnn12)c1ccc(Cl)cc1Cl]

In embodiments, the compound has the formula:

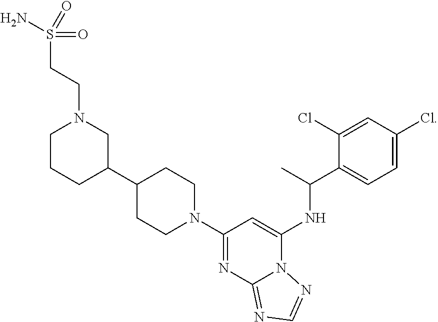 [SMILES: CC(Nc1cc(N2CCC(C3CCCN(CCS(N)(=O)=O)C3)CC2)nc2ncnn12)c1ccc(Cl)cc1Cl]

In embodiments, the compound has the formula:

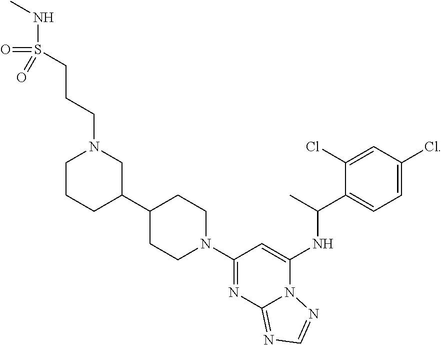 [SMILES: CNS(=O)(=O)CCCN1CCCC(C2CCN(c3cc(NC(C)c4ccc(Cl)cc4Cl)n4ncnc4n3)CC2)C1]

In embodiments, the compound has the formula:

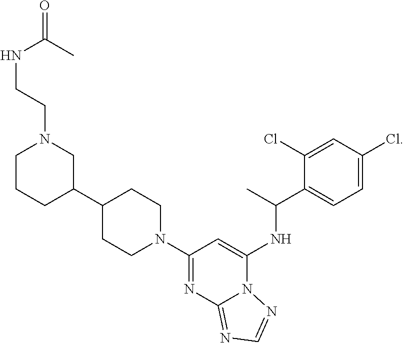 [SMILES: CC(=O)NCCN1CCCC(C2CCN(c3cc(NC(C)c4ccc(Cl)cc4Cl)n4ncnc4n3)CC2)C1]

In embodiments, the compound has the formula:

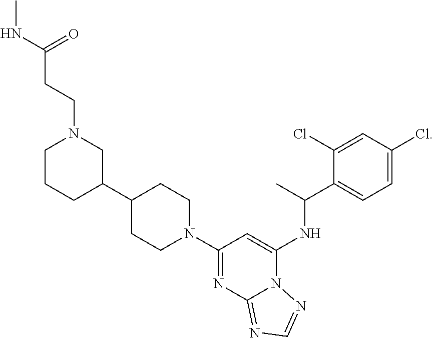 [SMILES: CNC(=O)CCN1CCCC(C2CCN(c3cc(NC(C)c4ccc(Cl)cc4Cl)n4ncnc4n3)CC2)C1]

In embodiments, the compound has the formula:

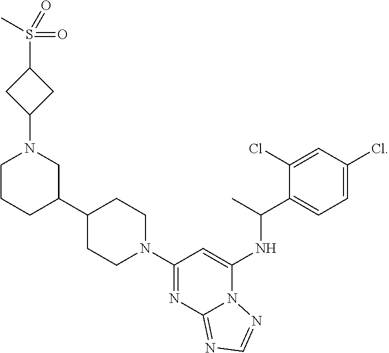 [SMILES: CC(Nc1cc(N2CCC(C3CCCN(C4CC(S(C)(=O)=O)C4)C3)CC2)nc2ncnn12)c1ccc(Cl)cc1Cl]

In embodiments, the compound has the formula:

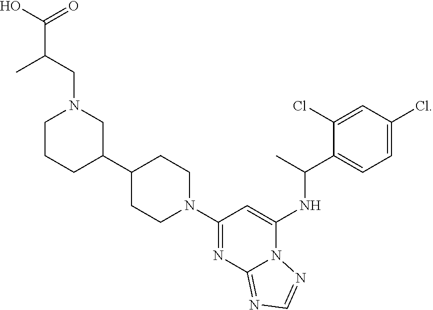 [SMILES: CC(CN1CCCC(C2CCN(c3cc(NC(C)c4ccc(Cl)cc4Cl)n4ncnc4n3)CC2)C1)C(=O)O]

In embodiments, the compound has the formula:

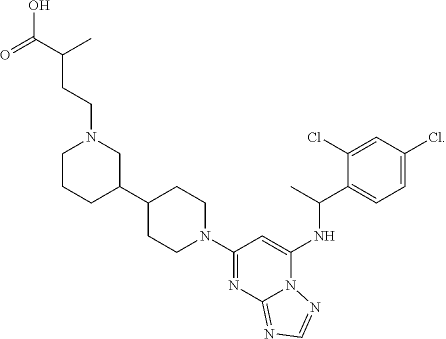 [SMILES: CC(CCN1CCCC(C2CCN(c3cc(NC(C)c4ccc(Cl)cc4Cl)n4ncnc4n3)CC2)C1)C(=O)O]

In embodiments, the compound has the formula:

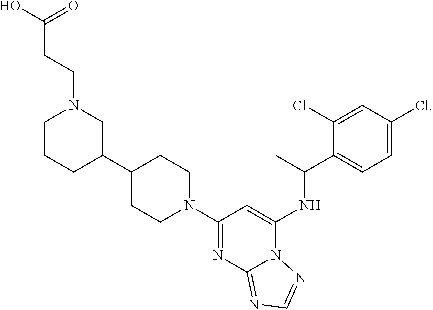 [SMILES: CC(Nc1cc(N2CCC(C3CCCN(CCC(=O)O)C3)CC2)nc2ncnn12)c1ccc(Cl)cc1Cl]

In embodiments, the compound has the formula:

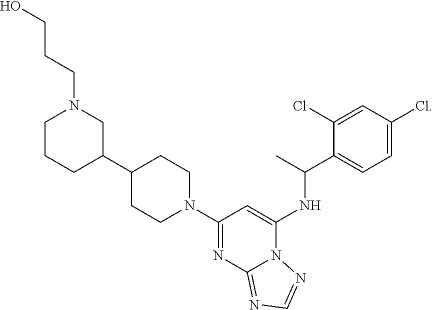 [SMILES: CC(Nc1cc(N2CCC(C3CCCN(CCCO)C3)CC2)nc2ncnn12)c1ccc(Cl)cc1Cl]

In embodiments, the compound has the formula:

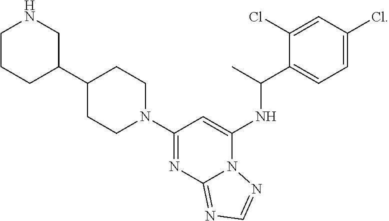 [SMILES: CC(Nc1cc(N2CCC(C3CCCNC3)CC2)nc2ncnn12)c1ccc(Cl)cc1Cl]

In embodiments, the compound has the formula:

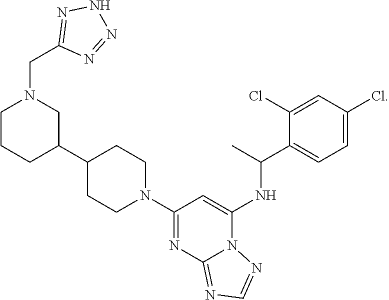 [SMILES: CC(Nc1cc(N2CCC(C3CCCN(Cc4nn[nH]n4)C3)CC2)nc2ncnn12)c1ccc(Cl)cc1Cl]

In embodiments, the compound has the formula:

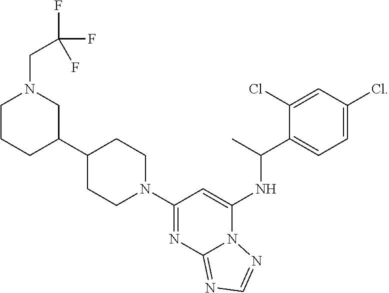 [SMILES: CC(Nc1cc(N2CCC(C3CCCN(CC(F)(F)F)C3)CC2)nc2ncnn12)c1ccc(Cl)cc1Cl]

In embodiments, the compound has the formula:

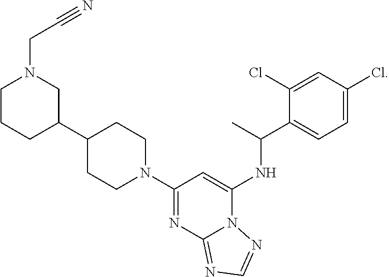 [SMILES: CC(Nc1cc(N2CCC(C3CCCN(CC#N)C3)CC2)nc2ncnn12)c1ccc(Cl)cc1Cl]

In embodiments, the compound has the formula:

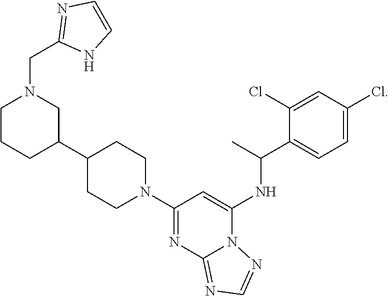 [SMILES: CC(Nc1cc(N2CCC(C3CCCN(Cc4ncc[nH]4)C3)CC2)nc2ncnn12)c1ccc(Cl)cc1Cl]

In embodiments, the compound has the formula

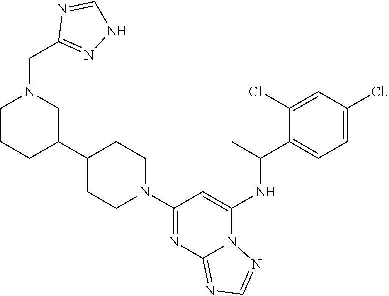 [SMILES: CC(Nc1cc(N2CCC(C3CCCN(Cc4nc[nH]n4)C3)CC2)nc2ncnn12)c1ccc(Cl)cc1Cl]

In embodiments, the compound has the formula:

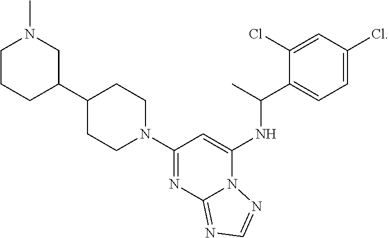 [SMILES: CC(Nc1cc(N2CCC(C3CCCN(C)C3)CC2)nc2ncnn12)c1ccc(Cl)cc1Cl]

In embodiments, the compound has the formula:

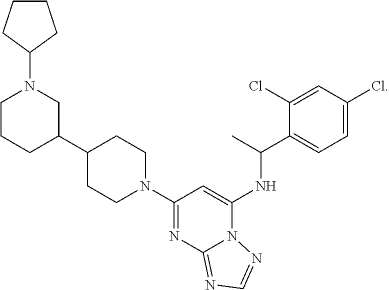 [SMILES: CC(Nc1cc(N2CCC(C3CCCN(C4CCCC4)C3)CC2)nc2ncnn12)c1ccc(Cl)cc1Cl]

In embodiments, the compound has the formula:

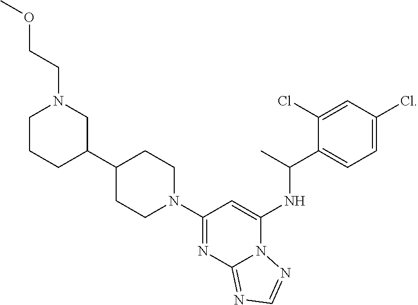 [SMILES: COCCN1CCCC(C2CCN(c3cc(NC(C)c4ccc(Cl)cc4Cl)n4ncnc4n3)CC2)C1]

In embodiments, the compound has the formula:

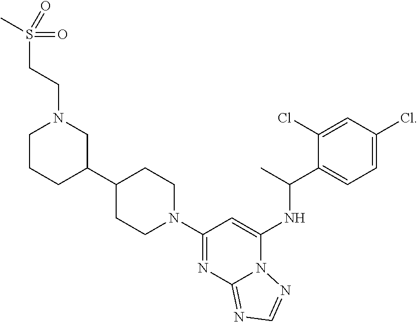 [SMILES: CC(Nc1cc(N2CCC(C3CCCN(CCS(C)(=O)=O)C3)CC2)nc2ncnn12)c1ccc(Cl)cc1Cl]

In embodiments, the compound has the formula:

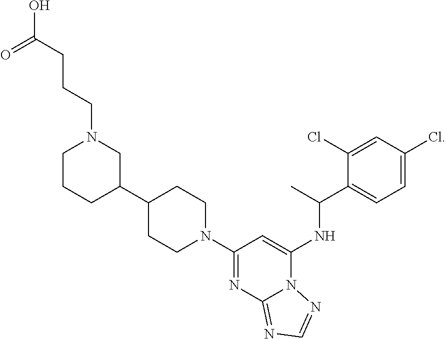 [SMILES: CC(Nc1cc(N2CCC(C3CCCN(CCCC(=O)O)C3)CC2)nc2ncnn12)c1ccc(Cl)cc1Cl]

In embodiments, the compound has the formula:

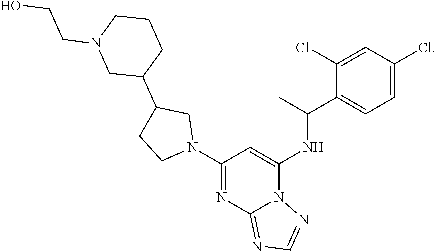 [SMILES: CC(Nc1cc(N2CCC(C3CCCN(CCO)C3)C2)nc2ncnn12)c1ccc(Cl)cc1Cl]

In embodiments, the compound has the formula:

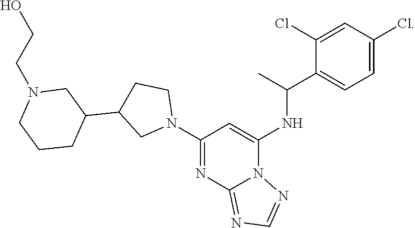 [SMILES: CC(Nc1cc(N2CCC(C3CCCN(CCO)C3)C2)nc2ncnn12)c1ccc(Cl)cc1Cl]

In embodiments, the compound has the formula

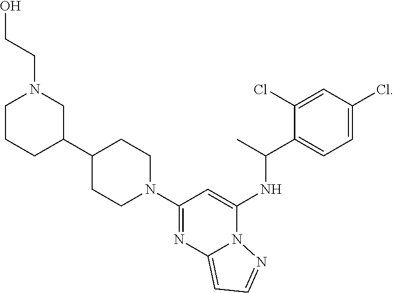 [SMILES: CC(Nc1cc(N2CCC(C3CCCN(CCO)C3)CC2)nc2ccnn12)c1ccc(Cl)cc1Cl]

In embodiments, the compound has the formula:

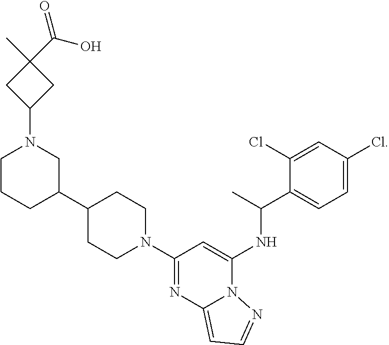 [SMILES: CC(Nc1cc(N2CCC(C3CCCN(C4CC(C)(C(=O)O)C4)C3)CC2)nc2ccnn12)c1ccc(Cl)cc1Cl]

In embodiments, the compound has the formula:

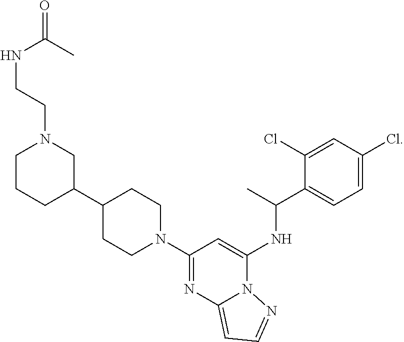 [SMILES: CC(=O)NCCN1CCCC(C2CCN(c3cc(NC(C)c4ccc(Cl)cc4Cl)n4nccc4n3)CC2)C1]

In embodiments, the compound has the formula:

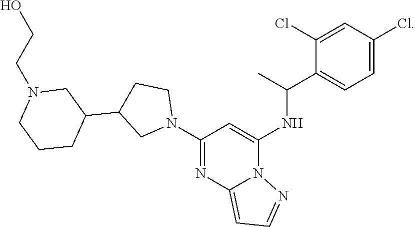 [SMILES: CC(Nc1cc(N2CCC(C3CCCN(CCO)C3)C2)nc2ccnn12)c1ccc(Cl)cc1Cl]

In embodiments, the compound has the formula:

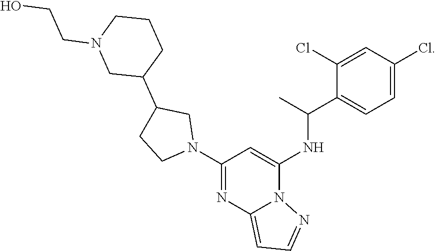 [SMILES: CC(Nc1cc(N2CCC(C3CCCN(CCO)C3)C2)nc2ccnn12)c1ccc(Cl)cc1Cl]

In embodiments, the compound has the formula:

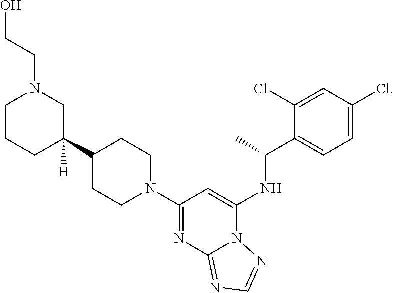 [SMILES: C[C@@H](Nc1cc(N2CCC([C@H]3CCCN(CCO)C3)CC2)nc2ncnn12)c1ccc(Cl)cc1Cl]

In embodiments, the compound has the formula:

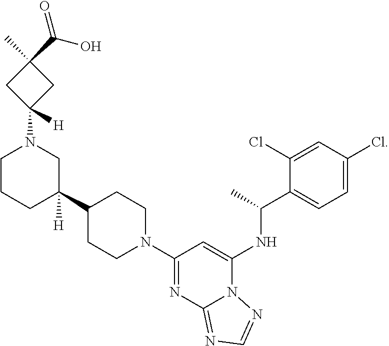 [SMILES: C[C@@H](Nc1cc(N2CCC([C@H]3CCCN([C@H]4C[C@](C)(C(=O)O)C4)C3)CC2)nc2ncnn12)c1ccc(Cl)cc1Cl]

In embodiments, the compound has the formula:

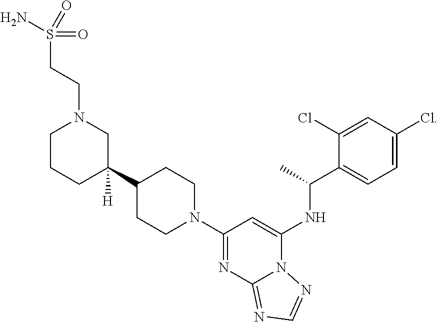 [SMILES: C[C@@H](Nc1cc(N2CCC([C@H]3CCCN(CCS(N)(=O)=O)C3)CC2)nc2ncnn12)c1ccc(Cl)cc1Cl]

In embodiments, the compound has the formula:

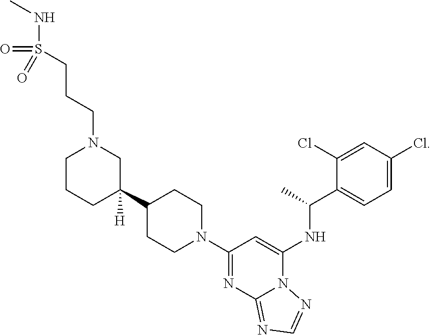 [SMILES: CNS(=O)(=O)CCCN1CCC[C@H](C2CCN(c3cc(N[C@H](C)c4ccc(Cl)cc4Cl)n4ncnc4n3)CC2)C1]

In embodiments, the compound has the formula:

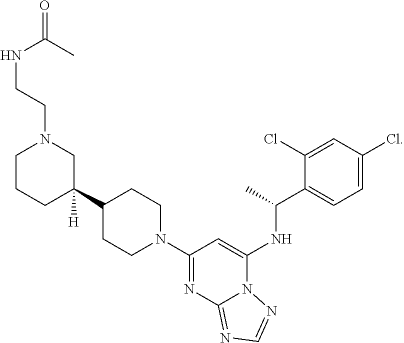 [SMILES: CC(=O)NCCN1CCC[C@H](C2CCN(c3cc(N[C@H](C)c4ccc(Cl)cc4Cl)n4ncnc4n3)CC2)C1]

In embodiments, the compound has the formula:

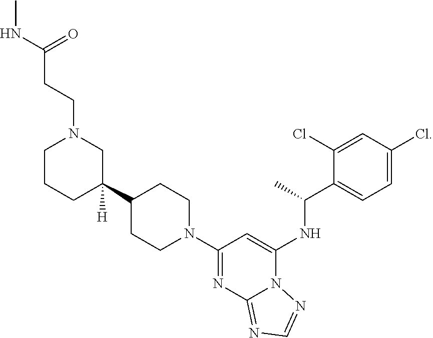 [SMILES: CNC(=O)CCN1CCC[C@H](C2CCN(c3cc(N[C@H](C)c4ccc(Cl)cc4Cl)n4ncnc4n3)CC2)C1]

In embodiments, the compound has the formula:

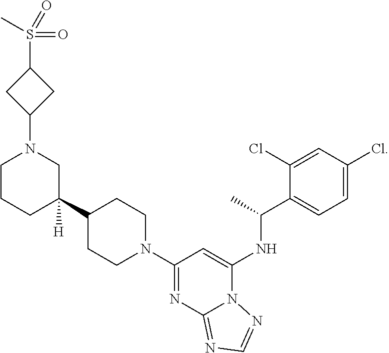 [SMILES: C[C@@H](Nc1cc(N2CCC([C@H]3CCCN(C4CC(S(C)(=O)=O)C4)C3)CC2)nc2ncnn12)c1ccc(Cl)cc1Cl]

In embodiments, the compound has the formula:

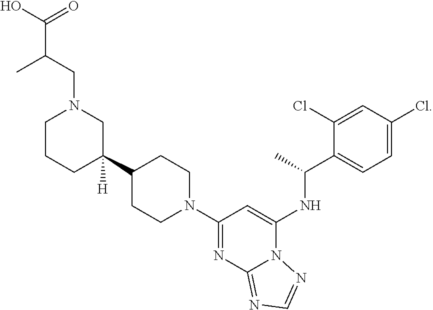 [SMILES: CC(CN1CCC[C@H](C2CCN(c3cc(N[C@H](C)c4ccc(Cl)cc4Cl)n4ncnc4n3)CC2)C1)C(=O)O]

In embodiments, the compound has the formula:

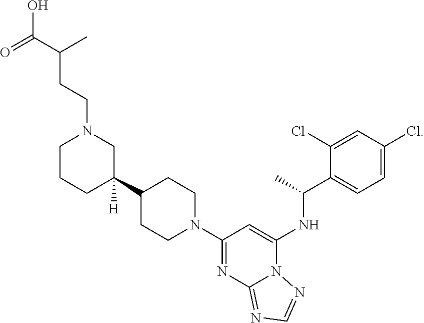 [SMILES: CC(CCN1CCC[C@H](C2CCN(c3cc(N[C@H](C)c4ccc(Cl)cc4Cl)n4ncnc4n3)CC2)C1)C(=O)O]

In embodiments, the compound has the formula:

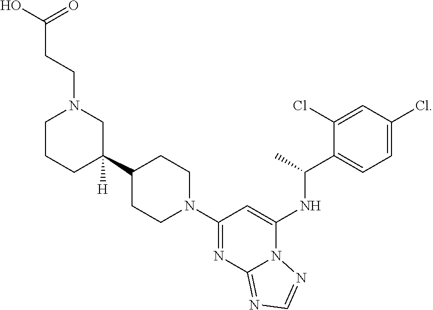 [SMILES: C[C@@H](Nc1cc(N2CCC([C@H]3CCCN(CCC(=O)O)C3)CC2)nc2ncnn12)c1ccc(Cl)cc1Cl]

In embodiments, the compound has the formula:

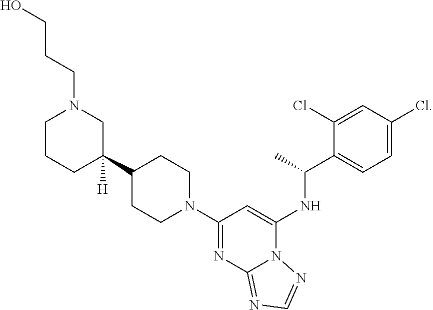 [SMILES: C[C@@H](Nc1cc(N2CCC([C@H]3CCCN(CCCO)C3)CC2)nc2ncnn12)c1ccc(Cl)cc1Cl]

In embodiments, the compound has the formula:

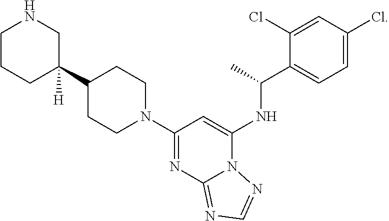 [SMILES: C[C@@H](Nc1cc(N2CCC([C@H]3CCCNC3)CC2)nc2ncnn12)c1ccc(Cl)cc1Cl]

In embodiments, the compound has the formula:

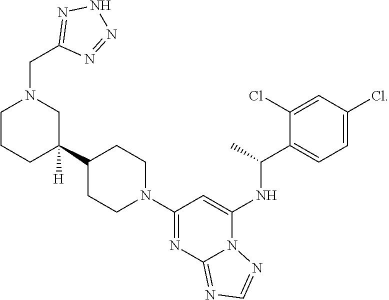 [SMILES: C[C@@H](Nc1cc(N2CCC([C@H]3CCCN(Cc4nn[nH]n4)C3)CC2)nc2ncnn12)c1ccc(Cl)cc1Cl]

In embodiments, the compound has the formula:

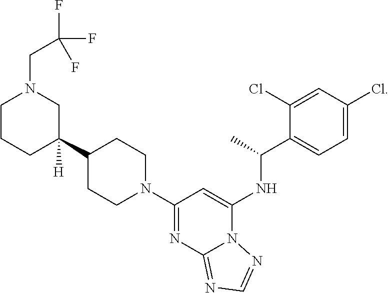 [SMILES: C[C@@H](Nc1cc(N2CCC([C@H]3CCCN(CC(F)(F)F)C3)CC2)nc2ncnn12)c1ccc(Cl)cc1Cl]

In embodiments, the compound has the formula:

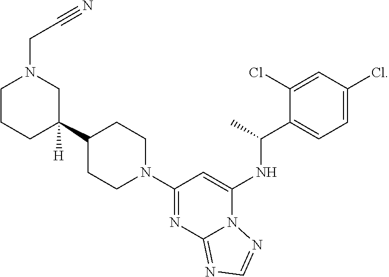 [SMILES: C[C@@H](Nc1cc(N2CCC([C@H]3CCCN(CC#N)C3)CC2)nc2ncnn12)c1ccc(Cl)cc1Cl]

In embodiments, the compound has the formula:

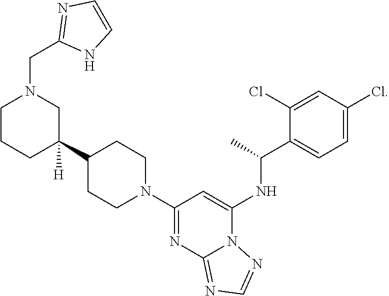 [SMILES: C[C@@H](Nc1cc(N2CCC([C@H]3CCCN(Cc4ncc[nH]4)C3)CC2)nc2ncnn12)c1ccc(Cl)cc1Cl]

In embodiments, the compound has the formula:

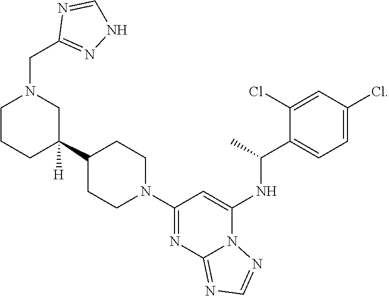 [SMILES: C[C@@H](Nc1cc(N2CCC([C@H]3CCCN(Cc4nc[nH]n4)C3)CC2)nc2ncnn12)c1ccc(Cl)cc1Cl]

In embodiments, the compound has the formula:

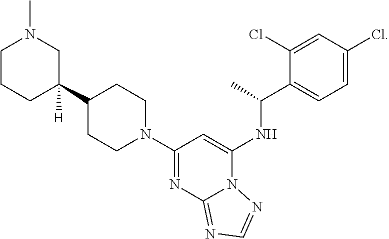 [SMILES: C[C@@H](Nc1cc(N2CCC([C@H]3CCCN(C)C3)CC2)nc2ncnn12)c1ccc(Cl)cc1Cl]

In embodiments, the compound has the formula:

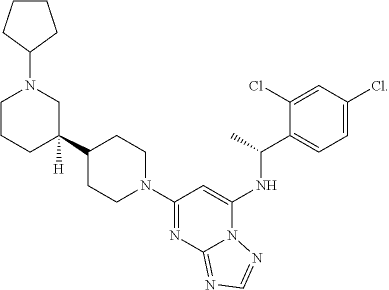 [SMILES: C[C@@H](Nc1cc(N2CCC([C@H]3CCCN(C4CCCC4)C3)CC2)nc2ncnn12)c1ccc(Cl)cc1Cl]

In embodiments, the compound has the formula:

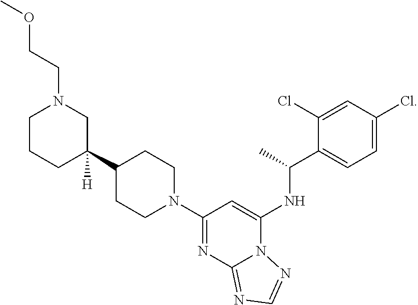 [SMILES: COCCN1CCC[C@H](C2CCN(c3cc(N[C@H](C)c4ccc(Cl)cc4Cl)n4ncnc4n3)CC2)C1]

In embodiments, the compound has the formula:

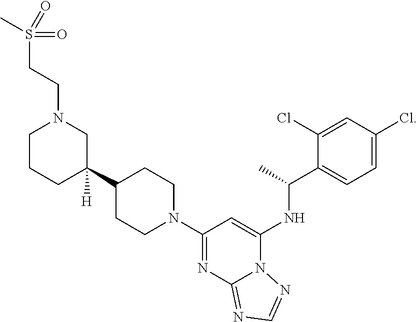 [SMILES: C[C@@H](Nc1cc(N2CCC([C@H]3CCCN(CCS(C)(=O)=O)C3)CC2)nc2ncnn12)c1ccc(Cl)cc1Cl]

In embodiments, the compound has the formula:

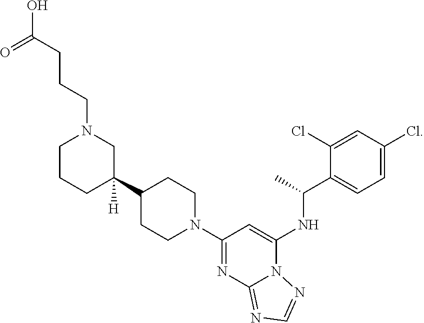 [SMILES: C[C@@H](Nc1cc(N2CCC([C@H]3CCCN(CCCC(=O)O)C3)CC2)nc2ncnn12)c1ccc(Cl)cc1Cl]

In embodiments, the compound has the formula:

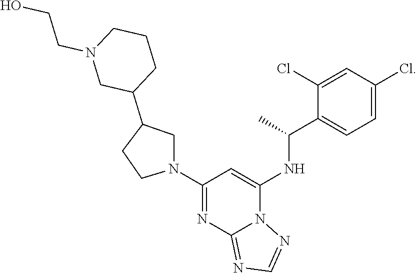 [SMILES: C[C@@H](Nc1cc(N2CCC(C3CCCN(CCO)C3)C2)nc2ncnn12)c1ccc(Cl)cc1Cl]

In embodiments, the compound has the formula:

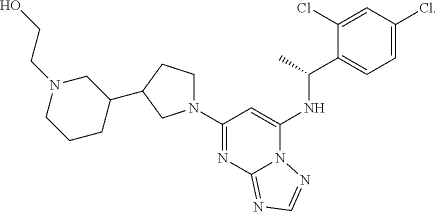 [SMILES: C[C@@H](Nc1cc(N2CCC(C3CCCN(CCO)C3)C2)nc2ncnn12)c1ccc(Cl)cc1Cl]

In embodiments, the compound has the formula:

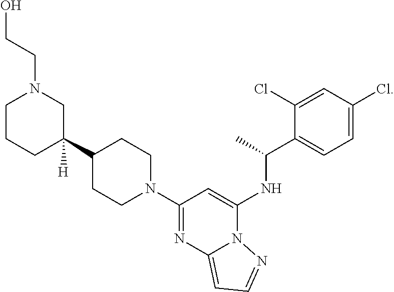 [SMILES: C[C@@H](Nc1cc(N2CCC([C@H]3CCCN(CCO)C3)CC2)nc2ccnn12)c1ccc(Cl)cc1Cl]

In embodiments, the compound has the formula:

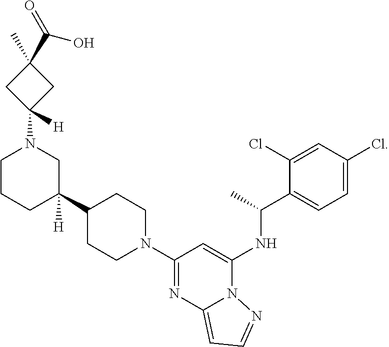 [SMILES: C[C@@H](Nc1cc(N2CCC([C@H]3CCCN([C@H]4C[C@](C)(C(=O)O)C4)C3)CC2)nc2ccnn12)c1ccc(Cl)cc1Cl]

In embodiments, the compound has the formula:

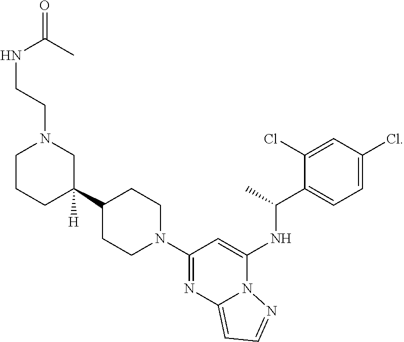 [SMILES: CC(=O)NCCN1CCC[C@H](C2CCN(c3cc(N[C@H](C)c4ccc(Cl)cc4Cl)n4nccc4n3)CC2)C1]

In embodiments, the compound has the formula:

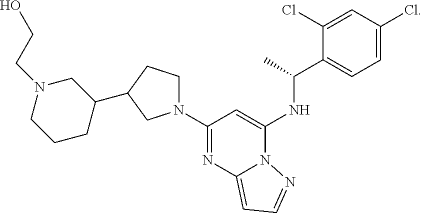 [SMILES: C[C@@H](Nc1cc(N2CCC(C3CCCN(CCO)C3)C2)nc2ccnn12)c1ccc(Cl)cc1Cl]

In embodiments, the compound has the formula:

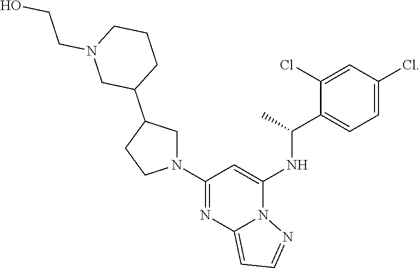 [SMILES: C[C@@H](Nc1cc(N2CCC(C3CCCN(CCO)C3)C2)nc2ccnn12)c1ccc(Cl)cc1Cl]

In embodiments, the compound has the formula:

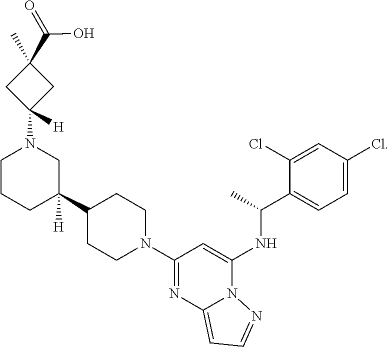 [SMILES: C[C@@H](Nc1cc(N2CCC([C@H]3CCCN([C@H]4C[C@](C)(C(=O)O)C4)C3)CC2)nc2ccnn12)c1ccc(Cl)cc1Cl]

In embodiments, the compound has the formula:

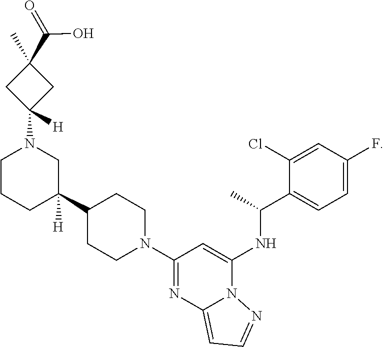 [SMILES: C[C@@H](Nc1cc(N2CCC([C@H]3CCCN([C@H]4C[C@](C)(C(=O)O)C4)C3)CC2)nc2ccnn12)c1ccc(F)cc1Cl]

In embodiments, the compound has the formula:

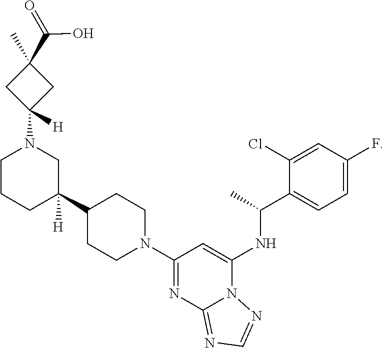 [SMILES: C[C@@H](Nc1cc(N2CCC([C@H]3CCCN([C@H]4C[C@](C)(C(=O)O)C4)C3)CC2)nc2ncnn12)c1ccc(F)cc1Cl]

In embodiments, the compound has the formula:

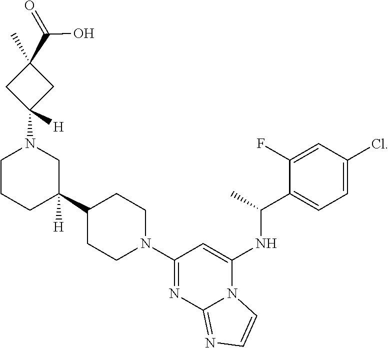 [SMILES: C[C@@H](Nc1cc(N2CCC([C@H]3CCCN([C@H]4C[C@](C)(C(=O)O)C4)C3)CC2)nc2nccn12)c1ccc(Cl)cc1F]

In embodiments, the compound has the formula:

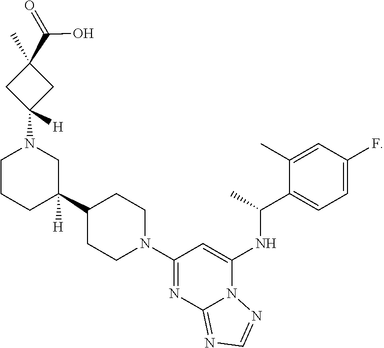 [SMILES: Cc1cc(F)ccc1[C@@H](C)Nc1cc(N2CCC([C@H]3CCCN([C@H]4C[C@](C)(C(=O)O)C4)C3)CC2)nc2ncnn12]

In embodiments, the compound has the formula:

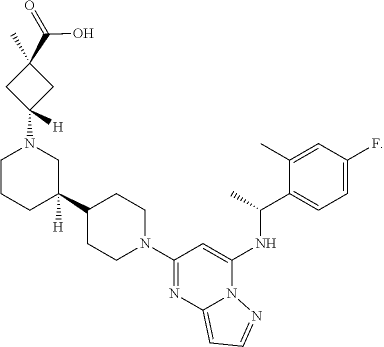 [SMILES: Cc1cc(F)ccc1[C@@H](C)Nc1cc(N2CCC([C@H]3CCCN([C@H]4C[C@](C)(C(=O)O)C4)C3)CC2)nc2ccnn12]

In embodiments, the compound has the formula:

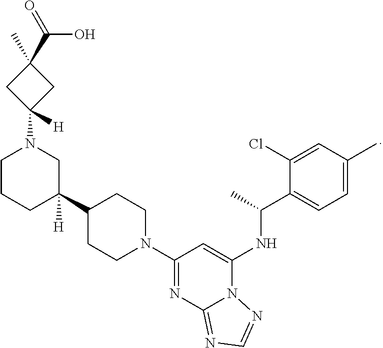 [SMILES: Cc1ccc([C@@H](C)Nc2cc(N3CCC([C@H]4CCCN([C@H]5C[C@](C)(C(=O)O)C5)C4)CC3)nc3ncnn23)c(Cl)c1]

In embodiments, the compound has the formula:

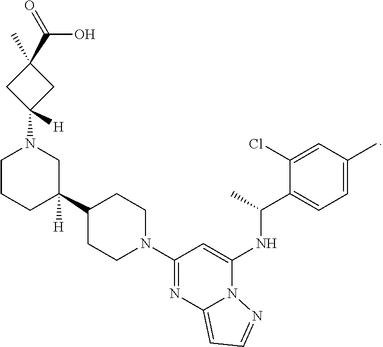 [SMILES: Cc1ccc([C@@H](C)Nc2cc(N3CCC([C@H]4CCCN([C@H]5C[C@](C)(C(=O)O)C5)C4)CC3)nc3ccnn23)c(Cl)c1]

In embodiments, the compound has the formula:

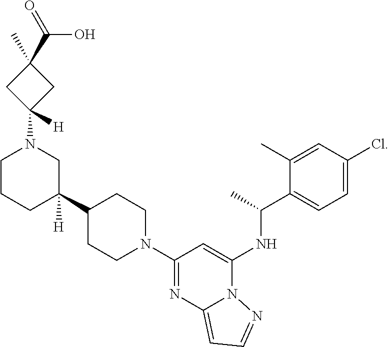 [SMILES: Cc1cc(Cl)ccc1[C@@H](C)Nc1cc(N2CCC([C@H]3CCCN([C@H]4C[C@](C)(C(=O)O)C4)C3)CC2)nc2ccnn12]

In embodiments, the compound has the formula:

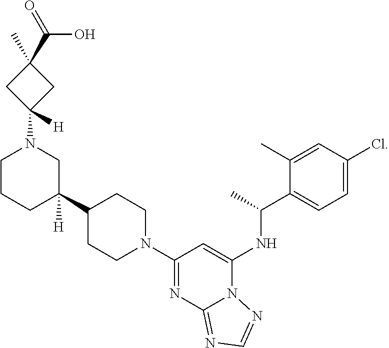 [SMILES: Cc1cc(Cl)ccc1[C@@H](C)Nc1cc(N2CCC([C@H]3CCCN([C@H]4C[C@](C)(C(=O)O)C4)C3)CC2)nc2ncnn12]

In embodiments, the compound has the formula:

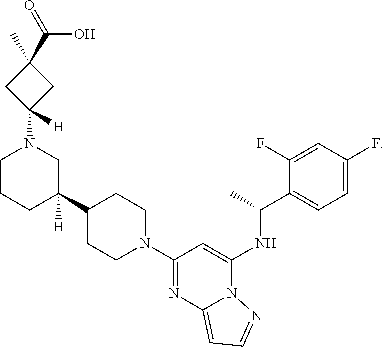 [SMILES: C[C@@H](Nc1cc(N2CCC([C@H]3CCCN([C@H]4C[C@](C)(C(=O)O)C4)C3)CC2)nc2ccnn12)c1ccc(F)cc1F]

In embodiments, the compound has the formula:

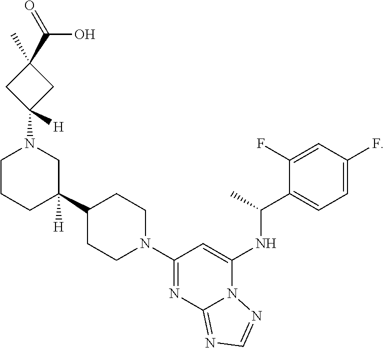 [SMILES: C[C@@H](Nc1cc(N2CCC([C@H]3CCCN([C@H]4C[C@](C)(C(=O)O)C4)C3)CC2)nc2ncnn12)c1ccc(F)cc1F]

In embodiments, the compound has the formula:

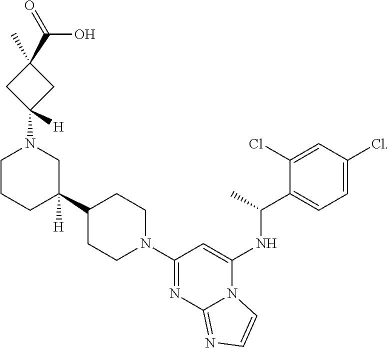 [SMILES: C[C@@H](Nc1cc(N2CCC([C@H]3CCCN([C@H]4C[C@](C)(C(=O)O)C4)C3)CC2)nc2nccn12)c1ccc(Cl)cc1Cl]

In embodiments, the compound has the formula:

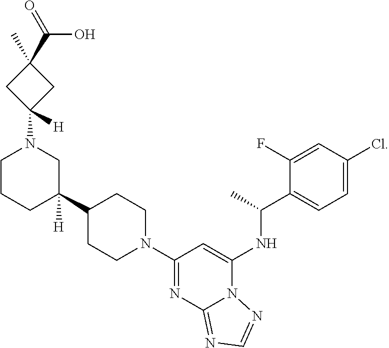 [SMILES: C[C@@H](Nc1cc(N2CCC([C@H]3CCCN([C@H]4C[C@](C)(C(=O)O)C4)C3)CC2)nc2ncnn12)c1ccc(Cl)cc1F]

In embodiments, the compound has the formula:

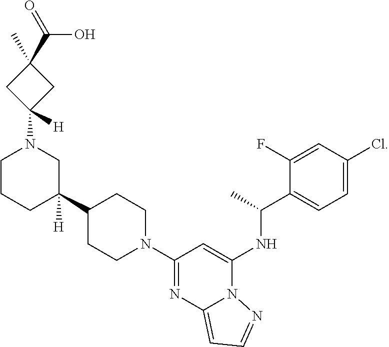 [SMILES: C[C@@H](Nc1cc(N2CCC([C@H]3CCCN([C@H]4C[C@](C)(C(=O)O)C4)C3)CC2)nc2ccnn12)c1ccc(Cl)cc1F]

In embodiments, the compound is useful as a comparator compound. In embodiments, the comparator compound can be used to assess the activity of a test compound as set forth in an assay described herein (e.g., in the examples section or tables).

In embodiments, the compound is a compound as described herein, including in embodiments. In embodiments the compound is a compound described herein (e.g., in the examples section, tables, or claims).

### III. Pharmaceutical Compositions

In an aspect is provided a pharmaceutical composition including a compound described herein, or a pharmaceutically acceptable salt thereof, and a pharmaceutically acceptable excipient. In embodiments, the pharmaceutical composition includes an effective amount of the compound. In embodiments, the pharmaceutical composition includes a therapeutically effective amount of the compound. In embodiments, the compound is a compound of formula (I), (II), (III), (IVa), (IVb), (IVc), (Va), (Vb), (Vc), (VI), (VIIa), (VIIb), (VIIc), (VIIIa), (VIIIb), or (VIIIc), including all embodiments thereof.

The compounds described herein (e.g., CCR4 inhibitors) may be in the form of compositions suitable for administration to a subject. In general, such compositions are “pharmaceutical compositions” including a compound (e.g., CCR4 inhibitor(s)) and one or more pharmaceutically acceptable or physiologically acceptable diluents, carriers or excipients. In embodiments, the compound described herein (e.g., CCR4 inhibitor) is present in a therapeutically acceptable amount. The pharmaceutical compositions may be used in the methods described herein. For example, the pharmaceutical compositions can be administered ex vivo or in vivo to a subject in order to practice the therapeutic and prophylactic methods and uses described herein.

The pharmaceutical compositions of the present invention can be formulated to be compatible with the intended method or route of administration; exemplary routes of administration are set forth herein.

The pharmaceutical compositions containing the active ingredient (e.g., an inhibitor of CCR4 function, compound described herein) may be in a form suitable for oral use, for example, as tablets, capsules, troches, lozenges, aqueous or oily suspensions, dispersible powders or granules, emulsions, hard or soft capsules, or syrups, solutions, microbeads or elixirs. Pharmaceutical compositions intended for oral use may be prepared according to any method known to the art for the manufacture of pharmaceutical compositions, and such compositions may contain one or more agents such as, for example, sweetening agents, flavoring agents, coloring agents and preserving agents in order to provide pharmaceutically elegant and palatable preparations. Tablets, capsules, and the like contain the active ingredient in admixture with non-toxic pharmaceutically acceptable excipients which are suitable for the manufacture thereof. These excipients may be, for example, diluents, such as calcium carbonate, sodium carbonate, lactose, calcium phosphate, or sodium phosphate; granulating and disintegrating agents, for example, corn starch, or alginic acid; binding agents, for example, starch, gelatin, or acacia, and lubricating agents, for example, magnesium stearate, stearic acid, or talc.

The tablets, capsules and the like suitable for oral administration may be uncoated or coated by known techniques to delay disintegration and absorption in the gastrointestinal tract and thereby provide a sustained action. For example, a time-delay material such as glyceryl monostearate or glyceryl distearate may be employed. They may also be coated by techniques known in the art to form osmotic therapeutic tablets for controlled release. Additional agents include biodegradable or biocompatible particles or a polymeric substance such as polyesters, polyamine acids, hydrogel, polyvinyl pyrrolidone, polyanhydrides, polyglycolic acid, ethylene-vinylacetate, methylcellulose, carboxymethylcellulose, protamine sulfate, or lactide/glycolide copolymers, polylactide/glycolide copolymers, or ethylenevinylacetate copolymers in order to control delivery of an administered composition. For example, the oral agent can be entrapped in microcapsules prepared by coacervation techniques or by interfacial polymerization, by the use of hydroxymethylcellulose or gelatin-microcapsules or poly(methylmethacrolate) microcapsules, respectively, or in a colloid drug delivery system. Colloidal dispersion systems include macromolecule complexes, nano-capsules, microspheres, microbeads, and lipid-based systems, including oil-in-water emulsions, micelles, mixed micelles, and liposomes. Methods for the preparation of the above-mentioned formulations will be apparent to those skilled in the art.

Formulations for oral use may also be presented as hard gelatin capsules wherein the active ingredient is mixed with an inert solid diluent, for example, calcium carbonate, calcium phosphate, kaolin, or microcrystalline cellulose, or as soft gelatin capsules wherein the active ingredient is mixed with water or an oil medium, for example, peanut oil, liquid paraffin, or olive oil.

Aqueous suspensions contain the active materials in admixture with excipients suitable for the manufacture thereof. Such excipients can be suspending agents, for example, sodium carboxymethylcellulose, methylcellulose, hydroxy-propylmethylcellulose, sodium alginate, polyvinyl-pyrrolidone, gum tragacanth, or gum acacia; dispersing or wetting agents, for example, a naturally-occurring phosphatide (e.g., lecithin), or condensation products of an alkylene oxide with fatty acids (e.g., polyoxy-ethylene stearate), or condensation products of ethylene oxide with long chain aliphatic alcohols (e.g., for heptadecaethyleneoxycetanol), or condensation products of ethylene oxide with partial esters derived from fatty acids and a hexitol (e.g., polyoxyethylene sorbitol monooleate), or condensation products of ethylene oxide with partial esters derived from fatty acids and hexitol anhydrides (e.g., polyethylene sorbitan monooleate). The aqueous suspensions may also contain one or more preservatives.

Oily suspensions may be formulated by suspending the active ingredient in a vegetable oil, for example, arachis oil, olive oil, sesame oil or coconut oil, or in a mineral oil such as liquid paraffin. The oily suspensions may contain a thickening agent, for example, beeswax, hard paraffin or cetyl alcohol. Sweetening agents, such as those set forth above, and flavoring agents may be added to provide a palatable oral preparation.

Dispersible powders and granules suitable for preparation of an aqueous suspension by the addition of water provide the active ingredient in admixture with a dispersing or wetting agent, and optionally one or more suspending agents and/or preservatives. Suitable dispersing or wetting agents and suspending agents are exemplified herein.

The pharmaceutical compositions of the present invention may also be in the form of oil-in-water emulsions. The oily phase may be a vegetable oil, for example olive oil or arachis oil, or a mineral oil, for example, liquid paraffin, or mixtures of these. Suitable emulsifying agents may be naturally occurring gums, for example, gum acacia or gum tragacanth; naturally occurring phosphatides, for example, soy bean, lecithin, and esters or partial esters derived from fatty acids; hexitol anhydrides, for example, sorbitan monooleate; and condensation products of partial esters with ethylene oxide, for example, polyoxyethylene sorbitan monooleate.

The pharmaceutical compositions may include a therapeutically effective amount of a CCR4 inhibitor described herein and one or more pharmaceutically and physiologically acceptable formulation agents. Suitable pharmaceutically acceptable or physiologically acceptable diluents, carriers or excipients include, but are not limited to, antioxidants (e.g., ascorbic acid and sodium bisulfate), preservatives (e.g., benzyl alcohol, methyl parabens, ethyl or n-propyl, p-hydroxybenzoate), emulsifying agents, suspending agents, dispersing agents, solvents, fillers, bulking agents, detergents, buffers, vehicles, diluents, and/or adjuvants. For example, a suitable vehicle may be physiological saline solution or citrate-buffered saline, possibly supplemented with other materials common in pharmaceutical compositions for parenteral administration. Neutral buffered saline or saline mixed with serum albumin are further exemplary vehicles. Those skilled in the art will readily recognize a variety of buffers that can be used in the pharmaceutical compositions and dosage forms contemplated herein. Typical buffers include, but are not limited to, pharmaceutically acceptable weak acids, weak bases, or mixtures thereof. As an example, the buffer components can be water soluble materials such as phosphoric acid, tartaric acids, lactic acid, succinic acid, citric acid, acetic acid, ascorbic acid, aspartic acid, glutamic acid, and salts thereof. Acceptable buffering agents include, for example, a Tris buffer; N-(2-Hydroxyethyl)piperazine-N′-(2-ethanesulfonic acid) (HEPES); 2-(N-Morpholino)ethanesulfonic acid (MES); 2-(N-Morpholino)ethanesulfonic acid sodium salt (MES); 3-(N-Morpholino)propanesulfonic acid (MOPS); and N-tris[Hydroxymethyl]methyl-3-aminopropanesulfonic acid (TAPS).

After a pharmaceutical composition has been formulated, it may be stored in sterile vials as a solution, suspension, gel, emulsion, solid, or dehydrated or lyophilized powder. Such formulations may be stored either in a ready-to-use form, a lyophilized form requiring reconstitution prior to use, a liquid form requiring dilution prior to use, or other acceptable form. In embodiments, the pharmaceutical composition is provided in a single-use container (e.g., a single-use vial, ampule, syringe, or autoinjector (similar to, e.g., an EpiPen®)), whereas a multi-use container (e.g., a multi-use vial) is provided in other embodiments.

Formulations can also include carriers to protect the composition against rapid degradation or elimination from the body, such as a controlled release formulation, including liposomes, hydrogels, prodrugs and microencapsulated delivery systems. For example, a time-delay material such as glyceryl monostearate or glyceryl stearate alone, or in combination with a wax, may be employed. Any drug delivery apparatus may be used to deliver a CCR4 inhibitor (e.g., compound described herein), including implants (e.g., implantable pumps) and catheter systems, slow injection pumps and devices, all of which are well known to the skilled artisan.

Depot injections, which are generally administered subcutaneously or intramuscularly, may also be utilized to release the compound (e.g., CCR4 inhibitor) disclosed herein over a defined period of time. Depot injections are usually either solid- or oil-based and generally comprise at least one of the formulation components set forth herein. One of ordinary skill in the art is familiar with possible formulations and uses of depot injections.

The pharmaceutical compositions may be in the form of a sterile injectable aqueous or oleagenous suspension. This suspension may be formulated according to the known art using those suitable dispersing or wetting agents and suspending agents mentioned herein. The sterile injectable preparation may also be a sterile injectable solution or suspension in a non-toxic parenterally-acceptable diluent or solvent, for example, as a solution in 1,3-butane diol. Acceptable diluents, solvents and dispersion media that may be employed include water, Ringer's solution, isotonic sodium chloride solution, Cremophor® EL (BASF, Parsippany, NJ) or phosphate buffered saline (PBS), ethanol, polyol (e.g., glycerol, propylene glycol, and liquid polyethylene glycol), and suitable mixtures thereof. In addition, sterile fixed oils are conventionally employed as a solvent or suspending medium; for this purpose, any bland fixed oil may be employed, including synthetic mono- or diglycerides. Moreover, fatty acids, such as oleic acid, find use in the preparation of injectables. Prolonged absorption of particular injectable formulations can be achieved by including an agent that delays absorption (e.g., aluminum monostearate or gelatin).

The present invention contemplates the administration of a compound described herein (e.g., CCR4 inhibitor) in the form of suppositories for rectal administration. The suppositories can be prepared by mixing the drug with a suitable non-irritating excipient, which is solid at ordinary temperatures but liquid at the rectal temperature and will therefore melt in the rectum to release the drug. Such materials include, but are not limited to, cocoa butter and polyethylene glycols.

The compounds (e.g., CCR4 inhibitor) described herein may be in the form of any other suitable pharmaceutical composition (e.g., sprays for nasal or inhalation use) currently known or developed in the future.

### IV. Methods of Use

In an aspect is provided a method of inhibiting C-C chemokine receptor type 4 (CCR4), including contacting CCR4 with a compound described herein, or a pharmaceutically acceptable salt thereof. In embodiments, the compound is a compound of formula (I), (II), (III), (IVa), (IVb), (IVc), (Va), (Vb), (Vc), (VI), (VIIa), (VIIb), (VIIc), (VIIIa), (VIIIb), or (VIIIc), including all embodiments thereof.

In an aspect is provided a method of treating or preventing a disease or disorder mediated by CCR4, including administering to a subject in need thereof a therapeutically effective amount of a compound described herein, or a pharmaceutically acceptable salt thereof. In embodiments, the compound is a compound of formula (I), (II), (III), (IVa), (IVb), (IVc), (Va), (Vb), (Vc), (VI), (VIIa), (VIIb), (VIIc), (VIIIa), (VIIIb), or (VIIIc), including all embodiments thereof.

In an aspect is provided a method of treating or preventing a disease or disorder mediated by CCR4, including administering to a subject in need thereof a therapeutically effective amount of a pharmaceutical composition described herein.

In embodiments, the disease or disorder is an immune disease or disorder or an inflammatory disease or disorder. In embodiments, the disease or disorder is inflammation. In embodiments, the method further includes co-administering an anti-inflammatory agent to the subject. In embodiments, the anti-inflammatory agent is thalidomide or a derivative thereof, a retinoid, dithranol, calcipotriol, a non-steroidal anti-inflammatory agent (NSAID), a cyclo-oxygenase inhibiting nitric oxide donor (CINOD), a glucocorticosteroid, methotrexate, leflunomide, hydroxychloroquine, d-penicillamine, auranofin, an analgesic, a diacerein, hyaluronic acid derivative, or a nutritional supplement.

In embodiments, the disease or disorder is traumatic brain injury, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome,vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo, asthma, allergic asthma, acne vulgaris, celiac disease, chronic prostatitis, inflammatory bowel disease, pelvic inflammatory disease, reperfusion injury, transplant rejection, interstitial cystitis, atherosclerosis, or atopic dermatitis.

In embodiments, the disease or disorder is a cardiovascular disease or disorder or a metabolic disease or disorder. In embodiments, the method further includes co-administering a cardiovascular disorder agent or a metabolic disorder agent to the subject. In embodiments, the cardiovascular disorder agent is a calcium channel blocker, a beta-adrenoceptor blocker, an angiotensin-converting enzyme (ACE) inhibitor, an angiotensin-2 receptor antagonist, a lipid lowering agent, a modulator of blood cell morphology, a thrombolytic, or an anticoagulant.

In embodiments, the disease or disorder is cancer. In embodiments, the cancer is colon cancer or pancreatic cancer. In embodiments, the method further includes co-administering a chemotherapeutic agent or anti-cancer agent to the subject. In embodiments, the chemotherapeutic agent or anti-cancer agent is an antiproliferative/antineoplastic drug, an antimetabolite, an antitumour antibiotic, an antimitotic agent, a topoisomerase inhibitor, a cytostatic agent, an oestrogen receptor down regulator, an antiandrogen, a LHRH antagonist or LHRH agonist, a progestogen, an aromatase inhibitor, an inhibitor of 5a-reductase, an agent which inhibits cancer cell invasion, an inhibitor of growth factor function, a farnesyl transferase inhibitor, a tyrosine kinase inhibitor, a serine/threonine kinase inhibitor, an inhibitor of the epidermal growth factor family, an inhibitor of the platelet-derived growth factor family, an inhibitor of the hepatocyte growth factor family; an antiangiogenic agent, a vascular damaging agent, an agent used in antisense therapy, an anti-ras antisense, an agent used in a gene therapy, an immunotherapeutic agent, or an antibody. In embodiments, the chemotherapeutic agent or anti-cancer agent is an inhibitor of the PD-L1/PD-1 pathway, an inhibitor of CTLA-4 or an agonistic antibody of CD137 (4.1BB). In embodiments, the chemotherapeutic agent or anti-cancer agent is an agent from Table 1. In embodiments, the method further includes co-administering a therapeutically effective amount of at least two of: a CCR4 inhibitor, an immune modulator agent, or an agent from Table 1, or any combination thereof.

In embodiments, the disease or disorder is inflammatory bowel disease. In embodiments, the disease or disorder is rheumatoid arthritis. In embodiments, the disease or disorder is psoriasis. In embodiments, the disease or disorder includes allergy-related disorders (e.g., hypersensitivity and anaphylactic responses); gastrointestinal disorders (e.g., Crohn's disease, ulcerative colitis, ileitis and enteritis); psoriasis and inflammatory dermatoses (e.g., dermatitis, eczema, atopic dermatitis, allergic contact dermatitis, dermatomyositis, urticaria and pruritus); vasculitis; scleroderma; asthma, COPD, and respiratory allergic diseases (e.g., allergic rhinitis and hypersensitivity lung diseases); autoimmune diseases, including arthritis (e.g., rheumatoid and psoriatic), multiple sclerosis, systemic lupus erythematosus, type I diabetes and glomerulonephritis; graft rejection (e.g., allograft rejection); transplant rejection (e.g., solid organ); cancers, such as leukemias, lymphomas and metastatic cancers, particularly solid tumors (e.g., gastric cancers); and other diseases in which inhibition of undesired inflammatory and/or immune responses is desired, such as atherosclerosis, neurodegenerative diseases (e.g., Alzheimer's disease), encephalitis, meningitis, hepatitis, nephritis, sepsis, sarcoidosis, allergic conjunctivitis, otitis, and sinusitis. In embodiments, the CCR4-mediated disease, disorder or condition is asthma, COPD, rhinitis, idiopathic pulmonary fibrosis, psoriasis and contact dermatitis. In embodiments, the disease or disorder is including pulmonary fibrosis, hepatic inflammation, asthma, atopic dermatitis, cancer (e.g., thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma), or granuloma development.

In embodiments, the disease or disorder is asthma, urticaria, rhinitis, or dermatitis. In embodiments, the disease or disorder is allergic asthma, autoimmune urticaria, allergic rhinitis, or contact dermatitis.

In embodiments, the administration of the compounds disclosed herein are for the treatment or prevention of immune-, inflammatory-, or cancer-related diseases, disorders, and conditions. Such diseases, disorders and conditions are described in detail elsewhere, as are other maladies that may be treated or prevented with compounds (e.g., CCR4 inhibitor) described herein.

It is frequently beneficial to improve one of more physical properties of the treatment modalities disclosed herein and/or the manner in which they are administered. Improvements of physical properties include, for example, methods of increasing water solubility, bioavailability, serum half-life, and/or therapeutic half-life; and/or modulating biological activity. Modifications known in the art include pegylation, Fc-fusion and albumin fusion. Although generally associated with large molecule agents (e.g., polypeptides), such modifications have recently been evaluated with particular small molecules. By way of example, Chiang, M. et al. (J. Am. Chem. Soc., 2014, 136(9):3370-73) describe a small molecule agonist of the adenosine 2a receptor conjugated to the immunoglobulin Fc domain. The small molecule-Fc conjugate retained potent Fc receptor and adenosine 2a receptor interactions and showed superior properties compared to the unconjugated small molecule. Covalent attachment of PEG molecules to small molecule therapeutics has also been described (Li, W. et al., Progress in Polymer Science, 2013 38:421-44).

In embodiments, compounds of the present invention are effective in the treatment and prevention of IBD (e.g., Crohn's disease and ulcerative colitis, both of which are chronic idiopathic diseases that can affect any part of the gastrointestinal tract, and are associated with many untoward effects, and patients with prolonged ulcerative colitis are at an increased risk of developing colon cancer). Current IBD treatments are aimed at controlling inflammatory symptoms, and while certain agents (e.g., corticosteroids, aminosalicylates and standard immunosuppressive agents (e.g., cyclosporine, azathioprine, and methotrexate)) have met with limited success, long-term therapy may cause liver damage (e.g., fibrosis or cirrhosis) and bone marrow suppression, and patients often become refractory to such treatments.

The compounds described herein can be used to increase or enhance an immune response; to improve immunization, including increasing vaccine efficacy; and to increase inflammation. Immune deficiencies associated with immune deficiency diseases, immunosuppressive medical treatment, acute and/or chronic infection, and aging can be treated using the compounds disclosed herein. The compounds described herein can also be used to stimulate the immune system of patients suffering from iatrogenically-induced immune suppression, including those who have undergone bone marrow transplants, chemotherapy, or radiotherapy.

In accordance with the present invention, a compound or pharmaceutical salt thereof can be used to treat or prevent a proliferative condition or disorder, including a cancer, for example, cancer of the uterus, cervix, breast, prostate, testes, gastrointestinal tract (e.g., esophagus, oropharynx, stomach, small or large intestines, colon, or rectum), kidney, renal cell, bladder, bone, bone marrow, skin, head or neck, liver, gall bladder, heart, lung, pancreas, salivary gland, adrenal gland, thyroid, brain (e.g., gliomas), ganglia, central nervous system (CNS) and peripheral nervous system (PNS), and cancers of the hematopoietic system and the immune system (e.g., spleen or thymus). In embodiments are methods of treating or preventing other cancer-related diseases, disorders or conditions, including, for example, immunogenic tumors, non-immunogenic tumors, dormant tumors, virus-induced cancers (e.g., epithelial cell cancers, endothelial cell cancers, squamous cell carcinomas and papillomavirus), adenocarcinomas, lymphomas, carcinomas, melanomas, leukemias, myelomas, sarcomas, teratocarcinomas, chemically-induced cancers, metastasis, and angiogenesis. In embodiments is included reducing tolerance to a tumor cell or cancer cell antigen, e.g., by modulating activity of a regulatory T-cell and/or a CD8+ T-cell (see, e.g., Ramirez-Montagut, et al. (2003) Oncogene 22:3180-87; and Sawaya, et al. (2003) New Engl. J. Med. 349:1501-09). In embodiments, the tumor or cancer is colon cancer, ovarian cancer, breast cancer, melanoma, lung cancer, glioblastoma, or leukemia. In embodiments, the cancer is gastric cancer. The use of the term(s) cancer-related diseases, disorders and conditions is meant to refer broadly to conditions that are associated, directly or indirectly, with cancer, and includes, e.g., angiogenesis and precancerous conditions such as dysplasia. In embodiments, the cancer is thyroid carcinoma, cholangiocarcinoma, pancreatic cancer, pancreatic adenocarcinoma, skin cutaneous melanoma, colon cancer, colon adenocarcinoma, rectum adenocarcinoma, stomach adenocarcinoma, esophageal carcinoma, head and neck squamous cell carcinoma, breast invasive carcinoma, lung adenocarcinoma, or lung squamous cell carcinoma.

In embodiments, a cancer can be metastatic or at risk of becoming metastatic, or may occur in a diffuse tissue, including cancers of the blood or bone marrow (e.g., leukemia). In embodiments, the compounds of the invention can be used to overcome T-cell tolerance.

In embodiments, provided are methods for treating a proliferative condition, cancer, tumor, or precancerous condition with a compound described herein and at least one additional therapeutic or diagnostic agent, examples of which are set forth elsewhere herein.

In embodiments, provided is a method for treating and/or preventing a proliferative condition, cancer, tumor, or precancerous disease, disorder or condition with a compound described herein.

In embodiments drawn to methods of treating cancer, the administration of a therapeutically effective amount of a compound described herein results in a cancer survival rate greater than the cancer survival rate observed by administering either agent alone. In further embodiments drawn to methods of treating cancer, the administration of a therapeutically effective amount of a compound described herein (e.g., CCR4 inhibitor) results in a reduction of tumor size or a slowing of tumor growth greater than reduction of tumor size or tumor growth observed following administration of either agent alone. In embodiments, the methods of treating cancer disclosed herein further include administering a chemotherapeutic agent or anticancer agent in combination with a compound described herein (e.g., of structural Formula (I), (II), (III), (IVa), (IVb), (IVc), (Va), (Vb), (Vc), (VI), (VIIa), (VIIb), (VIIc), (VIIIa), (VIIIb), or (VIIIc), including all embodiments thereof), or a pharmaceutically acceptable salt thereof. In embodiments, the chemotherapeutic agent or anticancer agent is an antiproliferative/antineoplastic drug, an antimetabolite, an antitumour antibiotic, an antimitotic agent, a topoisomerase inhibitor, a cytostatic agent, an oestrogen receptor down regulator, an antiandrogen, a LHRH antagonist or LHRH agonist, a progestogen, an aromatase inhibitor, an inhibitor of 5a-reductase, an agent which inhibits cancer cell invasion, an inhibitor of growth factor function, a farnesyl transferase inhibitor, a tyrosine kinase inhibitor, a serine/threonine kinase inhibitor, an inhibitor of the epidermal growth factor family, an inhibitor of the platelet-derived growth factor family, an inhibitor of the hepatocyte growth factor family; an antiangiogenic agent, a vascular damaging agent, an agent used in antisense therapy, an anti-ras antisense, an agent used in a gene therapy, an immunotherapeutic agent, oran antibody. In embodiments, the methods of treating cancer disclosed herein further include co-administering a therapeutically effective amount of at least two of: a CCR4 inhibitor, an inhibitor of the PD-L1/PD-1 pathway, an inhibitor of CTLA-4 or an agonistic antibody of CD137 (4.1BB). In embodiments, the methods of treating cancer disclosed herein further include co-administering a therapeutically effective amount of at least two of: a CCR4 inhibitor, an agent that may be an immune modulator or an agent from Table 1.

Inhibition of CCR4 activity may also represent an important strategy for the treatment or prevention of neurological, neuropsychiatric, neurodegenerative or other diseases, disorders and conditions having some association with the central nervous system, including disorders associated with impairment of cognitive function and/or motor function. Many of these diseases, disorders and conditions comprise an immune and/or inflammatory component. In embodiments, the disease or disorder is Parkinson's disease, extra pyramidal syndrome (EPS), dystonia, akathisia, tardive dyskinesia, restless leg syndrome, epilepsy, periodic limb movement in sleep, attention deficit disorders, depression, anxiety, dementia, Alzheimer's disease, Huntington's disease, multiple sclerosis, cerebral ischemia, hemorrhagic stroke, subarachnoid hemorrhage, or traumatic brain injury.

Embodiments of the present invention contemplate the administration of the compounds described herein to a subject for the treatment or prevention of any other disorder that may benefit from at least some level of CCR4 modulation. Such diseases, disorders and conditions may include, for example, asthma, chronic obstructive pulmonary disease (COPD) including chronic bronchitis and emphysema, idiopathic pulmonary fibrosis, atopic or contact dermatitis, urticaria, allergic rhinitis, nasal polyps, allergic conjunctivitis, thrombosis, reperfusion injury of the myocardium and brain, chronic glomerulonephritis, sepsis, adult respiratory distress syndrome, and pain. Additional diseases, disorders and conditions include allergic bronchopulmonary aspergillosis, allergic fungal sinusitis, severe asthma with fungal sensitization and diseases involving a pathogenic role for fungi including invasion or colonization (such as invasive aspergillosis, aspergilloma or candidiasis).

In embodiments, the disease or disorder includes cardiovascular (e.g., cardiac ischemia), metabolic (e.g., development of insulititis diabetes), hepatic (e.g., hepatic fibrosis, NASH, and NAFLD), ophthalmologic (e.g., diabetic retinopathy), and renal (e.g., renal failure) disorders.

Described herein is the administration of the compounds described herein, and compositions (e.g., pharmaceutical salts, pharmaceutical composition) thereof, in any appropriate manner. Suitable routes of administration include oral, parenteral (e.g., intramuscular, intravenous, subcutaneous (e.g., injection or implant), intraperitoneal, intracisternal, intraarticular, intraperitoneal, intracerebral (intraparenchymal) and intracerebroventricular), nasal, vaginal, sublingual, intraocular, rectal, topical (e.g., transdermal), buccal and inhalation. Depot injections, which are generally administered subcutaneously or intramuscularly, may also be utilized to release the compounds disclosed herein over a defined period of time. In embodiments, the administration is oral administration.

In an aspect provided are methods for treating and/or preventing certain cardiovascular- and/or metabolic-related diseases, disorders and conditions, as well as disorders associated therewith, with a compound described herein.

The compounds of the present invention may be administered to a subject in an amount that is dependent upon, for example, the goal of administration (e.g., the degree of resolution desired); the age, weight, sex, and health and physical condition of the subject to which the formulation is being administered; the route of administration; and the nature of the disease, disorder, condition or symptom thereof. The dosing regimen may also take into consideration the existence, nature, and extent of any adverse effects associated with the agent(s) being administered. Effective dosage amounts and dosage regimens can readily be determined from, for example, safety and dose-escalation trials, in vivo studies (e.g., animal models), and other methods known to the skilled artisan.

In general, dosing parameters dictate that the dosage amount be less than an amount that could be irreversibly toxic to the subject (the maximum tolerated dose (MTD)) and not less than an amount required to produce a measurable effect on the subject. Such amounts are determined by, for example, the pharmacokinetic and pharmacodynamic parameters associated with ADME, taking into consideration the route of administration and other factors.

An effective dose (ED) is the dose or amount of an agent that produces a therapeutic response or desired effect in some fraction of the subjects taking it. The “median effective dose” or ED50 of an agent is the dose or amount of an agent that produces a therapeutic response or desired effect in 50% of the population to which it is administered. Although the ED50 is commonly used as a measure of reasonable expectance of an agent's effect, it is not necessarily the dose that a clinician might deem appropriate taking into consideration all relevant factors. Thus, in some situations the effective amount is more than the calculated ED50, in other situations the effective amount is less than the calculated ED50, and in still other situations the effective amount is the same as the calculated ED50.

In addition, an effective dose of the compounds of the present invention may be an amount that, when administered in one or more doses to a subject, produces a desired result relative to a healthy subject. For example, for a subject experiencing a particular disorder, an effective dose may be one that improves a diagnostic parameter, measure, marker and the like of that disorder by at least about 5%, at least about 10%, at least about 20%, at least about 25%, at least about 30%, at least about 40%, at least about 50%, at least about 60%, at least about 70%, at least about 80%, at least about 90%, or more than 90%, where 100% is defined as the diagnostic parameter, measure, marker and the like exhibited by a normal subject.

In embodiments, the compounds described herein may be administered (e.g., orally) at dosage levels of about 0.01 mg/kg to about 50 mg/kg, or about 1 mg/kg to about 25 mg/kg, of subject body weight per day, one, two, three, four or more times a day, to obtain the desired therapeutic effect. For administration of an oral agent, the compositions can be provided in the form of tablets, capsules and the like containing from 0.05 to 1000 milligrams of the active ingredient, particularly 0.05, 0.1, 0.25, 0.5, 0.75, 1.0, 1.25, 1.5, 1.75, 2.0, 2.5, 5.0, 7.5, 10.0, 15.0, 20.0, 25.0, 50.0, 75.0, 100.0, 125.0, 150.0, 175.0, 200.0, 250.0, 300.0, 400.0, 500.0, 600.0, 750.0, 800.0, 900.0, and 1000.0 milligrams of the active ingredient. A pharmaceutically acceptable carrier(s), diluent(s) and/or excipient(s) may be present in an amount of from about 0.1 g to about 2.0 g.

In embodiments, the dosage of the desired compound is contained in a “unit dosage form”. The phrase “unit dosage form” refers to physically discrete units, each unit including a predetermined amount of the compound (e.g., CCR4 inhibitor), sufficient to produce the desired effect. It will be appreciated that the parameters of a unit dosage form will depend on the particular agent and the effect to be achieved.

### V. Kits

In an aspect is provided a kit including a compound described herein (e.g., a CCR4 inhibitor) or pharmaceutical compositions thereof. The kits may be in the form of a physical structure housing various components, as described below, and may be utilized, for example, in practicing the methods described above. In embodiments, the compound is a compound of formula (I), (II), (III), (IVa), (IVb), (IVc), (Va), (Vb), (Vc), (VI), (VIIa), (VIIb), (VIIc), (VIIIa), (VIIIb), or (VIIIc), including all embodiments thereof.

A kit may include one or more of the compounds disclosed herein (e.g., provided in a sterile container), which may be in the form of a pharmaceutical composition suitable for administration to a subject. The compounds described herein (e.g., CCR4 inhibitors) can be provided in a form that is ready for use (e.g., a tablet or capsule) or in a form requiring, for example, reconstitution or dilution (e.g., a powder) prior to administration. When the compound (e.g., CCR4 inhibitor) is in a form that needs to be reconstituted or diluted by a user, the kit may also include diluents (e.g., sterile water), buffers, pharmaceutically acceptable excipients, and the like, packaged with, or separately from, the compound. Each component of the kit may be enclosed within an individual container, and all of the various containers may be within a single package. A kit of the present invention may be designed for conditions necessary to properly maintain the components housed therein (e.g., refrigeration or freezing).

A kit may contain a label or packaging insert including identifying information for the components therein and instructions for their use (e.g., dosing parameters, clinical pharmacology of the active ingredient(s), including mechanism of action, pharmacokinetics and pharmacodynamics, adverse effects, contraindications, etc.). Labels or inserts can include manufacturer information such as lot numbers and expiration dates. The label or packaging insert may be, e.g., integrated into the physical structure housing the components, contained separately within the physical structure, or affixed to a component of the kit (e.g., an ampule, tube or vial).

Labels or inserts can additionally include, or be incorporated into, a computer readable medium, such as a disk (e.g., hard disk, card, memory disk), optical disk such as CD- or DVD-ROM/RAM, DVD, MP3, magnetic tape, or an electrical storage media such as RAM and ROM or hybrids of these such as magnetic/optical storage media, FLASH media or memory-type cards. In embodiments, the actual instructions are not present in the kit, but means for obtaining the instructions from a remote source, e.g., via the internet, are provided.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### NUMBERED EMBODIMENTS

Embodiment 1. A compound having structural Formula (I):

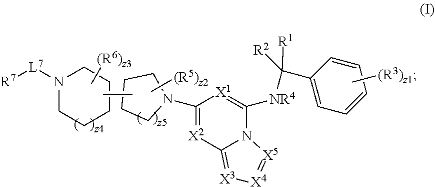

or a pharmaceutically acceptable salt thereof, wherein:
X¹ is CR⁸ or N;
X² is CR⁹ or N;
X³ is CR¹⁰ or N;
X⁴ is CR¹¹ or N;
X⁵ is CR¹² or N;
at least one of X¹, X², X³, X⁴, and X⁵ is N;
z1 is an integer from 0 to 5;
z2 is an integer from 0 to 4;
z3 is an integer from 0 to 11;
z4 is an integer from 0 to 2;
z5 is 1 or 2;
L⁷ is a bond, —O—, —S—, —NR^{7.2B}—, —C(O)—, —C(O)O—, —S(O)—, —S(O)₂—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹ is hydrogen, halogen, —CX^{1.1}₃, —CHX^{1.1}₂, —CH₂X^{1.1}, —CN, —N₃, —SOₙ₁R^{1A}, —SOᵥ₁NR^{1B}R^{1C}, —NHNR^{1B}R^{1C}, —ONR^{1B}R^{1C}, —NHC(O)NHNR^{1B}R^{1C}, —NHC(O)NR^{1B}R^{1C},
—N(O)ₘ₁, —NR^{1B}R^{1C}, —C(O)R^{1D}, —C(O)OR^{1D}, —C(O)NR^{1B}R^{1C}, —OR^{1A}, —NR^{1B}SO₂R^{1A},
—NR^{1B}C(O)R^{1D}—NR^{1B}C(O)OR^{1D}, —NR^{1B}OR^{1D}, —OCX^{1.1}₃, —OCHX^{1.1}₂, —OCH₂X^{1.1}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is hydrogen, halogen, —CX^{2.1}₃, —CHX^{2.1}₂, —CH₂X^{2.1}, —CN, —N₃, —SOₙ₂R^{2A},
—SOᵥ₂NR^{2B}R^{2C}, —NHNR^{2B}R^{2C}, —ONR^{2B}R^{2C}, —NHC(O)NHNR^{2B}R^{2C}, —NHC(O)NR^{2B}R^{2C},
—N(O)ₘ₂, —NR^{2B}R^{2C}, —C(O)R^{2D}, —C(O)OR^{2D}, —C(O)NR^{2B}R^{2C}, —OR^{2A}, —NR^{2B}SO₂R^{2A},
—NR^{2B}C(O)R^{2D}, —NR^{2B}C(O)OR^{2D}, —NR^{2B}OR^{2D}, —OCX^{2.1}₃, —OCHX^{2.1}₂, —OCH₂X^{2.1}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is independently halogen, —CX^{3.1}₃, —CHX^{3.1}₂, —CH₂X^{3.1}, —CN, —N₃, —SOₙ₃R^{3A},
—SOᵥ₃NR^{3B}R^{3C}, —NHNR^{3B}R^{3C}, —ONR^{3B}R^{3C}, —NHC(O)NHNR^{3B}R^{3C}, —NHC(O)NR^{3B}R^{3C},
—N(O)ₘ₃, —NR^{3B}R^{3C}, —C(O)R^{3D}, —C(O)OR^{3D}, —C(O)NR^{3B}R^{3C}, —OR^{3A}, —NR^{3B}SO₂R^{3A},
—NR^{3B}C(O)R^{3D}—NR^{3B}C(O)OR^{3D}, —NR^{3B}OR^{3D}, —OCX^{3.1}₃, —OCHX^{3.1}₂, —OCH₂X³¹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ is hydrogen, —CX^{4.1}₃, —CHX^{4.1}₂, —CH₂X⁴¹, —SOₙ₄R^{4A}, —SOᵥ₄NR^{4B}R^{4C},
—C(O)R^{4D}, —C(O)OR^{4D}, —C(O)NR^{4B}R^{4C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁵ is independently halogen, oxo, —CX^{5.1}₃, —CHX^{5.1}₂, —CH₂X^{5.1}, —CN, —N₃,
—SOₙ₅R^{5A}, —SOᵥ₅NR^{5B}R^{5C}, —NHNR^{5B}R^{5C}, —ONR^{5B}R^{5C}, —NHC(O)NHNR^{5B}R^{5C}, —NHC(O)NR^{5B}R^{5C}, —N(O)ₘ₅, —NR^{5B}R^{5C}, —C(O)R^{5D}, —C(O)OR^{5D}, —C(O)NR^{5B}R^{5C}, —OR^{5A},
—NR^{5B}SO₂R^{5A}, —NR^{5B}C(O)R^{5D}, —NR^{5B}C(O)OR^{5D}, —NR^{5B}OR^{5D}, —OCX^{5.1}₃, —OCHX^{5.1}₂,
—OCH₂X^{5.1}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁶ is independently halogen, oxo, —CX^{6.1}₃, —CHX^{6.1}₂, —CH₂X^{6.1}, —CN, —N₃,
—SOₙ₆R^{6A}, —SOᵥ₆NR^{6B}R^{6C}, —NHNR^{6B}R^{6C}, —ONR^{6B}R^{6C}, —NHC(O)NHNR^{6B}R^{6C}, —NHC(O)NR^{6B}R^{6C}, —N(O)ₘ₆, —NR^{6B}R^{6C}, —C(O)R^{6D}, —C(O)OR^{6D}, —C(O)NR^{6B}R^{6C}, —OR^{6A},
—NR^{6B}SO₂R^{6A}, —NR^{6B}C(O)R^{6D}, —NR^{6B}C(O)OR^{6D}, —NR^{6B}OR^{6D}, —OCX^{6.1}₃, —OCHX^{6.1}₂,
—OCH₂X^{6.1}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁷ is hydrogen, halogen, —CX^{7.1}₃, —CHX^{7.1}₂, —CH₂X^{7.1}, —CN, —N₃, —SOₙ₇R^{7A},
—SOᵥ₇NR^{7B}R^{7C}, —NHNR^{7B}R^{7C}, —ONR^{7B}R^{7C}, —NHC(O)NHNR^{7B}R^{7C}, —NHC(O)NR^{7B}R^{7C},
—N(O)ₘ₇, —NR^{7B}R^{7C}, —C(O)R^{7D}, —C(O)OR^{7D}, —C(O)NR^{7B}R^{7C}, OR^{7A}, —NR^{7B}SO₂R^{7A},
—NR^{7B}C(O)R^{7D}, —NR^{7B}C(O)OR^{7D}, —NR^{7B}OR^{7D}, —OCX^{7.1}₃, —OCHX^{7.1}₂, —OCH₂X^{7.1}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁸ is hydrogen, halogen, —CX^{8.1}₃, —CHX^{8.1}₂, —CH₂X^{8.1}, —CN, —N₃, —SOᵥ₈R^{8A},
—SOᵥ₈NR^{8B}R^{8C}, —NHNR^{8B}R^{8C}, —ONR^{8B}R^{8C}, —NHC(O)NHNR^{8B}R^{8C}, —NHC(O)NR^{8B}R^{8C},
—N(O)ₘ₈, —NR^{8B}R^{8C}, —C(O)R^{8D}, —C(O)OR^{8D}, —C(O)NR^{8B}R^{8C}, —OR^{8A}, —NR^{8B}SO₂R^{8A},
—NR^{8B}C(O)R^{8D}, —NR^{8B}C(O)OR^{8D}, —NR^{8B}OR^{8D}, —OCX^{8.1}₃, —OCHX^{8.1}₂, —OCH₂X^{8.1}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁹ is hydrogen, halogen, —CX^{9.1}₃, —CHX^{9.1}₂, —CH₂X^{9.1}, —CN, —N₃, —SOₙ₉R^{9A},
—SOᵥ₉NR^{9B}R^{9C}, —NHNR^{9B}R^{9C}, —ONR^{9B}R^{9C}, —NHC(O)NHNR^{9B}R^{9C}, —NHC(O)NR^{9B}R^{9C},
—N(O)ₘ₉, —NR^{9B}R^{9C}, —C(O)R^{9D}, —C(O)OR^{9D}, —C(O)NR^{9B}R^{9C}, —OR^{9A}, —NR^{9B}SO₂R^{9A}
—NR^{9B}C(O)R^{9D}, —NR^{9B}C(O)OR^{9D}, —NR^{9B}OR^{9D}, —OCX^{9.1}₃, —OCHX^{9.1}₂, —OCH₂X^{9.1}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or when X² is CR⁹ and X³ is CR¹⁰, then R⁹ and R¹⁰ may optionally be joined to form a substituted or unsubstituted cycloalkyl, substituted or unsubstituted heterocycloalkyl, substituted or unsubstituted aryl, or substituted or unsubstituted heteroaryl;
R¹⁰ is hydrogen, halogen, —CX^{10.1}₃—CHX^{10.1}₂—CH₂X¹⁰, —CN, —N₃, —SOₙ₁₀R_{10A}, —SOᵥ₁₀NR^{10B}R^{10C}, —NHNR^{10B}R^{10C}, —ONR^{10B}R^{10C}, —NHC(O)NHNR^{10B}R^{10C}, —NHC(O)NR^{10B}Roc, —N(O)ₘ₁₀, —NR^{10B}Roc, —C(O)R^{10D}, —C(O)OR^{10D}, —C(O)NR^{10B}R^{10C},
—OR^{10A}, —NR^{10B}SO₂R^{10A}, —NR^{10B}C(O)R^{10D}, —NR^{10B}C(O)OR^{10D}, —NR^{10B}OR^{10D}, —OCX^{10.1}₃,
—OCHX^{10.1}₂, —OCH₂X^{10.1}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or when X³ is CR¹⁰ and X⁴ is CR¹¹, then R¹⁰ and R¹¹ may optionally be joined to form a substituted or unsubstituted cycloalkyl, substituted or unsubstituted heterocycloalkyl, substituted or unsubstituted aryl, or substituted or unsubstituted heteroaryl;
R¹¹ is hydrogen, halogen, —CX^{11.1}₃, —CHX^{11.1}₂, —CH₂X^{11.1}, —CN, —N₃, —SOₙ₁₁R^{11A}, —SOᵥ₁₁NR^{11B}R^{11C}, —NHNR^{11B}R^{11C}, —ONR^{11B}R^{11C}, —NHC(O)NHNR^{11B}R^{11C}, —NHC(O)NR^{11B}R^{11C}, —N(O)ₘ₁₁, —NR^{11B}R^{11C}, —C(O)R^{11D}, —C(O)OR^{11D}, —C(O)NR^{11B}R^{11C}
—OR^{11A}, —NR^{11B}SO₂R^{11A}, —NR^{11B}C(O)R^{11D}, —NR^{11B}C(O)OR^{11D}, —NR^{11B}OR^{11D}, —OCX^{11.1}₃,
—OCHX^{11.1}₂, —OCH₂X^{11.1}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or when X⁴ is CR¹¹ and X⁵ is CR¹², then R¹¹ and R¹² may optionally be joined to form a substituted or unsubstituted cycloalkyl, substituted or unsubstituted heterocycloalkyl, substituted or unsubstituted aryl, or substituted or unsubstituted heteroaryl;
R¹² is hydrogen, halogen, —CX^{12.1}₃, —CHX^{12.1}₂, —CH₂X^{12.1}, —CN, —N₃, —SOₙ₁₂R^{12A}, —SOᵥ₁₂NR^{12B}R^{12C}, —NHNR^{12B}R^{12C}, —ONR^{12B}R^{12C}, —NHC(O)NHNR^{12B}R^{12C}, —NHC(O)NR^{12B}R^{12C}, —N(O)ₘ₁₂, —NR^{12B}R^{12C}, —C(O)R^{12D}, —C(O)OR^{12D}, —C(O)NR^{12B}R^{12C},
—OR^{12A}—NR¹²BSO₂R^{12A}, —NR¹²BC(O)R^{12D}, —NR¹²BC(O)OR^{12D}, —NR¹²BOR^{12D}, OCX^{12.13},
—OCHX^{12.1}₂, —OCH₂X^{12.1}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, R^{1D}, R^{2A}, R^{2B}, R^{2C}, R^{2D}, R^{3A}, R^{3B}, R^{3C}, R^{3D}, R^{4A}, R^{4B}, R^{4C}, R^{4D}, R^{5A}, R^{5B}, R^{5C}, R^{5D}, R^{6A}, R^{6B}, R^{6C}, R^{6D}, R^{7A}, R^{7B}, R^{7C}, R^{7D}, R^{7B}, R^{8A}, R^{8B}, R^{8C}, R^{8D}, R^{9A}, R^{9B}, R^{9C}, R^{9D}, R^{10A}, R^{10B}, R^{10C}, R^{10D}, R^{11A}, R^{11B}, R^{11C}, R^{11D}, R^{12A}, R^{12B}, R^{12C}, and R^{12D} are independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1B} and R^{1C} substituents bonded to the same nitrogen atom may optionally be joined to form a substituted or unsubstituted heterocycloalkyl or substituted or unsubstituted heteroaryl; R^{2B} and R^{2C} substituents bonded to the same nitrogen atom may optionally be joined to form a substituted or unsubstituted heterocycloalkyl or substituted or unsubstituted heteroaryl; R^{3B} and R^{3C} substituents bonded to the same nitrogen atom may optionally be joined to form a substituted or unsubstituted heterocycloalkyl or substituted or unsubstituted heteroaryl; R^{4B} and R^{4C} substituents bonded to the same nitrogen atom may optionally be joined to form a substituted or unsubstituted heterocycloalkyl or substituted or unsubstituted heteroaryl; R^{5B} and R^{5C} substituents bonded to the same nitrogen atom may optionally be joined to form a substituted or unsubstituted heterocycloalkyl or substituted or unsubstituted heteroaryl; R^{6B} and R^{6C} substituents bonded to the same nitrogen atom may optionally be joined to form a substituted or unsubstituted heterocycloalkyl or substituted or unsubstituted heteroaryl; R^{7B} and R^{7C} substituents bonded to the same nitrogen atom may optionally be joined to form a substituted or unsubstituted heterocycloalkyl or substituted or unsubstituted heteroaryl; R^{8B} and R^{8C} substituents bonded to the same nitrogen atom may optionally be joined to form a substituted or unsubstituted heterocycloalkyl or substituted or unsubstituted heteroaryl; R^{9B} and R^{9C} substituents bonded to the same nitrogen atom may optionally be joined to form a substituted or unsubstituted heterocycloalkyl or substituted or unsubstituted heteroaryl; R^{10B} and R^{10C} substituents bonded to the same nitrogen atom may optionally be joined to form a substituted or unsubstituted heterocycloalkyl or substituted or unsubstituted heteroaryl; R^{11B} and R^{11C} substituents bonded to the same nitrogen atom may optionally be joined to form a substituted or unsubstituted heterocycloalkyl or substituted or unsubstituted heteroaryl; R^{12B} and R^{12C} substituents bonded to the same nitrogen atom may optionally be joined to form a substituted or unsubstituted heterocycloalkyl or substituted or unsubstituted heteroaryl;
X^{1.1}, X^{2.1}, X^{3.1}, X^{4.1}, X^{5.1}, X^{6.1}, X^{7.1}, X^{8.1}, X^{9.1}, X^{10.1}, X^{11.1}, and X^{12.1} are independently —Cl, —Br, —I, or —F;
n1, n2, n3, n4, n5, n6, n7, n8, n9, n10, n11, and n12 are independently an integer from 0 to 4; and
m1, m2, m3, m5, m6, m7, m8, m9, m10, m11, m12, v1, v2, v3, v4, v5, v6, v7, v8, v9, v10, v11, and v12 are independently 1 or 2.

Embodiment 2. The compound of embodiment 1, or a pharmaceutically acceptable salt thereof, wherein z2 is 0.

Embodiment 3. The compound of embodiment 1 or embodiment 2, or a pharmaceutically acceptable salt thereof, wherein z4 is 1.

Embodiment 4. The compound of any one of embodiments 1 to 3, or a pharmaceutically acceptable salt thereof, wherein z1 is 2.

Embodiment 5. The compound of embodiment 1, or a pharmaceutically acceptable salt thereof, having the formula:

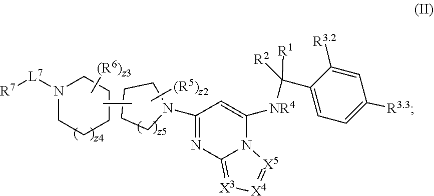

R^{3.2} is hydrogen, halogen, —CX^{3.2}₃, —CHX^{3.2}₂, —CH₂X^{3. 2}, —CN, —N₃, —SO_{n3.2}R^{3.2A},
—SO_{v3.2}NR^{3.2B}R^{3.2C}, —NHNR^{3.2B}R^{3.2C}, —ONR^{3.2B}R^{3.2C}, —NHC(O)NHNR^{3.2B}R^{3.2C}, —NHC(O)NR^{3.2B}R^{3.2C}, —N(O)_{m3.2}, —NR^{3.2B}R^{3.2C}, C(O)R^{3.2D}, —C(O)OR^{3.2D}, —C(O)NR^{3.2B}R^{3.2C},
—OR^{3.2A}, —NR^{3.2B}SO₂R^{3.2A}, —NR^{3.2B}C(O)R^{3.2D}, —NR^{3.2B}C(O)OR^{3.2D}, —NR^{3.2B}OR^{3.2D}, —OCX^{3.2}₃,
—OCHX^{3.2}₂, —OCH₂X^{3.2}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{3.3} is hydrogen, halogen, —CX^{3.3}, —CHX^{3.3}₂, —CH₂X^{3.3}, —CN, —N₃, —SO_{n3.3}R^{3.3A}
—SO_{v3.3}NR^{3.3B}R^{3.3C}, —NHNR^{3.3B}R^{3.3C}, —ONR^{3.3B}R^{3.3C}, —NHC(O)NHNR^{3.3B}R^{3.3C}, —NHC(O)NR^{3.3B}R^{3.3C}, —N(O)_{m3.3}, —NR^{3.3B}R^{3.3C}, —C(O)R^{3.3D}, —C(O)OR^{3.3D}, —C(O)NR^{3.3B}R^{3.3C},
—OR^{3.3A}, —NR^{3.3B}SO₂R^{3.3A}, —NR^{3.3B}C(O)R^{3.3D}, —NR^{3.3B}C(O)OR^{3.3D}, NR^{3.3B}OR^{3.3D}, —OCX^{3.3}₃,
—OCHX^{3.3}₂, —OCH₂X^{3.3}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{3.2A}, R^{3.2B}, R^{3.2C}, R^{3.2D}, R^{3.3A}, R^{3.3B}, R^{3.3C}, and R^{3.3D} are independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{3.2B} and R^{3.2c} substituents bonded to the same nitrogen atom may optionally be joined to form a substituted or unsubstituted heterocycloalkyl or substituted or unsubstituted heteroaryl; R^{3.3B} and R^{3.3c} substituents bonded to the same nitrogen atom may optionally be joined to form a substituted or unsubstituted heterocycloalkyl or substituted or unsubstituted heteroaryl;
n3.2 and n3.3 are independently an integer from 0 to 4;
m3.2, m3.3, v3.2, and v3.3 are independently 1 or 2; and
X^{3.2} and X^{3.3} are independently —Cl, —Br, —I, or —F.

Embodiment 6. The compound of embodiment 5, or a pharmaceutically acceptable salt thereof, having the formula:

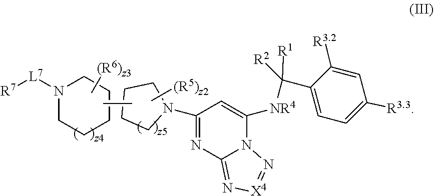

Embodiment 7. The compound of embodiment 6, or a pharmaceutically acceptable salt thereof, wherein
X⁴ is CR¹¹; and
R¹¹ is hydrogen, —CX^{11.1}₃, —CHX^{11.1}₂, —CH₂X^{11.1}, —CN, or substituted or unsubstituted C₁-C₄ alkyl.

Embodiment 8. The compound of embodiment 7, or a pharmaceutically acceptable salt thereof, wherein R¹¹ is hydrogen.

Embodiment 9. The compound of embodiment 5, or a pharmaceutically acceptable salt thereof, having the formula:

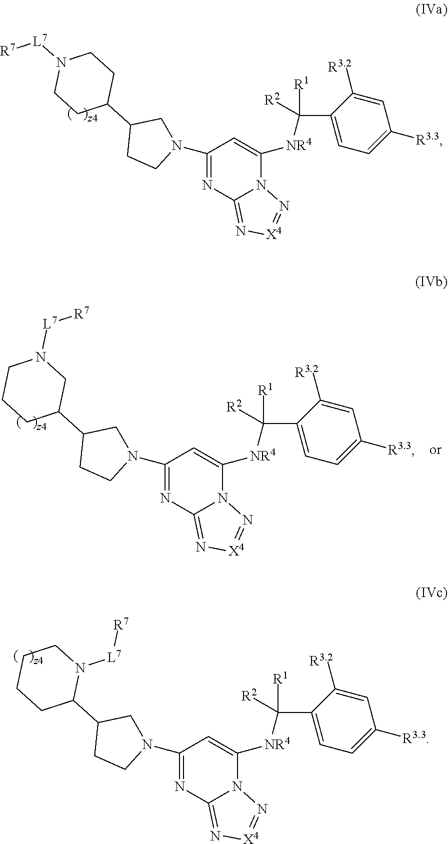

Embodiment 10. The compound of embodiment 5, or a pharmaceutically acceptable salt thereof, having the formula:

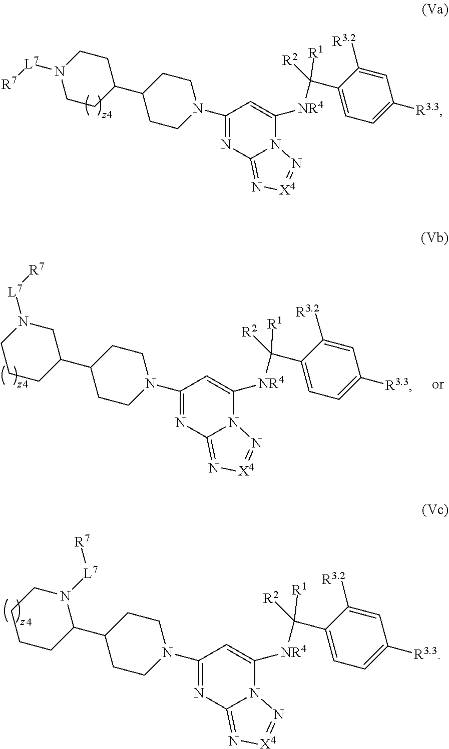

Embodiment 11. The compound of embodiment 5, or a pharmaceutically acceptable salt thereof, having the formula:

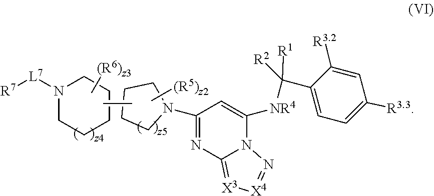

Embodiment 12. The compound of embodiment 11, or a pharmaceutically acceptable salt thereof, wherein
X³ is CR¹⁰;
X⁴ is CR¹¹;
R¹⁰ is hydrogen, —CX^{10.1}₃, —CHX^{10.1}₂—CH₂X^{10.1}, —CN, or substituted or unsubstituted C₁-C₄ alkyl; and
R¹¹ is hydrogen, —CX^{11.1}₃, —CHX^{11.1}₂, —CH₂X^{11.1}, —CN, or substituted or unsubstituted C₁-C₄ alkyl.

Embodiment 13. The compound of embodiment 12, or a pharmaceutically acceptable salt thereof, wherein R¹⁰ and R¹¹ are hydrogen.

Embodiment 14. The compound of embodiment 5, or a pharmaceutically acceptable salt thereof, having the formula:

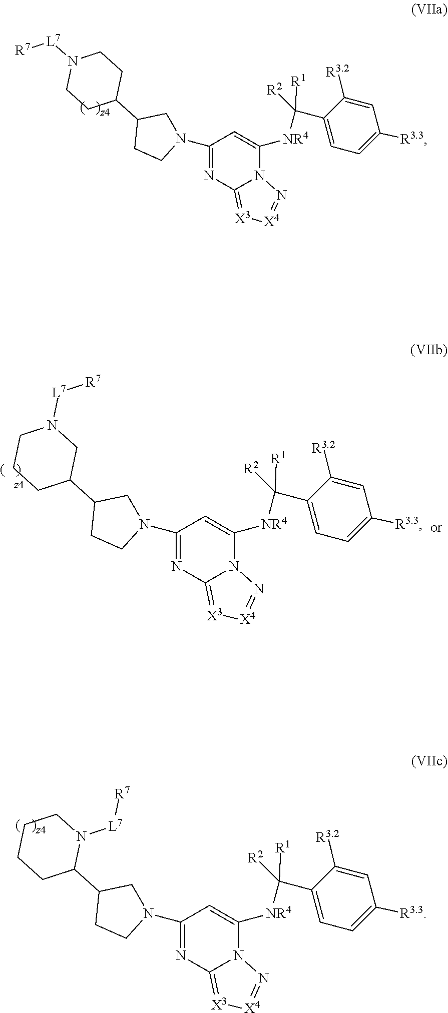

Embodiment 15. The compound of embodiment 5, or a pharmaceutically acceptable salt thereof, having the formula:

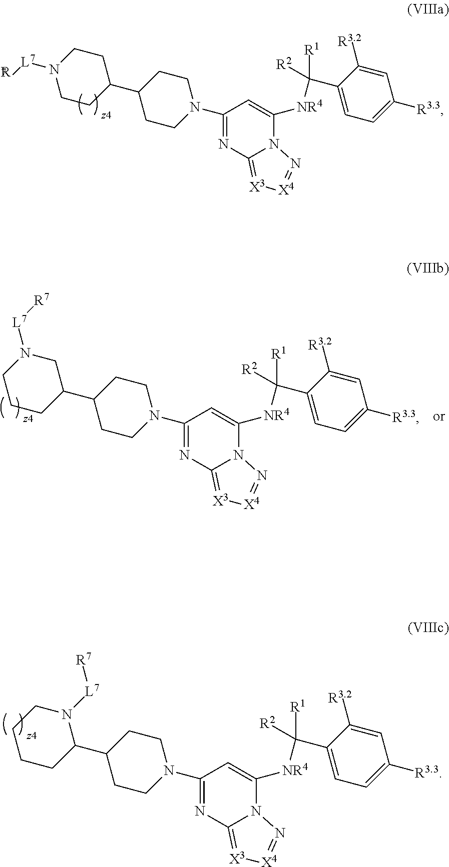

Embodiment 16. The compound of any one of embodiments 1 to 15, or a pharmaceutically acceptable salt thereof, wherein R⁴ is hydrogen.

Embodiment 17. The compound of any one of embodiments 1 to 16, or a pharmaceutically acceptable salt thereof, wherein R¹ is hydrogen, substituted or unsubstituted alkyl, or substituted or unsubstituted heteroalkyl.

Embodiment 18. The compound of any one of embodiments 1 to 16, or a pharmaceutically acceptable salt thereof, wherein R¹ is hydrogen or substituted or unsubstituted C₁-C₄ alkyl.

Embodiment 19. The compound of any one of embodiments 1 to 16, or a pharmaceutically acceptable salt thereof, wherein R¹ is hydrogen.

Embodiment 20. The compound of any one of embodiments 1 to 16, or a pharmaceutically acceptable salt thereof, wherein R¹ is unsubstituted methyl.

Embodiment 21. The compound of any one of embodiments 1 to 20, or a pharmaceutically acceptable salt thereof, wherein R² is hydrogen, substituted or unsubstituted alkyl, or substituted or unsubstituted heteroalkyl.

Embodiment 22. The compound of any one of embodiments 1 to 20, or a pharmaceutically acceptable salt thereof, wherein R² is hydrogen or substituted or unsubstituted C₁-C₄ alkyl.

Embodiment 23. The compound of any one of embodiments 1 to 20, or a pharmaceutically acceptable salt thereof, wherein R² is hydrogen.

Embodiment 24. The compound of any one of embodiments 1 to 20, or a pharmaceutically acceptable salt thereof, wherein R² is unsubstituted methyl.

Embodiment 25. The compound of any one of embodiments 5 to 10, or a pharmaceutically acceptable salt thereof, wherein R^{3.2} and R^{3.3} are independently halogen.

Embodiment 26. The compound of any one of embodiments 5 to 10, or a pharmaceutically acceptable salt thereof, wherein R^{3.2} and R^{3.3} are —Cl.

Embodiment 27. The compound of any one of embodiments 1 to 26, or a pharmaceutically acceptable salt thereof, wherein L⁷ is a bond, substituted or unsubstituted alkylene, or substituted or unsubstituted cycloalkylene.

Embodiment 28. The compound of any one of embodiments 1 to 26, or a pharmaceutically acceptable salt thereof, wherein L⁷ is a bond, unsubstituted C₁-C₆ alkylene, or substituted or unsubstituted C₃-C₆ cycloalkylene.

Embodiment 29. The compound of any one of embodiments 1 to 26, or a pharmaceutically acceptable salt thereof, wherein L⁷ is a bond, unsubstituted methylene, unsubstituted ethylene, unsubstituted n-propylene,

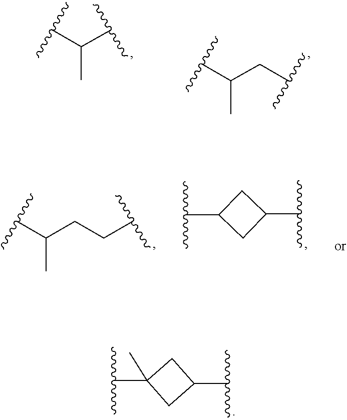

Embodiment 30. The compound of any one of embodiments 1 to 29, or a pharmaceutically acceptable salt thereof, wherein R⁷ is hydrogen, halogen, —CX^{7.1}₃, —CN, —SOₙ₇R^{7A}, —SOᵥ₇NR^{7B}R^{7C}, —NHC(O)NR^{7B}R^{7C}, —NR^{7B}R^{7C}, —C(O)R^{7D}, —C(O)OR^{7D}, —C(O)NR^{7B}R^{7C}, —OR^{7A}, —NR^{7B}SO₂R^{7A}, —NR^{7B}C(O)R^{7D}, —NR^{7B}C(O)OR^{7D}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 31. The compound of any one of embodiments 1 to 29, or a pharmaceutically acceptable salt thereof, wherein R⁷ is hydrogen, —CX^{7.1}₃, —CN, —OR^{7A}, —C(O)OR^{7D}, —C(O)NR^{7B}R^{7C}, —NR^{7B}C(O)R^{7D}, —SOₙ₇R^{7A}, —SOᵥ₇NR^{7B}R^{7C}, substituted or unsubstituted C₁-C₄ alkyl, unsubstituted 2 to 6 membered heteroalkyl, substituted or unsubstituted C₃-C₆ cycloalkyl, or substituted or unsubstituted 5 to 6 membered heteroaryl.

Embodiment 32. The compound of any one of embodiments 1 to 29, or a pharmaceutically acceptable salt thereof, wherein R⁷ is hydrogen, —CF₃, —CN, —OH, —OCH₃, —C(O)OH, —SO₂CH₃, —SO₂NH₂, —SO₂NHCH₃, —NHC(O)CH₃, —C(O)NHCH₃, unsubstituted methyl, unsubstituted cyclopentyl,

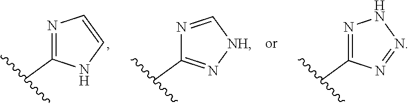 [SMILES: *c1ncc[nH]1 ; *c1nc[nH]n1 ; *c1nn[nH]n1]

Embodiment 33. The compound of embodiment 1 having the formula:

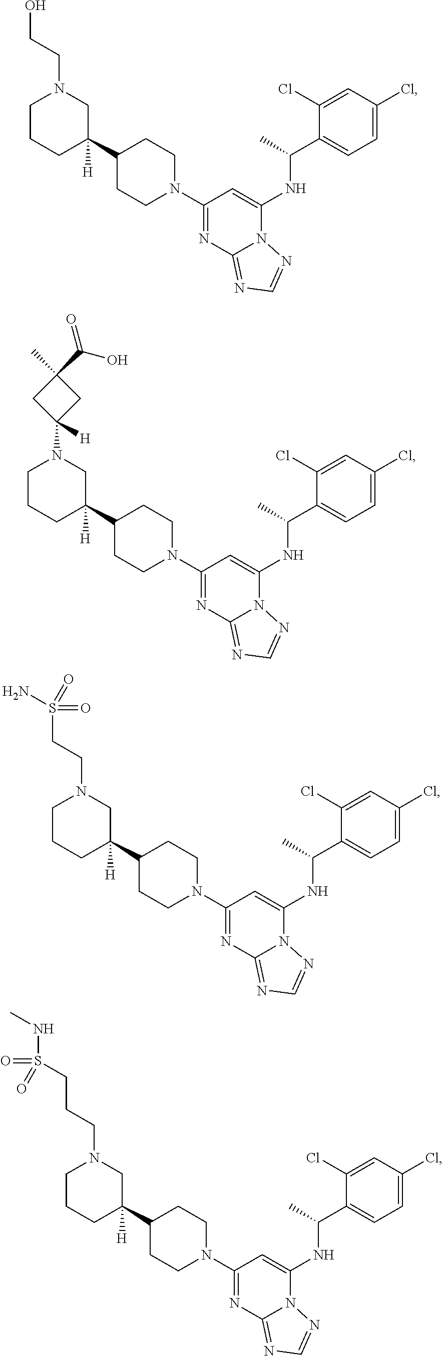 [SMILES: C[C@@H](Nc1cc(N2CCC([C@H]3CCCN(CCO)C3)CC2)nc2ncnn12)c1ccc(Cl)cc1Cl ; C[C@@H](Nc1cc(N2CCC([C@H]3CCCN([C@H]4C[C@](C)(C(=O)O)C4)C3)CC2)nc2ncnn12)c1ccc(Cl)cc1Cl ; C[C@@H](Nc1cc(N2CCC([C@H]3CCCN(CCS(N)(=O)=O)C3)CC2)nc2ncnn12)c1ccc(Cl)cc1Cl ; CNS(=O)(=O)CCCN1CCC[C@H](C2CCN(c3cc(N[C@H](C)c4ccc(Cl)cc4Cl)n4ncnc4n3)CC2)C1]

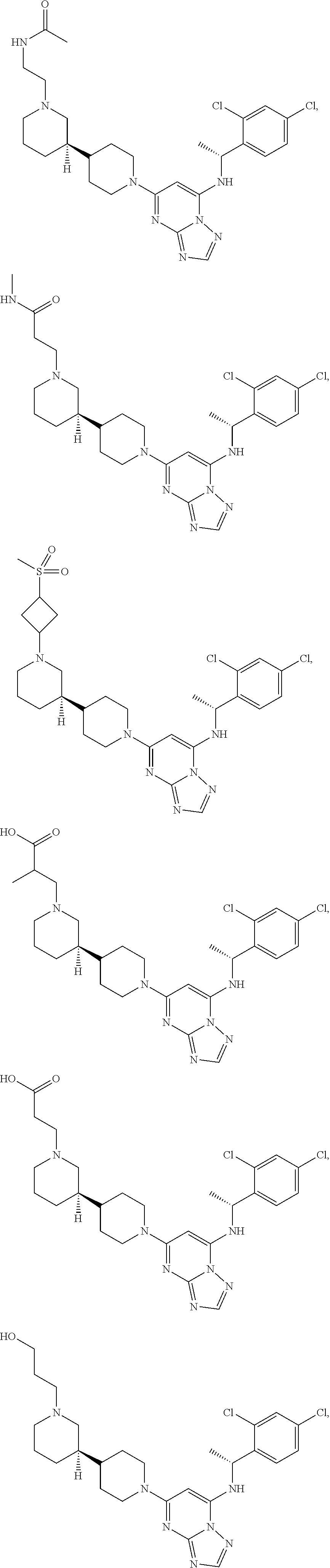 [SMILES: CC(=O)NCCN1CCC[C@H](C2CCN(c3cc(N[C@H](C)c4ccc(Cl)cc4Cl)n4ncnc4n3)CC2)C1 ; CNC(=O)CCN1CCC[C@H](C2CCN(c3cc(N[C@H](C)c4ccc(Cl)cc4Cl)n4ncnc4n3)CC2)C1 ; C[C@@H](Nc1cc(N2CCC([C@H]3CCCN(C4CC(S(C)(=O)=O)C4)C3)CC2)nc2ncnn12)c1ccc(Cl)cc1Cl ; CC(CN1CCC[C@H](C2CCN(c3cc(N[C@H](C)c4ccc(Cl)cc4Cl)n4ncnc4n3)CC2)C1)C(=O)O ; C[C@@H](Nc1cc(N2CCC([C@H]3CCCN(CCC(=O)O)C3)CC2)nc2ncnn12)c1ccc(Cl)cc1Cl ; C[C@@H](Nc1cc(N2CCC([C@H]3CCCN(CCCO)C3)CC2)nc2ncnn12)c1ccc(Cl)cc1Cl]

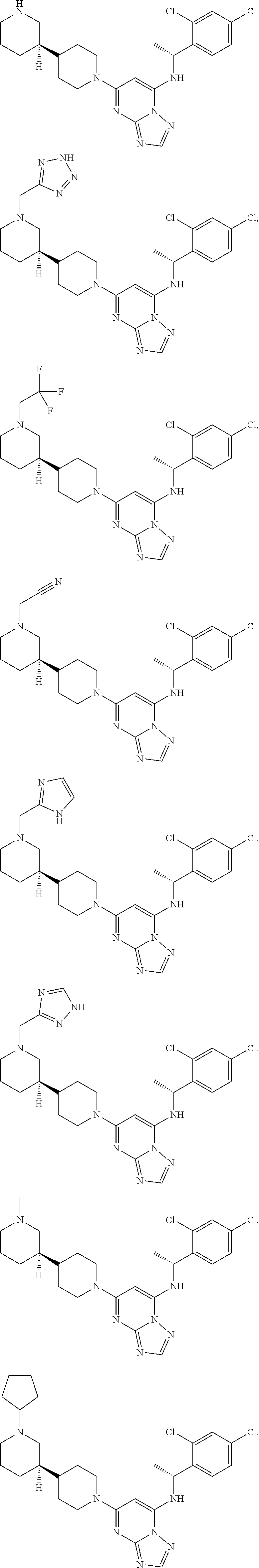

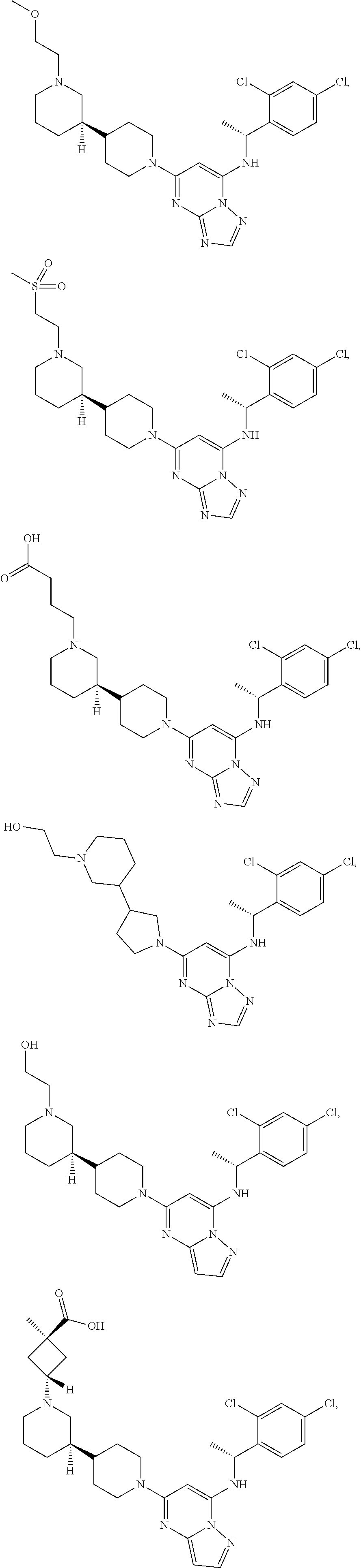 [SMILES: COCCN1CCC[C@H](C2CCN(c3cc(N[C@H](C)c4ccc(Cl)cc4Cl)n4ncnc4n3)CC2)C1 ; C[C@@H](Nc1cc(N2CCC([C@H]3CCCN(CCS(C)(=O)=O)C3)CC2)nc2ncnn12)c1ccc(Cl)cc1Cl ; C[C@@H](Nc1cc(N2CCC([C@H]3CCCN(CCCC(=O)O)C3)CC2)nc2ncnn12)c1ccc(Cl)cc1Cl ; C[C@@H](Nc1cc(N2CCC(C3CCCN(CCO)C3)C2)nc2ncnn12)c1ccc(Cl)cc1Cl ; C[C@@H](Nc1cc(N2CCC([C@H]3CCCN(CCO)C3)CC2)nc2ccnn12)c1ccc(Cl)cc1Cl ; C[C@@H](Nc1cc(N2CCC([C@H]3CCCN([C@H]4C[C@](C)(C(=O)O)C4)C3)CC2)nc2ccnn12)c1ccc(Cl)cc1Cl]

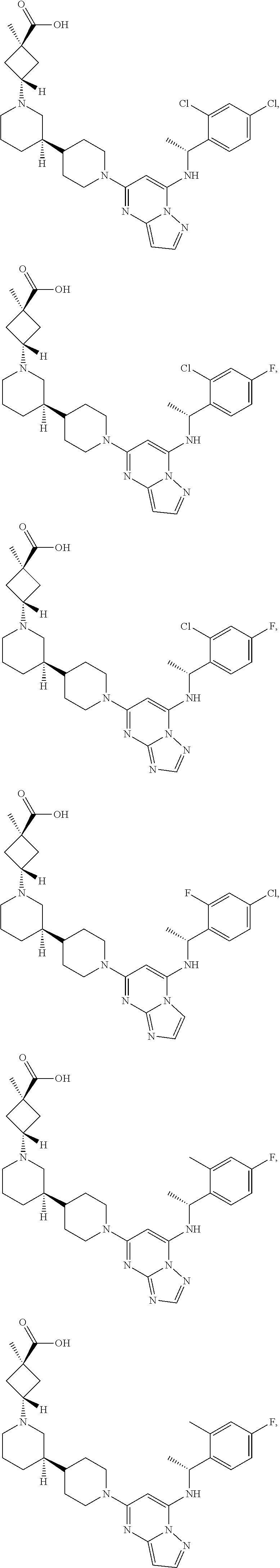

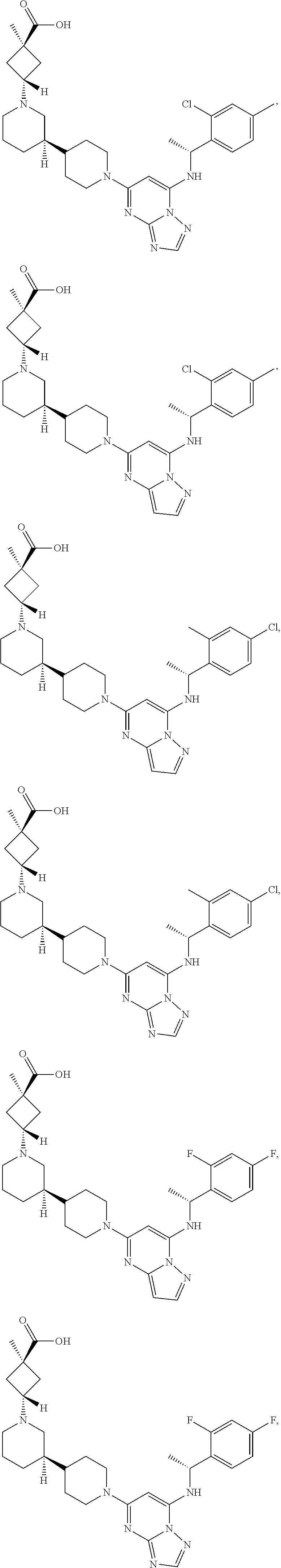

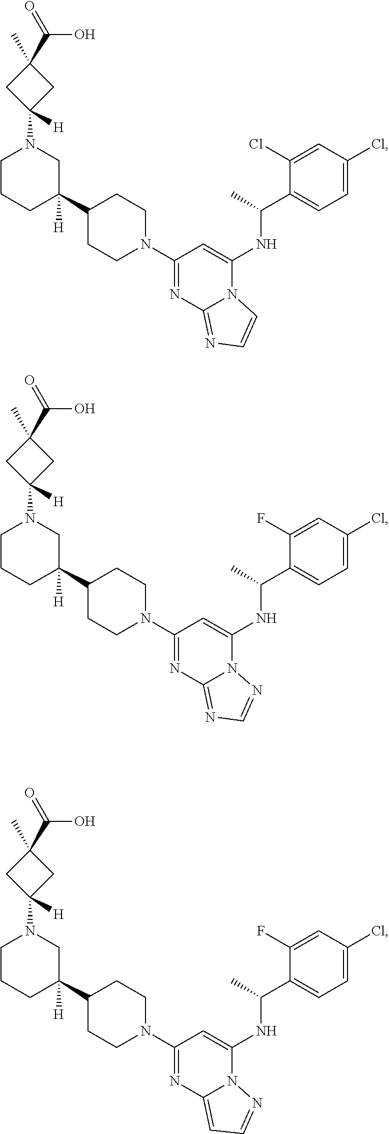 [SMILES: C[C@@H](Nc1cc(N2CCC([C@H]3CCCN([C@H]4C[C@](C)(C(=O)O)C4)C3)CC2)nc2nccn12)c1ccc(Cl)cc1Cl ; C[C@@H](Nc1cc(N2CCC([C@H]3CCCN([C@H]4C[C@](C)(C(=O)O)C4)C3)CC2)nc2ncnn12)c1ccc(Cl)cc1F ; C[C@@H](Nc1cc(N2CCC([C@H]3CCCN([C@H]4C[C@](C)(C(=O)O)C4)C3)CC2)nc2ccnn12)c1ccc(Cl)cc1F]

or a pharmaceutically acceptable salt thereof.

Embodiment 34. A pharmaceutical composition comprising a pharmaceutically acceptable excipient and the compound of any one of embodiments 1 to 33, or a pharmaceutically acceptable salt thereof.

Embodiment 35. A method of treating or preventing a disease or disorder mediated by CCR4, comprising administering to a subject in need thereof a therapeutically effective amount of the compound of any one of embodiments 1 to 33, or a pharmaceutically acceptable salt thereof.

Embodiment 36. A method of treating or preventing a disease or disorder mediated by CCR4, comprising administering to a subject in need thereof a therapeutically effective amount of the pharmaceutical composition of embodiment 34.

Embodiment 37. The method of embodiment 35 or embodiment 36, wherein the disease or disorder is an immune disease or disorder or an inflammatory disease or disorder.

Embodiment 38. The method of embodiment 37, further comprising co-administering an anti-inflammatory agent to the subject.

Embodiment 39. The method of embodiment 38, wherein the anti-inflammatory agent is thalidomide or a derivative thereof, a retinoid, dithranol, calcipotriol, a non-steroidal anti-inflammatory agent (NSAID), a cyclo-oxygenase inhibiting nitric oxide donor (CINOD), a glucocorticosteroid, methotrexate, leflunomide, hydroxychloroquine, d-penicillamine, auranofin, an analgesic, a diacerein, hyaluronic acid derivative, or a nutritional supplement.

Embodiment 40. The method of embodiment 35 or embodiment 36, wherein the disease or disorder is a cardiovascular disease or disorder or a metabolic disease or disorder.

Embodiment 41. The method of embodiment 40, further comprising co-administering a cardiovascular disorder agent or a metabolic disorder agent to the subject.

Embodiment 42. The method of embodiment 41, wherein the cardiovascular disorder agent is a calcium channel blocker, a beta-adrenoceptor blocker, an angiotensin-converting enzyme (ACE) inhibitor, an angiotensin-2 receptor antagonist, a lipid lowering agent, a modulator of blood cell morphology, a thrombolytic, or an anticoagulant.

Embodiment 43. The method of embodiment 35 or claim 36, wherein the disease or disorder is cancer.

Embodiment 44. The method of embodiment 43, further comprising co-administering a chemotherapeutic agent or anti-cancer agent to the subject.

Embodiment 45. The method of embodiment 44, wherein the chemotherapeutic agent or anti-cancer agent is an antiproliferative/antineoplastic drug, an antimetabolite, an antitumour antibiotic, an antimitotic agent, a topoisomerase inhibitor, a cytostatic agent, an oestrogen receptor down regulator, an antiandrogen, a LHRH antagonist or LHRH agonist, a progestogen, an aromatase inhibitor, an inhibitor of 5a-reductase, an agent which inhibits cancer cell invasion, an inhibitor of growth factor function, a farnesyl transferase inhibitor, a tyrosine kinase inhibitor, a serine/threonine kinase inhibitor, an inhibitor of the epidermal growth factor family, an inhibitor of the platelet-derived growth factor family, an inhibitor of the hepatocyte growth factor family; an antiangiogenic agent, a vascular damaging agent, an agent used in antisense therapy, an anti-ras antisense, an agent used in a gene therapy, an immunotherapeutic agent, or an antibody.

Embodiment 46. The method of embodiment 44, wherein the chemotherapeutic agent or anti-cancer agent is an inhibitor of the PD-L1/PD-1 pathway, an inhibitor of CTLA-4 or an agonistic antibody of CD137 (4.1BB).

Embodiment 47. The method of embodiment 44, wherein the chemotherapeutic agent or anti-cancer agent is an agent from Table 1.

Embodiment 48. The method of embodiment 44, further comprising co-administering a therapeutically effective amount of at least two of: a CCR4 inhibitor, an inhibitor of the PD-L1/PD-1 pathway, an inhibitor of CTLA-4 or an agonistic antibody of CD137 (4.1BB).

Embodiment 49. The method of embodiment 44, further comprising co-administering a therapeutically effective amount of at least two of: a CCR4 inhibitor, an immune modulator agent, or an agent from Table 1, or any combination thereof.

Embodiment 50. The method of embodiment 43, wherein the cancer is colon cancer or pancreatic cancer.

Embodiment 51. The method of embodiment 35 or embodiment 36, wherein the disease or disorder is allergy-related.

Embodiment 52. The method of embodiment 35 or embodiment 36, wherein the disease or disorder is inflammation.

Embodiment 53. The method of embodiment 35 or embodiment 36, wherein the disease or disorder is asthma, urticaria, rhinitis, or dermatitis.

Embodiment 54. The method of embodiment 53, wherein the disease or disorder is allergic asthma, autoimmune urticaria, allergic rhinitis, or contact dermatitis.

Embodiment 55. A method of inhibiting C-C chemokine receptor type 4 (CCR4), comprising contacting CCR4 with the compound of any one of embodiments 1 to 33, or a pharmaceutically acceptable salt thereof.

### EXAMPLES

### Synthesis Details

The following general schemes represent synthetic methods that may be used in the preparation of the compounds described herein, as well as common chemical intermediates generated in the preparation thereof. The skilled artisan will recognize that these schemes are representative only, and that in many instances alternative synthetic means may be employed.

The following examples are put forth so as to provide those of ordinary skill in the art with a complete disclosure and description of how to make and use the present invention, and are not intended to limit the scope of what the inventors regard as their invention, nor are they intended to represent that the experiments below were performed or that they are all of the experiments that may be performed. It is to be understood that exemplary descriptions written in the present tense were not necessarily performed, but rather that the descriptions can be performed to generate data and the like of a nature described therein. Efforts have been made to ensure accuracy with respect to numbers used (e.g., amounts, temperature, etc.), but some experimental errors and deviations should be accounted for.

Unless indicated otherwise, parts are parts by weight, molecular weight is weight average molecular weight, temperature is in degrees Celsius (° C.), and pressure is at or near atmospheric. Standard abbreviations are used, including the following: wt=wildtype; bp=base pair(s); kb=kilobase(s); nt=nucleotides(s); aa=amino acid(s); s or see=second(s); min=minute(s); h or hr=hour(s); ng=nanogram; g=microgram; mg=milligram; g=gram; kg=kilogram; dl or dL=deciliter; ¹ or L=microliter; m1 or mL=milliliter; 1 or L=liter; gM=micromolar; mM=millimolar; M=molar; kDa=kilodalton; i.m.=intramuscular(ly); i.p.=intraperitoneal(ly); SC or SQ=subcutaneous(ly); QD=daily; BID=twice daily; QW=weekly; QM=monthly; psi=pounds per square inch; HPLC=high performance liquid chromatography; BW=body weight; U=unit; ns=not statistically significant; HATU=(1-[Bis(dimethylamino)methylene]-1H-1,2,3-triazolo[4,5-b]pyridinium 3-oxid hexafluorophosphate); TFA=trifluoroacetic acid; MTBE=methyl t-butyl ether; DCM=dichloromethane; PBS=phosphate-buffered saline; IHC=immunohistochemistry; DMSO=dimethylsulfoxide; EtOAc=ethyl acetate; EtOH=ethanol; DMEM=Dulbeco's Modification of Eagle's Medium; EDTA=ethylenediaminetetraacetic acid; Me=methyl; Et=ethyl; S—singlet; D—doublet; dd—doublet of doublet; m—multiplet.

### General Synthetic Schemes

General synthesis of amino triazolopyrimidine (X¹═CR⁸, X²═N, X³═N, X⁴-CR¹¹, X⁵═N or X¹═N, X²═CR⁹, X³═N, X⁴═CR¹¹, X⁵═N, or X¹═CR, X²═N, X³═N, X⁴ ═N, X⁵═CR¹² or X¹═N, X²═CR⁹, X³═N, X⁴═N, X⁵═CR¹²), amino triazolopyridine (X¹═CR⁸, X²═CR⁹, X³═N, X⁴═CR¹¹, X⁵═N, or X¹═CR⁸, X²═CR⁹, X³═N, X⁴═N, X⁵═CR¹²), amino pyrazolopyrimidine (X¹═CR⁸, X²═N, X³═CR¹⁰, X⁴═CR¹¹, X⁵═N or X¹═N, X²CR⁹, X³═CR¹⁰, X⁴═CR¹¹, X⁵═N), amino pyrazolopyridine (X¹═CR⁸, X²═CR⁹, X³═CR¹⁰, X⁴═CR¹¹, X⁵═N), amino imidazolopyrimidine (X¹═CR⁸, X²═N, X³═N, X⁴═CR¹¹, X⁵CR¹² or X¹═N, X²═CR⁹, X³═N, X⁴═CR¹¹, X⁵═CR¹² or X¹═CR⁸, X²═N, X³═CR⁹, X⁴-N, X⁵═CR¹² or X¹═N, X²═CR⁹, X³═CR⁹, X⁴═N, X⁵═CR¹²) and amino imidazolopyridine (X¹═CR⁸, X²═CR⁹, X³═CR¹⁰, X⁴═N, X⁵═CR¹² or X¹═CR⁸, X²═CR⁹, X³═N, X⁴═CR¹¹, X═CR¹²):

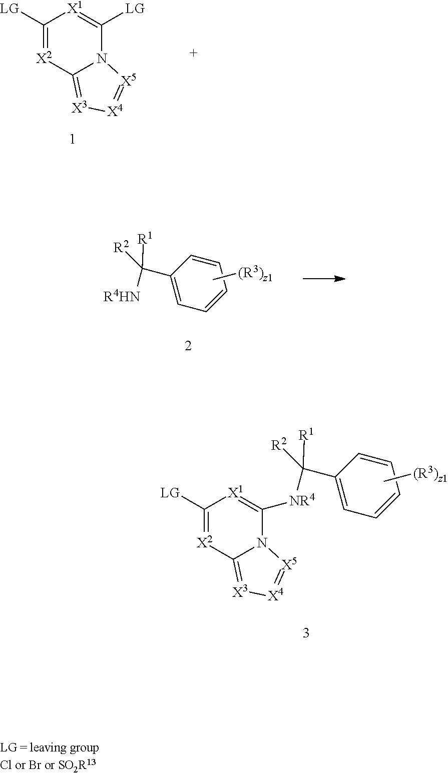

General Procedure A: To a solution of compounds of a heterocycle of general structure 1 and benzylic amine 2 in a solution such as acetonitrile, DMF, or DMSO is added a base such as triethyl amine or cesium fluoride and the reaction is stirred for 2-36 hours at a temperature between room temperature and 120° C. The solvent is removed under reduced pressure and the residue is purified by silica gel chromatography using a mixture of organic solvents that may include hexanes, ethyl acetate or dichloromethane to give the target compound 3.

General synthesis of diamino triazolopyrimidine (X¹═CR⁸, X²═N, X³═N, X⁴-CR¹¹, X⁵═N or X¹═N, X²═CR⁹, X³═N, X⁴ CR¹¹, X⁵═N, or X¹═CR, X²═N, X³═N, X⁴ ═N, X⁵ '² CR¹² or X¹═N, X²═CR⁹, X³═N, X⁴═N, X⁵ '² CR¹²), diamino triazolopyridine (X¹ ═CR⁸, X²═CR⁹, X³═N, X⁴═CR¹¹, X⁵═N, or X¹═CR⁸, X²═CR⁹, X³═N, X⁴═N, X⁵-CR¹²), diamino pyrazolopyrimidine (X¹═CR⁸, X²═N, X³═CR¹⁰, X⁴═CR¹¹, X⁵═N or X¹═N, X²═CR⁹, X³═CR¹⁰, X⁴═CR¹¹, X⁵═N), diamino pyrazolopyridine (X¹ '² CR⁸, X²═CR⁹, X³═CR¹⁰, X⁴═CR¹¹, X⁵═N), diamino imidazolopyrimidine (X¹═CR⁸, X²═N, X³═N, X⁴═CR¹, X⁵═CR¹² or X¹═N, X²═CR⁹, X³═N, X⁴═CR¹¹, X⁵═CR¹² or X¹═CR⁸, X²═N, X³═CR⁹, X⁴═N, X⁵ '² CR¹² or X¹═N, X²═CR⁹, X³═CR⁹, X⁴═N, X⁵ '² CR¹²) and diamino imidazolopyridine (X¹═CR⁸, X²═CR⁹, X³═CR¹⁰, X⁴═N, X⁵ '² CR¹² or X¹═CR⁸, X²═CR⁹, X³═N, X⁴═CR¹¹, X⁵═CR¹²):

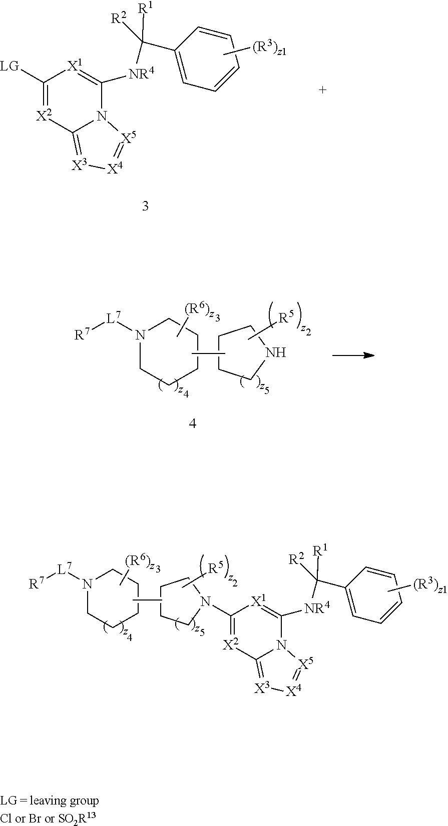

General Procedure B: To a solution of an amino heterocyclic compound of a general structure 3 and a heterocyclic amine of general structure 4 in solvent such as tert-butanol, acetonitrile, or DMSO is added a base such as sodium hydroxide, diisopropylethylamine, or cesium fluoride. The reaction mixture is heated to approximately 80° C. for 4-24 hours. The reaction mixture is neutralized to pH 4-5 and the resulting product is purified by reversed phase HPLC.

General Procedure C: Alternatively, a solution of an amino heterocyclic compound of a general structure 3 and a heterocyclic amine of general structure 4 in an organic solvent such as a mixture of 1,2-dimethoxyethane and tert-butanol is charged with argon and is treated with an organometallic catalyst such as Pd₂dba₃ a ligand such as BINAP and a base such as potassium tert-butoxide and heated to a temperature of approximately 100° C. for 4-24 hours. The mixture is then cooled to room temperature, filtered through a plug of celite with ethyl acetate and further with 20% methanol in dichloromethane and all volatiles are then removed under reduced pressure. The resulting residue can be purified by silica gel chromatography using a mixture of solvents that can include hexanes, ethyl acetate, dichloromethane, or methanol or by using reversed phase HPLC methods.

General synthesis of heterocyclic amine derivatives 4:

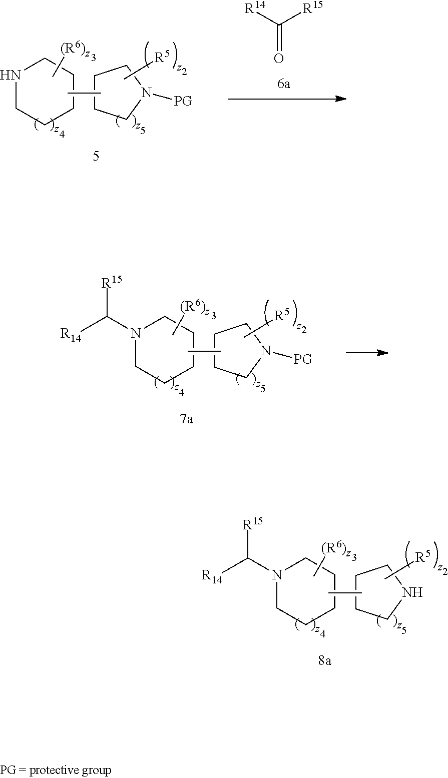

General Procedure D: To a solution of a ketone or aldehyde of general structure 6a in an organic solvent such as dichloroethane is added an amine of general structure 5 and an imine reducing agent such as sodium triacetoxyborohydride and the mixture is stirred for between 1 and 24 h. The reaction is treated with a weak aqueous base such as aqueous sodium carbonate and the mixture is extracted with an organic solvent such as ethyl acetate. The organic solvent is separated, treated with a drying agent such as sodium sulfate and the dried solution is evaporated to give amine of general structure 7a. The protective group, such as Boc, Cbz, or benzyl on compound of general structure 7a can be removed by exposing it to an acidic organic solution, for example HCl in dioxane or trifluoroacetic acid in DCM or can be removed using catalytic Pd. The mixture is stirred at room temperature for a time between 1 and 16 h. The reaction mixture can be concentrated or filtered through a celite pad and then concentrated under reduced pressure to give an amine of the general structure 8a that can be used in subsequent reactions without further purification.

General Procedure E: Alternatively, the amine of general structure 5 can be reacted with an alkyl halide 6b optionally in the presence sodium iodide, in the presence of a base such as sodium carbonate or diisopropylethylamine in a solvent such as DMF or acetonitrile. After stirring between 4 and 18 h, the reaction is diluted with water, and the mixture is extracted with an organic solvent such as ethyl acetate. The organic solvent is separated, treated with a drying agent such as sodium sulfate and the dried solution is evaporated to give an amine of general structure 7b. The protective group, such as Boc, Cbz, or benzyl on compound of general structure 7b can be removed by exposing it to an acidic organic solution, for example HCl in dioxane or trifluoroacetic acid in DCM or can be removed using catalytic Pd. The mixture is stirred at room temperature for a time between 1 and 16 h. The reaction mixture can be concentrated or filtered through a celite pad and then concentrated under reduced pressure to give an amine of the general structure 8b that can be used in subsequent reactions without further purification.

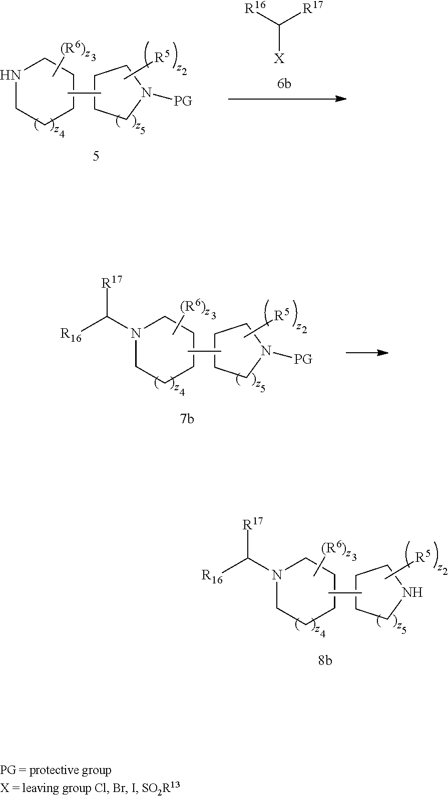

General Method F: Amines of general structure 5 can also be functionalized through a Micheal reaction with the appropriate Michael acceptors such as 6c where R¹⁸ contains an electron withdrawing group (EWG) that may include —NO₂, —CHO, —(C═O)R, —SO₂R, or —CN.

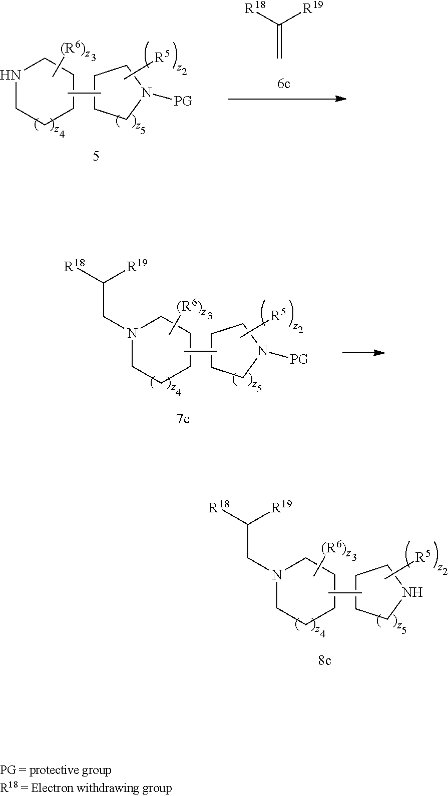

To a solution of amine of general structure 5 in dry organic solvent such as DCM is added a Michael acceptor of structure 7c. The reaction mixture is stirred either at room temperature or at 50° C. until complete conversion which can be monitored using TLC or LCMS. Upon completion, solvent is removed under reduced pressure. The resulting residue can be purified by silica gel chromatography using a mixture of organic solvents, for example a mixture of MeOH and DCM to give compounds of the general formula 7c. The protective group, such as Boc, Cbz, or benzyl on compound of general structure 7c can be removed by exposing it to an acidic organic solution, for example HCl in dioxane or trifluoroacetic acid in DCM or can be removed using catalytic Pd. The mixture is stirred at room temperature for a time between one and 16 h. The reaction mixture can be concentrated or filtered through a celite pad and then concentrated under reduced pressure to give an amine of the general structure 8c that can be used in subsequent reactions without further purification.

### Synthetic Examples

### Synthesis of Common Intermediate I:

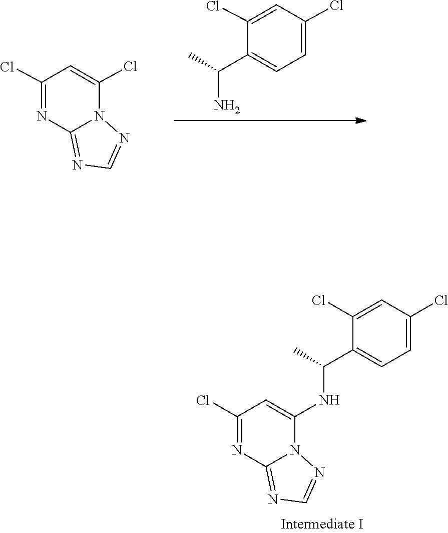 [SMILES: Clc1cc(Cl)n2ncnc2n1 ; C[C@@H](N)c1ccc(Cl)cc1Cl ; C[C@@H](Nc1cc(Cl)nc2ncnn12)c1ccc(Cl)cc1Cl]

(R)-5-chloro-N-(1-(2,4-dichlorophenyl)ethyl)-[1,2,4]triazolo[1,5-a]pyrimidin-7-amine. Prepared according to General Procedure A, at room temperature, commercially available 5,7-dichloro-[1,2,4]triazolo[1,5-a]pyrimidine (3.96 g, 20.95 mmol) and (1R)-1-(2,4-dichlorophenyl)ethanamine (3.98 g, 20.95 mmol) are dissolved in anhydrous DMSO (100 mL), cesium fluoride (4.77 g, 31.43 mmol) is added, and the reaction mixture is stirred at 100° C. for 3 hours. The reaction mixture was cooled to room temperature, added water, and extracted with EtOAc, then with EtOAc/hexanes (1:1). The organic layer was dried over anhydrous sodium sulfate, filtered, and concentrated. The crude was purified directly by normal-phase chromatography using a gradient of EtOAc in hexanes (0 to 100%) as eluent. The fractions containing the desired product were concentrated under reduced pressure until a constant weight was attained and afforded (R)-5-chloro-N-(1-(2,4-dichlorophenyl)ethyl)-[1,2,4]triazolo[1,5-a]pyrimidin-7-amine (5.749 g).

### Synthesis of Common Intermediate II:

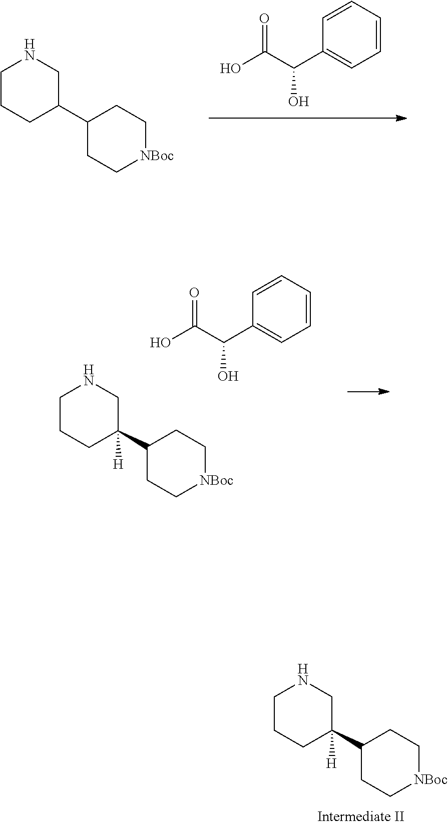 [SMILES: CC(C)(C)OC(=O)N1CCC(C2CCCNC2)CC1 ; O=C(O)[C@@H](O)c1ccccc1 ; O=C(O)[C@@H](O)c1ccccc1 ; CC(C)(C)OC(=O)N1CCC([C@H]2CCCNC2)CC1 ; CC(C)(C)OC(=O)N1CCC([C@H]2CCCNC2)CC1]

Step 1. Commercially available tert-butyl 4-(piperidin-3-yl)piperidine-1-carboxylate (25.6 g, 95.38 mmol) is dissolved in THF (76.8 mL) and then the solution is heated to 80° C. (mild reflux). L-(+)-mandelic acid (7.26 g, 47.69 mmol) was added dissolved in THF (7.26 mL), followed by MTBE (76.8 mL). The mixture is heated to mild reflux (−60° C.) for 30 minutes, then cooled to room temperature slowly over 8 h. A white solid is formed. The solid was filtered and rinsed with cold MTBE. The solid was dried under reduced pressure until a constant weight was attained and afforded tert-butyl 4-[(3R)-3-piperidyl]piperidine-1-carboxylate; (2S)-2-hydroxy-2-phenyl-acetic acid (19.53 g, 98:2 er by chiral HPLC on a Chiralpak IC-3 column, 4.6 mm×250 mm, 3 μM, eluting with 50% heptane (with 0.1% diethylamine), 50% ethanol at 1 mL/min, detecting at 210 nM, t_{R}^{=8.1}-9 min).

Step 2. tert-butyl 4-[(3R)-3-piperidyl]piperidine-1-carboxylate; (2S)-2-hydroxy-2-phenyl-acetic acid (3 g, 7.13 mmol) is dissolved in DCM (60 mL) and then washed with a saturated aqueous solution of sodium carbonate. The aqueous fraction was then extracted with DCM (2×60 mL). The final pH of the aqueous fraction was 11. The combined organic fractions were dried over anhydrous sodium sulfate, filtered and concentrated under reduced pressure to afford tert-butyl 4-[(3R)-3-piperidyl]piperidine-1-carboxylate (1.91 g). The crude was used as is for the next reactions with any further purification. LCMS [M+H]⁺: 269.2

### Compound Examples

### Example 1.5-((R)-[3,4′-bipiperidin]-1′-yl)-N-((R)-1-(2,4-dichlorophenyl)ethyl)-[1,2,4]triazolo[1,5-a]pyrimidin-7-amine

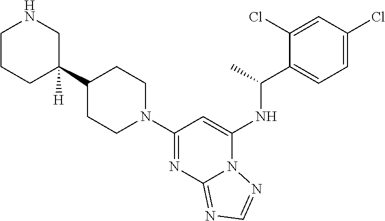 [SMILES: C[C@@H](Nc1cc(N2CCC([C@H]3CCCNC3)CC2)nc2ncnn12)c1ccc(Cl)cc1Cl]

Step 1. Synthesis of (R)-1-([3,4′-bipiperidin]-1-yl)-2,2,2-trifluoroethan-1-one. Intermediate 11(1.9 g, 7.08 mmol) and N,N-diisopropylethylamine (2.47 mL, 14.16 mmol) were dissolved in DCM (28 mL), then added trifluoroacetic anhydride (1.48 mL, 10.62 mmol) dropwise. The reaction mixture was stirred at room temperature for 18 h. The reaction mixture was washed with a saturated aqueous solution of sodium carbonate, then water. The organic layer is dried over anhydrous sodium sulfate, filtered, and concentrated. The crude product was dissolved in DCM (30 mL), then added HCl (8.6 mL, 4 N in 1,4-dioxane) dropwise. The reaction mixture was stirred at room temperature for 18 h, then concentrated under reduced pressure until a constant weight was attained and afforded the title compound (2.0 g) as the corresponding HCl salt. The crude was used as is for the next reactions with any further purification. LCMS [M+Na]⁺: 387.2.

Step 2. Synthesis of 1-((R)-1′-(7-(((R)-1-(2,4-dichlorophenyl)ethyl)amino)-[1,2,4]triazolo[1,5-a]pyrimidin-5-yl)-[3,4′-bipiperidin]-1-yl)-2,2,2-trifluoroethan-1-one. Prepared according to General Procedure B using intermediate I (500 mg, 1.46 mmol), (R)-1-([3,4′-bipiperidin]-1-yl)-2,2,2-trifluoroethan-1-one hydrochloride (610 mg, 2.04 mmol), and N,N-diisopropylethylamine (1.53 mL, 8.76 mmol) in MeCN (10 mL) at 85° C. for 18 h. The solvent was removed under reduced pressure. The crude was purified directly by normal-phase chromatography using a gradient of EtOAc in hexanes (0 to 100%) as eluent. The fractions containing the desired product were concentrated under reduced pressure until a constant weight was attained and afforded the title compound (682 mg). LCMS [M+H]⁺: 570.1.

Step 3. Synthesis of 5-((R)-[3,4′-bipiperidin]-1′-yl)-N-((R)-1-(2,4-dichlorophenyl)ethyl)-[1,2,4]triazolo[1,5-a]pyrimidin-7-amine. 1-((R)-1-(7-(((R)-1-(2,4-dichlorophenyl)ethyl)amino)-[1,2,4]triazolo[1,5-a]pyrimidin-5-yl)-[3,4′-bipiperidin]-1-yl)-2,2,2-trifluoroethan-1-one (682 mg, 1.20 mmol) was dissolved in 1,4-dioxane (10 mL), then added LiOH (1.2 mL, 3 N aqueous solution). The reaction mixture was heated to 50° C. for 18 h. The reaction mixture was cooled to room temperature, added water, and extracted with 10% MeOH in DCM. The organic layer was dried over anhydrous sodium sulfate, filtered, and concentrated. The product was concentrated under reduced pressure until a constant weight was attained and afforded the title compound (540 mg). ¹H NMR (400 MHz, DMSO-d6, TFA salt) δ8.35 (d, J=7.6 Hz, 1H), 8.32 (bs, 2H), 8.1 (s, 1H), 7.65 (d, J=1.3 Hz, 1H), 7.64 (d, J=8.5 Hz, 1H), 7.46 (dd, J=8.5, 1.3 Hz, 1H), 5.26 (s, 1H), 5.08 (q, J=7.1 Hz, 1H), 4.33-4.17 (m, 2H), 3.28-3.14 (m, 2H), 2.87-2.78 (m, 2H), 2.79-2.65 (m, 1H), 2.66-2.52 (m, 1H), 1.82.1.70 (m, 2H), 1.70.1.61 (m, 2H), 1.59 (d, J=6.8 Hz, 3H), 1.55.1.48 (m, 1H), 1.46.1.37 (m, 2H), 1.20-1.04 (m, 2H), 1.04-0.89 (m, 1H). LCMS [M+H]⁺: 474.2.

### Example 2. 5-((R)-1-((2H-tetrazol-5-yl)methyl)-[3,4′-bipiperidin]-1′-yl)-N-((R)-1-(2,4-dichlorophenyl)ethyl)-[1,2,4]triazolo[1,5-a]pyrimidin-7-amine

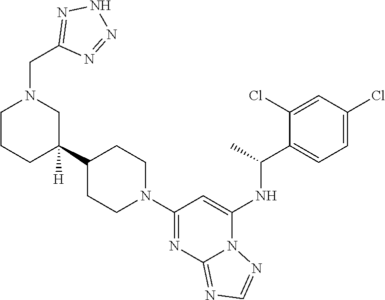 [SMILES: C[C@@H](Nc1cc(N2CCC([C@H]3CCCN(Cc4nn[nH]n4)C3)CC2)nc2ncnn12)c1ccc(Cl)cc1Cl]

Prepared according to General Procedure E using 5-((R)-[3,4′-bipiperidin]-1′-yl)-N-((R)-1-(2,4-dichlorophenyl)ethyl)-[1,2,4]triazolo[1,5-a]pyrimidin-7-amine (50 mg, 0.11 mol, Example 1, Step 3), 5-(chloromethyl)-2H-tetrazole (38 mg, 0.32 mmol), and N,N-diisopropylethylamine (0.20 mL, 1.2 mmol) in MeCN (1 mL) at 80° C. for 18 h. The crude product was purified by reversed phase HPLC (Phenomenex, Gemini-NX, 10 μm, 250×30 mm, C18 column; gradient of 0% MeCN/0.1% TFA-water to 100% MeCN/0.1% TFA) to afford the title compound (11 mg) as the corresponding TFA salt. ¹H NMR (400 MHz, Methanol-d4, TFA salt) δ8.70 (s, 1H), 7.56 (d, J=2.2 Hz, 1H), 7.54 (d, J=8.4 Hz, 1H), 7.37 (dd, J=8.5, 2.2 Hz, 1H), 5.43 (s, 1H), 5.24 (q, J=6.8 Hz, 1H), 4.75-4.67 (m, 2H), 4.55-4.29 (m, 1H), 3.72-3.65 (m, 1H), 3.65-3.58 (m, 1H), 3.06-2.96 (m, 2H), 2.96-2.84 (m, 2H), 2.06.1.97 (m, 1H), 1.94.1.85 (m, 1H), 1.84.1.77 (m, 4H), 1.77.1.71 (m, 1H), 1.68 (d, J=6.8 Hz, 3H), 1.65-1.56 (m, 1H), 1.34.1.18 (m, 2H), 1.18.1.03 (m, 1H). LCMS [M+H]⁺: 556.2.

### Example 3.2-((R)-1′-(7-(((R)-1-(2,4-dichlorophenyl)ethyl)amino)-[1,2,4]triazolo[1,5-a]pyrimidin-5-yl)-[3,4′-bipiperidin]-1-yl)acetonitrile

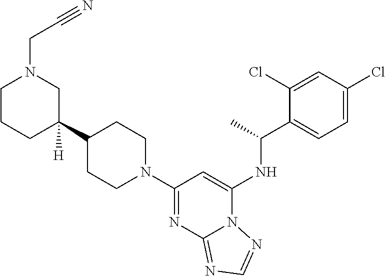 [SMILES: C[C@@H](Nc1cc(N2CCC([C@H]3CCCN(CC#N)C3)CC2)nc2ncnn12)c1ccc(Cl)cc1Cl]

Prepared according to General Procedure E using 5-((R)-[3,4′-bipiperidin]-1′-yl)-N-((R)-1-(2,4-dichlorophenyl)ethyl)-[1,2,4]triazolo[1,5-a]pyrimidin-7-amine (50 mg, 0.11 mol, Example 1, Step 3), 2-bromoacetonitrile (15 μL, 0.21 mmol), and N,N-diisopropylethylamine (0.20 mL, 1.2 mmol) in MeCN (1 mL) at 80° C. for 18 h. The crude product was purified by reversed phase HPLC (Phenomenex, Gemini-NX, 10 μm, 250×30 mm, C18 column; gradient of 0% MeCN/0.1% TFA-water to 100% MeCN/0.1% TFA) to afford the title compound (40 mg) as the corresponding TFA salt. ¹H NMR (400 MHz, Methanol-d4, TFA salt) δ8.70 (s, 1H), 7.57 (d, J=2.4 Hz, 1H), 7.53 (d, J=8.5 Hz, 1H), 7.37 (dd, J=8.4, 2.3 Hz, 1H), 5.24 (q, J=6.8 Hz, 1H), 4.55-4.24 (m, 2H), 4.20-4.02 (m, 2H), 3.30-3.18 (m, 2H), 3.07-2.91 (m, 2H), 2.83-2.43 (m, 3H), 2.01.1.91 (m, 1H), 1.90.1.87 (m, 1H), 1.86.1.82 (m, 2H), 1.82.1.77 (m, 1H), 1.68 (d, J=6.8 Hz, 3H), 1.65.1.49 (m, 2H), 1.32.1.18 (m, 1H), 1.18.1.03 (m, 2H). LCMS [M+H]⁺: 513.2.

### Example 4. 5-((R)-1-((1H-imidazol-2-yl)methyl)-[3,4′-bipiperidin]-1′-yl)-N-((R)-1-(2,4-dichlorophenyl)ethyl)-[1,2,4]triazolo[1,5-a]pyrimidin-7-amine

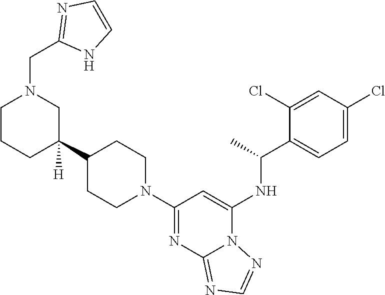 [SMILES: C[C@@H](Nc1cc(N2CCC([C@H]3CCCN(Cc4ncc[nH]4)C3)CC2)nc2ncnn12)c1ccc(Cl)cc1Cl]

Prepared according to General Procedure E using 5-((R)-[3,4′-bipiperidin]-1′-yl)-N-((R)-1-(2,4-dichlorophenyl)ethyl)-[1,2,4]triazolo[1,5-a]pyrimidin-7-amine (50 mg, 0.11 mol, Example 1, Step 3), 2-(bromomethyl)-1H-imidazole hydrobromide (51 mg, 0.21 mmol), and N,N-diisopropylethylamine (0.20 mL, 1.2 mmol) in MeCN (1 mL) at 80° C. for 18 h. The crude product was purified by reversed phase HPLC (Phenomenex, Gemini-NX, 10 μm, 250×30 mm, C18 column; gradient of 0% MeCN/0.1% TFA-water to 100% MeCN/0.1% TFA) to afford the title compound (25 mg) as the corresponding TFA salt. ¹H NMR (400 MHz, Methanol-d4, TFA salt) δ8.70 (s, 1H), 7.57-7.54 (m, 1H), 7.53-7.52 (m, 1H), 7.44-7.42 (m, 1H), 7.39-7.35 (m, 1H), 5.41 (s, 1H), 5.29-5.21 (m, 1H), 4.53-4.30 (m, 3H), 4.30-4.20 (m, 1H), 3.28-3.12 (m, 1H), 3.04-2.88 (m, 2H), 2.82-2.44 (m, 3H), 1.99.1.86 (m, 2H), 1.85.1.75 (m, 3H), 1.75.1.71 (m, 1H), 1.68 (d, J=6.8 Hz, 3H), 1.65.1.52 (m, 1H), 1.31.1.17 (m, 2H), 1.16-1.03 (m, 2H). LCMS [M+H]⁺: 554.2.

### Example 5. 5-((R)-1-((1H-1,2,4-triazol-3-yl)methyl)-[3,4′-bipiperidin]-1′-yl)-N-((R)-1-(2,4-dichlorophenyl)ethyl)-[1,2,4]triazolo[1,5-a]pyrimidin-7-amine

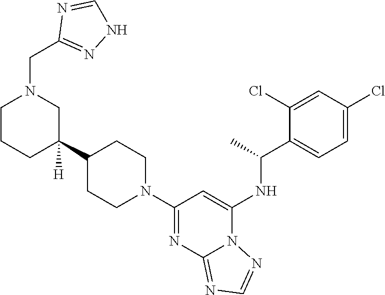 [SMILES: C[C@@H](Nc1cc(N2CCC([C@H]3CCCN(Cc4nc[nH]n4)C3)CC2)nc2ncnn12)c1ccc(Cl)cc1Cl]

Prepared according to General Procedure E using 5-((R)-[3,4′-bipiperidin]-1′-yl)-N-((R)-1-(2,4-dichlorophenyl)ethyl)-[1,2,4]triazolo[1,5-a]pyrimidin-7-amine (50 mg, 0.11 mol, Example 1, Step 3), 3-(chloromethyl)-1H-1,2,4-triazole hydrochloride (32 mg, 0.21 mmol), and N,N-diisopropylethylamine (0.20 mL, 1.2 mmol) in MeCN (1 mL) at 80° C. for 18 h. The crude product was purified by reversed phase HPLC (Phenomenex, Gemini-NX, 10 μm, 250×30 mm, C18 column; gradient of 0% MeCN/0.1% TFA-water to 100% MeCN/0.1% TFA) to afford the title compound (26 mg) as the corresponding TFA salt. ¹H NMR (400 MHz, Methanol-d4, TFA salt) δ8.69 (s, 1H), 8.59 (s, 1H), 7.56 (d, J=2.1 Hz, 1H), 7.54 (d, J=8.4 Hz, 1H), 7.37 (dd, J=8.4, 2.2 Hz, 1H), 5.42 (s, 1H), 5.22 (q, J=6.8 Hz, 1H), 4.50-4.42 (m, 2H), 4.42-4.32 (m, 1H), 3.71-3.63 (m, 1H), 3.63-3.55 (m, 1H), 3.37-3.32 (m, 1H), 3.28-3.25 (m, 1H), 3.06-2.91 (m, 2H), 2.91-2.81 (m, 2H), 2.06.1.96 (m, 1H), 1.94.1.85 (m, 1H), 1.84.1.70 (m, 3H), 1.68 (d, J=6.7 Hz, 3H), 1.65.1.56 (m, 1H), 1.33.1.18 (m, 2H), 1.18.1.05 (m, 1H). LCMS [M+H]⁺: 555.2.

### Example 6. N-((R)-1-(2,4-dichlorophenyl)ethyl)-5-((R)-1-(tetrahydro-2H-pyran-4-yl)-[3,4′-bipiperidin]-1′-yl)-[1,2,4]triazolo[1,5-a]pyrimidin-7-amine

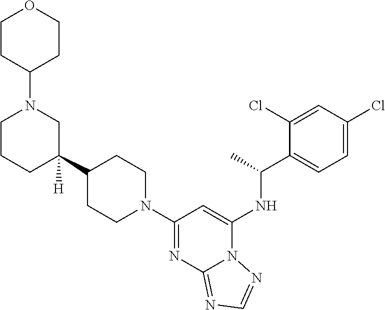 [SMILES: C[C@@H](Nc1cc(N2CCC([C@H]3CCCN(C4CCOCC4)C3)CC2)nc2ncnn12)c1ccc(Cl)cc1Cl]

Prepared according to General Procedure D using 5-((R)-[3,4′-bipiperidin]-1′-yl)-N-((R)-1-(2,4-dichlorophenyl)ethyl)-[1,2,4]triazolo[1,5-a]pyrimidin-7-amine (50 mg, 0.11 mol, Example 1, Step 3), tetrahydro-4H-pyran-4-one (29 μL, 0.32 mmol, and sodium triacetoxyborohydride (112 mg, 0.53 mmol) in THF (1 mL) at room temperature for 14 h. The crude product was purified by reversed phase HPLC (Phenomenex, Gemini-NX, 10 μm, 250×30 mm, C18 column; gradient of 0% MeCN/0.1% TFA-water to 100% MeCN/0.1% TFA) to afford the title compound (33 mg) as the corresponding TFA salt. ¹H NMR (400 MHz, Methanol-d4, TFA salt) δ8.71 (s, 1H), 7.56 (d, J=2.3 Hz, 1H), 7.54 (d, J=8.5 Hz, 1H), 7.37 (dd, J=8.5, 2.1 Hz, 1H), 5.43 (s, 1H), 5.24 (q, J=6.8 Hz, 1H), 4.63-4.21 (m, 2H), 4.12-4.03 (m, 2H), 3.58-3.47 (m, 2H), 3.49-3.38 (m, 3H), 3.08-2.95 (m, 2H), 2.96-2.83 (m, 1H), 2.83-2.72 (m, 1H), 2.11.1.97 (m, 3H), 1.94.1.86 (m, 1H), 1.87.1.78 (m, 3H), 1.81.1.70 (m, 2H), 1.68 (d, J=6.9 Hz, 3H), 1.67.1.57 (m, 1H), 1.36.1.20 (m, 2H), 1.21.1.07 (m, 1H). LCMS [M+H]⁺: 558.2.

### Example 7. N-((R)-1-(2,4-dichlorophenyl)ethyl)-5-((R)-1-(2,2,2-trifluoroethyl)-[3,4′-bipiperidin]-1′-yl)-[1,2,4]triazolo[1,5-a]pyrimidin-7-amine

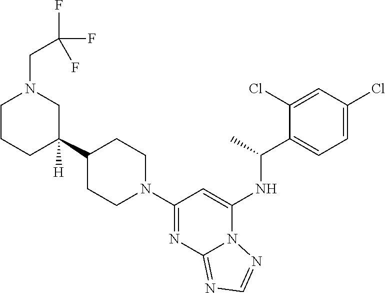 [SMILES: C[C@@H](Nc1cc(N2CCC([C@H]3CCCN(CC(F)(F)F)C3)CC2)nc2ncnn12)c1ccc(Cl)cc1Cl]

Prepared according to General Procedure E using 5-((R)-[3,4′-bipiperidin]-1′-yl)-N-((R)-1-(2,4-dichlorophenyl)ethyl)-[1,2,4]triazolo[1,5-a]pyrimidin-7-amine (50 mg, 0.11 mol, Example 1, Step 3), 2,2,2-trifluoroethyltrifluoromethanesulfonate (23 μL, 0.16 mmol), and N,N-diisopropylethylamine (55 μL, 0.32 mmol) in MeCN (1 mL) at 80° C. for 3 h. The crude product was purified by reversed phase HPLC (Phenomenex, Gemini-NX, 10 μm, 250×30 mm, C18 column; gradient of 0% MeCN/0.1% TFA-water to 100% MeCN/0.1% TFA) to afford the title compound (18 mg) as the corresponding TFA salt. ¹H NMR (400 MHz, Methanol-d4, TFA salt) δ8.69 (s, 1H), 7.56 (d, J=2.1 Hz, 1H), 7.54 (d, J=8.5 Hz, 1H), 7.37 (dd, J=8.4, 2.1 Hz, 1H), 5.42 (s, 1H), 5.24 (q, J=6.8 Hz, 1H), 4.55-4.28 (m, 2H), 3.83-3.62 (m, 2H), 3.38-3.33 (m, 1H), 3.29-3.25 (m, 1H), 3.06-2.91 (m, 2H), 2.85-2.69 (m, 2H), 2.68-2.55 (m, 1H), 2.04 (s, 2H), 1.93.1.85 (m, 1H), 1.85.1.71 (m, 2H), 1.68 (d, J=6.8 Hz, 3H), 1.63.1.52 (m, 2H), 1.31.1.17 (m, 1H), 1.17.1.03 (m, 1H). LCMS [M+H]⁺: 556.2.

### Example 8.2-((R)-1′-(7-(((R)-1-(2,4-dichlorophenyl)ethyl)amino)-[1,2,4]triazolo[1,5-a]pyrimidin-5-yl)-[3,4′-bipiperidin]-1-yl)ethan-1-ol

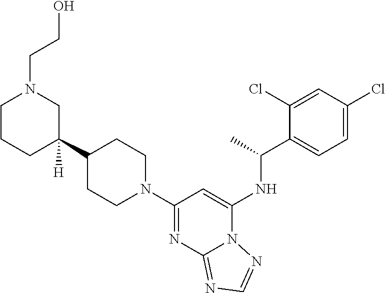 [SMILES: C[C@@H](Nc1cc(N2CCC([C@H]3CCCN(CCO)C3)CC2)nc2ncnn12)c1ccc(Cl)cc1Cl]

Step 1. Synthesis of (R)-2-([3,4′-bipiperidin]-1-yl)ethan-1-ol dihydrochloride. Prepared according to General Procedure D using intermediate II (200 mg, 0.75 mmol), (tert-butyldimethylsiloxy)acetaldehyde (390 mg, 2.24 mmol) and sodium triacetoxyborohydride (474 mg, 2.24 mmol) in DCE (5 mL) and afforded tert-butyl 4-[(3R)-1-[2-[tert-butyl(dimethyl)silyl]oxyethyl]-3-piperidyl]piperidine-1-carboxylate. The crude was then treated with HCl (0.93 mL, 4N in 1,4-dioxane) in DCM (4 mL) and afforded (R)-2-([3,4′-bipiperidin]-1-yl)ethan-1-ol dihydrochloride (211 mg). LCMS [M+H]⁺: 213.2.

Step 2. Synthesis of 2-((R)-1′-(7-(((R)-1-(2,4-dichlorophenyl)ethyl)amino)-[1,2,4]triazolo[1,5-a]pyrimidin-5-yl)-[3,4′-bipiperidin]-1-yl)ethan-1-ol. Prepared according to General Procedure B using intermediate I (200 mg, 0.58 mmol), (R)-2-([3,4′-bipiperidin]-1-yl)ethan-1-ol dihydrochloride (167 mg) and N,N-diisopropylethylamine (0.51 mL, 2.92 mmol) in acetonitrile (10 mL) at 80° C. for 2 h. The crude product was purified by reversed phase HPLC (Phenomenex, Gemini-NX, 10 μm, 250×30 mm, C18 column; gradient of 0% MeCN/0.1% TFA-water to 100% MeCN/0.1% TFA) to afford the title compound (182 mg) as the corresponding TFA salt. ¹H NMR (400 MHz, Methanol-d4, TFA salt) δ8.53 (s, 1H), 7.56 (dd, J=2.1, 0.7 Hz, 1H), 7.53 (d, J=8.5 Hz, 1H), 7.37 (dd, J=8.5, 2.1 Hz, 1H), 5.37 (s, 1H), 5.22 (q, J=6.8 Hz, 1H), 4.54-4.29 (m, 2H), 3.88 (t, J=5.1 Hz, 2H), 3.65-3.56 (m, 2H), 3.26-3.19 (m, 2H), 3.03-2.82 (m, 4H), 2.80-2.70 (m, 1H), 2.04.1.96 (m, 1H), 1.94.1.86 (m, 1H), 1.85.1.74 (m, 3H), 1.67 (d, J=6.8 Hz, 3H), 1.65.1.54 (m, 1H), 1.33.1.18 (m, 2H), 1.18-1.05 (m, 1H). LCMS [M+H]⁺: 518.2.

### Example 9. N-((R)-1-(2,4-dichlorophenyl)ethyl)-5-((R)-1-methyl-[3,4′-bipiperidin]-1′-yl)-[1,2,4]triazolo[1,5-a]pyrimidin-7-amine

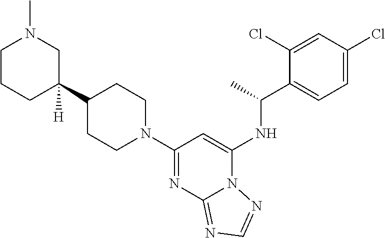 [SMILES: C[C@@H](Nc1cc(N2CCC([C@H]3CCCN(C)C3)CC2)nc2ncnn12)c1ccc(Cl)cc1Cl]

Step 1. Synthesis of (R)-1-methyl-3,4′-bipiperidine dihydrochloride. Prepared according to General Procedure D using intermediate II (150 mg, 0.56 mmol), formaldehyde (136 mg, 1.68 mmol) and sodium triacetoxyborohydride (355 mg, 1.68 mmol) in DCE (3 mL) and afforded tert-butyl (R)-1-methyl-[3,4′-bipiperidine]-1′-carboxylate. The crude was then treated with HCl (0.7 mL, 4N in 1,4-dioxane) in DCM (4 mL) and afforded (R)-1-methyl-3,4′-bipiperidine dihydrochloride (141 mg). LCMS [M+H]⁺: 183.2.

Step 2. Synthesis of N-((R)-1-(2,4-dichlorophenyl)ethyl)-5-((R)-1-methyl-[3,4′-bipiperidin]-1′-yl)-[1,2,4]triazolo[1,5-a]pyrimidin-7-amine. Prepared according to General Procedure B using intermediate I (50 mg, 0.15 mmol), (R)-1-methyl-3,4′-bipiperidine dihydrochloride (39 mg) and N,N-diisopropylethylamine (0.13 mL, 0.73 mmol) in acetonitrile (3 mL) at 80° C. for 2 h. The crude product was purified by reversed phase HPLC (Phenomenex, Gemini-NX, 10 μm, 250×30 mm, C18 column; gradient of 0% MeCN/0.1% TFA-water to 100% MeCN/0.1% TFA) to afford the title compound (82 mg) as the corresponding TFA salt. ¹H NMR (400 MHz, Methanol-d4, TFA salt) δ8.70 (s, 1H), 7.56 (d, J=2.1 Hz, 1H), 7.54 (d, J=8.5 Hz, 1H), 7.37 (dd, J=8.5, 2.1 Hz, 1H), 5.43 (s, 1H), 5.24 (q, J=6.8 Hz, 1H), 4.56-4.24 (m, 2H), 3.54-3.43 (m, 2H), 3.07-2.87 (m, 3H), 2.86 (s, 3H), 2.77-2.67 (m, 1H), 2.04.1.97 (m, 2H), 1.94.1.86 (m, 1H), 1.85.1.74 (m, 2H), 1.68 (d, J=6.8 Hz, 3H), 1.66.1.56 (m, 2H), 1.34.1.06 (m, 3H). LCMS [M+H]⁺: 488.2.

### Example 10. 5-((R)-1-cyclopentyl-[3,4′-bipiperidin]-1′-yl)-N-((R)-1-(2,4-dichlorophenyl)ethyl)-[1,2,4]triazolo[1,5-a]pyrimidin-7-amine

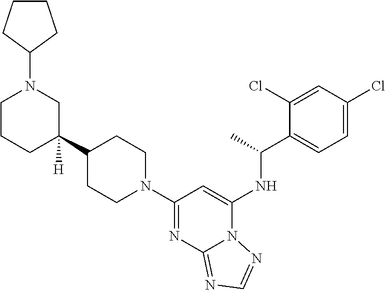 [SMILES: C[C@@H](Nc1cc(N2CCC([C@H]3CCCN(C4CCCC4)C3)CC2)nc2ncnn12)c1ccc(Cl)cc1Cl]

Step 1. Synthesis of (R)-1-cyclopentyl-3,4′-bipiperidine dihydrochloride. Prepared according to General Procedure D using intermediate II (150 mg, 0.56 mmol), cyclopentanone (141 mg, 1.68 mmol) and sodium triacetoxyborohydride (355 mg, 1.68 mmol) in DCE (3 mL) and afforded tert-butyl (R)-1-cyclopentyl-[3,4′-bipiperidine]-1′-carboxylate. The crude was then treated with HCl (0.7 mL, 4 N in 1,4-dioxane) in DCM (4 mL) and afforded (R)-1-cyclopentyl-3,4′-bipiperidine dihydrochloride (171 mg). LCMS [M+H]⁺: 237.2.

Step 2. Synthesis of 5-((R)-1-cyclopentyl-[3,4′-bipiperidin]-1′-yl)-N-((R)-1-(2,4-dichlorophenyl)ethyl)-[1,2,4]triazolo[1,5-a]pyrimidin-7-amine. Prepared according to General Procedure B using intermediate I (50 mg, 0.15 mmol), (R)-1-cyclopentyl-3,4′-bipiperidine dihydrochloride (47.4 mg) and N,N-diisopropylethylamine (0.13 mL, 0.73 mmol) in acetonitrile (3 mL) at 80° C. for 2 h. The crude product was purified by reversed phase HPLC (Phenomenex, Gemini-NX, 10 μm, 250×30 mm, C18 column; gradient of 0% MeCN/0.1% TFA-water to 100% MeCN/0.1% TFA) to afford the title compound (73 mg) as the corresponding TFA salt. ¹H NMR (400 MHz, Methanol-d4, TFA salt) δ8.69 (s, 1H), 7.56 (d, J=2.1 Hz, 1H), 7.53 (d, J=8.4 Hz, 1H), 7.37 (dd, J=8.4, 2.1 Hz, 1H), 5.42 (s, 1H), 5.24 (q, J=6.6 Hz, 1H), 4.59-4.23 (m, 2H), 3.62-3.53 (m, 1H), 3.53-3.44 (m, 2H), 3.08-2.91 (m, 2H), 2.89-2.78 (m, 1H), 2.78-2.68 (m, 1H), 2.26-2.09 (m, 3H), 2.06.1.98 (m, 1H), 1.95.1.77 (m, 6H), 1.76.1.70 (m, 3H), 1.68 (d, J=6.8 Hz, 3H), 1.66.1.58 (m, 2H), 1.37.1.19 (m, 2H), 1.19.1.06 (m, 1H). LCMS [M+H]⁺: 542.2.

### Example 11. N-((R)-1-(2,4-dichlorophenyl)ethyl)-5-((R)-1-(2-methoxyethyl)-[3,4′-bipiperidin]-1′-yl)-[1,2,4]triazolo[1,5-a]pyrimidin-7-amine

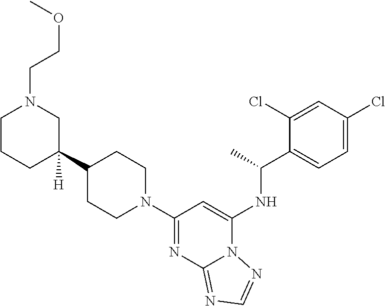 [SMILES: COCCN1CCC[C@H](C2CCN(c3cc(N[C@H](C)c4ccc(Cl)cc4Cl)n4ncnc4n3)CC2)C1]

Step 1. Synthesis of (R)-1-(2-methoxyethyl)-3,4′-bipiperidine dihydrochloride. Prepared according to General Procedure E using intermediate II (150 mg, 0.56 mmol), 1-chloro-2-methoxyethane (63 mg, 0.67 mmol) and N,N-diisopropylethylamine (0.29 mL, 1.68 mmol) in acetonitrile (3 mL) at 80° C. for 12 h and afforded tert-butyl (R)-1-(2-methoxyethyl)-[3,4′-bipiperidine]-1′-carboxylate. The crude was then treated with HCl (0.7 mL, 4 N in 1,4-dioxane) in DCM (4 mL) and afforded (R)-1-(2-methoxyethyl)-3,4′-bipiperidine dihydrochloride (167 mg). LCMS [M+H]⁺: 227.2.

Step 2. Synthesis of N-((R)-1-(2,4-dichlorophenyl)ethyl)-5-((R)-1-(2-methoxyethyl)-[3,4′-bipiperidin]-1′-yl)-[1,2,4]triazolo[1,5-a]pyrimidin-7-amine. Prepared according to General Procedure B using intermediate I (50 mg, 0.15 mmol), (R)-1-(2-methoxyethyl)-3,4′-bipiperidine dihydrochloride dihydrochloride (45.7 mg) and N,N-diisopropylethylamine (0.13 mL, 0.73 mmol) in acetonitrile (3 mL) at 80° C. for 2 h. The crude product was purified by reversed phase HPLC (Phenomenex, Gemini-NX, 10 μm, 250×30 mm, C18 column; gradient of 0% MeCN/0.1% TFA-water to 100% MeCN/0.1% TFA) to afford the title compound (88 mg) as the corresponding TFA salt. ¹H NMR (400 MHz, Methanol-d4, TFA salt) δ8.70 (s, 1H), 7.56 (d, J=2.1 Hz, 1H), 7.54 (d, J=8.5 Hz, 1H), 7.37 (dd, J=8.4, 2.1 Hz, 1H), 5.43 (s, 1H), 5.25 (q, J=6.8 Hz, 1H), 4.58-4.29 (m, 2H), 3.72 (t, J=5.5 Hz, 2H), 3.62-3.51 (m, 2H), 3.41 (s, 3H), 3.37-3.25 (m, 2H), 3.06-2.92 (m, 2H), 2.91-2.81 (m, 1H), 2.80-2.70 (m, 1H), 2.03.1.96 (m, 1H), 1.93.1.86 (m, 1H), 1.85.1.78 (m, 3H), 1.78.1.71 (m, 1H), 1.68 (d, J=6.8 Hz, 3H), 1.66.1.55 (m, 1H), 1.34.1.05 (m, 3H). LCMS [M+H]⁺: 532.1.

### Example 12. N-((R)-1-(2,4-dichlorophenyl)ethyl)-5-((R)-1-(2-(methylsulfonyl)ethyl)-[3,4′-bipiperidin]-1′-yl)-[1,2,4]triazolo[1,5-a]pyrimidin-7-amine

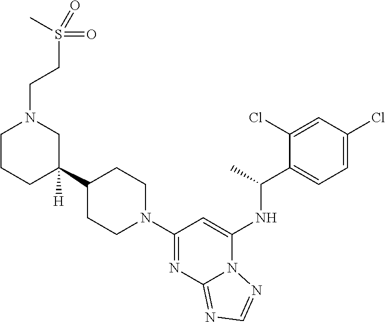 [SMILES: C[C@@H](Nc1cc(N2CCC([C@H]3CCCN(CCS(C)(=O)=O)C3)CC2)nc2ncnn12)c1ccc(Cl)cc1Cl]

Step 1. Synthesis of (R)-1-(2-(methylsulfonyl)ethyl)-3,4′-bipiperidine dihydrochloride. Prepared according to General Procedure F using intermediate II (150 mg, 0.56 mmol), mehyl vinyl sulfone (71.2 mg, 0.67 mmol) and N,N-diisopropylethylamine (0.29 mL, 1.68 mmol) in acetonitrile (3 mL) at 80° C. for 12 h and afforded tert-butyl (R)-1-(2-(methylsulfonyl)ethyl)-[3,4′-bipiperidine]-1′-carboxylate. The crude was then treated with HCl (0.7 mL, 4N in 1,4-dioxane) in DCM (4 mL) and afforded (R)-1-(2-(methylsulfonyl)ethyl)-3,4′-bipiperidine dihydrochloride (193 mg). LCMS [M+H]⁺: 275.2.

Step 2. Synthesis of N-((R)-1-(2,4-dichlorophenyl)ethyl)-5-((R)-1-(2-(methylsulfonyl)ethyl)-[3,4′-bipiperidin]-1′-yl)-[1,2,4]triazolo[1,5-a]pyrimidin-7-amine. Prepared according to General Procedure B using intermediate I (50 mg, 0.15 mmol), (R)-1-(2-(methylsulfonyl)ethyl)-3,4′-bipiperidine dihydrochloride (53.1 mg) and N,N-diisopropylethylamine (0.13 mL, 0.73 mmol) in acetonitrile (3 mL) at 80° C. for 2 h. The crude product was purified by reversed phase HPLC (Phenomenex, Gemini-NX, 10 μm, 250×30 mm, C18 column; gradient of 0% MeCN/0.1% TFA-water to 100% MeCN/0.1% TFA) to afford the title compound (72 mg) as the corresponding TFA salt. ¹H NMR (400 MHz, Methanol-d4, TFA salt) δ8.70 (s, 1H), 7.56 (d, J=2.1 Hz, 1H), 7.54 (d, J=8.5 Hz, 1H), 7.37 (dd, J=8.4, 2.1 Hz, 1H), 5.43 (s, 1H), 5.24 (q, J=6.8 Hz, 1H), 4.55-4.25 (m, 2H) 3.77-3.67 (m, 2H), 3.67-3.55 (m, 5H), 3.10 (s, 3H), 3.07-2.75 (m, 3H), 2.08.1.98 (m, 2H), 1.95.1.87 (m, 1H), 1.87.1.72 (m, 3H), 1.68 (d, J=6.8 Hz, 3H), 1.65.1.57 (m, 1H), 1.39.1.05 (m, 3H). LCMS [M+H]⁺: 580.2.

### Example 13. N-(2-((R)-1′-(7-(((R)-1-(2,4-dichlorophenyl)ethyl)amino)-[1,2,4]triazolo[1,5-a]pyrimidin-5-yl)-[3,4′-bipiperidin]-1-yl)ethyl)acetamide

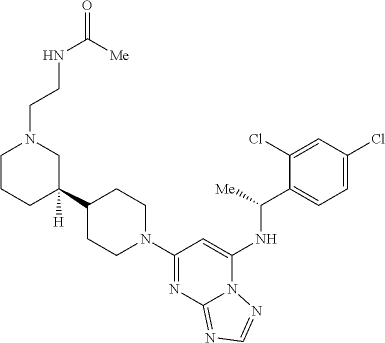 [SMILES: CC(=O)NCCN1CCC[C@H](C2CCN(c3cc(N[C@H](C)c4ccc(Cl)cc4Cl)n4ncnc4n3)CC2)C1]

Step 1. Synthesis of tert-butyl (R)-1-(2-(1,3-dioxoisoindolin-2-yl)ethyl)-[3,4′-bipiperidine]-1′-carboxylate. Prepared according to General Procedure D using Intermediate II (2.1 g, 5.0 mmol) 2-(1,3-dioxoisoindolin-2-yl)acetaldehyde (1.4 g, 7.5 mmol), and sodium triacetoxyborohydride (2.1 g, 10.0 mmol) in DCE (16.6 mL). The reaction mixture was stirred for 16 h then diluted with DCM, filtered through a pad of Celite, and concentrated under reduced pressure. The crude product was purified by normal-phase chromatography using a gradient of MeOH in DCM (0-20%) as eluent to afford tert-butyl (R)-1-(2-(1,3-dioxoisoindolin-2-yl)ethyl)-[3,4′-bipiperidine]-1′-carboxylate (1.9 g).

Step 2. Synthesis of tert-butyl (R)-1-(2-aminoethyl)-[3,4′-bipiperidine]-1′-carboxylate. A solution of tert-butyl (R)-1-(2-(1,3-dioxoisoindolin-2-yl)ethyl)-[3,4′-bipiperidine]-1′-carboxylate (1.9 g, 4.4 mmol) and hydrazine hydrate (0.85 mL, 17.6 mmol) in ethanol (22.0 mL) was heated to 80° C. and stirred for 90 min. White precipitate formed within 10 min. The reaction mixture was cooled to ambient temperature, diluted with ethanol, and then filtered through a Büchner funnel to remove the solids. The filtrate was concentrated under reduced pressure to give tert-butyl (R)-1-(2-aminoethyl)-[3,4′-bipiperidine]-1′-carboxylate (1.3 g).

Step 3. Synthesis of tert-butyl (R)-1-(2-acetamidoethyl)-[3,4′-bipiperidine]-1′-carboxylate. To a solution of crude tert-butyl (R)-1-(2-aminoethyl)-[3,4′-bipiperidine]-1′-carboxylate (500 mg, 1.6 mmol) and triethylamine (0.67 mL, 4.8 mmol) in DCM (8.0 mL) at 0° C. was added acetic anhydride (0.30 mL, 3.2 mmol). The reaction mixture was warmed to ambient temperature and stirred for 16 h, then diluted with water and extracted with DCM. The combined organic phase was dried over anhydrous sodium sulfate, filtered, and concentrated under reduced pressure to afford tert-butyl (R)-1-(2-acetamidoethyl)-[3,4′-bipiperidine]-1′-carboxylate (284 mg).

Step 4. Synthesis of (R)—N-(2-([3,4′-bipiperidin]-1-yl)ethyl)acetamide dihydrochloride. To a solution of crude tert-butyl (R)-1-(2-acetamidoethyl)-[3,4′-bipiperidine]-1′-carboxylate (284 mg, 0.80 mmol) in DCM (4.0 mL) was added a solution of HCl (1.0 mL, 4 N in 1,4-dioxane). The reaction mixture was stirred overnight, then concentrated under reduced pressure to afford crude (R)—N-(2-([3,4′-bipiperidin]-1-yl)ethyl)acetamide dihydrochloride (262 mg).

Step 5. Synthesis of N-(2-((R)-1′-(7-(((R)-1-(2,4-dichlorophenyl)ethyl)amino)-[1,2,4]triazolo[1,5-a]pyrimidin-5-yl)-[3,4′-bipiperidin]-1-yl)ethyl)acetamide. Prepared according to General Procedure A using intermediate I (50 mg, 0.15 mmol) and (R)—N-(2-([3,4′-bipiperidin]-1-yl)ethyl)acetamide dihydrochloride (52 mg, 0.16 mmol) and sodium hydroxide (0.44 mL, 3 M aqueous solution) in a 3:2 tert-butanol:water mixture (0.96 mL) at 80° C. for 18 h. The reaction mixture was cooled to ambient temperature, and the organic phase was separated from the aqueous phase. The aqueous phase was diluted with brine, then extracted with DCM. The combined organic extracts were concentrated under reduced pressure. The crude product was purified by reversed phase HPLC (Phenomenex, Gemini-NX, 10 μm, 250×30 mm, C18 column; gradient of 0% MeCN/0.1% TFA-water to 100% MeCN/0.1% TFA) to afford the title compound (54 mg) as the corresponding TFA salt. ¹H NMR (400 MHz, Methanol-d4, TFA salt) δ8.70 (s, 1H), 7.56 (d, J=2.1 Hz, 1H), 7.54 (d, J=8.4 Hz, 1H), 7.37 (dd, J=8.4, 2.1 Hz, 1H), 5.43 (s, 1H), 5.32-5.18 (m, 1H), 4.56-4.29 (m, 2H), 3.78-3.69 (m, 1H), 3.69-3.56 (m, 2H), 3.56-3.45 (m, 1H), 3.22 (t, J=5.8 Hz, 2H), 3.08-2.92 (m, 2H), 2.92-2.80 (m, 1H), 2.78-2.63 (m, 1H), 2.06-2.00 (m, 1H), 1.99 (s, 3H), 1.96.1.87 (m, 1H), 1.88.1.78 (m, 2H), 1.78-1.71 (m, 1H), 1.68 (d, J=6.8 Hz, 3H), 1.66.1.55 (m, 2H), 1.37.1.04 (m, 3H). LCMS [M+H]⁺: 559.1.

### Example 14. 3-((R)-1′-(7-(((R)-1-(2,4-dichlorophenyl)ethyl)amino)-[1,2,4]triazolo[1,5-a]pyrimidin-5-yl)-[3,4′-bipiperidin]-1-yl)propanoic acid

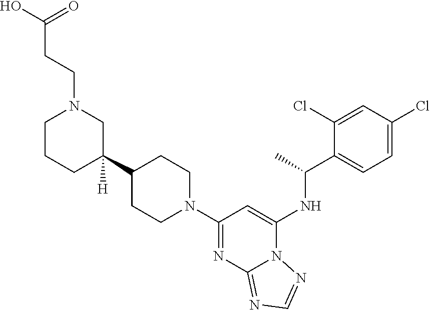 [SMILES: C[C@@H](Nc1cc(N2CCC([C@H]3CCCN(CCC(=O)O)C3)CC2)nc2ncnn12)c1ccc(Cl)cc1Cl]

Step 1. Synthesis of methyl (R)-3-([3,4′-bipiperidin]-1-yl)propanoate dihydrochloride. Prepared according to General Procedure F using intermediate II (300 mg, 1.12 mmol) and methyl acrylate (106 mg, 1.23 mmol) in DMF (5.6 mL). The reaction mixture was stirred at 50° C. until complete as monitored by LCMS. Upon completion, saturated aqueous sodium bicarbonate (30 mL) was added, and the product was extracted into diethyl ether (3×25 mL). The combined organic fractions were dried over magnesium sulfate, filtered, and concentrated under reduced pressure to give tert-butyl (R)-1-(3-methoxy-3-oxopropyl)-[3,4′-bipiperidine]-1′-carboxylate (353 mg). The crude was treated with HCl (2.8 mL, 4 N in 1,4-dioxane) in DCM (4.97 mL) and afforded methyl (R)-3-([3,4′-bipiperidin]-1-yl)propanoate dihydrochloride (360 mg).

Step 2. Synthesis of 3-((R)-1′-(7-(((R)-1-(2,4-dichlorophenyl)ethyl)amino)-[1,2,4]triazolo[1,5-a]pyrimidin-5-yl)-[3,4′-bipiperidin]-1-yl)propanoic acid. Prepared according to General Procedure B using intermediate I (80 mg, 0.23 mmol) in tert-butanol (1.05 mL), while separately methyl (R)-3-([3,4′-bipiperidin]-1-yl)propanoate dihydrochloride (76 mg, 0.23 mmol) was dissolved in water (0.35 mL) and added NaOH (0.7 mL, 3N aqueous solution). The two resulting mixtures were then combined and stirred at 80° C. for 3 h. The crude product was purified by reversed phase HPLC (Phenomenex, Gemini-NX, 10 μm, 250×30 mm, C18 column; gradient of 0% MeCN/0.1% TFA-water to 100% MeCN/0.1% TFA) to afford the title compound (44 mg) as the corresponding TFA salt. ¹H NMR (400 MHz, Methanol-d4, TFA salt) δ8.71 (s, 1H), 7.56 (d, J=2.1 Hz, 1H), 7.54 (d, J=8.5 Hz, 1H), 7.37 (dd, J=8.5, 2.1 Hz, 1H), 5.43 (s, 1H), 5.24 (q, J=6.9 Hz, 1H), 4.55-4.29 (m, 2H), 3.59-3.52 (m, 3H), 3.40 (t, J=7.1 Hz, 2H), 3.06-2.89 (m, 2H), 2.84 (t, J=7.2 Hz, 2H), 2.80-2.71 (m, 1H), 2.05.1.98 (m, 1H), 1.95.1.85 (m, 1H), 1.84.1.72 (m, 3H), 1.68 (d, J=6.8 Hz, 3H), 1.66.1.57 (m, 2H), 1.35-1.04 (m, 3H). LCMS [M+H]: 546.2.

### Example 15. 3-((R)-1′-(7-(((R)-1-(2,4-dichlorophenyl)ethyl)amino)-[1,2,4]triazolo[1,5-a]pyrimidin-5-yl)-[3,4′-bipiperidin]-1-yl)-2-methylpropanoic acid

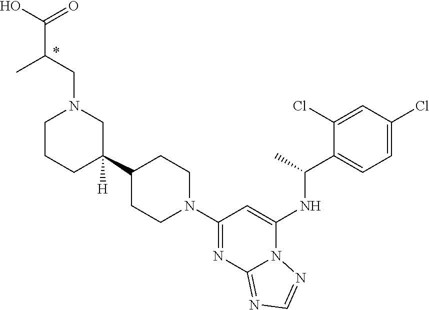 [SMILES: CC(CN1CCC[C@H](C2CCN(c3cc(N[C@H](C)c4ccc(Cl)cc4Cl)n4ncnc4n3)CC2)C1)C(=O)O]

Step 1. Synthesis of methyl 3-((R)-[3,4′-bipiperidin]-1-yl)-2-methylpropanoate dihydrochloride. Prepared according to General Procedure F using intermediate II (300 mg, 1.12 mmol) and methyl methacrylate (224 mg, 2.24 mmol) in DMF (5.6 mL). The reaction mixture was stirred at 50° C. until complete as monitored by LCMS. Upon completion, saturated aqueous sodium bicarbonate (30 mL) was added, and the product was extracted into diethyl ether (3×25 mL). The combined organic fractions were dried over magnesium sulfate, filtered, and concentrated under reduced to give tert-butyl (3R)-1-(3-methoxy-2-methyl-3-oxopropyl)-[3,4′-bipiperidine]-1′-carboxylate (289 mg). The crude was treated with HCl (2.2 mL, 4 N in 1,4-dioxane) in DCM (3.9 mL) and afforded methyl 3-((R)-[3,4′-bipiperidin]-1-yl)-2-methylpropanoate dihydrochloride (303 mg).

Step 2. Synthesis of 3-((R)-1′-(7-(((R)-1-(2,4-dichlorophenyl)ethyl)amino)-[1,2,4]triazolo[1,5-a]pyrimidin-5-yl)-[3,4′-bipiperidin]-1-yl)-2-methylpropanoic acid. Prepared according to General Procedure B using intermediate I (100 mg, 0.29 mmol) in tert-butanol (1.3 mL), while separately methyl 3-((R)-[3,4′-bipiperidin]-1-yl)-2-methylpropanoate dihydrochloride (99.6 mg, 0.29 mmol) was dissolved in water (0.43 mL) and added NaOH (0.85 mL, 3N aqueous solution). The two resulting mixtures were then combined and stirred at 80° C. for 3 h. The crude product was purified by reversed phase HPLC (Phenomenex, Gemini-NX, 10 μm, 250×30 mm, C18 column; gradient of 0% MeCN/0.1% TFA-water to 100% MeCN/0.1% TFA) to afford the title compound (36 mg, mixture of diastereomers) as the corresponding TFA salt. ¹H NMR (400 MHz, Methanol-d4, TFA salt) δ8.71 (s, 1H), 7.58-7.50 (m, 2H), 7.37 (dd, J=8.4, 1.6 Hz, 1H), 5.42 (s, 1H), 5.24 (q, J=6.8 Hz, 1H), 4.62-4.16 (m, 2H), 3.65-3.43 (m, 3H), 3.15-2.91 (m, 4H), 2.91-2.68 (m, 2H), 2.02.1.95 (m, 1H), 1.92.1.75 (m, 4H), 1.75.1.70 (m, 1H), 1.68 (d, J=6.8 Hz, 3H), 1.63.1.55 (m, 1H), 1.29 (d, J=7.0 Hz, 3H), 1.28.1.04 (m, 3H). LCMS [M+H]: 560.2.

### Example 16. 4-((R)-1′-(7-(((R)-1-(2,4-dichlorophenyl)ethyl)amino)-[1,2,4]triazolo[1,5-a]pyrimidin-5-yl)-[3,4′-bipiperidin]-1-yl)-2-methylbutanoic acid

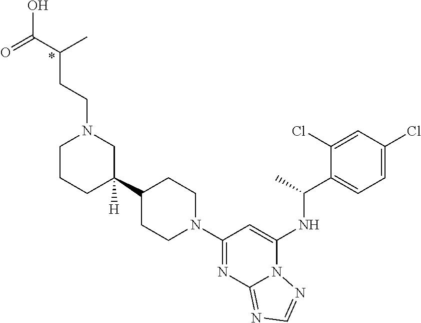 [SMILES: CC(CCN1CCC[C@H](C2CCN(c3cc(N[C@H](C)c4ccc(Cl)cc4Cl)n4ncnc4n3)CC2)C1)C(=O)O]

Step 1. Synthesis of methyl 4-((R)-[3,4′-bipiperidin]-1-yl)-2-methylbutanoate dihydrochloride. Prepared according to General Procedure E using intermediate II (300 mg, 1.12 mmol), methyl 4-bromo-2-methylbutanoate (240 mg, 1.23 mmol), and potassium carbonate (463 mg, 3.35 mmol) in MeCN (5.59 mL). The reaction mixture was stirred at 80° C. until complete as monitored by LCMS and afforded tert-butyl (3R)-1-(4-methoxy-3-methyl-4-oxobutyl)-[3,4′-bipiperidine]-1′-carboxylate (410 mg). The crude was treated with HCl (3.0 mL, 4 N in 1,4-dioxane) in DCM (5.35 mL) and afforded methyl 4-((R)-[3,4′-bipiperidin]-1-yl)-2-methylbutanoate dihydrochloride (369 mg).

Step 2. Synthesis of 4-((R)-1′-(7-(((R)-1-(2,4-dichlorophenyl)ethyl)amino)-[1,2,4]triazolo[1,5-a]pyrimidin-5-yl)-[3,4′-bipiperidin]-1-yl)-2-methylbutanoic acid. Prepared according to General Procedure B using intermediate I (100 mg, 0.29 mmol) in tert-butanol (1.3 mL), while separately methyl 4-((R)-[3,4′-bipiperidin]-1-yl)-2-methylbutanoate dihydrochloride (104 mg, 0.29 mmol) was dissolved in water (0.43 mL) and added NaOH (0.85 mL, 3N aqueous solution). The two resulting mixtures were then combined and stirred at 80° C. for 3 h. The crude product was purified by reversed phase HPLC (Phenomenex, Gemini-NX, 10 μm, 250×30 mm, C18 column; gradient of 0% MeCN/0.1% TFA-water to 100% MeCN/0.1% TFA) to afford the title compound (35 mg, mixture of diastereomers) as the corresponding TFA salt. ¹H NMR (400 MHz, Methanol-d4, TFA salt) δ8.70 (s, 1H), 7.56 (d, J=2.1 Hz, 1H), 7.54 (d, J=8.5 Hz, 1H), 7.37 (dd, J=8.5, 2.1 Hz, 1H), 5.42 (s, 1H), 5.24 (q, J=6.8 Hz, 1H), 4.59-4.20 (m, 2H), 3.55 (t, J=11.6 Hz, 2H), 3.18-3.12 (m, 2H), 3.05-2.92 (m, 2H), 2.88-2.79 (m, 1H), 2.78-2.69 (m, 1H), 2.57-2.48 (m, 1H), 2.13.1.96 (m, 2H), 1.93.1.84 (m, 2H), 1.84.1.73 (m, 3H), 1.68 (d, J=6.8 Hz, 3H), 1.65.1.56 (m, 2H), 1.24 (d, J=7.1 Hz, 3H), 1.19.1.04 (m, 3H). LCMS [M+H]: 574.2.

### Example 17.3-((R)-1′-(7-(((R)-1-(2,4-dichlorophenyl)ethyl)amino)-[1,2,4]triazolo[1,5-a]pyrimidin-5-yl)-[3,4′-bipiperidin]-1-yl)-N-methylpropanamide

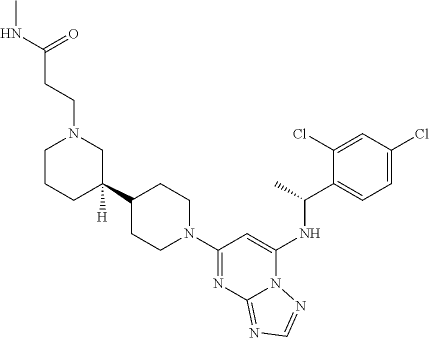 [SMILES: CNC(=O)CCN1CCC[C@H](C2CCN(c3cc(N[C@H](C)c4ccc(Cl)cc4Cl)n4ncnc4n3)CC2)C1]

Step 1. Synthesis of (R)-3-([3,4′-bipiperidin]-1-yl)-N-methylpropanamide dihydrochloride. Prepared according to General Procedure F using intermediate II (158 mg, 0.59 mmol), N-methylacrylamide (60 mg, 0.71 mmol) in DMF (2.9 mL). The reaction mixture was stirred at 65° C. until complete as monitored by LCMS. Upon completion, saturated aqueous sodium bicarbonate (20 mL) was added, and the product was extracted into diethyl ether (3×20 mL). The combined organics were dried over magnesium sulfate, filtered, and concentrated under reduced pressure to give tert-butyl (R)-1-(3-(methylamino)-3-oxopropyl)-[3,4′-bipiperidine]-1′-carboxylate (196 mg). The crude was treated with HCl (1.56 mL, 4 N in 1,4-dioxane) in DCM (4.97 mL) and afforded (R)-3-([3,4′-bipiperidin]-1-yl)-N-methylpropanamide dihydrochloride (202 mg).

Step 2. Synthesis of 3-((R)-1′-(7-(((R)-1-(2,4-dichlorophenyl)ethyl)amino)-[1,2,4]triazolo[1,5-a]pyrimidin-5-yl)-[3,4′-bipiperidin]-1-yl)-N-methylpropanamide. Prepared according to General Procedure B using intermediate I (100 mg, 0.29 mmol), (R)-3-([3,4′-bipiperidin]-1-yl)-N-methylpropanamide dihydrochloride (95.2 mg, 0.29 mmol) and N,N-diisopropylethylamine (0.25 mL, 1.45 mmol) in DMSO (1.42 mL). The crude product was purified by reversed phase HPLC (Phenomenex, Gemini-NX, 10 μm, 250×30 mm, C18 column; gradient of 0% MeCN/0.1% TFA-water to 100% MeCN/0.1% TFA) to afford the title compound (12.4 mg) as the corresponding TFA salt. ¹H NMR (400 MHz, Methanol-d4, TFA salt) δ8.71 (s, 1H), 7.58-7.50 (m, 2H), 7.37 (d, J=8.4 Hz, 1H), 5.43 (s, 1H), 5.24 (q, J=6.8 Hz, 1H), 4.57-4.21 (m, 2H), 3.62-3.52 (m, 2H), 3.42-3.34 (m, 2H), 3.05-2.91 (m, 3H), 2.89-2.79 (m, 1H), 2.74 (s, 3H), 2.70 (t, J=6.7 Hz, 2H), 2.07.1.97 (m, 1H), 1.95.1.85 (m, 1H), 1.85.1.71 (m, 3H) 1.68 (d, J=6.8 Hz, 3H), 1.66.1.57 (m, 2H), 1.35.1.21 (m, 2H), 1.19-1.04 (m, 1H). LCMS [M+H]: 559.2.

### Example 18. (1R,3r)-3-((R)-1′-(7-(((R)-1-(2,4-dichlorophenyl)ethyl)amino)-[1,2,4]triazolo[1,5-a]pyrimidin-5-yl)-[3,4′-bipiperidin]-1-yl)-1-methylcyclobutane-1-carboxylic acid

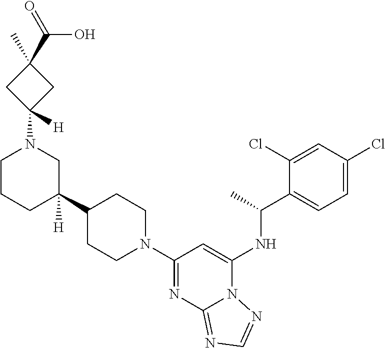 [SMILES: C[C@@H](Nc1cc(N2CCC([C@H]3CCCN([C@H]4C[C@](C)(C(=O)O)C4)C3)CC2)nc2ncnn12)c1ccc(Cl)cc1Cl]

Step 1. Synthesis of methyl (1R,3r)-3-((R)-[3,4′-bipiperidin]-1-yl)-1-methylcyclobutane-1-carboxylate dihydrochloride. Prepared according to General Procedure D using intermediate II (1.42 g, 5.29 mmol), 1-methyl-3-oxocyclobutane-1-carboxylic acid (0.881 g, 6.88 mmol) and diludine (1.74 g, 6.88 mmol) in toluene (51 mL) at 75° C. for 12 h. The mixture was cooled to room temperature and concentrated under reduced pressure. The residue was suspended in water (20 mL) and MTBE (20 mL) and filtered through celite. The aqueous fraction was washed with MTBE (2×25 mL) and concentrated under reduced pressure to give (1R,3r)-3-((R)-1′-(tert-butoxycarbonyl)-[3,4′-bipiperidin]-1-yl)-1-methylcyclobutane-1-carboxylic acid (1.41 g). A portion of the crude (150 mg, 0.39) was treated with HCl (1.56 mL, 4 N in 1,4-dioxane) in methanol (3 mL) and afforded methyl (1R,3r)-3-((R)-[3,4′-bipiperidin]-1-yl)-1-methylcyclobutane-1-carboxylate dihydrochloride (138 mg).

Step 2. Synthesis of (1R,3r)-3-((R)-1′-(7-(((R)-1-(2,4-dichlorophenyl)ethyl)amino)-[1,2,4]triazolo[1,5-a]pyrimidin-5-yl)-[3,4′-bipiperidin]-1-yl)-1-methylcyclobutane-1-carboxylic acid. Prepared according to General Procedure B using intermediate I (60 mg, 0.18 mmol), methyl (1R,3r)-3-((R)-[3,4′-bipiperidin]-1-yl)-1-methylcyclobutane-1-carboxylate dihydrochloride (64 mg, 0.18 mmol), and NaOH (0.53 mL, 3 N aqueous solution) in tert-butanol (1 mL) and water (1 mL) at 80° C. for 19 h. The crude product was purified by reversed phase HPLC (Phenomenex, Gemini-NX, 10 μm, 250×30 mm, C18 column; gradient of 0% MeCN/0.1% TFA-water to 100% MeCN/0.1% TFA) to afford the title compound (41 mg) as the corresponding TFA salt. ¹H NMR (400 MHz, DMSO-d6, TFA salt) δ9.49-9.18 (m, 1H), 8.60 (s, 1H), 8.55-8.40 (m, 1H), 7.67 (d, J=2.1 Hz, 1H), 7.65 (d, J=8.5 Hz, 1H), 7.47 (d, J=8.5 Hz, 1H), 5.36 (s, 1H), 5.16-5.06 (m, 1H), 4.34-4.21 (m, 2H), 3.71-3.60 (m, 1H), 3.34-3.27 (m, 1H), 3.25-3.18 (m, 1H), 2.93-2.80 (m, 2H), 2.69-2.58 (m, 3H), 2.58-2.52 (m, 1H), 2.22-2.10 (m, 2H), 1.92.1.81 (m, 1H), 1.76.1.68 (m, 1H), 1.68.1.62 (m, 2H), 1.59 (d, J=6.9 Hz, 3H), 1.57.1.46 (m, 3H), 1.31 (s, 3H), 1.20.1.09 (m, 1H), 1.08-0.96 (m, 2H). LCMS [M+H]⁺: 586.2.

### Example 19.2-((R)-1′-(7-(((R)-1-(2,4-dichlorophenyl)ethyl)amino)-[1,2,4]triazolo[1,5-a]pyrimidin-5-yl)-[3,4′-bipiperidin]-1-yl)ethane-1-sulfonamide

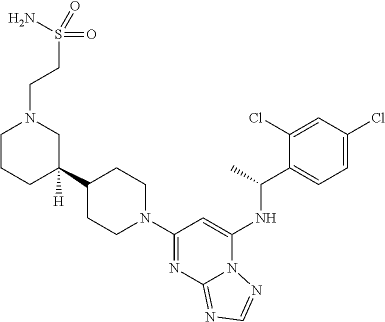 [SMILES: C[C@@H](Nc1cc(N2CCC([C@H]3CCCN(CCS(N)(=O)=O)C3)CC2)nc2ncnn12)c1ccc(Cl)cc1Cl]

Step 1. Synthesis of (R)-2-([3,4′-bipiperidin]-1-yl)ethane-1-sulfonamide dihydrochloride. Prepared according to General Procedure E using intermediate II (200 mg, 0.75 mmol), 2-chloroethanesulfonamide (107 mg, 0.75 mmol), sodium iodide (11.2 mg, 0.07 mmol), and sodium carbonate (158 mg, 1.5 mmol) in 1,4 dioxane (2.5 mL) at 80° C. for 18 h, and afforded tert-butyl (R)-1-(2-sulfamoylethyl)-[3,4′-bipiperidine]-1′-carboxylate. The crude was then treated with HCl (0.93 mL, 4 N in 1,4-dioxane) in DCM (4 mL) and afforded (R)-2-([3,4′-bipiperidin]-1-yl)ethane-1-sulfonamide dihydrochloride (250 mg).

Step 2. Synthesis of 2-((R)-1′-(7-(((R)-1-(2,4-dichlorophenyl)ethyl)amino)-[1,2,4]triazolo[1,5-a]pyrimidin-5-yl)-[3,4′-bipiperidin]-1-yl)ethane-1-sulfonamide. Prepared according to General Procedure B using intermediate I (50 mg, 0.15 mmol), (R)-2-([3,4′-bipiperidin]-1-yl)ethane-1-sulfonamide dihydrochloride (50.7 mg, 0.15 mmol) and potassium phosphate tribasic (154 mg, 0.73 mmol) in tert-butanol (1.2 mL) and water (0.4 mL) at 80° C. for 2 h. The crude product was purified by reversed phase HPLC (Phenomenex, Gemini-NX, 10 μm, 250×30 mm, C18 column; gradient of 0% MeCN/0.1% TFA-water to 100% MeCN/0.1% TFA) to afford the title compound (90 mg) as the corresponding TFA salt. ¹H NMR (400 MHz, Methanol-d4, TFA salt) δ8.70 (s, 1H), 7.55 (d, J=1.9 Hz, 1H), 7.52 (d, J=8.5 Hz, 1H), 7.36 (dd, J=8.5, 2.0 Hz, 1H), 5.42 (s, 1H), 5.24 (q, J=6.8 Hz, 1H), 4.64-4.21 (m, 2H), 3.64-3.56 (m, 6H), 3.08-2.87 (m, 3H), 2.86-2.75 (m, 1H), 2.07.1.97 (m, 1H), 1.93.1.86 (m, 1H), 1.86.1.71 (m, 4H), 1.68 (d, J=6.8 Hz, 3H), 1.66.1.56 (m, 1H), 1.37-0.99 (m, 3H). LCMS [M+H]⁺: 581.2.

### Example 20. 3-((R)-1′-(7-(((R)-1-(2,4-dichlorophenyl)ethyl)amino)-[1,2,4]triazolo[1,5-a]pyrimidin-5-yl)-[3,4′-bipiperidin]-1-yl)-N-methylpropane-1-sulfonamide

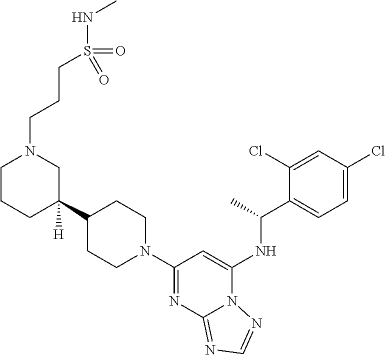 [SMILES: CNS(=O)(=O)CCCN1CCC[C@H](C2CCN(c3cc(N[C@H](C)c4ccc(Cl)cc4Cl)n4ncnc4n3)CC2)C1]

Step 1. Synthesis of (R)-3-([3,4′-bipiperidin]-1-yl)-N-methylpropane-1-sulfonamide dihydrochloride. Prepared according to General Procedure E using intermediate II (250 mg, 0.93 mmol), 3-chloro-N-methyl-propane-1-sulfonamide (160 mg, 0.93 mmol), sodium iodide (14 mg, 0.09 mmol), and sodium carbonate (197 mg, 1.8 mmol) in 1,4 dioxane (2.5 mL) at 80° C. for 18 h and afforded tert-butyl (R)-1-(3-(N-methylsulfamoyl)propyl)-[3,4′-bipiperidine]-1′-carboxylate. The crude was then treated with HCl (0.93 mL, 4 N in 1,4-dioxane) in DCM (4 mL) and afforded (R)-3-([3,4′-bipiperidin]-1-yl)-N-methylpropane-1-sulfonamide dihydrochloride (300 mg).

Step 2. Synthesis of 3-((R)-1′-(7-(((R)-1-(2,4-dichlorophenyl)ethyl)amino)-[1,2,4]triazolo[1,5-a]pyrimidin-5-yl)-[3,4′-bipiperidin]-1-yl)-N-methylpropane-1-sulfonamide. Prepared according to General Procedure B using intermediate I (50 mg, 0.15 mmol), (R)-3-([3,4′-bipiperidin]-1-yl)-N-methylpropane-1-sulfonamide dihydrochloride (54.8 mg, 0.15 mmol) and potassium phosphate tribasic (154 mg, 0.73 mmol) in tert-butanol (1.2 mL) and water (0.4 mL) at 80° C. for 2 h. The crude product was purified by reversed phase HPLC (Phenomenex, Gemini-NX, 10 μm, 250×30 mm, C18 column; gradient of 0% MeCN/0.1% TFA-water to 100% MeCN/0.1% TFA) to afford the title compound (100 mg) as the corresponding TFA salt. ¹H NMR (400 MHz, Methanol-d4, TFA salt) δ8.71 (s, 1H), 7.56 (d, J=2.4 Hz, 1H), 7.54 (d, J=8.4 Hz, 1H), 7.37 (dd, J=8.4, 2.4 Hz, 1H), 5.43 (s, 1H), 5.24 (q, J=6.8 Hz, 1H), 4.65-4.17 (m, 2H), 3.60-3.50 (m, 2H), 3.29-3.24 (m, 2H), 3.15 (t, J=7.2 Hz, 2H), 3.07-2.92 (m, 2H), 2.91 - 2.80 (m, 1H), 2.79-2.73 (m, 1H), 2.72 (s, 3H), 2.28-2.16 (m, 2H), 2.07.1.96 (m, 1H), 1.95-1.86 (m, 1H), 1.86.1.70 (m, 3H), 1.68 (d, J=6.9 Hz, 3H), 1.65.1.56 (m, 2H), 1.36.1.03 (m, 3H). LCMS [M+H]⁺: 609.2.

### Example 21. N-((R)-1-(2,4-dichlorophenyl)ethyl)-5-((R)-1-(3-(methylsulfonyl)cyclobutyl)-[3,4′-bipiperidin]-1′-yl)-[1,2,4]triazolo[1,5-a]pyrimidin-7-amine

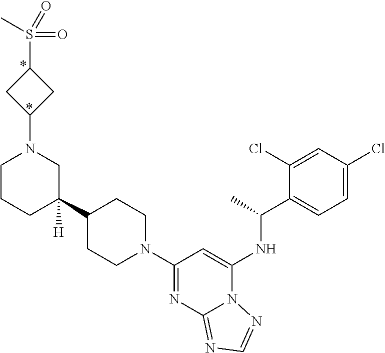 [SMILES: C[C@@H](Nc1cc(N2CCC([C@H]3CCCN(C4CC(S(C)(=O)=O)C4)C3)CC2)nc2ncnn12)c1ccc(Cl)cc1Cl]

Step 1. Synthesis of (R)-1-(3-(methylsulfonyl)cyclobutyl)-3,4′-bipiperidine 2,2,2-trifluoroacetic acid. Prepared according to General Procedure D using intermediate II (100 mg, 0.37 mmol), 3-(methylsulfonyl)cyclobutan-1-one (82.8 mg, 0.56 mmol) and sodium triacetoxyborohydride (158 mg, 0.75 mmol) in DCE (1.8 mL) at 80° C. for 18 h and afforded tert-butyl (R)-1-(3-(methylsulfonyl)cyclobutyl)-[3,4′-bipiperidine]-1′-carboxylate. The crude was then treated with trifluoroacetic acid (1 mL) in DCM (2 mL) and afforded (R)-1-(3-(methylsulfonyl)cyclobutyl)-3,4′-bipiperidine 2,2,2-trifluoroacetic acid (150 mg) as a mixture of diastereomers.

Step 2. Synthesis of N-((R)-1-(2,4-dichlorophenyl)ethyl)-5-((R)-1-(3-(methylsulfonyl)cyclobutyl)-[3,4′-bipiperidin]-1′-yl)-[1,2,4]triazolo[1,5-a]pyrimidin-7-amine. Prepared according to General Procedure B using intermediate I (50 mg, 0.15 mmol), (R)-1-(3-(methylsulfonyl)cyclobutyl)-3,4′-bipiperidine 2,2,2-trifluoroacetic acid (60.3 mg, 0.15 mmol) and potassium phosphate tribasic (154 mg, 0.73 mmol) in tert-butanol (1.2 mL) and water (0.4 mL) at 80° C. for 2 h. The crude product was purified by reversed phase HPLC (Phenomenex, Gemini-NX, 10 μm, 250×30 mm, C18 column; gradient of 0% MeCN/0.1% TFA-water to 100% MeCN/0.1% TFA) to afford the title compound (13 mg) as the corresponding TFA salt as a mixture of diastereomers. ¹H NMR (400 MHz, Methanol-d4, TFA salt) δ8.70 (s, 1H), 7.55 (d, J=2.2 Hz, 1H), 7.52 (d, J=8.5 Hz, 1H), 7.36 (dd, J=8.5, 2.3 Hz, 1H), 5.42 (s, 1H), 5.24 (q, J=7.0 Hz, 1H), 4.60-4.26 (m, 2H), 3.88-3.75 (m, 1H), 3.77-3.63 (m, 1H), 3.52-3.39 (m, 2H), 3.07-2.94 (m, 2H), 2.93 (s, 3H), 2.79-2.55 (m, 6H), 2.06.1.96 (m, 1H), 1.94.1.86 (m, 1H), 1.86.1.71 (m, 3H), 1.68 (d, J=7.0 Hz, 3H), 1.66.1.52 (m, 2H), 1.38.1.05 (m, 3H). LCMS [M+H]⁺: 606.2.

### Example 22. 3-((R)-1′-(7-(((R)-1-(2,4-dichlorophenyl)ethyl)amino)-[1,2,4]triazolo[1,5-a]pyrimidin-5-yl)-[3,4′-bipiperidin]-1-yl)propan-1-ol

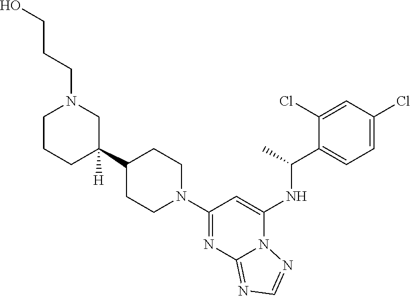 [SMILES: C[C@@H](Nc1cc(N2CCC([C@H]3CCCN(CCCO)C3)CC2)nc2ncnn12)c1ccc(Cl)cc1Cl]

Step 1. Synthesis of (R)-3-([3,4′-bipiperidin]-1-yl)propan-1-ol dihydrochloride. Prepared according to General Procedure F using intermediate II (175 mg, 0.65 mmol), and methyl acrylate (56 mg, 0.65 mmol) in DMF (3 mL) at room temperature, followed by reduction of the resulting ester with LiBH₄ (62 mg, 2.82 mmol) in THF/MeOH at room temperature. The crude was then treated with HCl (0.6 mL, 4 N in 1,4-dioxane) in DCM (2 mL) to afford (R)-3-([3,4′-bipiperidin]-1-yl)propan-1-ol dihydrochloride (130 mg).

Step 2. Synthesis of 3-((R)-1′-(7-(((R)-1-(2,4-dichlorophenyl)ethyl)amino)-[1,2,4]triazolo[1,5-a]pyrimidin-5-yl)-[3,4′-bipiperidin]-1-yl)propan-1-ol. Prepared according to General Procedure B using intermediate I (50 mg, 0.15 mmol), (R)-3-([3,4′-bipiperidin]-1-yl)propan-1-ol dihydrochloride (43.6 mg, 0.15 mmol) and potassium phosphate tribasic (154 mg, 0.73 mmol) in tert-butanol (1.2 mL) and water (0.4 mL) at 80° C. for 2 h. The crude product was purified by reversed phase HPLC (Phenomenex, Gemini-NX, 10 μm, 250×30 mm, C18 column; gradient of 0% MeCN/0.1% TFA-water to 100% MeCN/0.1% TFA) to afford the title compound (42 mg) as the corresponding TFA salt. ¹H NMR (400 MHz, Methanol-d4, TFA salt) δ8.72 (s, 1H), 7.54 (d, J=2.0 Hz, 1H), 7.53 (d, J=8.5 Hz, 1H), 7.37 (dd, J=8.5, 2.0 Hz, 1H), 5.42 (s, 1H), 5.24 (q, J=6.9 Hz, 1H), 4.72-4.11 (m, 2H), 3.73-3.64 (m, 2H), 3.60-3.52 (m, 2H), 3.29-3.15 (m, 2H), 3.07-2.91 (m, 2H), 2.84 (m, 1H), 2.72 (m, 1H), 2.06.1.86 (m, 4H), 1.86.1.71 (m, 3H), 1.68 (d, J=6.9 Hz, 3H), 1.65.1.56 (m, 2H), 1.43-0.99 (m, 3H). LCMS [M+H]⁺: 532.2.

### Example 23a/b. 2-(3-(1-(7-(((R)-1-(2,4-dichlorophenyl)ethyl)amino)-[1,2,4]triazolo[1,5-a]pyrimidin-5-yl)pyrrolidin-3-yl)piperidin-1-yl)ethan-1-ol

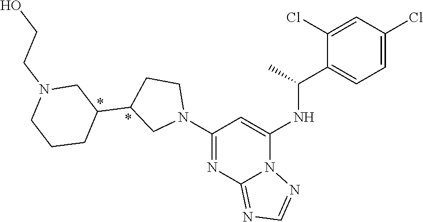 [SMILES: C[C@@H](Nc1cc(N2CCC(C3CCCN(CCO)C3)C2)nc2ncnn12)c1ccc(Cl)cc1Cl]

Step 1. Synthesis of 2-(3-(pyrrolidin-3-yl)piperidin-1-yl)ethan-1-ol dihydrochloride. Prepared according to General Procedure D using commercially available tert-butyl 3-(piperidin-3-yl)pyrrolidine-1-carboxylate (200 mg, 0.79 mmol), 2-((tert-butyldimethylsilyl)oxy)-acetaldehyde (137 mg, 0.79 mmol), and sodium triacetoxyborohydride (200 mg, 0.94 mmol) in THF (5 mL) at room temperature for 24 h. The reaction mixture was diluted in EtOAc and washed with a saturated aqueous solution of sodium carbonate. The organic layer was dried over anhydrous sodium sulfate, filtered, and concentrated. The crude intermediate was dissolved in DCM (5 mL), added HCl (2.0 mL, 4 N in 1,4-dioxane), and stirred at room temperature for 18 h. The crude product was concentrated under reduced pressure until a constant weight was attained and afforded 2-(3-(pyrrolidin-3-yl)piperidin-1-yl)ethan-1-ol dihydrochloride (206 mg). LCMS [M+H]⁺: 199.1.

Step 2. Synthesis of 2-(3-(1-(7-(((R)-1-(2,4-dichlorophenyl)ethyl)amino)-[1,2,4]triazolo[1,5-a]pyrimidin-5-yl)pyrrolidin-3-yl)piperidin-1-yl)ethan-1-ol. Prepared according to General Procedure B using intermediate I (50 mg, 0.15 mmol), 2-(3-(pyrrolidin-3-yl)piperidin-1-yl)ethan-1-ol dihydrochloride (44 mg, 0.16 mmol), and N,N-diisopropylethylamine (0.28 mL, 1.61 mmol) in acetonitrile (3 mL) and water (0.3 mL) at 80° C. for 21 h. The crude product was purified by reversed phase HPLC (Phenomenex, Gemini-NX, 10 μm, 250×30 mm, C18 column; gradient of 0% MeCN/0.1% TFA-water to 100% MeCN/0.1% TFA) to afford the title compound as 2 sets of diastereomers and as the corresponding TFA salts. Data for first eluting set of diastereomers (Peak 1, 15 mg). ¹H NMR (400 MHz, Methanol-d4, TFA salt) δ8.71 (s, 1H), 7.59-7.52 (m, 2H), 7.43-7.34 (m, 1H), 5.30-5.23 (m, 1H), 5.23-5.14 (m, 1H), 4.09-3.94 (m, 1H), 3.93-3.84 (m, 2H), 3.79-3.70 (m, 1H), 3.70-3.58 (m, 2H), 3.52-3.35 (m, 2H), 3.29-3.23 (m, 2H), 3.23-3.11 (m, 1H), 3.02-2.86 (m, 1H), 2.85-2.73 (m, 1H), 2.31-2.13 (m, 1H), 2.13.1.95 (m, 2H), 1.94.1.84 (m, 2H), 1.83.1.72 (m, 1H), 1.68 (d, J=6.8 Hz, 3H), 1.45.1.26 (m, 1H). LCMS [M+H]⁺: 504.2. Data for second eluting set of diastereomers (Peak 2, 18.5 mg). ¹H NMR (400 MHz, Methanol-d4, TFA salt) δ8.69 (s, 1H), 7.62-7.50 (m, 2H), 7.40-7.33 (m, 1H), 5.29-5.23 (m, 1H), 5.23-5.14 (m, 1H), 4.04-3.92 (m, 2H), 3.92-3.82 (m, 2H), 3.77-3.68 (m, 1H), 3.68-3.59 (m, 1H), 3.59-3.52 (m, 1H), 3.50-3.34 (m, 2H), 3.29-3.22 (m, 1H), 3.21-3.12 (m, 1H), 3.00-2.75 (m, 2H), 2.33-2.14 (m, 1H), 2.08.1.96 (m, 2H), 1.95.1.74 (m, 3H), 1.68 (d, J=6.8 Hz, 3H), 1.41-1.33 (m, 1H). LCMS [M+H]⁺: 504.2.

### Example 24. (1R,3r)-3-((R)-1′-(7-(((R)-1-(2,4-dichlorophenyl)ethyl)amino)pyrazolo[1,5-a]pyrimidin-5-yl)-[3,4′-bipiperidin]-1-yl)-1-methylcyclobutane-1-carboxylic acid

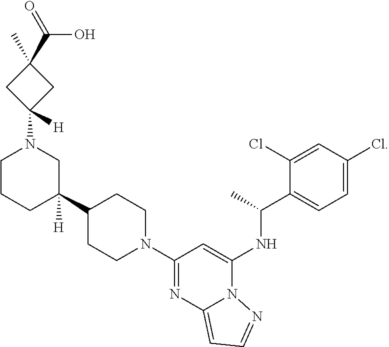 [SMILES: C[C@@H](Nc1cc(N2CCC([C@H]3CCCN([C@H]4C[C@](C)(C(=O)O)C4)C3)CC2)nc2ccnn12)c1ccc(Cl)cc1Cl]

Step 1. Preparation of (1R,3r)-3-((R)-1′-(tert-butoxycarbonyl)-[3,4′-bipiperidin]-1-yl)-1-methylcyclobutane-1-carboxylic acid. Intermediate 11(9.5 g, 35.4 mmol) was dissolved in dry toluene (235 mL) and added 1-methyl-3-oxocyclobutane-1-carboxylic acid (5.94 g, 46.4 mmol) and diludine (11.7 g, 46.4 mmol). The mixture was heated to 75° C. for 20 h. The reaction mixture was concentrated, then diluted with water (200 mL) and MTBE (200 mL), and sonicated to dissolve the precipitation. The reaction mixture was filtered through a celite pad. The aqueous layer of the filtrate was washed with MTBE (2×200 mL), then concentrated under reduced pressure to afford the title compound (13.05 g). LCMS [M+H]⁺: 381.1.

Step 2. Preparation of (1R,3r)-3-((R)-[3,4′-bipiperidin]-1-yl)-1-methylcyclobutane-1-carboxylic acid dihydrochloride. (1R,3r)-3-((R)-1′-(tert-Butoxycarbonyl)-[3,4′-bipiperidin]-1-yl)-1-methylcyclobutane-1-carboxylic acid (620 mg) was suspended in DCM (6 mL) and added HCl (4 M in 1,4-dioxane) (2.04 mL, 8.15 mmol). The mixture was stirred at room temperature for 2 h. The crude is concentrated under reduced pressure to afford the title compound (546 mg). LCMS [M+H]⁺: 281.1.

Step 3. Synthesis of (R)-5-chloro-N-(1-(2,4-dichlorophenyl)ethyl)pyrazolo[1,5-a]pyrimidin-7-amine. This compound was prepared according to General Procedure A using 5,7-dichloropyrazolo[1,5-a]pyrimidine (1 g, 5.32 mmol), (R)-1-(2,4-dichlorophenyl)ethan-1-amine (1.01 g, 5.32 mmol) and N,N-diisopropylethylamine (1.85 mL, 10.6 mmol) in MeCN (10.6 mL) at 80° C. for 18 h. The crude product was purified by silica column chromatography (0-50% EtOAc in hexanes) to provide the desired product (1.78 g). LCMS [M+H]⁺: 340.9

Step 4. Synthesis of (1R,3r)-3-((R)-1′-(7-(((R)-1-(2,4-dichlorophenyl)ethyl)amino)pyrazolo[1,5-a]pyrimidin-5-yl)-[3,4′-bipiperidin]-1-yl)-1-methylcyclobutane-1-carboxylic acid. This compound was prepared according to General Procedure B using (R)-5-chloro-N-(1-(2,4-dichlorophenyl)ethyl)pyrazolo[1,5-a]pyrimidin-7-amine (80 mg, 0.23 mmol), (1R,3r)-3-((R)-[3,4′-bipiperidin]-1-yl)-1-methylcyclobutane-1-carboxylic acid dihydrochloride (84.4 mg, 0.24 mmol) and potassium phosphate tribasic (249 mg, 1.17 mmol) in tert-butanol (3 mL) and water (1 mL) at 80° C. for 18 h. The crude product was purified by reversed phase HPLC (Phenomenex, Gemini-NX, 10 μm, 250×30 mm, C18 column; gradient of 0% MeCN/0.1% TFA-water to 100% MeCN/0.1% TFA) to afford the title compound (134 mg) as the corresponding TFA (trifluoroacetate) salt. ¹H NMR (400 MHz, Methanol-d4, TFA salt) δ7.99 (d, J=2.0 Hz, 1H), 7.60-7.57 (m, 1H), 7.55 (d, J=2.5 Hz, 1H), 7.39 (dd, J=8.5, 2.2 Hz, 1H), 6.24 (d, J=2.1 Hz, 1H), 5.35 (q, J=6.7 Hz, 1H), 5.22 (s, 1H), 4.11-4.01 (m, 2H), 3.81-3.68 (m, 1H), 3.51-3.43 (m, 1H), 3.43-3.36 (m, 1H), 3.25-3.13 (m, 2H), 2.87-2.77 (m, 2H), 2.73-2.62 (m, 1H), 2.62-2.52 (m, 1H), 2.27-2.17 (m, 2H), 2.07.1.99 (m, 1H), 1.97.1.83 (m, 3H), 1.72 (d, J=6.8 Hz, 3H), 1.70.1.61 (m, 4H), 1.42 (s, 3H), 1.33.1.17 (m, 2H). LCMS [M+H]⁺: 585.0.

### Example 25. (1R,3r)-3-((R)-1′-(7-(((R)-1-(2-chloro-4-fluorophenyl)ethyl)amino)pyrazolo[1,5-a]pyrimidin-5-yl)-[3,4′-bipiperidin]-1-yl)-1-methylcyclobutane-1-carboxylic acid

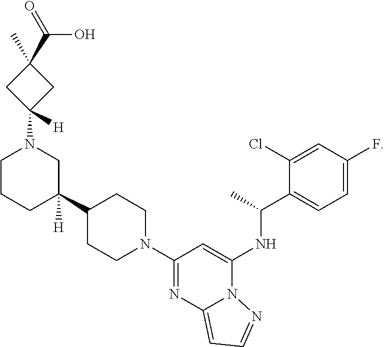 [SMILES: C[C@@H](Nc1cc(N2CCC([C@H]3CCCN([C@H]4C[C@](C)(C(=O)O)C4)C3)CC2)nc2ccnn12)c1ccc(F)cc1Cl]

Step 1. (R)-5-chloro-N-(1-(2-chloro-4-fluorophenyl)ethyl)pyrazolo[1,5-a]pyrimidin-7-amine was prepared according to General Procedure A using 5,7-dichloropyrazolo[1,5-a]pyrimidine (500 mg, 2.66 mmol), (R)-1-(2-chloro-4-fluorophenyl)ethan-1-amine (462 mg, 2.66 mmol) and N,N-diisopropylethylamine (0.926 mL, 5.32 mmol) in MeCN (5.3 mL) at 80° C. for 18 h. The crude product was purified by silica column chromatography (0-50% EtOAc in hexanes) to provide the desired product (857.7 mg). LCMS [M+H]⁺: 324.9

Step 2. (1R,3r)-3-((R)-1′-(7-(((R)-1-(2-chloro-4-fluorophenyl)ethyl)amino)pyrazolo[1,5-a]pyrimidin-5-yl)-[3,4′-bipiperidin]-1-yl)-1-methylcyclobutane-1-carboxylic acid was prepared according to General Procedure B using (R)-5-chloro-N-(1-(2-chloro-4-fluorophenyl)ethyl)pyrazolo[1,5-a]pyrimidin-7-amine (80 mg, 0.25 mmol), (1R,3r)-3-((R)-[3,4′-bipiperidin]-1-yl)-1-methylcyclobutane-1-carboxylic acid dihydrochloride (88.7 mg, 0.25 mmol) and potassium phosphate tribasic (261 mg, 1.23 mmol) in tert-butanol (3 mL) and water (1 mL) at 80° C. for 18 h. The crude product was purified by reversed phase HPLC (Phenomenex, Gemini-NX, 10 μm, 250×30 mm, C18 column; gradient of 0% MeCN/0.1% TFA-water to 100% MeCN/0.1% TFA) to afford the title compound (106.5 mg) as the corresponding TFA salt. ¹H NMR (400 MHz, Methanol-d4, TFA salt) δ7.99 (d, J=2.1 Hz, 1H), 7.65-7.57 (m, 1H), 7.34 (dd, J=8.6, 2.6 Hz, 1H), 7.20-7.11 (m, 1H), 6.24 (d, J=2.1 Hz, 1H), 5.36 (q, J=6.7 Hz, 1H), 5.25 (s, 1H), 4.09-4.02 (m, 2H), 3.81-3.68 (m, 1H), 3.51-3.43 (m, 1H), 3.43-3.35 (m, 1H), 3.27-3.12 (m, 2H), 2.87-2.77 (m, 2H), 2.72-2.63 (m, 1H), 2.62-2.51 (m, 1H), 2.27-2.17 (m, 2H), 2.09.1.98 (m, 1H), 1.97.1.83 (m, 3H), 1.72 (d, J=6.7 Hz, 3H), 1.70.1.61 (m, 4H), 1.42 (s, 3H), 1.35.1.17 (m, 2H). LCMS [M+H]⁺: 569.0.

### Example 26. (1R,3r)-3-((R)-1′-(7-(((R)-1-(2-chloro-4-fluorophenyl)ethyl)amino)-[1,2,4]triazolo[1,5-a]pyrimidin-5-yl)-[3,4′-bipiperidin]-1-yl)-1-methylcyclobutane-1-carboxylic acid

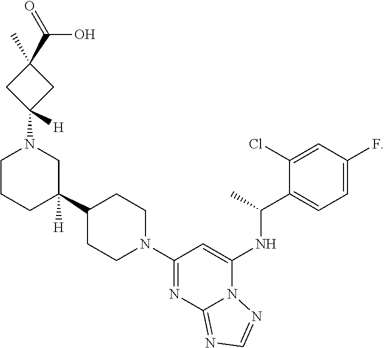 [SMILES: C[C@@H](Nc1cc(N2CCC([C@H]3CCCN([C@H]4C[C@](C)(C(=O)O)C4)C3)CC2)nc2ncnn12)c1ccc(F)cc1Cl]

Step 1. (R)-5-chloro-N-(1-(2-chloro-4-fluorophenyl)ethyl)-[1,2,4]triazolo[1,5-a]pyrimidin-7-amine was prepared according to General Procedure A using 5,7-dichloro-[1,2,4]triazolo[1,5-a]pyrimidine (500 mg, 2.65 mmol), (R)-1-(2-chloro-4-fluorophenyl)ethan-1-amine (459 mg, 2.65 mmol) and N,N-diisopropylethylamine (0.922 mL, 5.29 mmol) in MeCN (5.3 mL) at 80° C. for 8 h. The crude product was purified by silica column chromatography (0-50% EtOAc in hexanes) to provide the desired product (735.6 mg). LCMS [M+H]⁺: 326.0

Step 2. (1R,3r)-3-((R)-1′-(7-(((R)-1-(2-chloro-4-fluorophenyl)ethyl)amino)-[11,2,4]triazolo[1,5-a]pyrimidin-5-yl)-[3,4′-bipiperidin]-1-yl)-1-methylcyclobutane-1-carboxylic acid was prepared according to General Procedure B using (R)-5-chloro-N-(1-(2-chloro-4-fluorophenyl)ethyl)-[1,2,4]triazolo[1,5-a]pyrimidin-7-amine (80 mg, 0.25 mmol), (1R,3r)-3-((R)-[3,4′-bipiperidin]-1-yl)-1-methylcyclobutane-1-carboxylic acid dihydrochloride (88.4 mg, 0.25 mmol) and potassium phosphate tribasic (260 mg, 1.23 mmol) in tert-butanol (3 mL) and water (1 mL) at 80° C. for 8 h. The crude product was purified by reversed phase HPLC (Phenomenex, Gemini-NX, 10 μm, 250×30 mm, C18 column; gradient of 0% MeCN/0.1% TFA-water to 100% MeCN/0.1% TFA) to afford the title compound (80.1 mg) as the corresponding TFA salt. ¹H NMR (400 MHz, Methanol-d4, TFA salt) δ8.69 (s, 1H), 7.58 (dd, J=8.8, 5.9 Hz, 1H), 7.32 (dd, J=8.5, 2.6 Hz, 1H), 7.18-7.09 (m, 1H), 5.44 (s, 1H), 5.25 (q, J=6.8 Hz, 1H), 4.53-4.31 (m, 2H), 3.80-3.67 (m, 1H), 3.50-3.42 (m, 1H), 3.42-3.35 (m, 1H), 3.05-2.90 (m, 2H), 2.86-2.77 (m, 2H), 2.71-2.61 (m, 1H), 2.61-2.51 (m, 1H), 2.27-2.15 (m, 2H), 2.06.1.97 (m, 1H), 1.95.1.87 (m, 1H), 1.86.1.76 (m, 2H), 1.68 (d, J=6.8 Hz, 3H), 1.66.1.57 (m, 2H), 1.42 (s, 3H), 1.38.1.08 (m, 4H). LCMS [M+H]⁺: 570.0.

### Example 27. (1R,3r)-3-((R)-1′-(5-(((R)-1-(4-chloro-2-fluorophenyl)ethyl)amino)imidazo[1,2-a]pyrimidin-7-yl)-[3,4′-bipiperidin]-1-yl)-1-methylcyclobutane-1-carboxylic acid

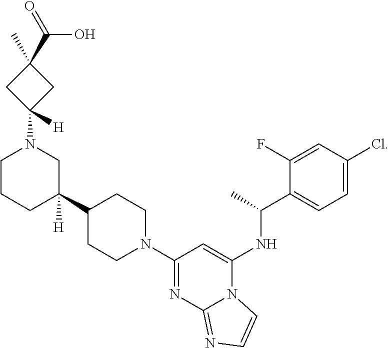 [SMILES: C[C@@H](Nc1cc(N2CCC([C@H]3CCCN([C@H]4C[C@](C)(C(=O)O)C4)C3)CC2)nc2nccn12)c1ccc(Cl)cc1F]

Step 1. (R)-7-chloro-N-(1-(4-chloro-2-fluorophenyl)ethyl)imidazo[1,2-a]pyrimidin-5-amine was prepared according to General Procedure A using 5,7-dichloroimidazo[1,2-a]pyrimidine (100 mg, 0.53 mmol), (R)-1-(4-chloro-2-fluorophenyl)ethan-1-amine hydrochloride (112 mg, 0.53 mmol), and cesium fluoride (121 mg, 0.80 mmol) in DMSO (2 mL) at 100° C. for 18 h. The reaction mixture was extracted with DCM and washed with a saturated aqueous solution of sodium carbonate. The organic layer was dried over anhydrous sodium sulfate, filtered, and concentrated under reduced pressure until a constant weight was attained and afforded the title compound (67 mg). The crude was used as is for the next reaction with further purification. LCMS [M+H]⁺: 325.1.

Step 2. (1R,3r)-3-((R)-1′-(5-(((R)-1-(4-chloro-2-fluorophenyl)ethyl)amino)imidazo[1,2-a]pyrimidin-7-yl)-[3,4′-bipiperidin]-1-yl)-1-methylcyclobutane-1-carboxylic acid was prepared according to General Procedure B using of (R)-7-chloro-N-(1-(4-chloro-2-fluorophenyl)ethyl)imidazo[1,2-a]pyrimidin-5-amine (67 mg, 0.21 mmol), (1R,3r)-3-((R)-[3,4′-bipiperidin]-1-yl)-1-methylcyclobutane-1-carboxylic acid dihydrochloride (73 mg, 0.21 mmol, Example 24, Step 2), and cesium fluoride (156 mg, 1.0 mmol) in DMSO (1.5 mL) at 120° C. for 24 h. The crude product was purified by reversed phase HPLC (Phenomenex, Gemini-NX, 10 μm, 250×30 mm, C18 column; gradient of 0% MeCN/0.1% TFA-water to 100% MeCN/0.1% TFA) to afford the title compound (8 mg) as the corresponding TFA salt. ¹H NMR (400 MHz, Methanol-d4, TFA salt) δ7.92 (d, J=2.8 Hz, 1H), 7.70 (d, J=8.0 Hz, 1H), 7.50-7.45 (m, 1H), 7.31-7.25 (m, 1H), 7.25-7.21 (m, 1H), 5.51 (s, 1H), 5.14 (q, J=6.8 Hz, 1H), 3.78-3.69 (m, 1H), 3.49-3.42 (m, 1H), 3.41-3.35 (m, 2H), 3.00-2.95 (m, 1H), 2.94-2.86 (m, 2H), 2.86-2.77 (m, 2H), 2.76-2.61 (m, 1H), 2.59-2.51 (m, 1H), 2.37-2.33 (m, 1H), 2.27-2.17 (m, 2H), 2.04.1.97 (m, 1H), 1.97.1.87 (m, 1H), 1.81.1.74 (m, 2H), 1.72 (d, J=6.8 Hz, 3H), 1.70.1.55 (m, 2H), 1.41 (s, 3H), 1.33.1.28 (m, 1H), 1.27.1.18 (m, 1H), 1.15.1.02 (m, 1H). LCMS [M+H]⁺: 569.3.

### Example 28. (1R,3r)-3-((R)-1′-(7-(((R)-1-(4-fluoro-2-methylphenyl)ethyl)amino)-[1,2,4]triazolo[1,5-a]pyrimidin-5-yl)-[3,4′-bipiperidin]-1-yl)-1-methylcyclobutane-1-carboxylic acid

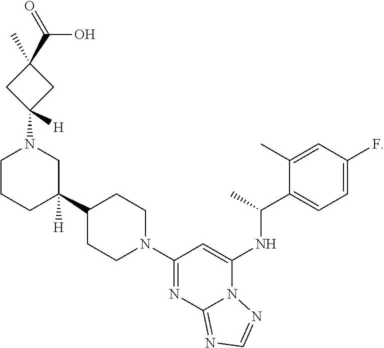 [SMILES: Cc1cc(F)ccc1[C@@H](C)Nc1cc(N2CCC([C@H]3CCCN([C@H]4C[C@](C)(C(=O)O)C4)C3)CC2)nc2ncnn12]

Step 1. (R)-5-chloro-N-(1-(4-fluoro-2-methylphenyl)ethyl)-[1,2,4]triazolo[1,5-a]pyrimidin-7-amine was prepared according to General Procedure A using 5,7-dichloro-[1,2,4]triazolo[1,5-a]pyrimidine (100 mg, 0.53 mmol (R)-1-(4-fluoro-2-methylphenyl)ethan-1-amine hydrochloride (100 mg, 0.53 mmol), and N,N-diisopropylethylamine (0.28 mL, 1.6 mmol) in MeCN (2 mL) at 80° C. for 1 h. The crude was purified directly by normal-phase chromatography using a gradient of EtOAc in hexanes (0 to 100%) as eluent. The fractions containing the desired product were concentrated under reduced pressure until a constant weight was attained and afforded the title compound (156 mg). LCMS [M+H]⁺: 306.1.

Step 2. (1R,3r)-3-((R)-1′-(7-(((R)-1-(4-fluoro-2-methylphenyl)ethyl)amino)-[1,2,4]triazolo[1,5-a]pyrimidin-5-yl)-[3,4′-bipiperidin]-1-yl)-1-methylcyclobutane-1-carboxylic acid was prepared according to General Procedure B using (R)-5-chloro-N-(1-(4-fluoro-2-methylphenyl)ethyl)-[1,2,4]triazolo[1,5-a]pyrimidin-7-amine (50 mg, 0.16 mmol), (1R,3r)-3-((R)-[3,4′-bipiperidin]-1-yl)-1-methylcyclobutane-1-carboxylic acid dihydrochloride (58 mg, 0.16 mmol, Example 24, Step 2), and N,N-diisopropylethylamine (0.14 mL, 0.82 mmol) in MeCN (1.5 mL) at 90° C. for 18 h. The crude product was purified by reversed phase HPLC (Phenomenex, Gemini-NX, 10 μm, 250×30 mm, C18 column; gradient of 0% MeCN/0.1% TFA-water to 100% MeCN/0.1% TFA) to afford the title compound (34 mg) as the corresponding TFA salt. ¹H NMR (400 MHz, DMSO-d6, TFA salt) δ12.64 (bs, 1H), 9.45-9.39 (m, 1H), 8.62 (s, 1H), 8.42 (d, J=7.4 Hz, 1H), 7.49-7.43 (m, 1H), 7.08-7.03 (m, 1H), 7.03-6.97 (m, 1H), 5.44 (s, 1H), 5.05 (q, J=7.0 Hz, 1H), 4.39-4.19 (m, 2H), 3.65 (q, J=8.4 Hz, 1H), 3.37-3.27 (m, 1H), 3.26-3.16 (m, 1H), 2.92-2.78 (m, 2H), 2.69-2.56 (m, 3H), 2.42 (s, 3H), 2.21-2.12 (m, 2H), 1.92.1.84 (m, 1H), 1.79.1.69 (m, 1H), 1.69.1.61 (m, 2H), 1.56 (d, J=6.7 Hz, 3H), 1.53.1.44 (m, 3H), 1.31 (s, 3H), 1.21-0.97 (m, 3H). LCMS [M+H]⁺: 550.3.

### Example 29. (1R,3r)-3-((R)-1′-(7-(((R)-1-(4-fluoro-2-methylphenyl)ethyl)amino) pyrazolo[1,5-a]pyrimidin-5-yl)-[3,4′-bipiperidin]-1-yl)-1-methylcyclobutane-1-carboxylic acid

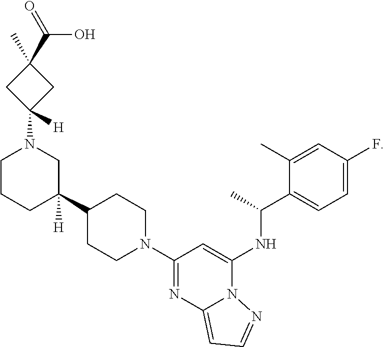 [SMILES: Cc1cc(F)ccc1[C@@H](C)Nc1cc(N2CCC([C@H]3CCCN([C@H]4C[C@](C)(C(=O)O)C4)C3)CC2)nc2ccnn12]

Step 1. (R)-5-chloro-N-(1-(4-fluoro-2-methylphenyl)ethyl)pyrazolo[1,5-a]pyrimidin-7-amine was prepared according to General Procedure A using 5,7-dichloropyrazolo[1,5-a]pyrimidine (100 mg, 0.53 mmol), (R)-1-(4-fluoro-2-methylphenyl)ethan-1-amine hydrochloride (101 mg, 0.53 mmol), and N,N-diisopropylethylamine (0.28 mL, 1.6 mmol) in MeCN (2 mL) at 80° C. for 1 h. The crude was purified directly by normal-phase chromatography using a gradient of EtOAc in hexanes (0 to 100%) as eluent. The fractions containing the desired product were concentrated under reduced pressure until a constant weight was attained and afforded the title compound (129 mg). LCMS [M+H]⁺: 305.1.

Step 2. (1R,3r)-3-((R)-1′-(7-(((R)-1-(4-fluoro-2-methylphenyl)ethyl)amino)pyrazolo[1,5-a]pyrimidin-5-yl)-[3,4′-bipiperidin]-1-yl)-1-methylcyclobutane-1-carboxylic acid was prepared according to General Procedure B using of (R)-5-chloro-N-(1-(4-fluoro-2-methylphenyl)ethyl)pyrazolo[1,5-a]pyrimidin-7-amine (50 mg, 0.16 mmol), (1R,3r)-3-((R)-[3,4′-bipiperidin]-1-yl)-1-methylcyclobutane-1-carboxylic acid dihydrochloride (58 mg, 0.16 mmol, Example 24, Step 2), and cesium fluoride (125 mg, 0.82 mmol) in DMSO (1.5 mL) at 120° C. for 3 d. The crude product was purified by reversed phase HPLC (Phenomenex, Gemini-NX, 10 μm, 250×30 mm, C18 column; gradient of 0% MeCN/0.1% TFA-water to 100% MeCN/0.1% TFA) to afford the title compound (25 mg) as the corresponding TFA salt. ¹H NMR (400 MHz, DMSO-d6, TFA salt) δ12.69 (bs, 1H), 9.42 (m, 1H), 7.94 (s, 1H), 7.50-7.44 (m, 1H), 7.10-7.03 (m, 1H), 7.04-6.96 (m, 1H), 6.07 (s, 1H), 5.27 (s, 1H), 5.14-5.04 (m, 1H), 4.13 (bs, 2H), 3.66 (q, J=8.4 Hz, 1H), 3.35-3.27 (m, 1H), 3.25-3.18 (m, 1H), 3.03-2.80 (m, 2H), 2.68-2.53 (m, 4H), 2.43 (s, 3H), 2.22-2.12 (m, 2H), 1.92.1.84 (m, 1H), 1.79.1.69 (m, 2H), 1.68-1.61 (m, 2H), 1.58 (d, J=6.7 Hz, 3H), 1.55.1.44 (m, 2H), 1.31 (s, 3H), 1.25.1.15 (m, 1H), 1.15.1.01 (m, 2H). LCMS [M+H]⁺: 549.4.

### Example 30. (1R,3r)-3-((R)-1′-(7-(((R)-1-(2-chloro-4-methylphenyl)ethyl)amino)-[1,2,4]triazolo[1,5-a]pyrimidin-5-yl)-[3,4′-bipiperidin]-1-yl)-1-methylcyclobutane-1-carboxylic acid

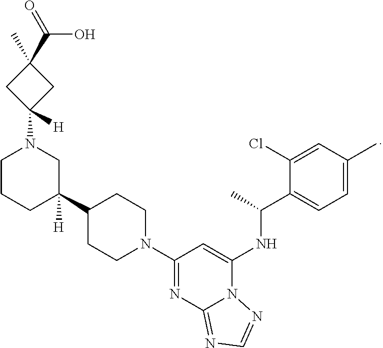 [SMILES: Cc1ccc([C@@H](C)Nc2cc(N3CCC([C@H]4CCCN([C@H]5C[C@](C)(C(=O)O)C5)C4)CC3)nc3ncnn23)c(Cl)c1]

Step 1. (R)-5-chloro-N-(1-(2-chloro-4-methylphenyl)ethyl)-[1,2,4]triazolo[1,5-a]pyrimidin-7-amine was prepared according to General Procedure A using 5,7-dichloro-[1,2,4]triazolo[1,5-a]pyrimidine (100 mg, 0.53 mmol), (R)-1-(2-chloro-4-methylphenyl)ethan-1-amine hydrochloride (10⁹ mg, 0.53 mmol), and N,N-diisopropylethylamine (0.28 mL, 1.6 mmol) in MeCN (2 mL) at 80° C. for 18 h. The crude was purified directly by normal-phase chromatography using a gradient of EtOAc in hexanes (0 to 100%) as eluent. The fractions containing the desired product were concentrated under reduced pressure until a constant weight was attained and afforded the title compound (157 mg). LCMS [M+H]⁺: 322.1.

Step 2. (1R,3r)-3-((R)-1′-(7-(((R)-1-(2-chloro-4-methylphenyl)ethyl)amino)-[11,2,4]triazolo[1,5-a]pyrimidin-5-yl)-[3,4′-bipiperidin]-1-yl)-1-methylcyclobutane-1-carboxylic acid was prepared according to General Procedure B using of (R)-5-chloro-N-(1-(2-chloro-4-methylphenyl)ethyl)-[1,2,4]triazolo[1,5-a]pyrimidin-7-amine (50 mg, 0.16 mmol), (1R,3r)-3-((R)-[3,4′-bipiperidin]-1-yl)-1-methylcyclobutane-1-carboxylic acid dihydrochloride (55 mg, 0.16 mmol, Example 24, Step 2), and N,N-diisopropylethylamine (0.19 mL, 1.1 mmol) in MeCN (1.5 mL) at 90° C. for 18 h. The crude product was purified by reversed phase HPLC (Phenomenex, Gemini-NX, 10 μm, 250×30 mm, C18 column; gradient of 0% MeCN/0.1% TFA-water to 100% MeCN/0.1% TFA) to afford the title compound (16 mg) as the corresponding TFA salt. ¹H NMR (400 MHz, Methanol-d4, TFA salt) δ8.66 (s, 1H), 7.41 (d, J=8.0 Hz, 1H), 7.31-7.26 (m, 1H), 7.19-7.12 (m, 1H), 5.44 (s, 1H), 5.21 (q, J=6.8 Hz, 1H), 4.57-4.28 (m, 2H), 3.80-3.67 (m, 1H), 3.51-3.42 (m, 1H), 3.42-3.34 (m, 1H), 3.04-2.89 (m, 2H), 2.86-2.77 (m, 2H), 2.71-2.61 (m, 1H), 2.61-2.51 (m, 1H), 2.32 (s, 3H), 2.25-2.14 (m, 2H), 2.08.1.97 (m, 1H), 1.96.1.87 (m, 1H), 1.84.1.80 (m, 1H), 1.79.1.76 (m, 1H), 1.74.1.69 (m, 1H), 1.66 (d, J=6.8 Hz, 3H), 1.63.1.56 (m, 2H), 1.42 (s, 3H), 1.38.1.26 (m, 1H), 1.26.1.09 (m, 2H). LCMS [M+H]⁺: 566.3.

### Example 31. (1R,3r)-3-((R)-1′-(7-(((R)-1-(2-chloro-4-methylphenyl)ethyl)amino)pyrazolo[1,5-a]pyrimidin-5-yl)-[3,4′-bipiperidin]-1-yl)-1-methylcyclobutane-1-carboxylic acid

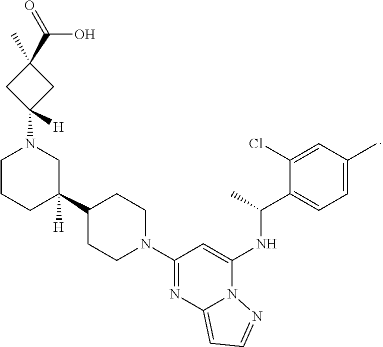 [SMILES: Cc1ccc([C@@H](C)Nc2cc(N3CCC([C@H]4CCCN([C@H]5C[C@](C)(C(=O)O)C5)C4)CC3)nc3ccnn23)c(Cl)c1]

Step 1. (R)-5-chloro-N-(1-(2-chloro-4-methylphenyl)ethyl)pyrazolo[1,5-a]pyrimidin-7-amine was prepared according to General Procedure A using 5,7-dichloropyrazolo[1,5-a]pyrimidine (100 mg, 0.53 mmol), (R)-1-(2-chloro-4-methylphenyl)ethan-1-amine hydrochloride (110 mg, 0.53 mmol), and N,N-diisopropylethylamine (0.28 mL, 1.6 mmol) in MeCN (2 mL) at 80° C. for 1.5 h. The crude was purified directly by normal-phase chromatography using a gradient of EtOAc in hexanes (0 to 100%) as eluent. The fractions containing the desired product were concentrated under reduced pressure until a constant weight was attained and afforded the title compound (169 mg). LCMS [M+H]⁺: 321.1.

Step 2. (1R,3r)-3-((R)-1′-(7-(((R)-1-(2-chloro-4-methylphenyl)ethyl)amino)pyrazolo[1,5-a]pyrimidin-5-yl)-[3,4′-bipiperidin]-1-yl)-1-methylcyclobutane-1-carboxylic acid was prepared according to General Procedure B using (R)-5-chloro-N-(1-(2-chloro-4-methylphenyl)ethyl)pyrazolo[1,5-a]pyrimidin-7-amine (50 mg, 0.16 mmol), (1R,3r)-3-((R)-[3,4′-bipiperidin]-1-yl)-1-methylcyclobutane-1-carboxylic acid dihydrochloride (55 mg, 0.16 mmol, Example 24, Step 2), and cesium fluoride (166 mg, 1.1 mmol) in DMSO (1.5 mL) at 120° C. for 18 h. The crude product was purified by reversed phase HPLC (Phenomenex, Gemini-NX, 10 μm, 250×30 mm, C18 column; gradient of 0% MeCN/0.1% TFA-water to 100% MeCN/0.1% TFA) to afford the title compound (3.5 mg) as the corresponding TFA salt. ¹H NMR (400 MHz, Methanol-d4, TFA salt) δ7.98 (dd, J=2.3, 1.6 Hz, 1H), 7.46-7.41 (m, 1H), 7.33-7.25 (m, 1H), 7.20-7.14 (m, 1H), 6.23 (d, J=2.1 Hz, 1H), 5.32 (q, J=6.7 Hz, 1H), 5.25 (s, 1H), 4.09-4.05 (m, 1H), 4.04-4.01 (m, 1H), 3.78-3.70 (m, 1H), 3.53-3.44 (m, 2H), 3.44-3.36 (m, 1H), 3.24-3.10 (m, 2H), 2.87-2.77 (m, 3H), 2.73-2.62 (m, 1H), 2.62-2.51 (m, 1H), 2.32 (s, 3H), 2.26-2.18 (m, 2H), 2.07.1.98 (m, 1H), 1.98-1.90 (m, 1H), 1.90.1.83 (m, 2H), 1.70 (d, J=6.8 Hz, 3H), 1.68.1.60 (m, 2H), 1.42 (s, 3H), 1.33.1.18 (m, 2H). LCMS [M+H]⁺: 565.4.

### Example 32. (1R,3r)-3-((R)-1′-(7-(((R)-1-(4-chloro-2-methylphenyl)ethyl)amino) pyrazolo[1,5-a]pyrimidin-5-yl)-[3,4′-bipiperidin]-1-yl)-1-methylcyclobutane-1-carboxylic acid

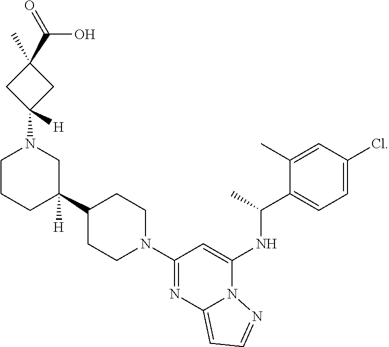 [SMILES: Cc1cc(Cl)ccc1[C@@H](C)Nc1cc(N2CCC([C@H]3CCCN([C@H]4C[C@](C)(C(=O)O)C4)C3)CC2)nc2ccnn12]

Step 1. (R)-5-chloro-N-(1-(4-chloro-2-methylphenyl)ethyl)pyrazolo[1,5-a]pyrimidin-7-amine was prepared according to General Procedure A using 5,7-dichloropyrazolo[1,5-a]pyrimidine (200 mg, 1.06 mmol), (R)-1-(4-chloro-2-methylphenyl)ethan-1-amine; hydrochloride (219 mg, 1.06 mmol), and N,N-diisopropylethylamine (0.37 mL, 2.13 mmol) in MeCN (7.1 mL) at room temperature for 3 h. The reaction mixture was diluted with water (150 mL) and saturated aqueous sodium carbonate (50 mL), then extracted with EtOAc (3×100 mL). The organic layer was washed with brine, dried over magnesium sulfate, filtered, and concentrated under reduced pressure until a constant weight was attained and afforded the title compound (323 mg). LCMS [M+H]⁺: 321.1.

Step 2. (1R,3r)-3-((R)-1′-(7-(((R)-1-(4-chloro-2-methylphenyl)ethyl)amino)pyrazolo[1,5-a]pyrimidin-5-yl)-[3,4′-bipiperidin]-1-yl)-1-methylcyclobutane-1-carboxylic acid was prepared according to General Procedure B using (R)-5-chloro-N-(1-(4-chloro-2-methylphenyl)ethyl)pyrazolo[1,5-a]pyrimidin-7-amine (100 mg, 0.31 mmol) and methyl (1R,3r)-3-((R)-[3,4′-bipiperidin]-1-yl)-1-methylcyclobutane-1-carboxylate dihydrochloride (110 mg, 0.31 mmol, Example 18, Step 1) in tert-butanol (1.97 mL), water (0.66 mL), and NaOH (0.93 mL, 3N aqueous solution) at 80° C. for 18 h. The crude product was purified by reversed phase HPLC (Phenomenex, Gemini-NX, 10 μm, 250×30 mm, C18 column; gradient of 0% MeCN/0.1% TFA-water to 100% MeCN/0.1% TFA) to afford the title compound (80 mg) as the corresponding TFA salt. ¹H NMR (400 MHz, Methanol-d4, TFA salt) δ7.99 (d, J=2.1 Hz, 1H), 7.38 (d, J=8.4 Hz, 1H), 7.29 (d, J=2.4 Hz, 1H), 7.22 (dd, J=8.3, 2.4 Hz, 1H), 6.24 (d, J=2.1 Hz, 1H), 5.16 (q, J=8.0 Hz, 1H), 5.10 (s, 1H), 4.06-3.99 (m, 1H), 3.99-3.91 (m, 1H), 3.80-3.69 (m, 1H), 3.51-3.44 (m, 1H), 3.43-3.36 (m, 1H), 3.23-3.10 (m, 2H), 2.88-2.77 (m, 3H), 2.72-2.61 (m, 1H), 2.61-2.50 (m, 1H), 2.48 (s, 3H), 2.27-2.18 (m, 3H), 2.08.1.98 (m, 1H), 1.97.1.88 (m, 1H), 1.87.1.79 (m, 2H), 1.70 (d, J=6.8 Hz, 3H), 1.68.1.61 (m, 2H), 1.41 (s, 3H), 1.28.1.15 (m, 2H). LCMS [M+H]⁺: 565.0.

### Example 33. (1R,3r)-3-((R)-1′-(7-(((R)-1-(4-chloro-2-methylphenyl)ethyl)amino)-[1,2,4]triazolo[1,5-a]pyrimidin-5-yl)-[3,4′-bipiperidin]-1-yl)-1-methylcyclobutane-1-carboxylic acid

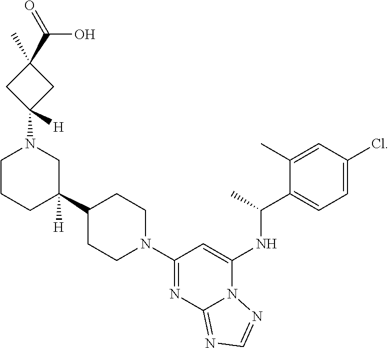 [SMILES: Cc1cc(Cl)ccc1[C@@H](C)Nc1cc(N2CCC([C@H]3CCCN([C@H]4C[C@](C)(C(=O)O)C4)C3)CC2)nc2ncnn12]

Step 1. (R)-5-chloro-N-(1-(4-chloro-2-methylphenyl)ethyl)-[1,2,4]triazolo[1,5-a]pyrimidin-7-amine was prepared according to General Procedure A using 5,7-dichloro-[1,2,4]triazolo[1,5-a]pyrimidine (200 mg, 1.06 mmol), (R)-1-(4-chloro-2-methylphenyl)ethan-1-amine; hydrochloride (219 mg, 1.06 mmol) and N,N-diisopropylethylamine in MeCN (7.1 mL) at room temperature for 3 h. The reaction mixture was diluted with water (150 mL) and saturated aqueous sodium carbonate (50 mL), then extracted with EtOAc (3×100 mL). The organic layer was combined, washed with brine, dried over magnesium sulfate, filtered, and concentrated under reduced pressure until a constant weight was attained and afforded the title compound (301 mg). LCMS [M+H]⁺: 322.1.

Step 2. (1R,3r)-3-((R)-1′-(7-(((R)-1-(4-chloro-2-methylphenyl)ethyl)amino)-[1,2,4]triazolo[1,5-a]pyrimidin-5-yl)-[3,4′-bipiperidin]-1-yl)-1-methylcyclobutane-1-carboxylic acid was prepared according to General Procedure B using ((R)-5-chloro-N-(1-(4-chloro-2-methylphenyl)ethyl)-[1,2,4]triazolo[1,5-a]pyrimidin-7-amine (100 mg, 0.31 mmol), methyl (1R,3r)-3-((R)-[3,4′-bipiperidin]-1-yl)-1-methylcyclobutane-1-carboxylate dihydrochloride (110 mg, 0.31 mmol, Example 18, Step 1) in tert-butanol (1.97 mL), water (0.66 mL), and NaOH (0.93 mL, 3N aqueous solution) at 80° C. for 18 h. The crude product was purified by reversed phase HPLC (Phenomenex, Gemini-NX, 10 μm, 250×30 mm, C18 column; gradient of 0% MeCN/0.1% TFA-water to 100% MeCN/0.1% TFA) to afford the title compound (137 mg) as the corresponding TFA salt. ¹H NMR (400 MHz, Methanol-d4, TFA salt) 8.69 (s, 1H), 7.37 (d, J=8.3 Hz, 1H), 7.27 (d, J=2.2 Hz, 1H), 7.19 (dd, J=8.3, 2.4 Hz, 1H), 5.31 (s, 1H), 5.05 (q, J=6.8 Hz, 1H), 4.55-4.17 (m, 2H), 3.77-3.66 (m, 1H), 3.49-3.42 (m, 1H), 3.41-3.35 (m, 1H), 3.03-2.87 (m, 2H), 2.86-2.75 (m, 2H), 2.69-2.60 (m, 1H), 2.58-2.49 (m, 1H), 2.47 (s, 3H), 2.32-2.20 (m, 3H), 2.04.1.95 (m, 1H), 1.93.1.85 (m, 1H), 1.80.1.71 (m, 3H), 1.66 (d, J=6.7 Hz, 3H), 1.64.1.55 (m, 1H), 1.40 (s, 3H), 1.33.1.13 (m, 2H), 1.13-0.97 (m, 1H). LCMS [M+H]⁺: 566.2.

### Example 34. (1R,3r)-3-((R)-1′-(7-(((R)-1-(2,4-difluorophenyl)ethyl)amino)pyrazolo[1,5-a]pyrimidin-5-yl)-[3,4′-bipiperidin]-1-yl)-1-methylcyclobutane-1-carboxylic acid

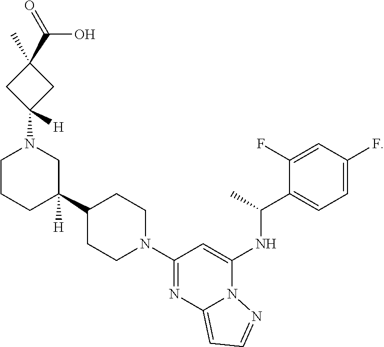 [SMILES: C[C@@H](Nc1cc(N2CCC([C@H]3CCCN([C@H]4C[C@](C)(C(=O)O)C4)C3)CC2)nc2ccnn12)c1ccc(F)cc1F]

Step 1. (R)-5-chloro-N-(1-(2,4-difluorophenyl)ethyl)pyrazolo[1,5-a]pyrimidin-7-amine was prepared according to General Procedure A using 5,7-dichloropyrazolo[1,5-a]pyrimidine (81.2 mg, 0.43 mmol), (R)-1-(2,4-difluorophenyl)ethan-1-amine (70.8 mg, 0.45 mmol), and N,N-diisopropylethylamine (0.22 mL, 1.26 mmol) in MeCN (2 mL) at 80° C. for 3 h. The reaction mixture was diluted with water (20 mL) and saturated aqueous sodium carbonate (20 mL), then extracted with EtOAc (3×25 mL). The organic layer was washed with brine, dried over sodium sulfate, filtered, and concentrated under reduced pressure until a constant weight was attained and afforded the title compound (136 mg). LCMS [M+H]⁺: 309.0.

Step 2. (1R,3r)-3-((R)-1′-(7-(((R)-1-(2,4-difluorophenyl)ethyl)amino)pyrazolo[1,5-a]pyrimidin-5-yl)-[3,4′-bipiperidin]-1-yl)-1-methylcyclobutane-1-carboxylic acid was prepared according to General Procedure B using (R)-5-chloro-N-(1-(2,4-difluorophenyl)ethyl)pyrazolo[1,5-a]pyrimidin-7-amine (67.0 mg, 0.21 mmol) and methyl (1R,3r)-3-((R)-[3,4′-bipiperidin]-1-yl)-1-methylcyclobutane-1-carboxylate dihydrochloride (77.7 mg, 0.22 mmol, Example 18, Step 1) in tert-butanol (1.50 mL), water (0.50 mL), and K₃PO₄ (319 mg, 1.50 mmol) at 80° C. for 16 h. The crude product was purified by reversed phase HPLC (Phenomenex, Gemini-NX, 10 μm, 250×30 mm, C18 column; gradient of 5% MeCN/0.1% TFA-water to 60% MeCN/0.1% TFA) to afford the title compound (23 mg) as the corresponding TFA salt. ¹H NMR (400 MHz, Methanol-d4, TFA salt) δ7.96 (d, J=1.8 Hz, 1H), 7.56 (m, 1H), 7.09-6.97 (m, 2H), 6.23 (d, J=2.1 Hz, 1H), 5.45 (s, 1H), 5.30 (q, J=6.8 Hz, 1H), 4.15-4.05 (m, 2H), 3.78-3.68 (m, 1H), 3.51-3.43 (m, 1H), 3.42-3.37 (m, 1H), 3.25-3.12 (m, 2H), 2.88-2.76 (m, 3H), 2.72-2.62 (m, 1H), 2.61-2.51 (m, 1H), 2.29-2.18 (m, 3H), 2.07.1.97 (m, 1H), 1.96.1.83 (m, 3H), 1.74 (d, J=6.8 Hz, 3H), 1.72.1.61 (m, 2H), 1.41 (s, 3H), 1.39-1.19 (m, 2H). LCMS [M+H]⁺: 553.3.

### Example 35. (1R,3r)-3-((R)-1′-(7-(((R)-1-(2,4-difluorophenyl)ethyl)amino)-[1,2,4]triazolo[1,5-a]pyrimidin-5-yl)-[3,4′-bipiperidin]-1-yl)-1-methylcyclobutane-1-carboxylic acid

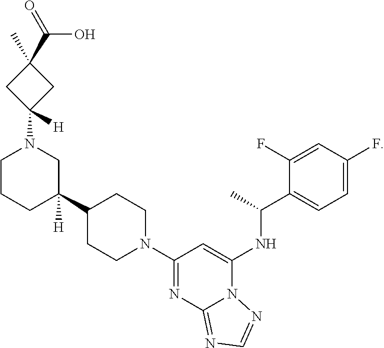 [SMILES: C[C@@H](Nc1cc(N2CCC([C@H]3CCCN([C@H]4C[C@](C)(C(=O)O)C4)C3)CC2)nc2ncnn12)c1ccc(F)cc1F]

Step 1. (R)-5-chloro-N-(1-(2,4-difluorophenyl)ethyl)-[1,2,4]triazolo[1,5-a]pyrimidin-7-amine was prepared according to General Procedure A using 5,7-dichloro-[1,2,4]triazolo[1,5-a]pyrimidine (79.0 mg, 0.42 mmol), (R)-1-(2,4-difluorophenyl)ethan-1-amine (70.7 mg, 0.45 mmol) and N,N-diisopropylethylamine (0.22 mL, 1.26 mmol) in MeCN (2 mL) at 80° C. for 4 h. The reaction mixture was diluted with water (20 mL) and saturated aqueous sodium carbonate (20 mL), then extracted with EtOAc (3×25 mL). The organic layer was combined, washed with brine, dried over sodium sulfate, filtered, and concentrated under reduced pressure until a constant weight was attained and afforded the title compound (126 mg). LCMS [M+H]⁺: 310.0.

Step 2. (1R,3r)-3-((R)-1′-(7-(((R)-1-(2,4-difluorophenyl)ethyl)amino)-[11,2,4]triazolo[1,5-a]pyrimidin-5-yl)-[3,4′-bipiperidin]-1-yl)-1-methylcyclobutane-1-carboxylic acid was prepared according to General Procedure B using (R)-5-chloro-N-(1-(2,4-difluorophenyl)ethyl)-[1,2,4]triazolo[1,5-a]pyrimidin-7-amine (70.0 mg, 0.23 mmol), methyl (1R,3r)-3-((R)-[3,4′-bipiperidin]-1-yl)-1-methylcyclobutane-1-carboxylate dihydrochloride (77.7 mg, 0.23 mmol, Example 18, Step 1) in tert-butanol (1.50 mL), water (0.50 mL), and K₃PO₄ (300 mg, 1.41 mmol) at 80° C. for 16 h. The crude product was purified by reversed phase HPLC (Phenomenex, Gemini-NX, 10 μm, 250×30 mm, C18 column; gradient of 5% MeCN/0.1% TFA-water to 60% MeCN/0.1% TFA) to afford the title compound (126 mg) as the corresponding TFA salt. ¹H NMR (400 MHz, Methanol-d4, TFA salt) δ8.69 (s, 1H), 7.59-7.49 (m, 1H), 7.08-6.95 (m, 2H), 5.64 (s, 1H), 5.18 (q, J=6.8 Hz, 1H), 4.65-4.29 (m, 2H), 3.80-3.66 (m, 1H), 3.50-3.42 (m, 1H), 3.41-3.34 (m, 1H), 3.06-2.90 (m, 3H), 2.88-2.76 (m, 2H), 2.72-2.60 (m, 1H), 2.59-2.50 (m, 1H), 2.30-2.17 (m, 3H), 2.07.1.96 (m, 1H), 1.95.1.88 (m, 1H), 1.85.1.76 (m, 2H), 1.71 (d, J=6.8 Hz, 3H), 1.68.1.58 (m, 1H), 1.40 (s, 3H), 1.39-1.11 (m, 3H). LCMS [M+H]⁺: 554.3.

### Example 36. (1R,3r)-3-((R)-1′-(5-(((R)-1-(2,4-dichlorophenyl)ethyl)amino) imidazo[1,2-a]pyrimidin-7-yl)-[3,4′-bipiperidin]-1-yl)-1-methylcyclobutane-1-carboxylic acid

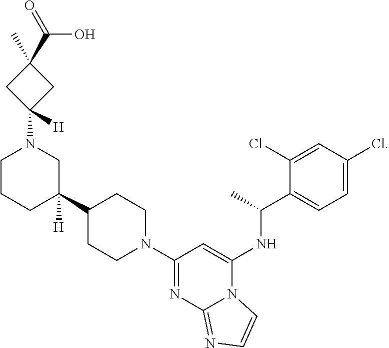 [SMILES: C[C@@H](Nc1cc(N2CCC([C@H]3CCCN([C@H]4C[C@](C)(C(=O)O)C4)C3)CC2)nc2nccn12)c1ccc(Cl)cc1Cl]

Step 1. (R)-7-chloro-N-(1-(2,4-dichlorophenyl)ethyl)imidazo[1,2-a]pyrimidin-5-amine was prepared according to General Procedure A using 5,7-dichloroimidazo[1,2-a]pyrimidine (57.4 mg, 0.30 mmol), (R)-1-(2,4-dichlorophenyl)ethan-1-amine (62.0 mg, 0.33 mmol) and N,N-diisopropylethylamine (0.16 mL, 0.92 mmol) in MeCN (1.5 mL) at 80° C. for 18 h. The reaction mixture was cooled to 0° C., solid was filtered, rinsed with cold MeCN, air and vacuum dried to afford the title compound (67 mg). LCMS [M+H]⁺: 341.1.

Step 2. (1R,3r)-3-((R)-1′-(5-(((R)-1-(2,4-dichlorophenyl)ethyl)amino)imidazo[1,2-a]pyrimidin-7-yl)-[3,4′-bipiperidin]-1-yl)-1-methylcyclobutane-1-carboxylic acid was prepared according to General Procedure B using (R)-7-chloro-N-(1-(2,4-dichlorophenyl)ethyl)imidazo[1,2-a]pyrimidin-5-amine (62.0 mg, 0.18 mmol), methyl (1R,3r)-3-((R)-[3,4′-bipiperidin]-1-yl)-1-methylcyclobutane-1-carboxylate dihydrochloride (66.1 mg, 0.19 mmol, Example 18, Step 1) in DMSO (1.6 mL), water (0.40 mL), and K₃PO₄ (257 mg, 1.21 mmol) at 100° C. for 20 h. The crude product was purified by reversed phase HPLC (Phenomenex, Gemini-NX, 10 μm, 250×30 mm, C18 column; gradient of 5% MeCN/0.1% TFA-water to 60% MeCN/0.1% TFA) to afford the title compound (17.8 mg) as the corresponding TFA salt. ¹H NMR (400 MHz, Methanol-d4, TFA salt) δ7.92 (d, J=2.8 Hz, 1H), 7.57 (d, J=2.1 Hz, 1H), 7.52-7.46 (m, 2H), 7.37 (dd, J=8.4, 2.1 Hz, 1H), 5.32 (s, 1H), 5.26-5.16 (m, 1H), 4.60-4.13 (m, 2H), 3.79-3.67 (m, 1H), 3.51-3.42 (m, 1H), 3.42-3.34 (m, 1H), 3.03-2.86 (m, 3H), 2.85-3.76 (m, 2H), 2.71-2.60 (m, 1H), 2.59-2.50 (m, 1H), 2.25-2.15 (m, 2H), 2.06.1.96 (m, 1H), 1.93.1.85 (m, 1H), 1.80.1.72 (m, 2H), 1.68 (d, J=6.8 Hz, 3H), 1.66.1.55 (m, 2H), 1.41 (s, 3H), 1.36.1.14 (m, 2H), 1.12.1.00 (m, 1H). LCMS [M+H]⁺: 585.2.

### Example 37. (1R,3r)-3-((R)-1′-(7-(((R)-1-(4-chloro-2-fluorophenyl)ethyl)amino)-[1,2,4]triazolo[1,5-a]pyrimidin-5-yl)-[3,4′-bipiperidin]-1-yl)-1-methylcyclobutane-1-carboxylic acid

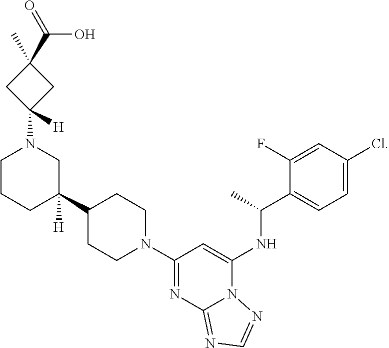 [SMILES: C[C@@H](Nc1cc(N2CCC([C@H]3CCCN([C@H]4C[C@](C)(C(=O)O)C4)C3)CC2)nc2ncnn12)c1ccc(Cl)cc1F]

Step 1. (R)-5-chloro-N-(1-(4-chloro-2-fluorophenyl)ethyl)-[1,2,4]triazolo[1,5-a]pyrimidin-7-amine was prepared according to General Procedure A using 5,7-dichloro[1,2,4]triazolo[1,5-a]pyrimidine (60 mg, 0.32 mmol), (R)-1-(4-chloro-2-fluorophenyl)ethan-1-amine hydrochloride (73 mg, 0.35 mmol), and N,N-diisopropylethylamine (0.17 mL, 0.95 mmol) in MeCN (1.4 mL) at 80° C. for 18 h. The crude product was purified by silica column chromatography (0-35% EtOAc in hexanes) to provide the desired product (100 mg). LCMS [M+H]⁺: 326.1.

Step 2. (1R,3r)-3-((R)-1′-(7-(((R)-1-(4-chloro-2-fluorophenyl)ethyl)amino)-[1,2,4]triazolo[1,5-a]pyrimidin-5-yl)-[3,4′-bipiperidin]-1-yl)-1-methylcyclobutane-1-carboxylic acid was prepared according to General Procedure B using (R)-5-chloro-N-(1-(4-chloro-2-fluorophenyl)ethyl)-[1,2,4]triazolo[1,5-a]pyrimidin-7-amine (100 mg, 0.31 mmol) and (1R,3r)-3-((R)-[3,4′-bipiperidin]-1-yl)-1-methylcyclobutane-1-carboxylic acid dihydrochloride (108 mg, 0.31 mmol, Example 24, Step 2), and K₃PO₄ (325 mg, 1.53 mmol) in tert-butanol (1.2 mL) and water (0.4 mL) at 80° C. for 18 h. The crude product was purified by reversed phase HPLC (Phenomenex, Gemini-NX, 10 μm, 250×30 mm, C18 column; gradient of 0% MeCN/0.1% TFA-water to 100% MeCN/0.1% TFA) to afford the title compound (50 mg) as the corresponding TFA salt. ¹H NMR (400 MHz, Methanol-d4, TFA salt) δ8.68 (s, 1H), 7.50 (t, J=8.3 Hz, 1H), 7.30-7.19 (m, 2H), 5.61 (s, 1H), 5.18 (q, J=6.9 Hz, 1H), 4.73-4.29 (m, 2H), 3.78-3.66 (m, 1H), 3.42 (m, 2H), 2.97 (m, 2H), 2.84-2.73 (m, 2H), 2.73-2.43 (m, 2H), 2.27 (m, 2H), 2.07.1.84 (m, 2H), 1.83 (s, 3H), 1.71 (m, 3H), 1.67.1.56 (m, 2H), 1.40 (d, J=4.7 Hz, 3H), 1.36.1.03 (m, 3H). LCMS [M+H]⁺: 570.0.

### Example 38. (1R,3r)-3-((R)-1′-(7-(((R)-1-(4-chloro-2-fluorophenyl)ethyl) amino)pyrazolo[1,5-a]pyrimidin-5-yl)-[3,4′-bipiperidin]-1-yl)-1-methylcyclobutane-1-carboxylic acid

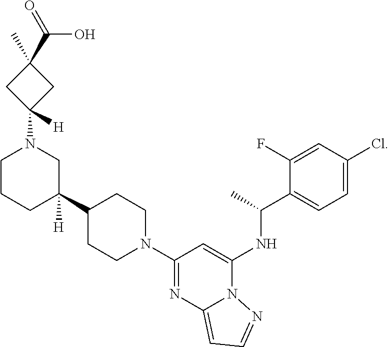 [SMILES: C[C@@H](Nc1cc(N2CCC([C@H]3CCCN([C@H]4C[C@](C)(C(=O)O)C4)C3)CC2)nc2ccnn12)c1ccc(Cl)cc1F]

Step 1. (R)-5-chloro-N-(1-(4-chloro-2-fluorophenyl)ethyl)pyrazolo[1,5-a]pyrimidin-7-amine was prepared according to General Procedure A using 5,7-dichloropyrazolo[1,5-a]pyrimidine (50 mg, 0.27 mmol), (R)-1-(4-chloro-2-fluorophenyl)ethan-1-amine hydrochloride (62 mg, 0.29 mmol) and N,N-diisopropylethylamine (0.14 mL, 0.79 mmol) in MeCN (1.3 mL) at 80° C. for 18 h. The crude product was purified by silica column chromatography (0-35% EtOAc in hexanes) to provide the desired product (60 mg). LCMS [M+H]⁺: 325.1.

Step 2. (1R,3r)-3-((R)-1′-(7-(((R)-1-(4-chloro-2-fluorophenyl)ethyl)amino)pyrazolo[1,5-a]pyrimidin-5-yl)-[3,4′-bipiperidin]-1-yl)-1-methylcyclobutane-1-carboxylic acid was prepared according to General Procedure B using (R)-5-chloro-N-(1-(4-chloro-2-fluorophenyl)ethyl)pyrazolo[1,5-a]pyrimidin-7-amine (60 mg, 0.19 mmol), (1R,3r)-3-((R)-[3,4′-bipiperidin]-1-yl)-1-methylcyclobutane-1-carboxylic acid dihydrochloride (65 mg, 0.19 mmol, Example 24, Step 2), and K₃PO₄ (196 mg, 0.92 mmol) in tert-butanol (1.2 mL) and water (0.4 mL) at 80° C. for 18 h. The crude product was purified by reversed phase HPLC (Phenomenex, Gemini-NX, 10 μm, 250×30 mm, C18 column; gradient of 0% MeCN/0.1% TFA-water to 100% MeCN/0.1% TFA) to afford the title compound (75 mg) as the corresponding TFA salt. ¹H NMR (400 MHz, Methanol-d4, TFA salt) δ7.96 (d, J=2.0 Hz, 1H), 7.52 (t, J=8.2 Hz, 1H), 7.33-7.19 (m, 2H), 6.24 (d, J=2.0 Hz, 1H), 5.43 (s, 1H), 5.30 (d, J=6.8 Hz, 1H), 4.15-4.03 (m, 2H), 3.79-3.66 (m, 1H), 3.50-3.36 (m, 2H), 3.26-3.13 (m, 2H), 2.85-2.75 (m, 2H), 2.72-2.61 (m, 1H), 2.61-2.50 (m, 1H), 2.36-2.22 (m, 2H), 2.06-1.97 (m, 1H), 1.97.1.82 (m, 4H), 1.75 (d, J=6.9 Hz, 3H), 1.73.1.61 (m, 3H), 1.40 (s, 3H), 1.36.1.17 (m, 2H). LCMS [M+H]⁺: 569.2.

### Example 39. Determination of CCR4 antagonist activity in Beta-Arrestin Cellular Assay

CCR4 Antagonistic Activity was assessed in Beta-Arrestin Cellular Assay. Compounds were assessed in CHO-K1 cells expressing the CCR4 receptor modified to enable β-glactosidase detection of β-arrestin agonist activity stimulated by CCL22 in a chemiluminescence assay. Cells were seeded in a total volume of 20 μL into white walled, 384-well microplates and incubated at 37° C. prior to testing. Cells were pre-incubated with test compound for 30 min at 37° C. followed by agonist challenge with CCL22 at the EC₈₀ concentration (15 nM) for 90 minutes. Assay signal was generated through a single addition of 12.5 or 15 μL (5000 v/v) of PathHunter Detection reagent cocktail, followed by a one-hour incubation at room temperature. Microplates were read following signal generation with a PerkinElmer Envision™ instrument for chemiluminescent signal detection. Antagonist activity was analyzed using CBIS data analysis suite (ChemInnovation, CA). Antagonist activity was characterized as percentage inhibition calculated using the following formula:

$$\% Inhibition = 100 \% \times ( 1 - ( mean RLU of test sample - mean RLU of vehicle control ) / \\ ( mean RLU of {EC}_{80} control - mean RLU of vehicle control ) ) .$$

Using a concentration response curve, the potency of the compounds of Examples 1 through 38, as measured by their CCR4 antagonist IC₅₀ values in the β-arrestin assay, was determined and the results are shown in Table 2 below. An IC₅₀ of >500 nM is designated as Potency C, an IC₅₀ of between 500 nM and 100 nM is designated as Potency B and an IC₅₀ of <100 nM is designated as Potency A.

TABLE 2

| Example | Potency |
|---|---|
| 1 | A |
| 2 | A |
| 3 | A |
| 4 | A |
| 5 | A |
| 6 | A |
| 7 | C |
| 8 | A |
| 9 | A |
| 10 | A |
| 11 | A |
| 12 | A |
| 13 | A |
| 14 | A |
| 15 | A |
| 16 | A |
| 17 | A |
| 18 | B |
| 19 | A |
| 20 | A |
| 21 | A |
| 22 | A |
| 23a | A |
| 23b | A |
| 24 | A |
| 25 | A |
| 26 | A |
| 27 | A |
| 28 | B |
| 29 | A |
| 30 | A |
| 31 | A |
| 32 | A |
| 33 | B |
| 34 | A |
| 35 | B |
| 36 | A |
| 37 | A |
| 38 | A |

1. A compound having structural Formula (I): 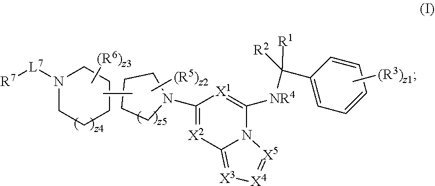
or a pharmaceutically acceptable salt thereof, wherein:
X¹ is CR⁸ or N;
X² is CR⁹ or N;
X³ is CR¹⁰ or N;
X⁴ is CR¹¹ or N;
X⁵ is CR¹² or N;
at least one of X¹, X², X³, X⁴, and X⁵ is N;
z1 is an integer from 0 to 5;
z2 is an integer from 0 to 4;
z3 is an integer from 0 to 11;
z4 is an integer from 0 to 2;
z5 is 1 or 2;
L⁷ is a bond, —O—, —S—, —NR^{7.2B}—, —C(O)—, —C(O)O—, —S(O)—, —S(O)₂—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹ is hydrogen, halogen, —CX^{11.3}, —CHX¹², —CH₂X¹¹, —CN, —N₃, —SOₙ₁R^{1A}, —SOᵥ₁NR^{1B}R^{1C}, —NHNR^{1B}R^{1C}, —ONR^{1B}R^{1C}, —NHC(O)NHNR^{1B}R^{1C}, —NHC(O)NR^{1B}R^{1C}, —N(O)ₘ₁, —NR^{1B}R^{1C}, —C(O)R^{1D}, —C(O)OR^{1D}, —C(O)NR^{1B}R^{1C}, —OR^{1A}, —NR^{1B}SO₂R^{1A}, —NR^{1B}C(O)R^{1D}, —NR^{1B}C(O)OR^{1D}, —NR^{1B}OR^{1D}, —OCX^{1.1}₃, —OCHX^{1.1}₂, —OCH₂X^{1.1}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is hydrogen, halogen, —CX^{2.1}₃, —CHX^{2.1}₂, —CH₂X^{2.1}, —CN, —N₃, —SOₙ₂R^{2A}, —SOᵥ₂NR^{2B}R^{2C}, —NHNR^{2B}R^{2C}, —ONR^{2B}R^{2C}, —NHC(O)NHNR^{2B}R^{2C}, —NHC(O)NR^{2B}R^{2C}, —N(O)ₘ₂, —NR^{2B}R^{2C}, —C(O)R^{2D}, —C(O)OR^{2D}, —C(O)NR^{2B}R^{2C}, OR^{2A}, —N^{2B}SO₂R^{2A}, —NR^{2B}C(O)R^{2D}, —NR^{2B}C(O)OR^{2D}, —NR^{2B}OR^{2D}, —OCX^{2.1}₃, —OCHX^{2.1}₂, —OCH₂X^{2.1}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is independently halogen, —CX^{3.1}₃, —CHX^{3.1}₂, —CH₂X^{3.1}, —CN, —N₃, —SOₙ₃R^{3A}, —SOᵥ₃NR^{3B}R^{3C}, —NHNR^{3B}R^{3C}, —ONR^{3B}R^{3C}, —NHC(O)NHNR^{3B}R^{3C}, —NHC(O)NR^{3B}R^{3C}, —N(O)ₘ₃, —NR^{3B}R^{3C}, —C(O)R^{3D}, —C(O)OR^{3D}, —C(O)NR^{3B}R^{3C}, —OR^{3A}, —NR^{3B}SO₂R^{3A}, —NR^{3B}C(O)R^{3D}, NR^{3B}C(O)OR^{3D}, —NR^{3B}OR^{3D}, —OCX^{3.1}₃, —OCHX^{3.1}₂, —OCH₂X^{3.1}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ is hydrogen, —CX^{4.1}₃, —CHX^{4.1}₂, —CH₂X^{4.1}, —SOₙ₄R^{4A}, —SOᵥ₄NR^{4B}R^{4C}, —C(O)R^{4D}, —C(O)OR^{4D}, —C(O)NR^{4B}R^{4C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁵ is independently halogen, oxo, —CX^{5.1}₃, —CHX^{5.1}₂, —CH₂X⁵¹, —CN, —N₃, —SOₙ₅R^{5A}, —SOᵥ₅NR^{5B}R^{5C}, —NHNR^{5B}R^{5C}, —ONR^{5B}R^{5C}, —NHC(O)NHNR^{5B}R^{5C}, —NHC(O)NR^{5B}R^{5C}, —N(O)ₘ₅, —NR^{5B}R^{5C}, —C(O)R^{5D}, —C(O)OR^{5D}, —C(O)NR^{5B}R^{5C}, —OR^{5A}, —NR^{5B}SO₂R^{5A}, —NR^{5B}C(O)R^{5D}, —NR^{5B}C(O)OR^{5D}, —NR^{5B}OR^{5D}, —OCX^{5.1} ₃, —OCHX^{5.1}₂, —OCH₂X^{5.1}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁶ is independently halogen, oxo, —CX^{6.1}₃, —CHX^{6.1}₂, —CH₂X^{6.1}, —CN, —N₃, —SOₙ₆R^{6A}, —SOᵥ₆NR^{6B}R^{6C}, —NHNR^{6B}R^{6C}, —ONR^{6B}R^{6C}, —NHC(O)NHNR^{6B}R^{6C}, —NHC(O)NR^{6B}R^{6C}, —N(O)ₘ₆, —NR^{6B}R^{6C}, —C(O)R^{6D}, —C(O)OR^{6D}, —C(O)NR^{6B}R^{6C}, —OR^{6A}, —NR^{6B}SO₂R^{6A}, —NR^{6B}C(O)R^{6D}, —NR^{6B}C(O)OR^{6D}, —NR^{6B}OR^{6D}, —OCX^{6.1}₃, —OCHX^{6.1}₂, —OCH₂X^{6.1}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁷ is hydrogen, halogen, —CX^{7.1}₃, —CHX^{7.1}₂, —CH₂X^{7.1}, —CN, —N₃, —SOₙ₇R^{7A}, —SOᵥ₇NR^{7B}R^{7C}, —NHNR^{7B}R^{7C}, —ONR^{7B}R^{7C}, —NHC(O)NHNR^{7B}R^{7C}, —NHC(O)NR^{7B}R^{7C}, —N(O)ₘ₇, —NR^{7B}R^{7C}, —C(O)R^{7D}, —C(O)OR^{7D}, —C(O)NR^{7B}R^{7C}, —OR^{7A}, —N^{7B}SO₂R^{7A}, —NR^{7B}C(O)R^{7D}, —NR^{7B}C(O)OR^{7D}, —NR^{7B}OR^{7D}, —OCX^{7.1}₃, —OCHX^{7.1}₂, —OCH₂X^{7.1}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁸ is hydrogen, halogen, —CX^{8.1}₃, —CHX^{8.1}₂, —CH₂X^{8.1}, —CN, —N₃, —SOₙ₈R^{8A}, —SOᵥ₈NR^{8B}R^{8C}, —NHNR^{8B}R^{8C}, —ONR^{8B}R^{8C}, —NHC(O)NHNR^{8B}R^{8C}, —NHC(O)NR^{8B}R^{8C}, —N(O)ₘ₈, —NR^{8B}R^{8C}, —C(O)R^{8D}, —C(O)OR^{8D}, —C(O)NR^{8B}R^{8C}, —OR^{8A}, —NR^{8B}SO₂R^{8A}, —NR^{8B}C(O)R^{8D}, —NR^{8B}C(O)OR^{8D}, —NR^{8B}OR^{8D}, —OCX^{8.1}₃, —OCHX^{8.1}₂, —OCH₂X^{8.1}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁹ is hydrogen, halogen, —CX^{9.1}₃, —CHX^{9.1}₂, —CH₂X^{9.1}, —CN, —N₃, —SOₙ₉R^{9A}, —SOᵥ₉NR^{9B}R^{9C}, —NHNR^{9B}R^{9C}, —ONR^{9B}R^{9C}, —NHC(O)NHNR^{9B}R^{9C}, —NHC(O)NR^{9B}R^{9C}, —N(O)ₘ₉, —NR^{9B}R^{9C}, —C(O)R^{9D}, —C(O)OR^{9D}, —C(O)NR^{9B}R^{9C}, —OR^{9A}, —N^{9B}SO₂R^{9A}, —NR^{9B}C(O)R^{9D}, —NR^{9B}C(O)OR^{9D}, —NR^{9B}OR^{9D}, —OCX^{9.1}₃, —OCHX^{9.1}₂, —OCH₂X^{9.1}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or when X² is CR⁹ and X³ is CR¹⁰, then R⁹ and R¹⁰ may optionally be joined to form a substituted or unsubstituted cycloalkyl, substituted or unsubstituted heterocycloalkyl, substituted or unsubstituted aryl, or substituted or unsubstituted heteroaryl;
R¹⁰ is hydrogen, halogen, —CX^{10.1}₃, —CHX^{10.1}₂, —CH₂X¹⁰, —CN, —N₃, —SOₙ₁₀R^{10A}, —SOᵥ₁₀NR^{10B}R^{10C}, —NHNR^{10B}R^{10C}, —ONR^{10B}R^{10C}, —NHC(O)NHNR^{10B}R^{10C}, —NHC(O)NR^{10B}R^{10C}, —N(O)ₘ₁₀, —NR^{10B}R^{10C}, —C(O)R^{10D}, —C(O)OR^{10D}, —C(O)NR^{10B}R^{10C}, —OR^{10A}, —NR^{10B}SO₂R^{10A}, —NR^{10B}C(O)R^{10D}, —NR^{10B}C(O)OR^{10D}, —NR^{10B}OR^{10D}, —OCX^{10.1}₃, —OCHX^{10.1}₂, —OCH₂X^{10.1}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or when X³ is CR¹⁰ and X⁴ is CR¹¹, then R¹⁰ and R¹¹ may optionally be joined to form a substituted or unsubstituted cycloalkyl, substituted or unsubstituted heterocycloalkyl, substituted or unsubstituted aryl, or substituted or unsubstituted heteroaryl;
R¹¹ is hydrogen, halogen, —CX^{11.1}₃, —CHX^{11.1}₂, —CH₂X^{11.1}, —CN, —N₃, —SOₙ₁₁R^{11A}, —SOᵥ₁₁NR^{11B}R^{11C}, —NHNR^{11B}R^{11C}, —ONR^{11B}R^{11C}, —NHC(O)NHNR^{11B}R^{11C}, —NHC(O)NR^{11B}R^{11C}, —N(O)ₘ₁₁, —NR^{11B}R^{11C}, —C(O)R^{11D}D—C(O)OR^{11D}, —C(O)NR^{11B}R^{11C}, —OR^{11A}, —NR^{11B}SO₂R^{11A}, —NR^{11B}C(O)R^{11D}, —NR^{11B}C(O)OR^{11D}, —NR^{11B}OR^{1D}, —OCX^{11.1}₃, —OCHX^{11.1}₂, —OCH₂X^{11.1}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or when X⁴ is CR¹¹ and X⁵ is CR¹², then R¹¹ and R¹² may optionally be joined to form a substituted or unsubstituted cycloalkyl, substituted or unsubstituted heterocycloalkyl, substituted or unsubstituted aryl, or substituted or unsubstituted heteroaryl;
R¹² is hydrogen, halogen, —CX^{12.1}₃, —CHX^{12.1}₂, —CH₂X^{12.1}, —CN, —N₃, —SOₙ₁₂R^{12A}, —SOᵥ₁₂NR^{12B}R^{12C}, NHNR^{12B}R^{12C}, —ONR^{12B}R^{12C}, —NHC(O)NHNR^{12B}R^{12C}, —NHC(O)NR^{12B}R^{12C}, —N(O)ₘ₁₂, —NR^{12B}R^{12C}, —C(O)R^{12D}, —C(O)OR^{12D}, C(O)NR^{12B}R^{12C}, —OR^{12A}, —NR^{12B}SO₂R^{12A}, —NR^{12B}C(O)R^{12D}, —NR^{12B}C(O)OR^{12D}, —NR^{12B}OR^{12D}, —OCX^{12.1}₃, —OCHX^{12.1}₂, —OCH₂X^{12.1}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, R^{1D}, R^{2A}, R^{2B}, R^{2C}, R^{2D}, R^{3A}, R^{3B}, R^{3C}, R^{3D}, R^{4A}, R^{4B}, R^{4C}, R^{4D}, R^{5A}, R^{5B}, R^{5C}, R^{5D}, R^{6A}, R^{6B}, R^{6C}, R^{6D}, R^{7A}, R^{7B}, R^{7C}, R^{7D}, R^{7B}, R^{8A}, R^{8B}, R^{8C}, R^{8D}, R^{9A}, R^{9B}, R^{9C}, R^{9D}, R^{10A}, R^{10B}, R^{10C}, R^{10D}, R^{11A}, R^{11B}, R^{11C}, R^{11D}, R^{12A}, R^{12B}, R^{12C}, and R^{12D} are independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1B} and R^{1C} substituents bonded to the same nitrogen atom may optionally be joined to form a substituted or unsubstituted heterocycloalkyl or substituted or unsubstituted heteroaryl; R^{2B} and R^{2e} substituents bonded to the same nitrogen atom may optionally be joined to form a substituted or unsubstituted heterocycloalkyl or substituted or unsubstituted heteroaryl; R^{3B} and R^{3C} substituents bonded to the same nitrogen atom may optionally be joined to form a substituted or unsubstituted heterocycloalkyl or substituted or unsubstituted heteroaryl; R^{4B} and R^{4C} substituents bonded to the same nitrogen atom may optionally be joined to form a substituted or unsubstituted heterocycloalkyl or substituted or unsubstituted heteroaryl; R^{5B} and R^{5C} substituents bonded to the same nitrogen atom may optionally be joined to form a substituted or unsubstituted heterocycloalkyl or substituted or unsubstituted heteroaryl; R^{6B} and R^{C} substituents bonded to the same nitrogen atom may optionally be joined to form a substituted or unsubstituted heterocycloalkyl or substituted or unsubstituted heteroaryl; R^{7B} and R^{7C} substituents bonded to the same nitrogen atom may optionally be joined to form a substituted or unsubstituted heterocycloalkyl or substituted or unsubstituted heteroaryl; R^{8B} and R^{8C} substituents bonded to the same nitrogen atom may optionally be joined to form a substituted or unsubstituted heterocycloalkyl or substituted or unsubstituted heteroaryl; R^{9B} and R^{9C} substituents bonded to the same nitrogen atom may optionally be joined to form a substituted or unsubstituted heterocycloalkyl or substituted or unsubstituted heteroaryl; R^{10B} and R^{10c} substituents bonded to the same nitrogen atom may optionally be joined to form a substituted or unsubstituted heterocycloalkyl or substituted or unsubstituted heteroaryl; R^{11B} and R^{11C} substituents bonded to the same nitrogen atom may optionally be joined to form a substituted or unsubstituted heterocycloalkyl or substituted or unsubstituted heteroaryl; R^{12B} and R^{12C} substituents bonded to the same nitrogen atom may optionally be joined to form a substituted or unsubstituted heterocycloalkyl or substituted or unsubstituted heteroaryl;
X^{1.1}, X^{2.1}, X^{3.1}, X^{4.1}, X^{5.1}, X^{6.1}, X^{7.1}, X^{8.1}, X^{9.1}, X^{10.1}, X^{11.1}, and X^{12.1} are independently —Cl, —Br, —I, or —F;
n1, n2, n3, n4, n5, n6, n7, n8, n9, n10, n11, and n12 are independently an integer from 0 to 4; and
m1, m2, m3, m5, m6, m7, m8, m9, m10, m11, m12, v1, v2, v3, v4, v5, v6, v7, v8, v9, v10, v11, and v12 are independently 1 or 2.

2. The compound of claim 1, or a pharmaceutically acceptable salt thereof, wherein z2 is 0.

3. The compound of claim 1 or claim 2, or a pharmaceutically acceptable salt thereof, wherein z4 is 1.

4. The compound of one of claims 1 to 3, or a pharmaceutically acceptable salt thereof, wherein z1 is 2.

5. The compound of claim 1, or a pharmaceutically acceptable salt thereof, having the formula: 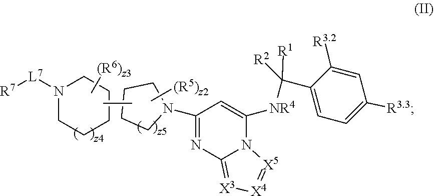
R^{3.2} is hydrogen, halogen, —CX^{3.2}₃, —CHX^{3.2}₂, —CH₂X^{3.2}, —CN, —N₃, —SO_{n3.2}R^{3.2A}, —SO_{v3.2}NR^{3.2B}R^{3.2C}, —NNR^{3.2B}R^{3.2C}, —ONR^{3.2B}R^{3.2C}, —NHC(O)NHNR^{3.2B}R^{3.2C}, —NHC(O)NR^{3.2B}R^{3.2C}, —N(O)_{m3.2}, —NR^{3.2B}R^{3.2C}, —C(O)R^{3.2D}, —C(O)OR^{3.2D}, —C(O)NR^{3.2B}R^{3.2C}, —OR^{3.2A}, —NR^{3.2B}SO₂R^{3.2A}, —NR^{3.2B}C(O)R^{3.2D}, —NR^{3.2B}C(O)OR^{3.2D}, —NR^{3.2B}OR^{3.2D}, —OCX^{3.2}₃, —OCHX^{3.2}₂, —OCH₂X^{3.2}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{3.3} is hydrogen, halogen, —CX^{3.33}, —CHX^{3.32}, —CH₂X^{3.3}, —CN, —N₃, —SO_{n3.3}R^{3.3A}, —SO_{v3.3}NR^{3.3B}R^{3.3C}, —NHNR^{3.3B}R^{3.3C}, —ONR^{3.3B}R^{3.3C}, —NHC(O)NHNR^{3.3B}R^{3.3C}, —NHC(O)NR^{3.3B}R^{3.3C}, —N(O)_{m3.3}, —NR^{3.3B}R^{3.3C}, —C(O)R^{3.3D}, —C(O)OR^{3.3D}, —C(O)NR^{3.3B}R^{3.3C}, —OR^{3.3A}, —NR^{3.3B}SO₂R^{3.3}A —NR^{3.3B}c(o)R^{3.3D}, —NR^{3.3B}C(O)OR^{3.3D}, —NR^{3.3B}OR^{3.3D}, —OCX^{3.3}₃, —OCHX^{3.3}₂, —OCH₂X^{3.3}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{3.2A}, R^{3.2B}, R^{3.2C}, R^{3.2D}, R^{3.3}A, R^{3.3B}, R^{3.3C}, and R^{3.3D} are independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{3.2B} and R^{3.2c} substituents bonded to the same nitrogen atom may optionally be joined to form a substituted or unsubstituted heterocycloalkyl or substituted or unsubstituted heteroaryl; R^{3.3B} and R^{3.3c} substituents bonded to the same nitrogen atom may optionally be joined to form a substituted or unsubstituted heterocycloalkyl or substituted or unsubstituted heteroaryl;
n3.2 and n3.3 are independently an integer from 0 to 4;
m3.2, m3.3, v3.2, and v3.3 are independently 1 or 2; and
X^{3.2} and X³³ are independently —Cl, —Br, —I, or —F.

6. The compound of claim 5, or a pharmaceutically acceptable salt thereof, having the formula

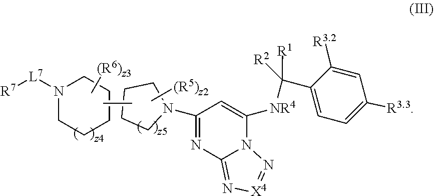

7. The compound of claim 6, or a pharmaceutically acceptable salt thereof, wherein
X⁴ is CR¹¹; and
R¹¹ is hydrogen, —CX^{11.1}₃, —CHX^{11.1}₂, —CH₂X^{11.1}, —CN, or substituted or unsubstituted C1-C4 alkyl.

8. The compound of claim 7, or a pharmaceutically acceptable salt thereof, wherein R¹¹ is hydrogen.

9. The compound of claim 5, or a pharmaceutically acceptable salt thereof, having the formula:

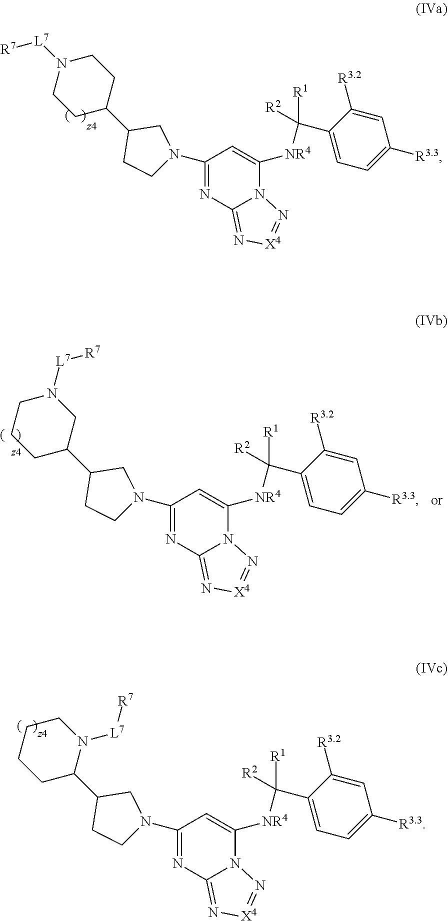

10. The compound of claim 5, or a pharmaceutically acceptable salt thereof, having the formula:

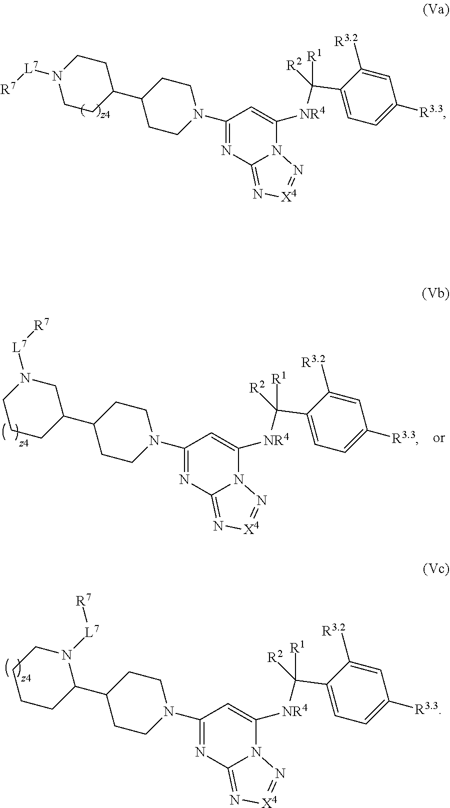

11. The compound of claim 5, or a pharmaceutically acceptable salt thereof, having the formula:

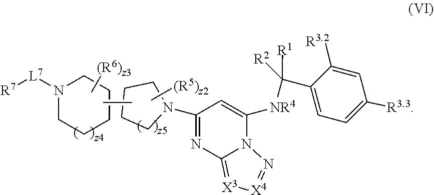

12. The compound of claim 11, or a pharmaceutically acceptable salt thereof, wherein
X³ is CR¹⁰;
X⁴ is CR¹¹;
R¹⁰ is hydrogen, —CX^{10.1}₃, —CHX^{10.1}₂, —CH₂X^{10.1}, —CN, or substituted or unsubstituted C₁-C₄ alkyl; and
R¹¹ is hydrogen, —CX^{11.1}₃, —CHX^{11.1}₂, —CH₂X^{11.1}, —CN, or substituted or unsubstituted C₁-C₄ alkyl.

13. The compound of claim 12, or a pharmaceutically acceptable salt thereof, wherein R¹⁰ and R¹¹ are hydrogen.

14. The compound of claim 5, or a pharmaceutically acceptable salt thereof, having the formula:

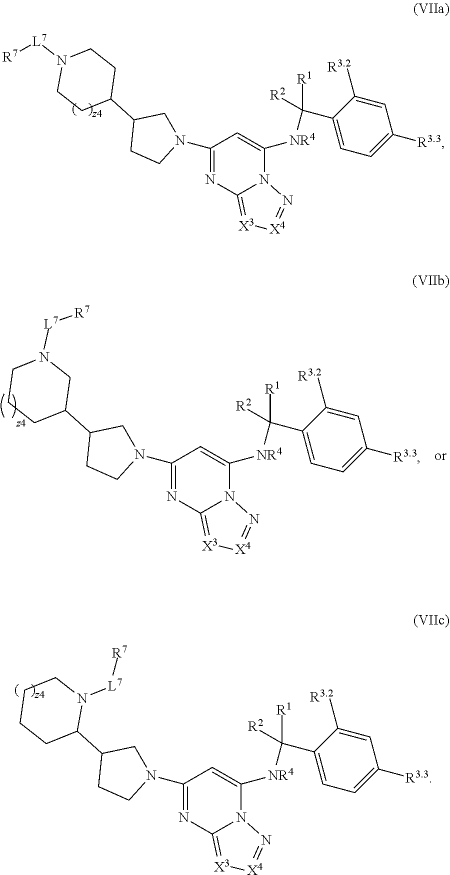

15. The compound of claim 5, or a pharmaceutically acceptable salt thereof, having the formula:

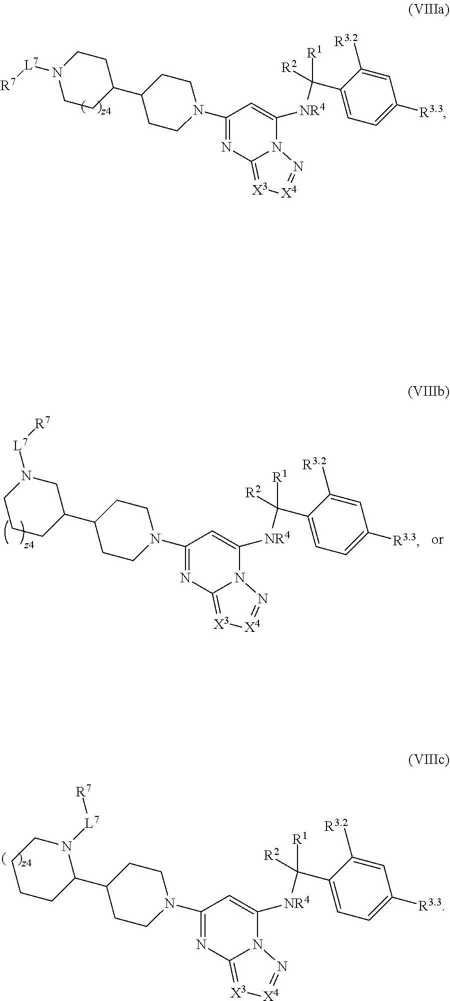

16. The compound of claim 1, or a pharmaceutically acceptable salt thereof, wherein R⁴ is hydrogen.

17. The compound of claim 1, or a pharmaceutically acceptable salt thereof, wherein R¹ is hydrogen, substituted or unsubstituted alkyl, or substituted or unsubstituted heteroalkyl.

18. The compound of claim 1, or a pharmaceutically acceptable salt thereof, wherein R¹ is hydrogen or substituted or unsubstituted C1-C4 alkyl.

19. The compound of claim 1, or a pharmaceutically acceptable salt thereof, wherein R¹ is hydrogen.

20. The compound of claim 1, or a pharmaceutically acceptable salt thereof, wherein R¹ is unsubstituted methyl.

21. The compound of claim 1, or a pharmaceutically acceptable salt thereof, wherein R² is hydrogen, substituted or unsubstituted alkyl, or substituted or unsubstituted heteroalkyl.

22. The compound of claim 1, or a pharmaceutically acceptable salt thereof, wherein R² is hydrogen or substituted or unsubstituted C₁-C₄ alkyl.

23. The compound of claim 1, or a pharmaceutically acceptable salt thereof, wherein R² is hydrogen.

24. The compound of claim 1, or a pharmaceutically acceptable salt thereof, wherein R² is unsubstituted methyl.

25. The compound of claim 5, or a pharmaceutically acceptable salt thereof, wherein R^{3.2} and R^{3.3} are independently halogen.

26. The compound of claim 5, or a pharmaceutically acceptable salt thereof, wherein R^{3.2} and R are —Cl.

27. The compound of claim 1, or a pharmaceutically acceptable salt thereof, wherein L⁷ is a bond, substituted or unsubstituted alkylene, or substituted or unsubstituted cycloalkylene.

28. The compound of claim 1, or a pharmaceutically acceptable salt thereof, wherein L⁷ is a bond, unsubstituted C1-C6 alkylene, or substituted or unsubstituted C3-C6 cycloalkylene.

29. The compound of claim 1, or a pharmaceutically acceptable salt thereof, wherein L7 is a bond, unsubstituted methylene, unsubstituted ethylene, unsubstituted n-propylene,

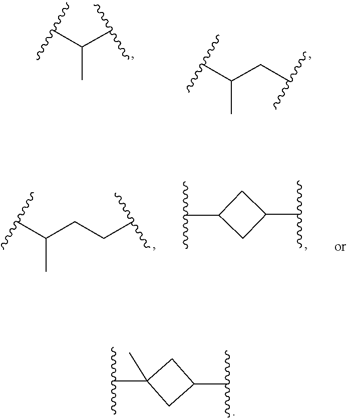

30. The compound of claim 1, or a pharmaceutically acceptable salt thereof, wherein R⁷ is hydrogen, halogen, —CX^{7.1}₃, —CN, —SOₙ₇R^{7A}, —SOᵥ₇NR^{7B}R^{7C}, —NHC(O)NR^{7B}R^{7C}, W^{7B}R^{7C}, —C(O)R^{7D}, —C(O)OR^{7D}, —C(O)NR^{7B}R^{7C}, —OR^{7A}, —NR^{7B}SO₂R^{7A}, —NR^{7B}C(O)R^{7D}, —NR^{7B}C(O)OR^{7D}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

31. The compound of claim 1, a pharmaceutically acceptable salt thereof, wherein R⁷ is hydrogen, —CX^{7.1}₃, —CN, —OR^{7A}, —C(O)OR^{7D}, —C(O)NR^{7B}R^{7C}, —NR^{7B}C(O)R^{7D}, —SOₙ₇R^{7A}, —SOᵥ₇NR^{7B}R^{7C}, substituted or unsubstituted C₁-C₄ alkyl, unsubstituted 2 to 6 membered heteroalkyl, substituted or unsubstituted C₃-C₆ cycloalkyl, or substituted or unsubstituted 5 to 6 membered heteroaryl.

32. The compound of claim 1, or a pharmaceutically acceptable salt thereof, wherein R⁷ is hydrogen, —CF₃, —CN, —OH, —OCH₃, —C(O)OH, —SO₂CH₃, —SO₂NH₂, —SO₂NHCH₃, —NHC(O)CH₃, —C(O)NHCH₃, unsubstituted methyl, unsubstituted cyclopentyl,

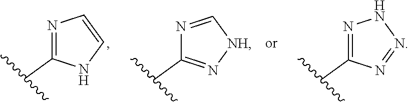 [SMILES: *c1ncc[nH]1 ; *c1nc[nH]n1 ; *c1nn[nH]n1]

33. The compound of claim 1 having the formula: 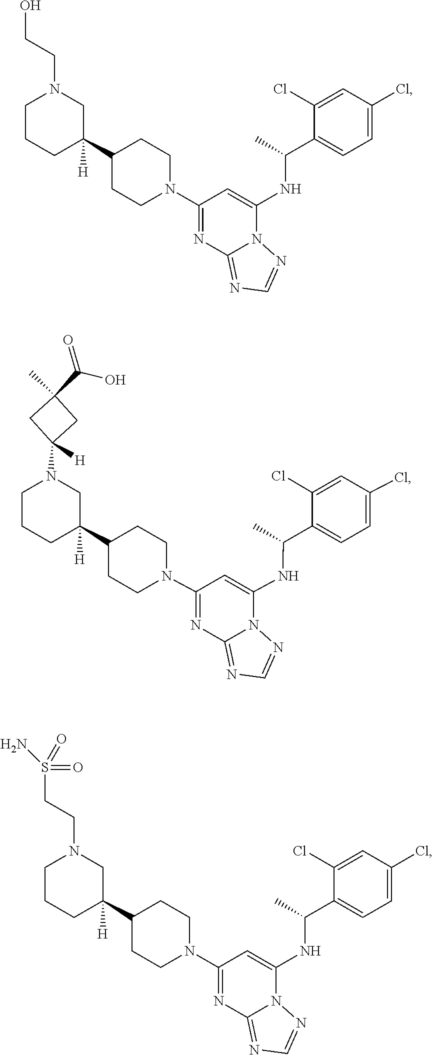 [SMILES: C[C@@H](Nc1cc(N2CCC([C@H]3CCCN(CCO)C3)CC2)nc2ncnn12)c1ccc(Cl)cc1Cl ; C[C@@H](Nc1cc(N2CCC([C@H]3CCCN([C@H]4C[C@](C)(C(=O)O)C4)C3)CC2)nc2ncnn12)c1ccc(Cl)cc1Cl ; C[C@@H](Nc1cc(N2CCC([C@H]3CCCN(CCS(N)(=O)=O)C3)CC2)nc2ncnn12)c1ccc(Cl)cc1Cl] 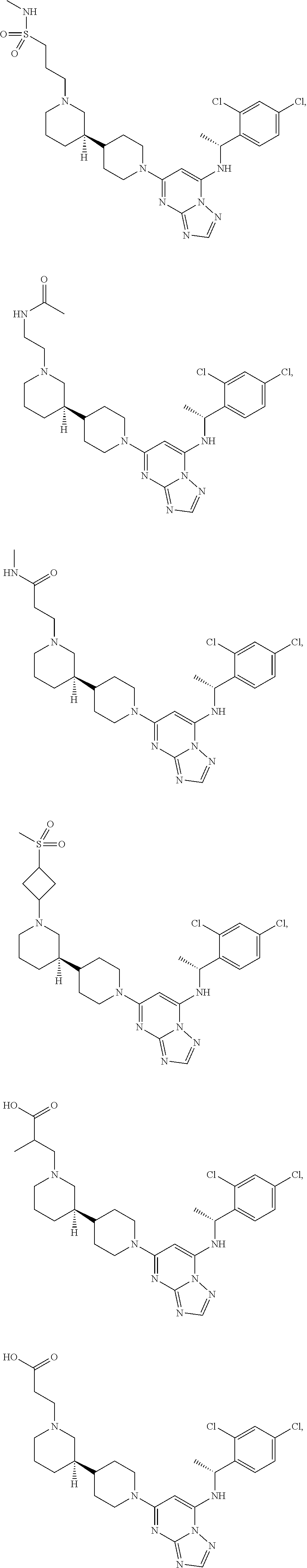 [SMILES: CNS(=O)(=O)CCCN1CCC[C@H](C2CCN(c3cc(N[C@H](C)c4ccc(Cl)cc4Cl)n4ncnc4n3)CC2)C1 ; CC(=O)NCCN1CCC[C@H](C2CCN(c3cc(N[C@H](C)c4ccc(Cl)cc4Cl)n4ncnc4n3)CC2)C1 ; CNC(=O)CCN1CCC[C@H](C2CCN(c3cc(N[C@H](C)c4ccc(Cl)cc4Cl)n4ncnc4n3)CC2)C1 ; C[C@@H](Nc1cc(N2CCC([C@H]3CCCN(C4CC(S(C)(=O)=O)C4)C3)CC2)nc2ncnn12)c1ccc(Cl)cc1Cl ; CC(CN1CCC[C@H](C2CCN(c3cc(N[C@H](C)c4ccc(Cl)cc4Cl)n4ncnc4n3)CC2)C1)C(=O)O ; C[C@@H](Nc1cc(N2CCC([C@H]3CCCN(CCC(=O)O)C3)CC2)nc2ncnn12)c1ccc(Cl)cc1Cl] 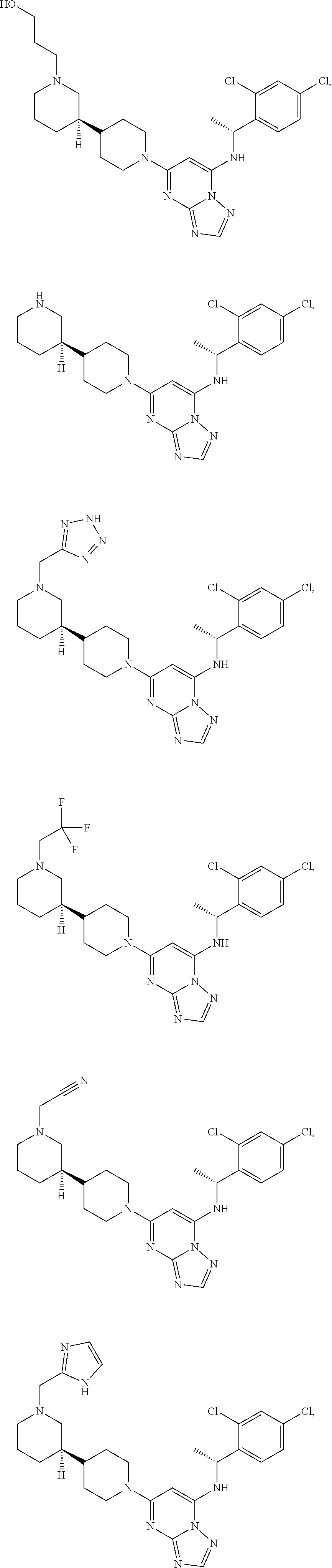 [SMILES: C[C@@H](Nc1cc(N2CCC([C@H]3CCCN(CCCO)C3)CC2)nc2ncnn12)c1ccc(Cl)cc1Cl ; C[C@@H](Nc1cc(N2CCC([C@H]3CCCNC3)CC2)nc2ncnn12)c1ccc(Cl)cc1Cl ; C[C@@H](Nc1cc(N2CCC([C@H]3CCCN(Cc4nn[nH]n4)C3)CC2)nc2ncnn12)c1ccc(Cl)cc1Cl ; C[C@@H](Nc1cc(N2CCC([C@H]3CCCN(CC(F)(F)F)C3)CC2)nc2ncnn12)c1ccc(Cl)cc1Cl ; C[C@@H](Nc1cc(N2CCC([C@H]3CCCN(CC#N)C3)CC2)nc2ncnn12)c1ccc(Cl)cc1Cl ; C[C@@H](Nc1cc(N2CCC([C@H]3CCCN(Cc4ncc[nH]4)C3)CC2)nc2ncnn12)c1ccc(Cl)cc1Cl] 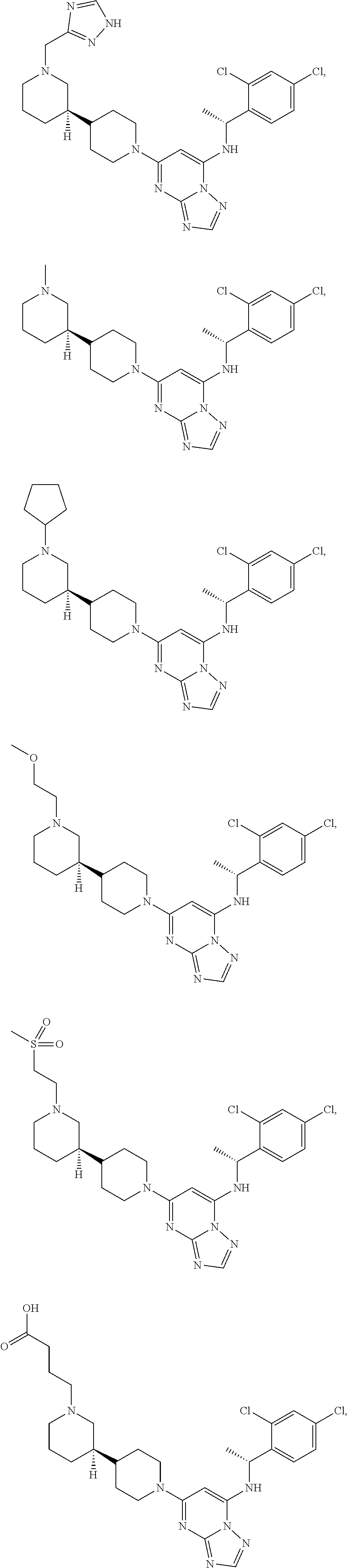 [SMILES: C[C@@H](Nc1cc(N2CCC([C@H]3CCCN(Cc4nc[nH]n4)C3)CC2)nc2ncnn12)c1ccc(Cl)cc1Cl ; C[C@@H](Nc1cc(N2CCC([C@H]3CCCN(C)C3)CC2)nc2ncnn12)c1ccc(Cl)cc1Cl ; C[C@@H](Nc1cc(N2CCC([C@H]3CCCN(C4CCCC4)C3)CC2)nc2ncnn12)c1ccc(Cl)cc1Cl ; COCCN1CCC[C@H](C2CCN(c3cc(N[C@H](C)c4ccc(Cl)cc4Cl)n4ncnc4n3)CC2)C1 ; C[C@@H](Nc1cc(N2CCC([C@H]3CCCN(CCS(C)(=O)=O)C3)CC2)nc2ncnn12)c1ccc(Cl)cc1Cl ; C[C@@H](Nc1cc(N2CCC([C@H]3CCCN(CCCC(=O)O)C3)CC2)nc2ncnn12)c1ccc(Cl)cc1Cl] 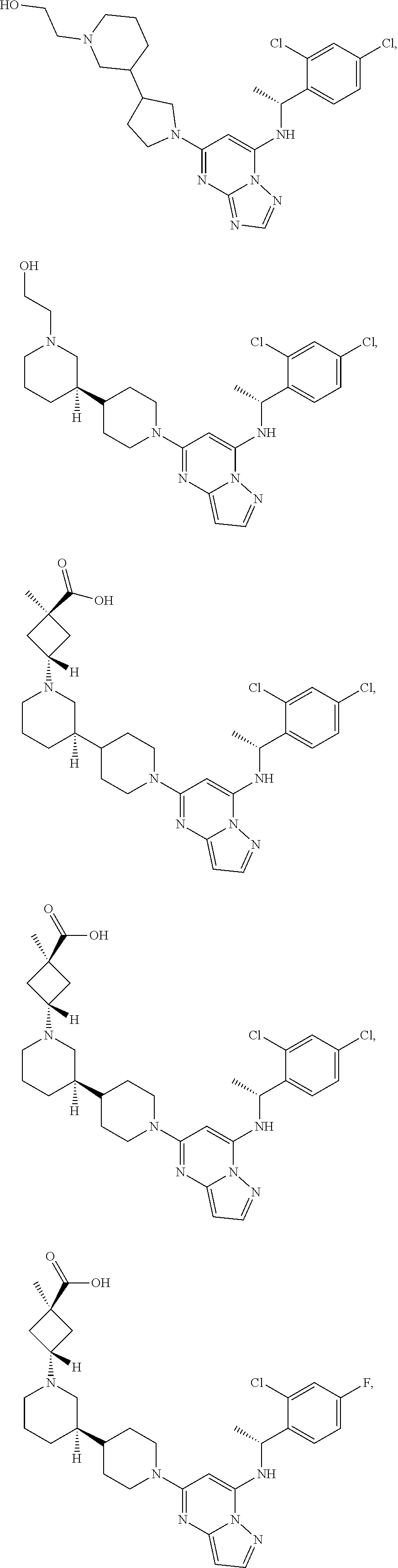 [SMILES: C[C@@H](Nc1cc(N2CCC(C3CCCN(CCO)C3)C2)nc2ncnn12)c1ccc(Cl)cc1Cl ; C[C@@H](Nc1cc(N2CCC([C@H]3CCCN(CCO)C3)CC2)nc2ccnn12)c1ccc(Cl)cc1Cl ; C[C@@H](Nc1cc(N2CCC([C@H]3CCCN([C@H]4C[C@](C)(C(=O)O)C4)C3)CC2)nc2ccnn12)c1ccc(Cl)cc1Cl ; C[C@@H](Nc1cc(N2CCC([C@H]3CCCN([C@H]4C[C@](C)(C(=O)O)C4)C3)CC2)nc2ccnn12)c1ccc(Cl)cc1Cl ; C[C@@H](Nc1cc(N2CCC([C@H]3CCCN([C@H]4C[C@](C)(C(=O)O)C4)C3)CC2)nc2ccnn12)c1ccc(F)cc1Cl] 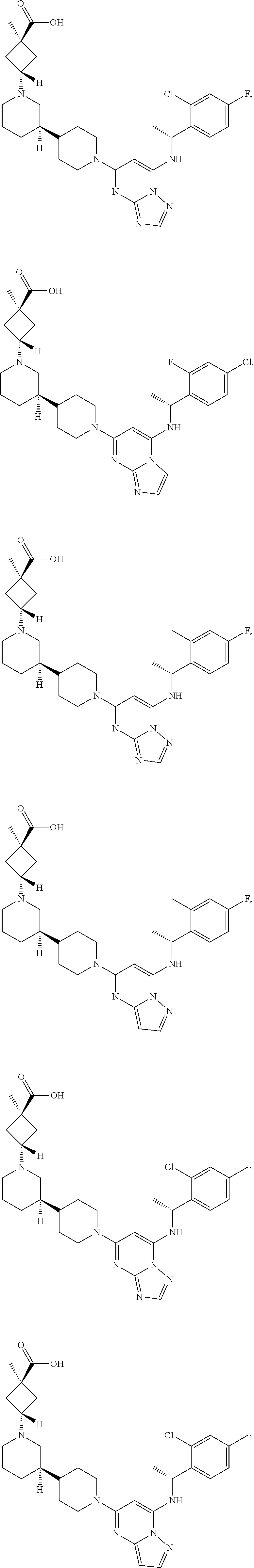 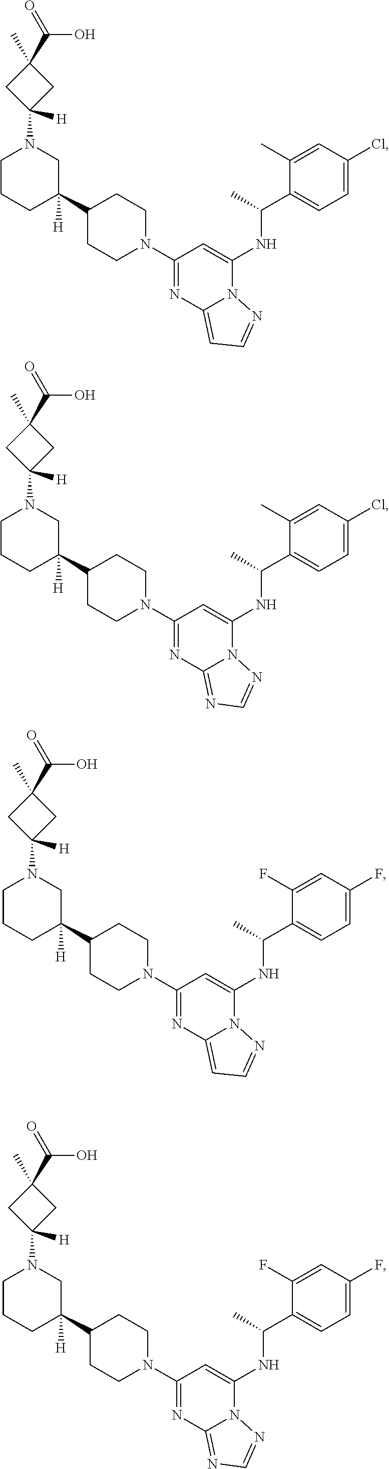 [SMILES: Cc1cc(Cl)ccc1[C@@H](C)Nc1cc(N2CCC([C@H]3CCCN([C@H]4C[C@](C)(C(=O)O)C4)C3)CC2)nc2ccnn12 ; Cc1cc(Cl)ccc1[C@@H](C)Nc1cc(N2CCC([C@H]3CCCN([C@H]4C[C@](C)(C(=O)O)C4)C3)CC2)nc2ncnn12 ; C[C@@H](Nc1cc(N2CCC([C@H]3CCCN([C@H]4C[C@](C)(C(=O)O)C4)C3)CC2)nc2ccnn12)c1ccc(F)cc1F ; C[C@@H](Nc1cc(N2CCC([C@H]3CCCN([C@H]4C[C@](C)(C(=O)O)C4)C3)CC2)nc2ncnn12)c1ccc(F)cc1F] 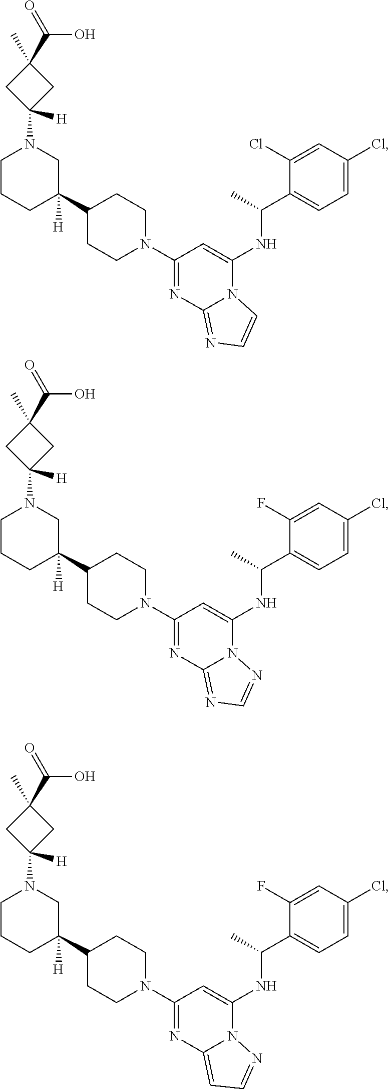 [SMILES: C[C@@H](Nc1cc(N2CCC([C@H]3CCCN([C@H]4C[C@](C)(C(=O)O)C4)C3)CC2)nc2nccn12)c1ccc(Cl)cc1Cl ; C[C@@H](Nc1cc(N2CCC([C@H]3CCCN([C@H]4C[C@](C)(C(=O)O)C4)C3)CC2)nc2ncnn12)c1ccc(Cl)cc1F ; C[C@@H](Nc1cc(N2CCC([C@H]3CCCN([C@H]4C[C@](C)(C(=O)O)C4)C3)CC2)nc2ccnn12)c1ccc(Cl)cc1F]
or a pharmaceutically acceptable salt thereof.

34. A pharmaceutical composition comprising a pharmaceutically acceptable excipient and the compound of claim 1, or a pharmaceutically acceptable salt thereof.

35. A method of treating or preventing a disease or disorder mediated by CCR4, comprising administering to a subject in need thereof a therapeutically effective amount of the compound of claim 1, or a pharmaceutically acceptable salt thereof.

36. A method of treating or preventing a disease or disorder mediated by CCR4, comprising administering to a subject in need thereof a therapeutically effective amount of the pharmaceutical composition of claim 34.

37. The method of claim 35, wherein the disease or disorder is an immune disease or disorder or an inflammatory disease or disorder.

38. The method of claim 37, further comprising co-administering an anti-inflammatory agent to the subject.

39. The method of claim 38, wherein the anti-inflammatory agent is thalidomide or a derivative thereof, a retinoid, dithranol, calcipotriol, a non-steroidal anti-inflammatory agent (NSAID), a cyclo-oxygenase inhibiting nitric oxide donor (CINOD), a glucocorticosteroid, methotrexate, leflunomide, hydroxychloroquine, d-penicillamine, auranofin, an analgesic, a diacerein, hyaluronic acid derivative, or a nutritional supplement.

40. The method of claim 35, wherein the disease or disorder is a cardiovascular disease or disorder or a metabolic disease or disorder.

41. The method of claim 40, further comprising co-administering a cardiovascular disorder agent or a metabolic disorder agent to the subject.

42. The method of claim 41, wherein the cardiovascular disorder agent is a calcium channel blocker, a beta-adrenoceptor blocker, an angiotensin-converting enzyme (ACE) inhibitor, an angiotensin-2 receptor antagonist, a lipid lowering agent, a modulator of blood cell morphology, a thrombolytic, or an anticoagulant.

43. The method of claim 35, wherein the disease or disorder is cancer.

44. The method of claim 43, further comprising co-administering a chemotherapeutic agent or anti-cancer agent to the subject.

45. The method of claim 44, wherein the chemotherapeutic agent or anti-cancer agent is an antiproliferative/antineoplastic drug, an antimetabolite, an antitumour antibiotic, an antimitotic agent, a topoisomerase inhibitor, a cytostatic agent, an oestrogen receptor down regulator, an antiandrogen, a LHRH antagonist or LHRH agonist, a progestogen, an aromatase inhibitor, an inhibitor of 5α-reductase, an agent which inhibits cancer cell invasion, an inhibitor of growth factor function, a farnesyl transferase inhibitor, a tyrosine kinase inhibitor, a serine/threonine kinase inhibitor, an inhibitor of the epidermal growth factor family, an inhibitor of the platelet-derived growth factor family, an inhibitor of the hepatocyte growth factor family; an antiangiogenic agent, a vascular damaging agent, an agent used in antisense therapy, an anti-ras antisense, an agent used in a gene therapy, an immunotherapeutic agent, or an antibody.

46. The method of claim 44, wherein the chemotherapeutic agent or anti-cancer agent is an inhibitor of the PD-L1/PD-1 pathway, an inhibitor of CTLA-4 or an agonistic antibody of CD137 (4.1BB).

47. The method of claim 44, wherein the chemotherapeutic agent or anti-cancer agent is an agent from Table 1.

48. The method of claim 44, wherein the chemotherapeutic agent or anti-cancer agent is an inhibitor of the PD-L1/PD-1 pathway, an inhibitor of CTLA-4 or an agonistic antibody of CD137 (4.1BB).

49. The method of claim 44, wherein the chemotherapeutic agent or anti-cancer agent is an immune modulator agent, or an agent from Table 1, or any combination thereof.

50. The method of claim 43, wherein the cancer is colon cancer or pancreatic cancer.

51. The method of claim 35, wherein the disease or disorder is allergy-related.

52. The method of claim 35, wherein the disease or disorder is inflammation.

53. The method of claim 35, wherein the disease or disorder is asthma, urticaria, rhinitis, or dermatitis.

54. The method of claim 53, wherein the disease or disorder is allergic asthma, autoimmune urticaria, allergic rhinitis, or contact dermatitis.

55. A method of inhibiting C-C chemokine receptor type 4 (CCR4), comprising contacting CCR4 with the compound of claim 1, or a pharmaceutically acceptable salt thereof.
